(19) United States
(12) Patent Application Publication
(10) Pub. No.: US 2007/0161677 A1
(43) Pub. Date: Jul. 12, 2007

(54) Bisarylurea derivatives

(75) Inventor: Hans-Peter Buchstaller, Griesheim (DE)

Correspondence Address: BAKER & DANIELS LLP, 805 15TH STREET, NW, SUITE 700, WASHINGTON, DC, 20005, US

(73) Assignee: Merck Patent GmbH, Darmstadt (DE)

(21) Appl. No.: 10/587,292

(22) PCT Filed: Jan. 17, 2005
(86) PCT No.: PCT/EP05/00387

§ 371 Date: Jul. 25, 2006

(30) Foreign Application Priority Data
Jan. 30, 2004 (EP) 04002092.7

(51) Int. Cl. A61K 31/444; A61K 31/4412; A61K 31/17; C07D 213/62; C07D 401/02

(52) U.S. Cl. 514/332; 514/346; 514/585; 514/596; 546/261; 546/291; 564/26; 564/48

(57) ABSTRACT

The present invention relates to bisarylurea derivatives of formula (I), the use of the compounds of formula (I) as inhibitors of raf-kinase, the use of the compounds of formula (I) for the manufacture of a pharmaceutical composition and a method of treatment, comprising administering said pharmaceutical composition to a patient.

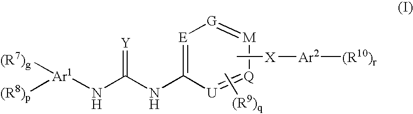

The present invention relates to bisarylurea derivatives, bisarylurea derivatives as medicaments, bisarylurea derivatives as inhibitors of raf-kinase, the use of bisarylurea derivatives for the manufacture of a pharmaceutical, a method for producing a pharmaceutical composition containing said bisarylurea derivatives, the pharmaceutical composition obtainable by said method and a method of treatment, comprising administering said pharmaceutical composition.

Protein phosphorylation is a fundamental process for the regulation of cellular functions. The coordinated action of both protein kinases and phosphatases controls the levels of phosphorylation and, hence, the activity of specific target proteins. One of the predominant roles of protein phosphorylation is in signal transduction, where extracellular signals are amplified and propagated by a cascade of protein phosphorylation and dephosphorylation events, e.g. in the p21^{ras}/raf pathway.

The p21^{ras} gene was discovered as an oncogene of the Harvey (rasH) and Kirsten (rasK) rat sarcoma viruses. In humans, characteristic mutations in the cellular ras gene (c-ras) have been associated with many different types of cancers. These mutant alleles, which render Ras constitutively active, have been shown to transform cells, such as the murine cell line NIH 3T3, in culture.

The p21^{ras} oncogene is a major contributor to the development and progression of human solid cancers and is mutated in 30% of all human cancers (Bolton et al. (1994) Ann. Rep. Med. Chem., 29, 165-74; Bos. (1989) Cancer Res., 49, 4682-9). Oncogenic Ras mutations have been identified for example in lung cancer, colorectal cancer, pancreas, thyroid cancer, melanoma, bladder tumours, liver tumour, kidney tumor, dermatological tumours and haematological tumors (Ddjei et al. (2001), J. Natl. Cancer Inst. 93(14), 1062-74; Midgley, R. S. and Kerr, D. J. (2002) Critical Rev. One/hematol 44, 109-120; Downward, J. (2003), Nature reviews 3, 11-22). In its normal, unmutated form, the ras protein is a key element of the signal transduction cascade directed by growth factor receptors in almost all tissues (Avruch et al. (1994) Trends Biochem. Sci., 19, 279-83).

Biochemically, ras is a guanine nucleotide binding protein, and cycling between a GTP-bound activated and a GDP-bound resting form is strictly controlled by ras endogenous GTPase activity and other regulatory proteins. The ras gene product binds to guanine triphosphate (GTP) and guanine diphosphate (GDP) and hydrolyzes GTP to GDP. It is the GTP-bound state of Ras that is active. In the ras mutants in cancer cells, the endogenous GTPase activity is alleviated and, therefore, the protein delivers constitutive growth signals to downstream effectors such as the enzyme raf kinase. This leads to the cancerous growth of the cells which carry these mutants (Magnuson et al. (1994) Semin. Cancer Biol., 5, 247-53). The ras proto-oncogene requires a functionally intact c-raf1 proto-oncogene in order to transduce growth and differentiation signals initiated by receptor and non-receptor tyrosine kinases in higher eukaryotes.

Activated Ras is necessary for the activation of the c-raf-1 proto-oncogene, but the biochemical steps through which Ras activates the Raf-1 protein (Ser/Thr) kinase are now well characterized. It has been shown that inhibiting the effect of active ras by inhibiting the raf kinase signaling pathway by administration of deactivating antibodies to raf kinase or by co-expression of dominant negative raf kinase or dominant negative MEK, the substrate of raf kinase, leads to the reversion of transformed cells to the normal growth phenotype see: Daum et al. (1994) Trends Biochem. Sci., 19, 474-80; Fridman et al. (1994) J Biol. Chem., 269, 30105-8. Kolch et al. (1991) Nature, 349, 426-28) and for review Weinstein-Oppenheimer et al. Pharm. & Therap. (2000), 88, 229-279.

Similarly, inhibition of raf kinase (by antisense oligodeoxynucleotides) has been correlated in vitro and in vivo with inhibition of the growth of a variety of human tumor types (Monia et al., Nat. Med. 1996, 2, 668-75; Geiger et al. (1997), Clin. Cancer Res. 3(7): 1179-85; Lau et al. (2002), Antisense Nucl. Acid. Drug Dev. 12(1): 11-20; McPhillips et al. (2001), Br. J. Cancer 85(11): 1753-8).

Raf serine- and threonine-specific protein kinases are cytosolic enzymes that stimulate cell growth in a variety of cell systems (Rapp, U. R., et al. (1988) in The oncogene handbook; T. Curran, E. P. Reddy, and A. Skalka (ed.) Elsevier Science Publishers; The Netherlands, pp. 213-253; Rapp, U. R., et al. (1988) Cold Spring Harbor Sym. Quant. Biol. 53:173-184; Rapp, U. R., et al. (1990) Inv Curr. Top. Microbiol. Amunol. Potter and Melchers (eds), Berlin, Springer-Verlag 166:129-139).

Three isozymes have been characterized:

c-Raf (also named Raf-1, c-raf-1 or c-raf1) (Bonner, T. I., et al. (1986) Nucleic Acids Res. 14:1009-1015). A-Raf (Beck, T. W., et al. (1987) Nucleic Acids Res. 15:595-609), and B-Raf (Qkawa, S., et al. (1998) Mol. Cell. Biol. 8:2651-2654; Sithanandam, G. et a. (1990) Oncogene: 1775). These enzymes differ in their expression in various tissues. Raf-1 is expressed in all organs and in all cell lines that have been examined, and A- and B-Raf are expressed in urogenital and brain tissues, respectively (Storm, S. M. (1990) Oncogene 5:345-351).

Raf genes are proto-oncogenes: they can initiate malignant transformation of cells when expressed in specifically altered forms. Genetic changes that lead to oncogenic activation generate a constitutively active protein kinase by removal or interference with an N-terminal negative regulatory domain of the protein (Heidecker, G., et al. (1990) Mol. Cell. Biol. 10:2503-2512; Rapp, U. R., et al. (1987) in Oncogenes and cancer S. A. Aaronson, J. Bishop, T. Sugimura, M. Terada, K. Toyoshima, and P. K. Vogt (ed). Japan Scientific Press, Tokyo). Microinjection into NIH 3T3 cells of oncogenically activated but not wild-type versions of the Raf-protein prepared with Escherichia coli expression vectors results in morphological transformation and stimulates DNA synthesis (Rapp, U. R., et al. (1987) in Oncogenes and cancer; S. A. Aaronson, J. Bishop, T. Sugimura, M. Terada, K. Toyoshima, and P. K. Vogt (ed.) Japan Scientific Press, Tokyo; Smith, M. R., et al (1990) Mol. Cell. Biol. 10:3828-3833). Activating mutants of B-Raf have been identified in a wide range of human cancers e.g. colon, ovarien, melanomas and sarcomas (Davies, H., et al. (2002), Nature 417 949-945. Published online Jun. 9, 2002, 10.1038/nature00766). The preponderant mutation is a single phosphomimetic substitution in the kinase activation domain (V599E), leading to constitutive kinase activity and transformation of NIH3T3 cells.

Thus, activated Raf-1 is an intracellular activator of cell growth. Raf-1 protein serine kinase in a candidate downstream effector of mitogen signal transduction, since Raf oncogenes overcome growth arrest resulting from a block of cellular ras activity due either to a cellular mutation (ras revertant cells) or microinjection of anti-ras antibodies (Rapp, U. R., et al. (1988) in The Oncogene Handbook, T. Curran, E. P. Reddy, and A. Skalka (ed.), Elsevier Science Publishers; The Netherlands, pp. 213-253; Smith, M. R., et al. (1986) Nature (London) 320:540-543).

c-Raf function is required for transformation by a variety of membrane-bound oncogenes and for growth stimulation by mitogens contained in serums (Smith, M. R., et al. (1986) Nature (London) 320:540-543). Raf-1 protein serine kinase activity is regulated by mitogens via phosphorylation (Morrison, D. K., et al. (1989) Cell 58:648-657), which also effects sub cellular distribution (Olah, Z., et al. (1991) Exp. Brain Res. 84:403; Rapp, U. R., et al. (1988) Cold Spring Harbor Sym. Quant. Biol. 53:173-184. Raf-1 activating growth factors include platelet-derived growth factor (PDGF) (Morrison, D. K., et al. (1988) Proc. Natl. Acad. Sci. USA 85:8855-8859), colony-stimulating factor (Baccarini, M., et al. (1990) EMBO J. 9:3649-3657), insulin (Blackshear, P. J., et al. (1990) J. Biol. Chem. 265:12115-12118), epidermal growth factor (EGF) (Morrison, R. K., et al. (1988) Proc. Natl. Acad. Sci. USA 85:8855-8859), interleukin 2 (Turner, B. C., et al (1991) Proc. Natl. Acad. Sci. USA 88:1227), and interleukin 3 and granulocytemacrophage colony-stimulating factor (Carroll, M. P., et al (1990) J. Biol. Chem. 265:19812-19817).

Upon mitogen treatment of cells, the transiently activated Raf-1 protein serine kinase translocates to the perinuclear area and the nucleus (Olah, Z., et al. (1991) Exp. Brain Res. 84:403; Rapp, U. R., et al. (1988) Cold Spring Harbor Sym. Quant. Biol. 53:173-184). Cells containing activated Raf are altered in their pattern of gene expression (Heidecker, G., et al. (1989) in Genes and signal transduction in multistage carcinogenesis, N. Colburn (ed.), Marcel Dekker, Inc., New York, pp. 339-374), and Raf oncogenes activate transcription from Ap-I/PEA3-dependent promoters in transient transfection assays (Jamal, S., et al (1990) Science 344:463-466; Kaibuchi, K., et al (1989) J. Biol. Chem. 264:20855-20858; Wasylyk, C., et al. (1989) Mol. Cell. Biol. 9:2247-2250).

There are at least two independent pathways for Raf-1 activation by extracellular mitogens: one involving protein kinase C (KC) and a second initiated by protein tyrosine kinases (Blackshear, P. J., et al. (1990) J. Biol. Chem. 265:12131-12134; Kovacina, K. S., et al (1990) J. Biol. Chem. 265:12115-12118; Morrison, D. K., et al. (1988) Proc. Natl. Acad. Sci. USA 85:8855-8859; Siegel, J. N., et al (1990) J. Biol. Chem. 265:18472-18480; Turner, B. C., et al (1991) Proc. Natl. Acad. Sci. USA 88:1227). In either case, activation involves Raf-1 protein phosphorylation. Raf-1 phosphorylation may be a consequence of a kinase cascade amplified by autophosphorylation or may be caused entirely by autophosphorylation initiated by binding of a putative activating ligand to the Raf-1 regulatory domain, analogous to PKC activation by diacylglycerol (Nishizuka, Y. (1986) Science 233:305-312).

The process of angiogenesis is the development of new blood vessels, generally capillaries, from pre-existing vasculature. Angiogenesis is defined as involving (i) activation of endothelial cells; (ii) increased vascular permeability; (iii) subsequent dissolution of the basement membrane and extravisation of plasma components leading to formation of a provisional fibrin gel extracellular matrix; (iv) proliferation and mobilization of endothelial cells; (v) reorganization of mobilized endothelial cells to form functional capillaries; (vi) capillary loop formation; and (vii) deposition of basement membrane and recruitment of perivascular cells to newly formed vessels.

Normal angiogenesis is activated during tissue growth, from embryonic development through maturity, and then enters a period of relative quiescence during adulthood.

Normal angiogensesis is also activated during wound healing, and at certain stages of the female reproductive cycle. Inappropriate or pathological angiogenesis has been associated with several disease states including various retinopathies; ischemic disease; atherosclerosis; chronic inflammatory disorders; rheumatoid arthritis, and cancer. The role of angiogenesis in disease states is discussed, for instance, in Fan et al, Trends in Pharmacol Sci. 16:54 66; Shawver et al, DOT Vol. 2, No. 2 Feb. 1997; Folkmann, 1995, Nature Medicine 1:27-31.

In cancer the growth of solid tumors has been shown to be angiogenesis dependent. (See Folkmann, J., J. Nat'l. Cancer Inst., 1990, 82, 4-6.) Consequently, the targeting of pro-angiogenic pathways is a strategy being widely pursued in order to provide new therapeutics in these areas of great, unmet medical need.

Raf is involved in angiogenic processes. Endothelial growth factors (e.g. vascular endothelial growth factor VEGF or basic fibroblast growth factor bFGF) activates receptor tyrosine kinases (e.g. VEGFR-2) and signal through the Ras/Raf/Mek/Erk kinase cascade and protects endothelial cells from apoptosis (Alavi et al. (2003), Science 301, 94-96; Hood, J. D. et al. (2002), Science 296, 2404; Mikula, M. et al. (2001), EMBO J. 20, 1952; Hauser, M. et al. (2001), EMBO J. 20, 1940; Wojnowski et al. (1997), Nature Genet. 16, 293). Activation of VEGFR-2 by VEGF is a critical step in the signal transduction pathway that initiates tumor angiogenesis. VEGF expression may be constitutive to tumor cells and can also be upregulated in response to certain stimuli. One such stimuli is hypoxia, where VEGF expression is upregulated in both tumor and associated host tissues. The VEGF ligand activates VEGFR-2 by binding with its extracellular VEGF binding site. This leads to receptor dimerization of VEGFRs and autophosphorylation of tyrosine residues at the intracellular kinase domain of VEGFR-2. The kinase domain operates to transfer a phosphate from ATP to the tyrosine residues, thus providing binding sites for signaling proteins downstream of VEGFR-2 leading ultimately to initiation of angiogenesis (McMahon, G., The Oncologist, Vol. 5, No. 90001, 3-10, Apr. 2000).

Mice with a targeted disruption in the Braf gene die of vascular defects during development (Wojnowski, L. et al. 1997, Nature genetics 16, page 293-296). These mice show defects in the formation of the vascular system and in angiogenesis e.g. enlarged blood vessels and increased apoptotic death of differentiated endothelial cells.

For the identification of a signal transduction pathway and the detection of cross talks with other signaling pathways suitable models or model systems have been generated by various scientists, for example cell culture models (e.g. Khwaja et al., EMBO, 1997, 16, 2783-93) and transgenic animal models (e.g. White et al., Oncogene, 2001, 20, 7064-7072). For the examination of particular steps in the signal transduction cascade, interfering compounds can be used for signal modulation (e.g. Stephens et al., Biochemical J., 2000, 351, 95-105). The compounds according to the invention may also be useful as reagents for the examination of kinase dependent signal transduction pathways in animal and/or cell culture models or any of the clinical disorders listed throughout this application.

The measurement of kinase activity is a well known technique feasible for each person skilled in the art. Generic test systems for kinase activity detection with substrates, for example histone (e.g. Alessi et al., FEBS Lett. 1996, 399, 3, page 333-8) or myelin basic protein are well described in the literature (e.g. Campos-González, R. and Glenney, Jr., J. R. 1992 J. Biol. Chem. 267, Page 14535).

For the identification of kinase inhibitors various assay systems are available (see for example Walters et al., Nature Drug Discovery 2003, 2; page 259-266). For example, in scintillation proximity assays (e.g. Sorg et al., J. of. Biomolecular Screening, 2002, 7, 11-19) or flashplate assays the radioactive phosphorylation of a protein or peptide as substrate with γATP can be measured. In the presence of an inhibitory compound no signal or a decreased radioactive signal is detectable. Furthermore homogeneous time-resolved fluorescence resonance energy transfer (HTR-FRET), and fluorescence polarization (FP) technologies are useful for assay methods (for example Sills et al., J. of Biomolecular Screening, 2002, 191-214).

Other non-radioactive ELISA based assay methods use specific phospho-antibodies (AB). The phospho-AB binds only the phosphorylated substrate. This binding is detectable with a secondary peroxidase conjugated antibody, measured for example by chemiluminescence (for example Ross et al., Biochem. J., 2002, 366, 977-981).

The present invention provides compounds generally described as bisarylurea derivatives, including both aryl and/or heteroaryl derivatives which are preferably kinase inhibitors and more preferably inhibitors of the enzyme raf kinase. Since the enzyme is a downstream effector of p21^{ras}, the inhibitors preferably are useful in pharmaceutical compositions for human or veterinary use where inhibition of the raf kinase pathway is indicated, e.g., in the treatment of tumors and/or cancerous cell growth mediated by raf kinase. In particular, the compounds preferably are useful in the treatment of human or animal solid cancers, e.g. murine cancer, since the progression of these cancers is dependent upon the ras protein signal transduction cascade and therefore susceptible to treatment by interruption of the cascade, i.e., by inhibiting raf kinase. Accordingly, the compound of Formula I or a pharmaceutically acceptable salt thereof can be administered for the treatment of diseases mediated by the raf kinase pathway especially cancers, preferably solid cancers, such as, for example, carcinomas (e.g., of the lungs, pancreas, thyroid, bladder or colon), myeloid disorders (e.g., myeloid leukemia) or adenomas (e.g., villous colon adenoma), pathological angiogenesis and metastatic cell migration. Furthermore the compounds preferably are useful in the treatment of complement activation dependent chronic inflammation (Niculescu et al. (2002) Immunol. Res., 24:191-199) and HIV-1 (human immunodeficiency virus type1) induced immunodeficiency (Popik et al. (1998) J Virol, 72: 6406-6413) and infection disease, Influenza A virus (Pleschka, S. et al. (2001), Nat. Cell. Biol, 3(3):301-5) and Helicobacter pylori infection (Wessler, S. et al. (2002), FASEB J., 16(3): 417-9).

Therefore, subject of the present invention are bisarylurea derivatives of formula I 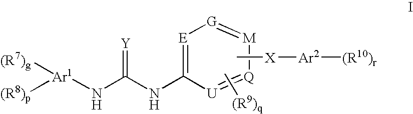
wherein
Ar¹, Ar² are selected independently from one another from aromatic hydrocarbons containing 6 to 14 carbon atoms and ethylenical unsaturated or aromatic heterocyclic residues containing 3 to 10 carbon atoms and one or two heteroatoms, independently selected from N, O and S,
E, G, M, Q and U are selected, independently from one another, from carbon atoms and nitrogen atoms, with the proviso that one or more of E, G, M, Q and U are carbon atoms and that X is bonded to a carbon atom,
R⁷ is independently selected from a group consisting of Het, OHet, N(R¹¹)Het, (CR⁵R⁶)ₖHet, O(CR⁵R⁶)ₖHet, N(R¹¹)(CR⁵R⁶)ₖHet, (CR⁵R⁶)ₖNR¹¹R¹², (CR⁵R⁶)ₖOR¹³, O(CR⁵R⁶)ₖNR¹¹R¹², NR¹¹(CR⁵R⁶)ₖNR¹¹R¹², O(CR⁵R⁶)ₖR¹³, NR¹¹(CR⁵R⁶)ₖR¹³, O(CR⁵R⁶)ₖOR¹³, NR¹¹(CR⁵R⁶)ₖOR¹³, (CR⁵R⁶)ₙO(CR⁵R⁶)ₖNR¹¹R¹², O(CR⁵R⁶)ₙO(CR⁵R⁶)ₖNR¹¹R¹², NR¹¹(CR⁵R⁶)ₙO(CR⁵R⁶)ₖNR¹¹R¹², (CR⁵R⁶)ₙNR¹¹(CR⁵R⁶)ₖNR¹¹R¹², O(CR⁵R⁶)ₙNR¹¹(CR⁵R⁶)ₖNR¹¹R¹², NR¹¹(CR⁵R⁶)ₙNR¹²(CR⁵R⁶)ₖNR¹¹R¹², (CR⁵R⁶)ₙO(CR⁵R⁶)ₖOR¹¹, O(CR⁵R⁶)ₙO(CR⁵R⁶)ₖOR¹¹, NR¹¹(CR⁵R⁶)ₙO(CR⁵R⁶)ₖOR¹², (CR⁵R⁶)ₙNR¹¹(CR⁵R⁶)ₖOR¹², O(CR⁵R⁶)ₙNR¹¹(CR⁵R⁶)ₖOR¹² and NR¹²(CR⁵R⁶)ₙNR¹¹(CR⁵R⁶)ₖOR¹² wherein
R⁵, R⁶ are in each case independently from one another selected from H and A;
R⁸, R⁹ and R¹⁰ are independently selected from a group consisting of H, A, cycloalkyl comprising 3 to 7 carbon atoms, Hal, CH₂Hal, CH(Hal)₂, C(Hal)₃, NO₂, (CH₂)ₙCN, (CH₂)ₙNR¹¹R¹², (CH₂)ₙO(CH₂)ₖNR¹¹R¹², (CH₂)ₙNR¹¹(CH₂)ₖNR¹¹R¹², (CH₂)ₙO(CH₂)ₖOR¹¹, (CH₂)ₙNR¹¹(CH₂)ₖOR¹², (CH₂)ₙCOOR¹³, (CH₂)COR¹³, (CH₂)ₙCONR¹¹R¹², (CH₂)ₙNR¹¹COR¹³, (CH₂)ₙNR¹¹CONR¹¹R¹², (CH₂)ₙNR¹¹SO₂A, (CH₂)ₙSO₂NR¹¹R¹², (CH₂)ₙS(O)ᵤR¹³, (CH₂)ₙOC(O)R¹³, (CH₂)ₙCOR¹³, (CH₂)ₙSR¹¹, CH═N—OA, CH₂CH═N—OA, (CH₂)ₙNHOA, (CH₂)ₙCH═N—R¹¹, (CH₂)ₙOC(O)NR¹¹R¹², (CH₂)ₙNR¹¹COOR¹³, (CH₂)ₙN(R¹¹)CH₂CH₂OR¹³, (CH₂)ₙN(R¹¹)CH₂CH₂OCF₃, (CH₂)ₙN(R¹¹)C(R¹³)HCOOR¹², (CH₂)ₙN(R¹¹)C(R¹³)HCOR¹¹, (CH₂)ₙN(R¹¹)CH₂CH₂N(R¹²)CH₂COOR¹¹, (CH₂)ₙN(R¹¹)CH₂CH₂NR¹¹R¹², CH═CHCOOR¹³, CH═CHCH₂NR¹¹R¹², CH═CHCH₂NR¹¹R¹², CH═CHCH₂OR¹³, (CH₂)ₙN(COOR¹³)COOR¹⁴, (CH₂)ₙN(CONH₂)COOR¹³, (CH₂)ₙN(CONH₂)CONH₂, (CH₂)ₙN(CH₂COOR¹³)COOR¹⁴, (CH₂)ₙN(CH₂CONH₂)COOR¹³, (CH₂)ₙN(CH₂CONH₂)CONH₂, (CH₂)ₙCHR¹³COR¹⁴, (CH₂)ₙCHR¹³COOR¹⁴, (CH₂)ₙCHR¹³CH₂OR¹⁴, (CH₂)ₙOCN and (CH₂)ₙNCO, wherein
R¹¹, R¹² are independently selected from a group consisting of H, A, (CH₂)ₘAr³ and (CH₂)ₘHet, or in NR¹¹R¹²,
R¹¹ and R¹² form, together with the N-atom they are bound to, a 5-, 6- or 7-membered heterocyclus which optionally contains 1 or 2 additional hetero atoms, selected from N, O and S; whereby said heterocyclic residue optionally is substituted by one or more substituent, selected from A, R¹³, ═O, ═S and ═N—R¹⁴,
R¹³, R¹⁴ are independently selected from a group consisting of H, Hal, A, (CH₂)ₘAr⁴ and (CH₂)ₘHet,
A is selected from the group consisting of alkyl, alkenyl, cycloalkyl, alkylenecycloalkyl, alkoxy, alkoxyalkyl and saturated heterocyclyl, preferably from the group consisting of alkyl, alkenyl, cycloalkyl, alkylenecycloalkyl, alkoxy and alkoxyalkyl,
Ar³, Ar⁴ are independently from one another aromatic hydrocarbon residues comprising 5 to 12 and preferably 5 to 10 carbon atoms which are optionally substituted by one or more substituents, selected from a group consisting of A, Hal, NO₂, CN, OR¹⁵, NR¹⁵R¹⁶, COOR¹⁵, CONR¹⁵R¹⁶, NR¹⁵COR¹⁶, NR¹⁵CONR¹⁵R¹⁶, NR¹⁶SO₂A, COR¹⁵, SO₂NR¹⁵R¹⁶, S(O)ᵤA and OOCR¹⁵,
Het is a saturated, unsaturated or aromatic heterocyclic residue which preferably contains 1 to 3 heteroatoms, more preferably 1 or 2 heteroatoms, the heteroatoms being preferably selected from N, O and S, more preferably from N and O; whereby said heterocyclic residue is optionally substituted by one or more substituents, selected from a group consisting of A, R¹³, ═O, ═S, ═N—R¹⁴, Hal, NO₂, CN, OR¹⁵, NR¹⁵R¹⁶, COR¹⁵, CONR¹⁵R¹⁶, NR¹⁵COR¹⁶, NR¹⁵CONR¹⁵R¹⁶, NR¹⁶SO₂A, COR¹⁵, SO₂NR¹⁵R¹⁶, S(O)ᵤA and OOCR¹⁵,
R¹⁵, R¹⁶ are independently selected from a group consisting of H, A, and (CH₂)ₘAr⁶, wherein
Ar⁶ is a 5- or 6-membered aromatic hydrocarbon which is optionally substituted by one or more substituents selected from a group consisting of methyl, ethyl, propyl, 2-propyl, tert.-butyl, Hal, CN, OH, NH₂ and CF₃,
k, n and m are independently of one another 0, 1, 2, 3, 4, or 5,
X represents a bond or is (CR¹¹R¹²)ₕ, or (CHR¹¹)ₕ-Q-(CHR¹²)ᵢ, wherein
Q is selected from a group consisting of O, S, N—R¹⁵, (CHal₂)ⱼ, (O—CHR¹⁸)ⱼ, (CHR¹⁸—O)ⱼ, CR¹⁸═CR¹⁹, (O—CHR¹⁸CHR¹⁹)ⱼ, (CHR¹⁸CHR¹⁹—O)ⱼ, C═O, C═S, C═NR¹⁵, CH(OR¹⁵), C(OR¹⁵)(OR²⁰), C(═O)O, OC(═O), OC(═O)O, C(═O)N(R¹⁵), N(R¹⁵)C(═O), OC(═O)N(R¹⁵), N(R¹⁵)C(═O)O, CH═N—O, CH═N—NR¹⁵, OC(O)NR¹⁵, NR¹⁵C(O)O, S═O, SO₂, SO₂NR¹⁵ and NR¹⁵SO₂, wherein
h, i are independently from each other 0, 1, 2, 3, 4, 5, or 6, and
j is 1, 2, 3, 4, 5, or 6,
Y is selected from O, S, NR²¹, C(R²²)—NO₂, C(R²²)—CN and C(CN)₂, wherein
R²¹ is independently selected from the meanings given for R¹³, R¹⁴ and
R²² is independently selected from the meanings given for R¹¹, R¹²,
g is 1, 2 or 3, preferably 1 or 2,
p, r are independently from one another 0, 1, 2, 3, 4 or 5,
q is 0, 1, 2, 3 or 4, preferably 0, 1 or 2,
u is 0, 1, 2 or 3, preferably 0, 1 or 2,
   and
Hal is independently selected from a group consisting of F, Cl, Br and I;
   and the pharmaceutically acceptable derivatives, solvates, salts and stereoisomers thereof, including mixtures thereof in all ratios, and more preferred the salts and/or solvates thereof, and especially preferred the physiologically acceptable salts and/or solvates thereof.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, the term “alkyl” preferably refers to a straight or branched chain hydrocarbon having from one to twelve carbon atoms, optionally substituted with substituents selected from the group consisting of C₁-C₆ alkyl, C₁-C₆ alkoxy, C₁-C₆ alkylsulfanyl, C₁-C₆ alkylsulfenyl, C₁-C₆ alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or C₁-C₆ perfluoroalkyl, multiple degrees of substitution being allowed. Examples of “alkyl” as used herein include, but are not limited to, methyl, ethyl, n-propyl, isopropyl, n-butyl, isobutyl, t-butyl, n-pentyl, isopentyl, and the like.

As used herein, the term “C₁-C₆ alkyl” preferably refers to an alkyl group as defined above containing at least 1, and at most 6, carbon atoms. Examples of branched or straight chained “C₁-C₆ alkyl” groups useful in the present invention include, but are not limited to, methyl, ethyl, n-propyl, isopropyl, isobutyl, n-butyl, t-butyl, n-pentyl and isopentyl.

As used herein, the term “alkylene” preferably refers to a straight or branched chain divalent hydrocarbon radical having from one to ten carbon atoms, optionally substituted with substituents selected from the group which includes lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl, optionally substituted by alkyl, nitro, cyano, halogen and lower perfluoroalkyl, multiple degrees of substitution being allowed. Examples of “alkylene” as used herein include, but are not limited to, methylene, ethylene, n-propylene, n-butylene and the like.

As used herein, the term “C₁-C₆ alkylene” preferably refers to an alkylene group, as defined above, which contains at least 1, and at most 6, carbon atoms respectively. Examples of “C₁-C₆ alkylene” groups useful in the present invention include, but are not limited to, methylene, ethylene and n-Propylene.

As used herein, the term “halogen” or “hal” preferably refers to fluorine (F), chlorine (Cl), bromine (Br) or iodine (I).

As used herein, the term “C₁-C₆ haloalkyl” preferably refers to an alkyl group as defined above containing at least 1, and at most 6, carbon atoms substituted with at least one halogen, halogen being as defined herein. Examples of branched or straight chained “C₁-C₆ haloalkyl” groups useful in the present invention include, but are not limited to, methyl, ethyl, propyl, isopropyl, isobutyl and n-butyl substituted independently with one or more halogens, e.g., fluoro, chloro, bromo and iodo.

As used herein, the term “cycloalkyl” or “C₃-C₇ cycloalkyl” preferably refers to a non-aromatic cyclic hydrocarbon ring having from three to seven carbon atoms and which optionally includes a C₁-C₆ alkyl linker through which it may be attached. The C₁-C₆ alkyl group is as defined above. Exemplary “C₃-C₇ cycloalkyl” groups include, but are not limited to, cyclopropyl, cyclobutyl, cyclopentyl, cyclohexyl and cycloheptyl. The term “cycloalkyl”, as used herein preferably also includes saturated heterocyclic groups, which are preferably selected from the cycloalkyl-groups as defined above, wherein one or two carbon atoms are replaced by hetero atoms, selected from the group consisting of O, N and S, which optionally is substituted by one or more substituents, preferably selected from alkyl, ═O, ═S and substituted or unsubstituted imino groups.

As used herein, the term “C₃-C₇ cycloalkylene” preferably refers to a non-aromatic alicyclic divalent hydrocarbon radical having from three to seven carbon atoms, optionally substituted with substituents selected from the group which includes lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, lower perfluoroalkyl, multiple degrees of substitution being allowed. Examples of “cycloalkylene” as used herein include, but are not limited to, cyclopropyl-1,1-diyl, cyclopropyl-1,2-diyl, cyclobutyl-1,2-diyl, cyclopentyl-1,3-diyl, cyclohexyl-1,4-diyl, cycloheptyl-1,4-diyl, or cyclooctyl-1,5-diyl, and the like.

As used herein, the term “heterocyclic” or the term “heterocyclyl” preferably refers to a three to twelve-membered heterocyclic ring having one or more degrees of unsaturation containing one or more heteroatomic substitutions selected from S, SO, SO₂, O or N, optionally substituted with substituents selected from the group consisting of C₁-C₆ alkyl, C₁-C₆ haloalkyl, C₁-C₆ alkoxy, C₁-C₆ alkylsulfanyl, C₁-C₆ haloalkylsulfanyl, C₁-C₆ alkylsulfenyl, C₁-C₆ alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or C₁-C₆ perfluoroalkyl, multiple degrees of substitution being allowed. Such a ring may be optionally fused to one or more other “heterocyclic” ring(s) or cycloalkyl ring(s). Examples of “heterocylic” moieties include, but are not limited to, tetrahydrofuran, pyran, 1,4-dioxane, 1,3-dioxane, pyrrolidine, piperidine, morpholine, tetrahydrothiopyran, tetrahydrothiophene, and the like.

As used herein, the term “heterocyclylene” preferably refers to a three to twelve-membered heterocyclic ring diradical having one or more degrees of unsaturation containing one or more heteroatoms selected from S, SO, SO₂, O or N, optionally substituted with substituents selected from the group which includes lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, lower perfluoroalkyl, multiple degrees of substitution being allowed. Such a ring may be optionally fused to one or more benzene rings or to one or more of another “heterocyclic” rings or cycloalkyl rings. Examples of “heterocyclylene” include, but are not limited to, tetrahydrofuran-2,5-diyl, morpholine-2,3-diyl, pyran-2,4-diyl, 1,4-dioxane-2,3-diyl, 1,3-dioxane-2,4-diyl, piperidine-2,4-diyl, piperidine-1,4-diyl, pyrrolidine-1,3-diyl, morpholine-2,4-diyl, and the like.

As used herein, the term “aryl” preferably refers to an optionally substituted benzene ring or to an optionally substituted benzene ring system fused to one or more optionally substituted benzene rings to form, for example, anthracene, phenanthrene, or napthalene ring systems. Exemplary optional substituents include C₁-C₆ alkyl, C₁-C₆ alkoxy, C₁-C₆ alkylsulfanyl, C₁-C₆ alkylsulfenyl, C₁-C₆ alkylsulfonyl, oxo, hydroxy, mercapto, amino optionally substituted by alkyl, carboxy, tetrazolyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, nitro, cyano, halogen, C₁-C₆ perfluoroalkyl, heteroaryl, or aryl, multiple degrees of substitution being allowed. Examples of “aryl” groups include, but are not limited to Phenyl, 2-naphthyl, 1-naphthyl, biphenyl, as well as substituted derivatives thereof. As used herein, the term “arylene” preferably refers to a benzene ring diradical or to a benzene ring system diradical fused to one or more optionally substituted benzene rings, optionally substituted with substituents selected from the group which includes lower alkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, nitro, cyano, halogen, lower perfluoroalkyl, heteroaryl and aryl, multiple degrees of substitution being allowed. Examples of “arylene” include, but are not limited to benzene-1,4-diyl, naphthalene-1,8-diyl, anthracene-1,4-diyl, and the like.

As used herein, the term “aralkyl” preferably refers to an aryl or heteroaryl group, as defined herein, attached through a C₁-C₆ alkyl linker, wherein C₁-C₆ alkyl is as defined herein. Examples of “aralkyl” include, but are not limited to, benzyl, phenylpropyl, 2-pyridylmethyl, 3-isoxazolylmethyl, 5-methyl-3-isoxazolylmethyl and 2-imidazolylethyl.

As used herein, the term “heteroaryl” preferably refers to a monocyclic five to seven-membered aromatic ring, or to a fused bicyclic aromatic ring system comprising two of such monocyclic five to seven-membered aromatic rings. These heteroaryl rings contain one or more nitrogen, sulfur and/or oxygen heteroatoms, where N—Oxides and sulfur Oxides and dioxides are permissible heteroatom substitutions and may be optionally substituted with up to three members selected from a group consisting of C₁-C₆ alkyl, C₁-C₆ haloalkyl, C₁-C₆ alkoxy, C₁-C₆ alkylsulfanyl, C₁-C₆ haloalkylsulfanyl, C₁-C₆ alkylsulfenyl, C₁-C₆ alkylsulfonyl, oxo, hydroxy, mercapto, amino optionally substituted by alkyl, carboxy, tetrazolyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, nitro, cyano, halogen, C₁-C₆ perfluoroalkyl, heteroaryl or aryl, multiple degrees of substitution being allowed. Examples of “heteroaryl” groups used herein include furanyl, thiophenyl, pyrrolyl, imidazolyl, pyrazolyl, triazolyl, tetrazolyl, thiazolyl, oxazolyl, isoxazolyl, oxadiazolyl, oxo-pyridyl, thiadiazolyl, isothiazolyl, pyridyl, pyridazyl, pyrazinyl, pyrimidyl, quinolinyl, isoquinolinyl, benzofuranyl, benzothiophenyl, indolyl, indazolyl, and substituted versions thereof.

As used herein, the term “heteroarylene” preferably refers to a five- to seven-membered aromatic ring diradical, or to a polycyclic heterocyclic aromatic ring diradical, containing one or more nitrogen, oxygen, or sulfur heteroatoms, where N—Oxides and sulfur monoxides and sulfur dioxides are permissible heteroaromatic substitutions, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, nitro, cyano, halogen, lower perfluoroalkyl, heteroaryl, or aryl, multiple degrees of substitution being allowed. For polycyclic aromatic ring system diradicals, one or more of the rings may contain one or more heteroatoms. Examples of “heteroarylene” used herein are furan-2,5-diyl, thiophene-2,4-diyl, 1,3,4-oxadiazole-2,5-diyl, 1,3,4-thiadiazole-2,5-diyl, 1,3-thiazole-2,5-diyl, pyridine-2,4-diyl, pyridine-2,3-diyl, pyridine-2,5-diyl, pyrimidine-2,4-diyl, quinoline-2,3-diyl, and the like.

As used herein, the term “alkoxy” preferably refers to the group RₐO—, where Rₐ is alkyl as defined above and the term “C₁-C₆ alkoxy” preferably refers to an alkoxy group as defined herein wherein the alkyl moiety contains at least 1 and at most 6 carbon atoms. Exemplary C₁-C₆ alkoxy groups useful in the present invention include, but are not limited to methoxy, ethoxy, n-propoxy, isopropoxy, n-butoxy and t-butoxy.

As used herein, the term “haloalkoxy” preferably refers to the group RₐO—, where Rₐ is haloalkyl as defined above and the term “C₁-C₆ haloalkoxy” preferably refers to an haloalkoxy group as defined herein wherein the haloalkyl moiety contains at least 1 and at most 6 carbon atoms. Exemplary C₁-C₆ haloalkoxy groups useful in the present invention include, but are not limited to, methoxy, ethoxy, n-propoxy, isopropoxy, n-butoxy and t-butoxy substituted with one or more halo groups, for instance trifluoromethoxy.

As used herein the term “aralkoxy” preferably refers to the group R_{C}R_{B}O—, where R_{B} is alkyl and R_{C} is aryl as defined above.

As used herein the term “aryloxy” preferably refers to the group R_{C}O—, where R_{C} is aryl as defined above.

As used herein, the term “alkylsulfanyl” preferably refers to the group R_{A}S—, where R_{A} is alkyl as defined above and the term “C₁-C₆ alkylsulfanyl” preferably refers to an alkylsulfanyl group as defined herein wherein the alkyl moiety contains at least 1 and at most 6 carbon atoms.

As used herein, the term “haloalkylsulfanyl” preferably refers to the group R_{D}S—, where R_{D} is haloalkyl as defined above and the term “C₁-C₆ haloalkylsulfanyl” preferably refers to a haloalkylsulfanyl group as defined herein wherein the alkyl moiety contains at least 1 and at most 6 carbon atoms.

As used herein, the term “alkylsulfenyl” preferably refers to the group R_{A}S(O)—, where R_{A} is alkyl as defined above and the term “C₁-C₆ alkylsulfenyl” preferably refers to an alkylsulfenyl group as defined herein wherein the alkyl moiety contains at least 1 and at most 6 carbon atoms.

As used herein, the term “alkylsulfonyl” preferably refers to the group R_{A}SO₂— where R_{A} is alkyl as defined above and the term “C₁-C₆ alkylsulfonyl” preferably refers to an alkylsulfonyl group as defined herein wherein the alkyl moiety contains at least 1 and at most 6 carbon atoms.

As used herein, the term “oxo” preferably refers to the group ═O.

As used herein, the term “mercapto” preferably refers to the group —SH.

As used herein, the term “carboxy” preferably refers to the group —COOH.

As used herein, the term “cyano” preferably refers to the group —CN.

As used herein, the term “cyanoalkyl” preferably refers to the group —R_{B}CN, wherein R_{B} is alkylen as defined above. Exemplary “cyanoalkyl” groups useful in the present invention include, but are not limited to, cyanomethyl, cyanoethyl and cyanoisopropyl.

As used herein, the term “aminosulfonyl” preferably refers to the group —SO₂NH₂.

As used herein, the term “carbamoyl” preferably refers to the group —C(O)NH₂.

As used herein, the term “sulfanyl” shall refer to the group —S—.

As used herein, the term “sulfenyl” shall refer to the group —S(O)—.

As used herein, the term “sulfonyl” shall refer to the group —S(O)₂— or —SO₂—.

As used herein, the term “acyl” preferably refers to the group R_{F}C(O)—, where R_{F} is alkyl, cycloalkyl or heterocyclyl as defined herein.

As used herein, the term “aroyl” preferably refers to the group R_{C}C(O)—, where R_{C} is aryl as defined herein.

As used herein, the term “heteroaroyl” preferably refers to the group R_{E}C(O)—, where R_{E} is heteroaryl as defined herein.

As used herein, the term “alkoxycarbonyl” preferably refers to the group R_{A}OC(O)—, where R_{A} is alkyl as defined herein.

As used herein, the term “acyloxy” preferably refers to the group R_{F}C(O)O—, where R_{F} is alkyl, cycloalkyl, or heterocyclyl as defined herein.

As used herein, the term “aroyloxy” preferably refers to the group R_{C}C(O)O—, where R_{C} is aryl as defined herein.

As used herein, the term “heteroaroyloxy” preferably refers to the group R_{E}C(O)O—, where R_{E} is heteroaryl as defined herein.

As used herein, the term “carbonyl” or “carbonyl moiety” preferably refers to the group C═O.

As used herein, the term “thiocarbonyl” or “thiocarbonyl moiety” preferably refers to the group C═S.

As used herein, the term “amino”, “amino group” or “imino moiety” preferably refers to the group NR_{G}R_{G′}, wherein R_{G} and R_{G′}, are preferably selected, independently from one another, from the group consisting of hydrogen, halogen, alkyl, haloalkyl, alkenyl, cycloalkyl, alkylenecycloalkyl, cyanoalkyl, aryl, aralkyl, heteroaryl, acyl and aroyl. If both R_{G} and R_{G′} are hydrogen, NR_{G}R_{G′} is also referred to as “unsubstituted amino moiety” or “unsubstituted amino group”. If R_{G} and/or R_{G′} are other than hydrogen, NR_{G}R_{G′} is also referred to as “substituted amino moiety” or “substituted amino group”.

As used herein, the term “imino” or “imino moiety” preferably refers to the group C═NR_{G}, wherein R_{G} is preferably selected from the group consisting of hydrogen, halogen, alkyl, haloalkyl, alkenyl, cycloalkyl, alkylenecycloalkyl, cyanoalkyl, aryl, aralkyl, heteroaryl, acyl and aroyl. If R_{G} is hydrogen, C═NR_{G} is also referred to as “unsubstituted imino moiety”. If R_{G} is a residue other than hydrogen, C═NR_{G} is also referred to as “substituted imino moiety”.

As used herein, the term “ethene-1,1-diyl moiety” preferably refers to the group C═CR_{K}R_{L}, wherein R_{K} and R_{L} are preferably selected, independently from one another, from the group consisting of hydrogen, halogen, alkyl, haloalkyl, alkenyl, cycloalkyl, nitro, alkylenecycloalkyl, cyanoalkyl, aryl, aralkyl, heteroaryl, acyl and aroyl. If both hydrogen R_{K} and R_{L} are hydrogen, C═CR_{K}R_{L} is also referred to as “unsubstituted ethene-1,1-diyl moiety”. If one of R_{K} and R_{L} or both are a residue other than hydrogen, C═CR_{K}R_{L} is also referred to as “substituted ethene-1,1-diyl moiety”.

As used herein, the terms “group”, “residue” and “radical” or “groups”, “residues” and “radicals” are usually used as synonyms, respectively, as it is common practice in the art.

As used herein, the term “optionally” means that the subsequently described event(s) may or may not occur, and includes both event(s), which occur, and events that do not occur.

As used herein, the term “physiologically functional derivative” preferably refers to any pharmaceutically acceptable derivative of a compound of the present invention, for example, an ester or an amide, which upon administration to a mammal is capable of providing (directly or indirectly) a compound of the present invention or an active metabolite thereof. Such derivatives are clear to those skilled in the art, without undue experimentation, and with reference to the teaching of Burger's Medicinal Chemistry And Drug Discovery, 5th Edition, Vol 1: Principles and Practice, which is incorporated herein by reference to the extent that it teaches physiologically functional derivatives.

As used herein, the term “solvate” preferably refers to a complex of variable stoichiometry formed by a solute (in this invention, a compound of formula I or a salt or physiologically functional derivative thereof) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water.

As used herein, the term “substituted” preferably refers to substitution with the named substituent or substituents, multiple degrees of substitution being allowed unless otherwise stated.

Certain of the compounds described herein may contain one or more chiral atoms, or may otherwise be capable of existing as two or more stereoisomers, which are usually enantiomers and/or diastereomers. Accordingly, the compounds of this invention include mixtures of stereoisomers, especially mixtures of enantiomers, as well as purified stereoisomers, especially purified enantiomers, or stereoisomerically enriched mixtures, especially enantiomerically enriched mixtures. Also included within the scope of the invention are the individual isomers of the compounds represented by formulae I above as well as any wholly or partially equilibrated mixtures thereof. The present invention also covers the individual isomers of the compounds represented by the formulas above as mixtures with isomers thereof in which one or more chiral Centers are inverted. Also, it is understood that all tautomers and mixtures of tautomers of the compounds of formulae I are included within the scope of the compounds of formulae I and preferably the formulae and subformulae corresponding thereto.

Racemates obtained can be resolved into the isomers mechanically or chemically by methods known per se. Diastereomers are preferably formed from the racemic mixture by reaction with an optically active resolving agent. Examples of suitable resolving agents are optically active acids, such as the D and L forms of tartaric acid, diacetyltartaric acid, dibenzoyltartaric acid, mandelic acid, malic acid, lactic acid or the various optically active camphorsulfonic acids, such as β-camphorsulfonic acid. Also advantageous is enantiomer resolution with the aid of a column filled with an optically active resolving agent (for example dinitrobenzoylphenylglycine); an example of a suitable eluent is a hexane/isopropanol/acetonitrile mixture.

The diastereomer resolution can also be carried out by standard purification processes, such as, for example, chromatography or fractional crystallization.

It is of course also possible to obtain optically active compounds of the formula I by the methods described above by using starting materials which are already optically active.

Unless indicated otherwise, it is to be understood that reference to compounds of formula I preferably includes the reference to the compounds of formula I′ and I″. Unless indicated otherwise, it is to be understood that reference to the compounds of formula I, I′ and I″ preferably includes the reference to the sub formulae corresponding thereto, for example the sub formulae I.1 to I.20 and preferably formulae Ia to Iz and Iaa to Iuu. It is also understood that the following embodiments, including uses and compositions, although recited with respect to formula I are preferably also applicable to formulae I′, I″ and sub formulae I.1 to I.20 and preferably formulae Ia to Iz and Iaa to Iuu.

Even more preferred are compounds of formula I

wherein

Ar¹, Ar² are selected independently from one another from aromatic hydrocarbons containing 6 to 10 and especially 6 carbon atoms and ethylenical unsaturated or aromatic heterocyclic residues containing 3 to 8 and especially 4 to 6 carbon atoms and one or two heteroatoms, independently selected from N, O and S and especially selected from N and O,
R⁷ is independently selected from a group consisting of Het, OHet, N(R¹¹)Het, (CR⁵R⁶)ₖHet, O(CR⁵R⁶)ₖHet, N(R¹¹)(CR⁵R⁶)ₖHet, (CR⁵R⁶)ₖNR¹¹R¹², (CR⁵R⁶)ₖOR¹³, O(CR⁵R⁶)ₖNR¹¹R¹², NR¹¹(CR⁵R⁶)ₖNR¹¹R¹², O(CR⁵R⁶)ₖR¹³, NR¹¹(CR⁵R⁶)ₖR¹³, O(CR⁵R⁶)ₖOR¹³, NR¹¹(CR⁵R⁶)ₖOR¹³, O(CR⁵R⁶)ₙO(CR⁵R⁶)ₖNR¹¹R¹², NR¹¹(CR⁵R⁶)ₙO(CR⁵R⁶)ₖNR¹¹R¹², O(CR⁵R⁶)ₙNR¹¹(CR⁵R⁶)ₖNR¹¹R¹², NR¹¹(CR⁵R⁶)ₙNR¹²(CR⁵R⁶)ₖNR¹¹R¹², O(CR⁵R⁶)ₙO(CR⁵R⁶)ₖOR¹¹, NR¹¹(CR⁵R⁶)ₙO(CR⁵R⁶)ₖOR¹², O(CR⁵R⁶)ₙNR¹¹(CR⁵R⁶)ₖOR¹² and NR¹²(CR⁵R⁶)ₙNR¹¹(CR⁵R⁶)ₖOR¹², wherein
R⁵, R⁶ are in each case independently from one another selected from H and A, and
n and/or k independently are 0, 1, 2, 3 or 4, preferably 1, 2, 3 or 4, and even more preferred is 2 or 3;
R⁸, R⁹ and R¹⁰ are independently selected from a group consisting of H, A, cycloalkyl comprising 3 to 7 carbon atoms, Hal, CH₂Hal, CH(Hal)₂, C(Hal)₃, NO₂, (CH₂)ₙCN, (CH₂)ₙNR¹¹R¹², (CH₂)ₙO(CH₂)ₖNR¹¹R¹², (CH₂)ₙNR¹¹(CH₂)ₖNR¹¹R¹², (CH₂)ₙO(CH₂)ₖOR¹¹, (CH₂)ₙNR¹¹(CH₂)ₖOR¹², (CH₂)ₙCOR¹³, (CH₂)ₙCOOR¹³, (CH₂)ₙCONR¹¹R¹², (CH₂)ₙNR¹¹COR¹³, (CH₂)ₙNR¹¹CONR¹¹R¹², (CH₂)ₙNR¹¹SO₂A, (CH₂)ₙSO₂NR¹¹R¹², (CH₂)ₙS(O)ᵤR¹³, (CH₂)ₙOC(O)R¹³, (CH₂)ₙCOR¹³, (CH₂)ₙSR¹¹, (CH₂)ₙNHOA, (CH₂)ₙNR¹¹COOR¹³, (CH₂)ₙN(R¹¹)CH₂CH₂OR¹³, (CH₂)ₙN(R¹¹)CH₂CH₂OCF₃, (CH₂)ₙN(R¹¹)C(R¹³)HCOOR¹², (CH₂)ₙN(R¹¹)C(R¹³)HCOR¹¹, (CH₂)ₙN(COOR¹³)COOR¹⁴, (CH₂)ₙN(CONH₂)COOR¹³, (CH₂)ₙN(CONH₂)CONH₂, (CH₂)ₙN(CH₂COOR¹³)COOR¹⁴, (CH₂)ₙN(CH₂CONH₂)COOR¹³, (CH₂)ₙN(CH₂CONH₂)CONH₂, (CH₂)ₙCHR¹³COR¹⁴, (CH₂)ₙCHR¹³COOR¹⁴ and (CH₂)ₙCHR¹³CH₂OR¹⁴, wherein
n and/or k independently are 0, 1, 2, 3 or 4, preferably 0, 1, 2 or 3, and even more preferred are 0 or 2;
X represents a bond or is (CR¹¹R¹²)ₕ, or (CHR¹¹)ₕ-Q-(CHR¹²)ᵢ, wherein
Q is selected from a group consisting of O, S, N—R¹⁵, (CHal₂)ⱼ, (O—CHR¹⁸), (CHR¹⁸—O), CR¹⁸═CR¹⁹, (O—CHR¹⁸CHR¹⁹)ⱼ, (CHR¹⁸CHR¹⁹—O)ⱼ, C═O, C═NR¹⁵, CH(OR¹⁵), C(OR¹⁵)(OR²⁰), C(═O)N(R¹⁵), N(R¹⁵)C(═O), CH═N—NR¹⁵, S═O, SO₂, SO₂NR¹⁵ and NR¹⁵SO₂, wherein
h, i are independently from each other 0, 1, 2, 3, 4, 5 or 6, preferably 0, 1, 2 or 3 and
j is 1, 2, 3, 4, 5 or 6, preferably 1, 2, 3 or 4,
g is 1 or 2, preferably 1,
p is 1, 2 or 3, preferably 1 or 2, and
r is 0, 1, 2, or 3, preferably 0, 1 or 2;
   and the pharmaceutically acceptable derivatives, solvates, salts and stereoisomers thereof, including mixtures thereof in all ratios, and more preferred the salts and/or solvates thereof, and especially preferred the physiologically acceptable salts and/or solvates thereof.

Subject of the present invention are especially compounds of formula I in which one or more substituents or groups, preferably the major part of the substituents or groups has a meaning which is indicated as preferred, more preferred, even more preferred or especially preferred.

In compounds of formula I, E, G, M, Q and U constitute, together with the carbon atom that E and U are bound to, a bivalent 6-membered aromatic or nitrogen containing heteroaromatic ring. Preferably, one or more of E, G, M, Q and U, more preferably two or more of E, G, M, Q and U and especially three or more of E, G, M, Q and U are carbon atoms. Especially preferred, none or one of E, G, M, Q and U is a nitrogen atom. Especially preferred, E, G, M, Q and U constitute, together with the carbon atom that E and U are bound to, a 6-membered aromatic or nitrogen containing heteroaromatic ring, selected from the group consisting of phenylen, pyridinylen and pyrimydylen, wherein X is preferably bonded to a carbon atom. The substituents R⁹ are preferably bound to a carbon atom.

More preferred as compounds of formula I are compounds of formula I′, 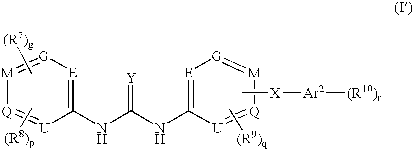
wherein each of E, G, M, Q and U is independently from one another selected from carbon atoms and nitrogen atoms, with the proviso that in each of the E, G, M, Q and U containing 6-membered rings, one or more of E, G, M, Q and U are carbon atoms, and the further proviso that X and preferably substituents (R⁷)_{g} and (R⁸)ₚ are bonded to a carbon atom, respectively. More preferably, in the E, G, M, Q and U containing 6-membered ring one or more times substituted by R⁷, U is CR⁷, where R⁷ is as defined above/below. Accordingly, especially preferred as compounds of formula I and compounds of formula I′ are compounds of formula I″, 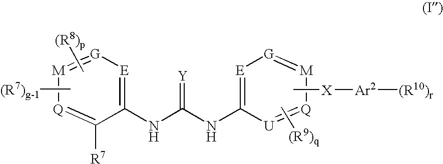
wherein each residue R⁵⁷ is independently selected from the meanings given above/below.

In compounds of formula I, the term alkyl preferably refers to an unbranched or branched alkyl residue, preferably an unbranched alkyl residue comprising 1, 2, 3, 4, 5, 6, 7, 8, 9 or 10, preferably 1, 2, 3, 4, 5 or 6, more preferred 1, 2, 3 or 4 and especially 1 or 2 carbon atoms, or a branched alkyl residue comprising 3, 4, 5, 6, 7, 8, 9 or 10, preferably 3, 4, 5 or 6 more preferred 3 or 4 carbon atoms. The alkyl residues can be optionally substituted, especially by one or more halogen atoms, for example up to perhaloalkyl, by one or more hydroxy groups or by one or more amino groups, all of which can optionally be substituted by alkyl. If an alkyl residue is substituted by halogen, it usually comprises 1, 2, 3, 4 or 5 halogen atoms, depending on the number of carbon atoms of the alkyl residue. For example, a methyl group can comprise, 1, 2 or 3 halogen atoms, an ethyl group (an alkyl residue comprising 2 carbon atoms) can comprise 1, 2, 3, 4 or 5 halogen atoms. If an alkyl residue is substituted by hydroxy groups, it usually comprises one or two, preferably one hydroxy groups. If the hydroxy group is substituted by alkyl, the alkyl substituent comprises preferably 1 to 4 carbon atoms and is preferably unsubstituted or substituted by halogen and more preferred unsubstituted. If an alkyl residue is substituted by amino groups, it usually comprises one or two, preferably one amino groups. If the amino group is substituted by alkyl, the alkyl substituent comprises preferably 1 to 4 carbon atoms and is preferably unsubstituted or substituted by halogen and more preferred unsubstituted. According to compounds of formula I, alkyl is preferably selected from the group consisting of methyl, ethyl, trifluoro methyl, pentafluoro ethyl, isopropyl, tert.-butyl, 2-amino ethyl, N-methyl-2-amino ethyl, N,N-dimethyl-2-amino ethyl, N-ethyl-2-amino ethyl, N,N-diethyl-2-amino ethyl, 2-hydroxy ethyl, 2-methoxy ethyl and 2-ethoxy ethyl, further preferred of the group consisting of 2-butyl, n-pentyl, neo-nentyl, isopentyl, hexyl and n-decyl, more preferred of methyl, ethyl, trifluoro methyl, isopropyl and tert.-butyl.

In compounds of formula I, alkenyl is preferably selected from the group consisting of allyl, 2- or 3-butenyl, isobutenyl, sec-butenyl, furthermore preferably 4-pentenyl, isopentenyl and 5-hexenyl.

In compounds of formula I, alkylene is preferably unbranched and is more preferably methylene or ethylene, furthermore preferably propylene or butylene.

In compounds of formula I, alkylenecycloalkyl preferably has 5 to 10 carbon atoms and is preferably methylenecyclopropyl, methylenencyclobutyl, furthermore preferably methylenecyclopentyl, methylenecyclohexyl or methylenecycloheptyl, furthermore alternatively ethylenecyclopropyl, ethylenecyclobutyl, ethylenecyclopentyl, ethylenecyclohexyl or ethylenencycloheptyl, propylenecyclopentyl, propylenecyclohexyl, butylenecyclopentyl or butylenecyclohexyl.

In compounds of formula I, the term “alkoxy” preferably comprises groups of formula O-alkyl, where alkyl is an alkyl group as defined above. More preferred, alkoxy is selected from group consisting of methoxy, ethoxy, n-propoxy, isopropoxy, 2-butoxy, tert.-butoxy and halogenated, especially perhalogenated, derivatives thereof. Preferred perhalogenated derivatives are selected from the group consisting of O—CCl₃, O—CF₃, O—C₂Cl₅, O—C₂F₅, O—C(CCl₃)₃ and O—C(CF₃)₃.

In compounds of formula I, the term “alkoxyalkyl” preferably comprises branched and unbranched residues, more preferred unbranched residues, of formula CᵤH₂ᵤ₊₁—O—(CH₂)ᵥ, wherein u and v are independently from each other 1 to 6. Especially preferred is u=1 and v 1 to 4.

In compounds of formula I the term “alkoxyalkyl” includes alkoxyalkyl groups as defined above, wherein one or more of the hydrogen atoms are substituted by halogen, for example up to perhalo alkoxyalkyl.

In compounds of formula I, cycloalkyl preferably has 3-7 carbon atoms and is preferably cyclopropyl or cyclobutyl, furthermore preferably cyclopentyl or cyclohexyl, furthermore also cycloheptyl, particularly preferably cyclopentyl. The term “cycloalkyl”, as used herein preferably also includes saturated heterocyclic groups, wherein one or two carbon atoms are substituted by hetero atoms, selected from the group consisting of O, NH, NA and S, wherein A is as defined as above/below. Cycloalkyl residues as defined herein can optionally be substituted, the substituents preferably selected from A, R¹³, ═O, ═S, ═N—R¹⁴, CN and hal.

In compounds of formula I, Ar³ to Ar⁶ are preferably selected independently from one another from phenyl, naphthyl and biphenyl which is optionally substituted by one or more substituents, selected from the group consisting of A, Hal, NO₂, CN, OR¹⁵, NR¹⁵R¹⁶, COOR¹⁵, CONR¹⁵R¹⁶, NR¹⁵COR¹⁶, NR¹⁵CONR¹⁵R¹⁶, NR¹⁶SO₂A, COR¹⁵, SO₂NR¹⁵R¹⁶, S(O)ᵤA and OOCR¹⁵.

In compounds of formula I, Het is preferably an optionally substituted aromatic heterocyclic residue and even more preferred and optionally substituted saturated heterocyclic residue. In substituted saturated heterocyclic residues, the substituents are preferably selected from A, R¹³, ═O, ═S, ═N—R¹⁴, CN and hal. Even more preferred, Het is selected from the group consisting of 1-piperidyl, 4-piperidyl, 1-methyl-piperidin-4-yl, 1-piperazyl, 1-(4-methyl)-piperazyl, 4-methylpiperazin-1-yl amine, 1-(4-(2-hydroxyethyl))-piperazyl, 4-morpholinyl, 1-pyrrolidinyl, 2-pyrrolidinyl, 3-pyrrolidinyl, 1-pyrazolidinyl 1-(2-methyl)-pyrazolidinyl, 1-imidazolidinyl or 1-(3-methyl)-imidazolidinyl, thiophen-2-yl, thiophen-3-yl, 2-pyridyl, 3-pyridyl, 4-pyridyl, 2-oxazolyl, 4-oxazolyl, 5-oxazolyl, 2-thiazolyl, 4-thiazolyl, 5-thiazolyl, chinolinyl, isochinolinyl, 2-pyridazyl, 4-pyridazyl, 2-pyrimidyl, 4-pyrimidyl, 5-pyrimidyl, 2-pyrazinyl and 3-pyrazinyl. Further preferred, Het as defined above is optionally substituted by one or more substituents preferably selected from A, R¹³, ═O, ═S, ═N—R¹⁴, CN and hal. More preferred, Het is either unsubstituted or substituted once or twice by ═O.

In compounds of formula I, saturated heterocyclyl is preferably a substituted or unsubstituted saturated heterocyclic residue, more preferred an unsubstituted saturated heterocyclic residue, preferably selected from the saturated groups given above in the definition of Het. Further preferred, saturated heterocyclyl as defined above is optionally substituted by one or more substituents preferably selected from A, R¹³, ═O, ═S, ═N—R¹⁴, CN and hal. More preferred, saturated heterocyclyl is either unsubstituted or substituted once or twice by ═O.

In compounds of formula I, aromatic hydrocarbons containing 6 to 14 carbon atoms and ethylenical unsaturated or aromatic heterocyclic residues containing 3 to 10 carbon atoms and one or two heteroatoms, independently selected from N, O and S, are preferably selected from the definitions given herein for aryl, heteroaryl and/or Het. Heteroaryl is more preferably furanyl, thiophenyl, pyrrolyl, imidazolyl, pyrazolyl, triazolyl, tetrazolyl, thiazolyl, oxazolyl, isoxazolyl, oxadiazolyl, oxo-pyridyl, thiadiazolyl, isothiazolyl, pyridyl, pyridazyl, pyrazinyl, pyrimidyl, quinolinyl, isoquinolinyl, benzofuranyl, benzothiophenyl, indolyl, indazolyl and even more preferably pyridinyl, pyrimidyl, chinolinyl, isochinolinyl, thiophenyl, thiadiazolyl, benzothiadiazolyl, oxazolyl, isoxazolyl, pyrazolyl and/or imidazolyl. Aryl more preferably refers to an optionally substituted benzene ring or to an optionally substituted benzene ring system fused to one or more optionally substituted benzene rings to form, for example, anthracene, phenanthrene, or napthalene ring systems. Even more preferably, aryl is selected from the group consisting of phenyl, 2-naphthyl, 1-naphthyl, biphenyl.

In compounds of formula I, Ar¹ is preferably selected from the group consisting of phenyl, pyridinyl, pyrimidyl, chinolinyl, isochinolinyl, thiophenyl, thiadiazolyl, benzothiadiazolyl, oxazolyl, isoxazolyl, pyrazolyl and imidazolyl, and especially from phenyl, pyridinyl, chinolinyl, isochinolinyl, thiophenyl, benzothiadiazolyl, oxazolyl, isoxazolyl and oxazolyl. Especially preferred, Ar¹ is phenyl or pyridinyl.

In compounds of formula I, Ar² is preferably selected from the group consisting of phenyl, pyridinyl, pyrimidyl, chinolinyl, isochinolinyl, thiophenyl, thiadiazolyl, benzothiadiazolyl, oxazolyl, isoxazolyl, pyrazolyl and imidazolyl, even more preferably from phenyl, pyridinyl and pyrimidyl and especially preferred from phenyl and pyridinyl.

Especially preferred are bisarylurea derivatives as described above/below, wherein
R⁷ is independently selected from a group consisting of Het, OHet, N(R¹¹)Het, (CR⁵R⁶)ₖHet, O(CR⁵R⁶)ₖHet, N(R¹¹)(CR⁵R⁶)ₖHet, (CR⁵R⁶)ₖNR¹¹R¹², (CR⁵R⁶)ₖOR¹³, O(CR⁵R⁶)ₖNR¹¹R¹², NR¹¹(CR⁵R⁶)ₖNR¹¹R¹², O(CR⁵R⁶)ₖR¹³, NR¹¹(CR⁵R⁶)ₖR¹³, O(CR⁵R⁶)ₖOR¹³, NR¹¹(CR⁵R⁶)ₖOR¹³, and more preferably from OHet, N(R¹¹)Het, (CR⁵R⁶)ₖHet, O(CR⁵R⁶)ₖHet, N(R¹¹)(CR⁵R⁶)ₖHet, O(CR⁵R⁶)ₖNR¹¹R¹², NR¹¹(CR⁵R⁶)ₖNR¹¹R¹², O(CR⁵R⁶)ₖOR¹³ and NR¹¹(CR⁵R⁶)ₖOR¹³, wherein
n and k are independently from one another 1, 2, 3 or 4.

If R⁵ and/or R⁶ is A, then A is preferably selected, independently from one another in each case, from the group consisting of alkyl, cycloalkyl, alkoxy, alkoxyalkyl and saturated heterocyclyl, more preferably from the group consisting of alkyl, cycloalkyl, alkoxy and alkoxyalkyl, and especially is alkyl.

Preferably, the sum of h and i in one residue exceeds 0.

Preferably, the sum of n and k in one residue exceeds 0.

In R⁷, n and/or k are preferably not 0.

In R⁷, (CR⁵R⁶)ₙ and/or (CR⁵R⁶)ₖ is preferably linear or branched alkylen, preferably linear or branched C₁-C₄ alkylen, which is optionally substituted as described above/below and preferably is unsubstituted.

Another preferred aspect of the instant invention relates to compounds of formula I, wherein n is 0 in the residues R⁸, R⁹ and/or R¹⁰ and especially in R¹⁰.

Another preferred aspect of the instant invention relates to compounds of formula I, wherein in the residues R⁷, n is 1, 2 or 3 and especially is 2.

Another preferred aspect of the instant invention relates to compounds of formula I, wherein X represents a bridging group, selected from (CR¹¹R¹²)ₕ or (CHR¹¹)ₕ-Q-(CHR¹²)ᵢ.

The invention relates in particular to compounds of the formula I in which at least one of said radicals has one of the preferred meanings given above.

Some more preferred groups of compounds may be expressed by the following sub-formulae I.1) to I.20), which correspond to the formula I and in which radicals not denoted in greater detail are as defined in the formula I, but in which
I.1) Ar¹ is phenyl, pyridinyl, pyrimidyl, chinolinyl, isochinolinyl, thiophenyl, thiadiazolyl, benzothiadiazolyl, oxazolyl, isoxazolyl, pyrazolyl or imidazolyl, preferably phenyl, pyridinyl, chinolinyl, isochinolinyl, thiophenyl, benzothiadiazolyl, oxazolyl, isoxazolyl or oxazolyl, even more preferably phenyl or pyridinyl;
I.2) Ar¹ is phenyl, pyridinyl, pyrimidyl, chinolinyl, isochinolinyl, thiophenyl, thiadiazolyl, benzothiadiazolyl, oxazolyl, isoxazolyl, pyrazolyl or imidazolyl, preferably phenyl, pyridinyl, chinolinyl, isochinolinyl, thiophenyl, benzothiadiazolyl, oxazolyl, isoxazolyl or oxazolyl, even more preferably phenyl or pyridinyl, and
   p is 1, 2 or 3;
I.3) Ar¹ is phenyl, pyridinyl, pyrimidyl, chinolinyl, isochinolinyl, thiophenyl, thiadiazolyl, benzothiadiazolyl, oxazolyl, isoxazolyl, pyrazolyl or imidazolyl, preferably phenyl, pyridinyl, chinolinyl, isochinolinyl, thiophenyl, benzothiadiazolyl, oxazolyl, isoxazolyl or oxazolyl, even more preferably phenyl or pyridinyl,
   p is 1, 2 or 3, and
   R⁸ is selected from the group consisting of alkyl comprising 1 to 4 carbon atoms, alkoxy comprising 1 to 4 carbon atoms, Hal, CH₂Hal, CH(Hal)₂, perhaloalkyl comprising 1 to 4 carbon atoms, NO₂, (CH₂)ₙCN, (CH₂)ₙNR¹¹R¹², (CH₂)ₙO(CH₂)ₖNR¹¹R¹², (CH₂)ₙNR¹¹(CH₂)ₖNR¹¹R¹², (CH₂)ₙO(CH₂)ₖOR¹¹, (CH₂)ₙNR¹¹(CH₂)ₖOR¹², (CH₂)ₙCOR¹³, (CH₂)ₙCOOR¹³, (CH₂)ₙCONR¹¹R¹², (CH₂)ₙSO₂NR¹¹R¹² and (CH₂)ₙS(O)ᵤR¹³;
I.4) Ar¹ is phenyl, pyridinyl, pyrimidyl, chinolinyl, isochinolinyl, thiophenyl, thiadiazolyl, benzothiadiazolyl, oxazolyl, isoxazolyl, pyrazolyl or imidazolyl, preferably phenyl, pyridinyl, chinolinyl, isochinolinyl, thiophenyl, benzothiadiazolyl, oxazolyl, isoxazolyl or oxazolyl, even more preferably phenyl or pyridinyl,
   p is 1, 2 or 3,
   R⁸ is selected from the group consisting of alkyl comprising 1 to 4 carbon atoms, alkoxy comprising 1 to 4 carbon atoms, Hal, CH₂Hal, CH(Hal)₂, perhaloalkyl comprising 1 to 4 carbon atoms, NO₂, (CH₂)ₙCN, (CH₂)ₙNR¹¹R¹², (CH₂)ₙO(CH₂)ₖNR¹¹R¹², (CH₂)ₙNR¹¹(CH₂)ₖNR¹¹R¹², (CH₂)ₙO(CH₂)ₖOR¹¹, (CH₂)ₙNR¹¹(CH₂)ₖOR¹², (CH₂)ₙCOR¹³, (CH₂)ₙCOOR¹³, (CH₂)ₙCONR¹¹R¹², (CH₂)ₙSO₂NR¹¹R¹² and (CH₂)ₙS(O)ᵤR¹³;
I.5) Ar¹ is phenyl, pyridinyl, pyrimidyl, chinolinyl, isochinolinyl, thiophenyl, thiadiazolyl, benzothiadiazolyl, oxazolyl, isoxazolyl, pyrazolyl or imidazolyl, preferably phenyl, pyridinyl, chinolinyl, isochinolinyl, thiophenyl, benzothiadiazolyl, oxazolyl, isoxazolyl or oxazolyl, even more preferably phenyl or pyridinyl,
   p is 1, 2 or 3,
   R⁸ is selected from the group consisting of alkyl comprising 1 to 4 carbon atoms, alkoxy comprising 1 to 4 carbon atoms, Hal, CH₂Hal, CH(Hal)₂, perhaloalkyl comprising 1 to 4 carbon atoms, NO₂, (CH₂)ₙCN, (CH₂)ₙNR¹¹R¹², (CH₂)ₙO(CH₂)ₖNR¹¹R¹², (CH₂)ₙNR¹¹(CH₂)ₖNR¹¹R¹², (CH₂)ₙO(CH₂)ₖOR¹¹, (CH₂)ₙNR¹¹(CH₂)ₖOR¹², (CH₂)ₙCOR¹³, (CH₂)ₙCOOR¹³, (CH₂)ₙCONR¹¹R¹², (CH₂)ₙSO₂NR¹¹R¹² and (CH₂)ₙS(O)ᵤR¹³, wherein
   n is 0 or 1;
I.6) Ar¹ is phenyl, pyridinyl, pyrimidyl, chinolinyl, isochinolinyl, thiophenyl, thiadiazolyl, benzothiadiazolyl, oxazolyl, isoxazolyl, pyrazolyl or imidazolyl, preferably phenyl, pyridinyl, chinolinyl, isochinolinyl, thiophenyl, benzothiadiazolyl, oxazolyl, isoxazolyl or oxazolyl, even more preferably phenyl or pyridinyl,
   p is 1, 2 or 3,
   R⁸ is selected from the group consisting of alkyl comprising 1 to 4 carbon atoms, alkoxy comprising 1 to 4 carbon atoms, Hal, CH₂Hal, CH(Hal)₂, perhaloalkyl comprising 1 to 4 carbon atoms, NO₂, (CH₂)ₙCN, (CH₂)ₙNR¹¹R¹², (CH₂)ₙO(CH₂)ₖNR¹¹R¹², (CH₂)ₙNR¹¹N(CH₂)ₖNR¹¹R¹², (CH₂)ₙO(CH₂)ₖOR¹¹, (CH₂)ₙNR¹¹(CH₂)ₖOR¹², (CH₂)ₙCOR¹³, (CH₂)ₙCOOR¹³, (CH₂)ₙCONR¹¹R¹², (CH₂)ₙSO₂NR¹¹R¹² and (CH₂)ₙS(O)ᵤR¹³, wherein
   n is 0 or 1, and
   u is 0;
I.7) Ar¹ is phenyl, pyridinyl, pyrimidyl, chinolinyl, isochinolinyl, thiophenyl, thiadiazolyl, benzothiadiazolyl, oxazolyl, isoxazolyl, pyrazolyl or imidazolyl, preferably phenyl, pyridinyl, chinolinyl, isochinolinyl, thiophenyl, benzothiadiazolyl, oxazolyl, isoxazolyl or oxazolyl, even more preferably phenyl or pyridinyl,
   p is 1, 2 or 3,
   R⁸ is selected from the group consisting of alkyl comprising 1 to 4 carbon atoms, alkoxy comprising 1 to 4 carbon atoms, Hal, CH₂Hal, CH(Hal)₂, perhaloalkyl comprising 1 to 4 carbon atoms, NO₂, (CH₂)ₙCN, (CH₂)ₙNR¹¹R¹², (CH₂)ₙO(CH₂)ₖNR¹¹R¹², (CH₂)ₙNR¹¹(CH₂)ₖNR¹¹R¹², (CH₂)ₙO(CH₂)ₖOR¹¹, (CH₂)ₙNR¹¹(CH₂)ₖOR¹², (CH₂)ₙCOR¹³, (CH₂)ₙCOOR¹³, (CH₂)ₙCONR¹¹R¹², (CH₂)ₙSO₂NR¹¹R¹² and (CH₂)ₙS(O)ᵤR¹³, wherein
   n is 0 or 1,
   u is 0, and
   q is 0 or 1, and
   X is selected from the group consisting of O, S, NR¹¹, CHOR¹¹, CH₂, CH₂CH₂, OCH₂, CH₂O, OCH₂CH₂, CH₂CH₂O, preferably O, S and CH₂ and especially O and S;
I.8) Ar¹ is phenyl, pyridinyl, pyrimidyl, chinolinyl, isochinolinyl, thiophenyl, thiadiazolyl, benzothiadiazolyl, oxazolyl, isoxazolyl, pyrazolyl or imidazolyl, preferably phenyl, pyridinyl, chinolinyl, isochinolinyl, thiophenyl, benzothiadiazolyl, oxazolyl, isoxazolyl or oxazolyl, even more preferably phenyl or pyridinyl,
   p is 1, 2 or 3,
   R⁸ is selected from the group consisting of alkyl comprising 1 to 4 carbon atoms, alkoxy comprising 1 to 4 carbon atoms, Hal, CH₂Hal, CH(Hal)₂, perhaloalkyl comprising 1 to 4 carbon atoms, NO₂, (CH₂)ₙCN, (CH₂)ₙNR¹¹R¹², (CH₂)ₙO(CH₂)ₖNR¹¹R¹², (CH₂)ₙNR¹¹(CH₂)ₖNR¹¹R¹², (CH₂)ₙO(CH₂)ₖOR¹¹, (CH₂)ₙNR¹¹(CH₂)ₖOR¹², (CH₂)ₙCOR¹³, (CH₂)ₙCOOR¹³, (CH₂)ₙCONR¹¹R¹², (CH₂)ₙSO₂NR¹¹R¹² and (CH₂)ₙS(O)ᵤR¹³, wherein
   n is 0 or 1,
   u is 0, and
   q is 0 or 1, and
   X is selected from the group consisting of O, S, NR¹¹, CHOR¹¹, CH₂, CH₂CH₂, OCH₂, CH₂O, OCH₂CH₂, CH₂CH₂O, preferably O, S and CH₂ and especially O and S,
   Ar² is phenyl, pyridinyl or pyrimidyl, and especially is phenyl or pyridinyl;
I.9) Ar¹ is phenyl, pyridinyl, pyrimidyl, chinolinyl, isochinolinyl, thiophenyl, thiadiazolyl, benzothiadiazolyl, oxazolyl, isoxazolyl, pyrazolyl or imidazolyl, preferably phenyl, pyridinyl, chinolinyl, isochinolinyl, thiophenyl, benzothiadiazolyl, oxazolyl, isoxazolyl or oxazolyl, even more preferably phenyl or pyridinyl,
   p is 1, 2 or 3,
   R⁸ is selected from the group consisting of alkyl comprising 1 to 4 carbon atoms, alkoxy comprising 1 to 4 carbon atoms, Hal, CH₂Hal, CH(Hal)₂, perhaloalkyl comprising 1 to 4 carbon atoms, NO₂, (CH₂)ₙCN, (CH₂)ₙNR¹¹R¹², (CH₂)ₙO(CH₂)ₖNR¹¹R¹², (CH₂)ₙNR¹¹(CH₂)ₖNR¹¹R¹², (CH₂)ₙO(CH₂)ₖOR¹¹, (CH₂)ₙNR¹¹(CH₂)ₖOR¹², (CH₂)ₙCOR¹³, (CH₂)ₙCOOR¹³, (CH₂)ₙCONR¹¹R¹², (CH₂)ₙSO₂NR¹¹R¹² and (CH₂)ₙS(O)ᵤR¹³, wherein
   n is 0 or 1,
   u is 0, and
   q is 0 or 1, and
   X is selected from the group consisting of O, S, NR¹¹, CHOR¹¹, CH₂, CH₂CH₂, OCH₂, CH₂O, OCH₂CH₂, CH₂CH₂O, preferably O, S and CH₂ and especially O and S,
   Ar² is phenyl, pyridinyl or pyrimidyl, and especially is phenyl or pyridinyl; and
   R¹⁰ is selected from the group consisting of H, alkyl comprising 1 to 4 carbon atoms, alkoxy comprising 1 to 4 carbon atoms, Hal, CH₂Hal, CH(Hal)₂, perhaloalkyl comprising 1 to 4 carbon atoms, NO₂, (CH₂)ₙCN, (CH₂)ₙNR¹¹R¹², (CH₂)ₙO(CH₂)ₖNR¹¹R¹², (CH₂)ₙNR¹¹(CH₂)ₖNR¹¹R¹², (CH₂)ₙO(CH₂)ₖOR¹¹, (CH₂)ₙNR¹¹(CH₂)ₖOR¹², (CH₂)ₙCOR¹³, (CH₂)ₙCOOR¹³, (CH₂)ₙCONR¹¹R¹², (CH₂)ₙSO₂NR¹¹R¹² and (CH₂)ₙS(O)ᵤR¹³, preferably alkyl comprising 1 to 4 carbon atoms, (CH₂)ₙNR¹¹R¹², (CH₂)ₙO(CH₂)ₖNR¹¹R¹², (CH₂)ₙCOR¹³, (CH₂)ₙCOOR¹³, (CH₂)ₙCONR¹¹R¹² and especially (CH₂)ₙCONR¹¹R¹²;
I.10) Ar¹ is phenyl, pyridinyl, pyrimidyl, chinolinyl, isochinolinyl, thiophenyl, thiadiazolyl, benzothiadiazolyl, oxazolyl, isoxazolyl, pyrazolyl or imidazolyl, preferably phenyl, pyridinyl, chinolinyl, isochinolinyl, thiophenyl, benzothiadiazolyl, oxazolyl, isoxazolyl or oxazolyl, even more preferably phenyl or pyridinyl,
   p is 1, 2 or 3,
   R⁸ is selected from the group consisting of alkyl comprising 1 to 4 carbon atoms, alkoxy comprising 1 to 4 carbon atoms, Hal, CH₂Hal, CH(Hal)₂, perhaloalkyl comprising 1 to 4 carbon atoms, NO₂, (CH₂)ₙCN, (CH₂)ₙNR¹¹R¹², (CH₂)ₙO(CH₂)ₖNR¹¹R¹², (CH₂)ₙCOR¹³, (CH₂)ₙCOOR¹³, (CH₂)ₙCONR¹¹R¹², (CH₂)ₙSO₂NR¹¹R¹² and (CH₂)ₙS(O)ᵤR¹³ wherein
   n is 0 or 1,
   u is 0, and
   q is 0 or 1, and
   X is selected from the group consisting of O, S, NR¹¹, CHOR¹¹, CH₂, CH₂CH₂, OCH₂, CH₂O, OCH₂CH₂, CH₂CH₂O, preferably O, S and CH₂ and especially O and S,
   Ar² is phenyl, pyridinyl or pyrimidyl, and especially is phenyl or pyridinyl; and
   R¹⁰ is selected from the group consisting of H, alkyl comprising 1 to 4 carbon atoms, alkoxy comprising 1 to 4 carbon atoms, Hal, CH₂Hal, CH(Hal)₂, perhaloalkyl comprising 1 to 4 carbon atoms, NO₂, (CH₂)ₙCN, (CH₂)ₙNR¹¹R¹², (CH₂)ₙO(CH₂)ₖNR¹¹R¹², (CH₂)ₙNR¹¹(CH₂)ₖNR¹¹R¹², (CH₂)ₙO(CH₂)ₖOR¹¹, (CH₂)ₙNR¹¹(CH₂)ₖOR¹², (CH₂)ₙCOR¹³, (CH₂)ₙCOOR¹³, (CH₂)ₙCONR¹¹R¹², (CH₂)ₙSO₂NR¹¹R¹² and (CH₂)ₙS(O)ᵤR¹³, preferably alkyl comprising 1 to 4 carbon atoms, (CH₂)ₙNR¹¹R¹², (CH₂)ₙO(CH₂)ₖNR¹¹R¹², (CH₂)ₙCOR¹³, (CH₂)ₙCOOR¹³, (CH₂)ₙCONR¹¹R¹² and especially (CH₂)ₙCONR¹¹R¹², wherein
   n is 0, 1 or 2, preferably 0 or 1;
I.11) Ar¹ is phenyl, pyridinyl, pyrimidyl, chinolinyl, isochinolinyl, thiophenyl, thiadiazolyl, benzothiadiazolyl, oxazolyl, isoxazolyl, pyrazolyl or imidazolyl, preferably phenyl, pyridinyl, chinolinyl, isochinolinyl, thiophenyl, benzothiadiazolyl, oxazolyl, isoxazolyl or oxazolyl, even more preferably phenyl or pyridinyl,
   p is 1, 2 or 3,
   R⁸ is selected from the group consisting of alkyl comprising 1 to 4 carbon atoms, alkoxy comprising 1 to 4 carbon atoms, Hal, CH₂Hal, CH(Hal)₂, perhaloalkyl comprising 1 to 4 carbon atoms, NO₂, (CH₂)ₙCN, (CH₂)ₙNR¹¹R¹², (CH₂)ₙO(CH₂)ₖNR¹¹R¹², (CH₂)ₙNR¹¹(CH₂)ₖNR¹¹R¹², (CH₂)ₙO(CH₂)ₖOR¹¹, (CH₂)ₙNR¹¹(CH₂)ₖOR¹², (CH₂)ₙCOR¹³, (CH₂)ₙCOOR¹³, (CH₂)ₙCONR¹¹R¹², (CH₂)ₙSO₂NR¹¹R¹² and (CH₂)ₙS(O)ᵤR¹³, wherein
   n is 0 or 1,
   u is 0, and
   q is 0 or 1, and
   X is selected from the group consisting of O, S, NR¹¹, CHOR¹¹, CH₂, CH₂CH₂, OCH₂, CH₂O, OCH₂CH₂, CH₂CH₂O, preferably O, S and CH₂ and especially O and S,
   Ar² is phenyl, pyridinyl or pyrimidyl, and especially is phenyl or pyridinyl; and
   R¹⁰ is selected from the group consisting of H, alkyl comprising 1 to 4 carbon atoms, alkoxy comprising 1 to 4 carbon atoms, Hal, CH₂Hal, CH(Hal)₂, perhaloalkyl comprising 1 to 4 carbon atoms, NO₂, (CH₂)ₙCN, (CH₂)ₙNR¹¹R¹², (CH₂)ₙO(CH₂)ₖNR¹¹R¹², (CH₂)ₙNR¹¹(CH₂)ₖNR¹¹R¹², (CH₂)ₙO(CH₂)ₖOR¹¹, (CH₂)ₙNR¹¹(CH₂)ₖOR¹², (CH₂)ₙCOR¹³, (CH₂)ₙCOOR¹³, (CH₂)ₙCONR¹¹R¹², (CH₂)ₙSO₂NR¹¹R¹² and (CH₂)ₙS(O)ᵤR¹³, preferably alkyl comprising 1 to 4 carbon atoms, (CH₂)ₙNR¹¹R¹², (CH₂)ₙO(CH₂)ₖNR¹¹R¹², (CH₂)ₙCOR¹³, (CH₂)ₙCOOR¹³, (CH₂)ₙCONR¹¹R¹² and especially (CH₂)ₙCONR¹¹R¹², wherein
   n is 0, 1 or 2, preferably 0 or 1 and
   r is 0, 1 or 2, preferably 0 or 1;
I.12) p is 1, 2 or 3,
   R⁸ is selected from the group consisting of alkyl comprising 1 to 4 carbon atoms, alkoxy comprising 1 to 4 carbon atoms, Hal, CH₂Hal, CH(Hal)₂, perhaloalkyl comprising 1 to 4 carbon atoms, NO₂, (CH₂)ₙCN, (CH₂)ₙNR¹¹R¹², (CH₂)ₙO(CH₂)ₖNR¹¹R¹², (CH₂)ₙNR¹¹(CH₂)ₖNR¹¹R¹², (CH₂)ₙO(CH₂)ₖOR¹¹, (CH₂)ₙNR¹¹(CH₂)ₖOR¹², (CH₂)ₙCOR¹³, (CH₂)ₙCOOR¹³, (CH₂)ₙCONR¹¹R¹², (CH₂)ₙSO₂NR¹¹R¹² and (CH₂)ₙS(O)ᵤR¹³, wherein
   n is 0 or 1,
   u is 0, and
   q is 0 or 1, and
   X is selected from the group consisting of O, S, NR¹¹, CHOR¹¹, CH₂, CH₂CH₂, OCH₂, CH₂O, OCH₂CH₂, CH₂CH₂O, preferably O, S and CH₂ and especially O and S,
   Ar² is phenyl, pyridinyl or pyrimidyl, and especially is phenyl or pyridinyl; and
   R¹⁰ is selected from the group consisting of H, alkyl comprising 1 to 4 carbon atoms, alkoxy comprising 1 to 4 carbon atoms, Hal, CH₂Hal, CH(Hal)₂, perhaloalkyl comprising 1 to 4 carbon atoms, NO₂, (CH₂)ₙCN, (CH₂)ₙNR¹¹R¹², (CH₂)ₙO(CH₂)ₖNR¹¹R¹², (CH₂)ₙNR¹¹(CH₂)ₖNR¹¹R¹², (CH₂)ₙO(CH₂)ₖOR¹¹, (CH₂)ₙNR¹¹(CH₂)ₖOR¹², (CH₂)ₙCOR¹³, (CH₂)ₙCOOR¹³, (CH₂)ₙCONR¹¹R¹², (CH₂)ₙSO₂NR¹¹R¹² and (CH₂)ₙS(O)ᵤR¹³, preferably alkyl comprising 1 to 4 carbon atoms, (CH₂)ₙNR¹¹R¹², (CH₂)ₙO(CH₂)ₖNR¹¹R¹², (CH₂)ₙCOR¹³, (CH₂)ₙCOOR¹³, (CH₂)ₙCONR¹¹R¹² and especially (CH₂)ₙCONR¹¹R¹², wherein
   n is 0, 1 or 2, preferably 0 or 1 and
   r is 0, 1 or 2, preferably 0 or 1;
I.13) R⁸ is selected from the group consisting of alkyl comprising 1 to 4 carbon atoms, alkoxy comprising 1 to 4 carbon atoms, Hal, CH₂Hal, CH(Hal)₂, perhaloalkyl comprising 1 to 4 carbon atoms, NO₂, (CH₂)ₙCN, (CH₂)ₙNR¹¹R¹², (CH₂)ₙO(CH₂)ₖNR¹¹R¹², (CH₂)ₙNR¹¹(CH₂)ₖNR¹¹R¹², (CH₂)ₙO(CH₂)ₖOR¹¹, (CH₂)ₙNR¹¹(CH₂)ₖOR¹², (CH₂)ₙCOR¹³, (CH₂)ₙCOOR¹³, (CH₂)ₙCONR¹¹R¹², (CH₂)ₙSO₂NR¹¹R¹² and (CH₂)ₙS(O)ᵤR¹³, wherein
   n is 0 or 1,
   u is 0, and
   q is 0 or 1, and
   X is selected from the group consisting of O, S, NR¹¹, CHOR¹¹, CH₂, CH₂CH₂, OCH₂, CH₂O, OCH₂CH₂, CH₂CH₂O, preferably O, S and CH₂ and especially O and S,
   Ar² is phenyl, pyridinyl or pyrimidyl, and especially is phenyl or pyridinyl; and
   R¹⁰ is selected from the group consisting of H, alkyl comprising 1 to 4 carbon atoms, alkoxy comprising 1 to 4 carbon atoms, Hal, CH₂Hal, CH(Hal)₂, perhaloalkyl comprising 1 to 4 carbon atoms, NO₂, (CH₂)ₙCN, (CH₂)ₙNR¹¹R¹², (CH₂)ₙO(CH₂)ₖNR¹¹R¹², (CH₂)ₙNR¹¹(CH₂)ₖNR¹¹R¹², (CH₂)ₙO(CH₂)ₖOR¹¹, (CH₂)ₙNR¹¹(CH₂)ₖOR¹², (CH₂)ₙCOR¹³, (CH₂)ₙCOOR¹³, (CH₂)ₙCONR¹¹R¹², (CH₂)ₙSO₂NR¹¹R¹² and (CH₂)ₙS(O)ᵤR¹³, preferably alkyl comprising 1 to 4 carbon atoms, (CH₂)ₙNR¹¹R¹², (CH₂)ₙO(CH₂)ₖNR¹¹R¹², (CH₂)ₙCOR¹³, (CH₂)ₙCOOR¹³, (CH₂)ₙCONR¹¹R¹² and especially (CH₂)ₙCONR¹¹R¹², wherein
   n is 0, 1 or 2, preferably 0 or 1 and
   r is 0, 1 or 2, preferably 0 or 1;
I.14) R⁸ is selected from the group consisting of alkyl comprising 1 to 4 carbon atoms, alkoxy comprising 1 to 4 carbon atoms, Hal, CH₂Hal, CH(Hal)₂, perhaloalkyl comprising 1 to 4 carbon atoms, NO₂, (CH₂)ₙCN, (CH₂)ₙNR¹¹R¹², (CH₂)ₙO(CH₂)ₖNR¹¹R¹², (CH₂)ₙNR¹¹(CH₂)ₖNR¹¹R¹², (CH₂)ₙO(CH₂)ₖOR¹¹, (CH₂)ₙNR¹¹(CH₂)ₖOR¹², (CH₂)ₙCOR¹³, (CH₂)ₙCOOR¹³, (CH₂)ₙCONR¹¹R¹², (CH₂)ₙSO₂NR¹¹R¹² and (CH₂)ₙS(O)ᵤR¹³, wherein
   u is 0, and
   q is 0 or 1, and
   X is selected from the group consisting of O, S, NR¹¹, CHOR¹¹, CH₂, CH₂CH₂, OCH₂, CH₂O, OCH₂CH₂, CH₂CH₂O, preferably O, S and CH₂ and especially O and S,
   Ar² is phenyl, pyridinyl or pyrimidyl, and especially is phenyl or pyridinyl; and
   R¹⁰ is selected from the group consisting of H, alkyl comprising 1 to 4 carbon atoms, alkoxy comprising 1 to 4 carbon atoms, Hal, CH₂Hal, CH(Hal)₂, perhaloalkyl comprising 1 to 4 carbon atoms, NO₂, (CH₂)ₙCN, (CH₂)ₙNR¹¹R¹², (CH₂)ₙO(CH₂)ₖNR¹¹R¹², (CH₂)ₙNR¹¹(CH₂)ₖNR¹¹R¹², (CH₂)ₙO(CH₂)ₖOR¹¹, (CH₂)ₙNR¹¹(CH₂)ₖOR¹², (CH₂)ₙCOR¹³, (CH₂)ₙCOOR¹³, (CH₂)ₙCONR¹¹R¹², (CH₂)ₙSO₂NR¹¹R¹² and (CH₂)ₙS(O)ᵤR¹³, preferably alkyl comprising 1 to 4 carbon atoms, (CH₂)ₙNR¹¹R¹², (CH₂)ₙO(CH₂)ₖNR¹¹R¹², (CH₂)ₙCOR¹³, (CH₂)ₙCOOR¹³, (CH₂)ₙCONR¹¹R¹² and especially (CH₂)ₙCONR¹¹R¹², wherein
   n is 0, 1 or 2, preferably 0 or 1 and
   r is 0, 1 or 2, preferably 0 or 1;
I.15) R⁸ is selected from the group consisting of alkyl comprising 1 to 4 carbon atoms, alkoxy comprising 1 to 4 carbon atoms, Hal, CH₂Hal, CH(Hal)₂, perhaloalkyl comprising 1 to 4 carbon atoms, NO₂, (CH₂)ₙCN, (CH₂)ₙNR¹¹R¹², (CH₂)ₙO(CH₂)ₖNR¹¹R¹², (CH₂)ₙNR¹¹(CH₂)ₖNR¹¹R¹², (CH₂)ₙO(CH₂)ₖOR¹¹, (CH₂)ₙNR¹¹(CH₂)ₖOR¹², (CH₂)ₙCOR¹³, (CH₂)ₙCOOR¹³, (CH₂)ₙCONR¹¹R¹², (CH₂)ₙSO₂NR¹¹R¹² and (CH₂)ₙS(O)ᵤR¹³ wherein
   q is 0 or 1, and
   X is selected from the group consisting of O, S, NR¹¹, CHOR¹¹, CH₂, CH₂CH₂, OCH₂, CH₂O, OCH₂CH₂, CH₂CH₂O, preferably O, S and CH₂ and especially O and S,
   Ar² is phenyl, pyridinyl or pyrimidyl, and especially is phenyl or pyridinyl; and
   R¹⁰ is selected from the group consisting of H, alkyl comprising 1 to 4 carbon atoms, alkoxy comprising 1 to 4 carbon atoms, Hal, CH₂Hal, CH(Hal)₂, perhaloalkyl comprising 1 to 4 carbon atoms, NO₂, (CH₂)ₙCN, (CH₂)ₙNR¹¹R¹², (CH₂)ₙO(CH₂)ₖNR¹¹R¹², (CH₂)ₙNR¹¹(CH₂)ₖNR¹¹R¹², (CH₂)ₙO(CH₂)ₖOR¹¹, (CH₂)ₙNR¹¹(CH₂)ₖOR¹², (CH₂)ₙCOR¹³, (CH₂)ₙCOOR¹³, (CH₂)ₙCONR¹¹R¹², (CH₂)ₙSO₂NR¹¹R¹² and (CH₂)ₙS(O)ᵤR¹³, preferably alkyl comprising 1 to 4 carbon atoms, (CH₂)ₙNR¹¹R¹², (CH₂)ₙO(CH₂)ₖNR¹¹R¹², (CH₂)ₙCOR¹³, (CH₂)ₙCOOR¹³, (CH₂)ₙCONR¹¹R¹² and especially (CH₂)ₙCONR¹¹R¹², wherein
   n is 0, 1 or 2, preferably 0 or 1 and
   r is 0, 1 or 2, preferably 0 or 1;
I.16) q is 0 or 1, and
   X is selected from the group consisting of O, S, NR¹¹, CHOR¹¹, CH₂, CH₂CH₂, OCH₂, CH₂O, OCH₂CH₂, CH₂CH₂O, preferably O, S and CH₂ and especially O and S,
   Ar² is phenyl, pyridinyl or pyrimidyl, and especially is phenyl or pyridinyl; and
   R¹⁰ is selected from the group consisting of H, alkyl comprising 1 to 4 carbon atoms, alkoxy comprising 1 to 4 carbon atoms, Hal, CH₂Hal, CH(Hal)₂, perhaloalkyl comprising 1 to 4 carbon atoms, NO₂, (CH₂)ₙCN, (CH₂)ₙNR¹¹R¹², (CH₂)ₙO(CH₂)ₖNR¹¹R¹², (CH₂)ₙNR¹¹(CH₂)ₖNR¹¹R¹², (CH₂)ₙO(CH₂)ₖOR¹¹, (CH₂)ₙNR¹¹(CH₂)ₖOR¹², (CH₂)ₙCOR¹³, (CH₂)ₙCOOR¹³, (CH₂)ₙCONR¹¹R¹², (CH₂)ₙSO₂NR¹¹R¹² and (CH₂)ₙS(O)ᵤR¹³, preferably alkyl comprising 1 to 4 carbon atoms, (CH₂)ₙNR¹¹R¹², (CH₂)ₙO(CH₂)ₖNR¹¹R¹², (CH₂)ₙCOR¹³, (CH₂)ₙCOOR¹³, (CH₂)ₙCONR¹¹R¹² and especially (CH₂)ₙCONR¹¹R¹², wherein
   n is 0, 1 or 2, preferably 0 or 1 and
   r is 0, 1 or 2, preferably 0 or 1;
I.17) X is selected from the group consisting of O, S, NR¹¹, CHOR¹¹, CH₂, CH₂CH₂, OCH₂, CH₂O, OCH₂CH₂, CH₂CH₂O, preferably O, S and CH₂ and especially O and S,
   Ar² is phenyl, pyridinyl or pyrimidyl, and especially is phenyl or pyridinyl; and
   R¹⁰ is selected from the group consisting of H, alkyl comprising 1 to 4 carbon atoms, alkoxy comprising 1 to 4 carbon atoms, Hal, CH₂Hal, CH(Hal)₂, perhaloalkyl comprising 1 to 4 carbon atoms, NO₂, (CH₂)ₙCN, (CH₂)ₙNR¹¹R¹², (CH₂)ₙO(CH₂)ₖNR¹¹R¹², (CH₂)ₙNR¹¹(CH₂)ₖNR¹¹R¹², (CH₂)ₙO(CH₂)ₖOR¹¹, (CH₂)ₙNR¹¹(CH₂)ₖOR¹², (CH₂)ₙCR¹³, (CH₂)ₙCOOR¹³, (CH₂)ₙCONR¹¹R¹², (CH₂)ₙSO₂NR¹¹R¹² and (CH₂)ₙS(O)ᵤR¹³, preferably alkyl comprising 1 to 4 carbon atoms, (CH₂)ₙNR¹¹R¹², (CH₂)ₙO(CH₂)ₖNR¹¹R¹², (CH₂)ₙCOR¹³, (CH₂)ₙCOOR¹³, (CH₂)ₙCONR¹¹R¹² and especially (CH₂)ₙCONR¹¹R¹², wherein
   n is 0, 1 or 2, preferably 0 or 1 and
   r is 0, 1 or 2, preferably 0 or 1;
I.18) Ar² is phenyl, pyridinyl or pyrimidyl, and especially is phenyl or pyridinyl; and
   R¹⁰ is selected from the group consisting of alkyl comprising 1 to 4 carbon atoms, alkoxy comprising 1 to 4 carbon atoms, Hal, CH₂Hal, CH(Hal)₂, perhaloalkyl comprising 1 to 4 carbon atoms, NO₂, (CH₂)ₙCN, (CH₂)ₙNR¹¹R¹², (CH₂)ₙO(CH₂)ₖNR¹¹R¹², (CH₂)ₙNR¹¹(CH₂)ₖNR¹¹R¹², (CH₂)ₙO(CH₂)ₖOR¹¹, (CH₂)ₙNR¹¹(CH₂)ₖOR¹², (CH₂)ₙCOR¹³, (CH₂)ₙCOOR¹³, (CH₂)ₙCONR¹¹R¹², (CH₂)ₙSO₂NR¹¹R¹² and (CH₂)ₙS(O)ᵤR¹³, preferably alkyl comprising 1 to 4 carbon atoms, (CH₂)ₙNR¹¹R¹², (CH₂)ₙO(CH₂)ₖNR¹¹R¹², (CH₂)ₙCOR¹³, (CH₂)ₙCOOR¹³, (CH₂)ₙCONR¹¹R¹² and especially (CH₂)ₙCONR¹¹R¹²,
   n is 0, 1 or 2, preferably 0 or 1 and
   r is 0, 1 or 2, preferably 0 or 1;
I.19) R¹⁰ is selected from the group consisting of H, alkyl comprising 1 to 4 carbon atoms, alkoxy comprising 1 to 4 carbon atoms, Hal, CH₂Hal, CH(Hal)₂, perhaloalkyl comprising 1 to 4 carbon atoms, NO₂, (CH₂)ₙCN, (CH₂)ₙNR¹¹R¹², (CH₂)ₙO(CH₂)ₖNR¹¹R¹², (CH₂)ₙNR¹¹(CH₂)ₖNR¹¹R¹², (CH₂)ₙO(CH₂)ₖOR¹¹, (CH₂)ₙNR¹¹(CH₂)ₖOR¹², (CH₂)ₙCOR¹³, (CH₂)ₙCOOR¹³, (CH₂)ₙCONR¹¹R¹², (CH₂)ₙSO₂NR¹¹R¹² and (CH₂)ₙS(O)ᵤR¹³, preferably alkyl comprising 1 to 4 carbon atoms, (CH₂)ₙNR¹¹R¹², (CH₂)ₙO(CH₂)ₖNR¹¹R¹², (CH₂)ₙCOR¹³, (CH₂)ₙCOOR¹³, (CH₂)ₙCONR¹¹R¹² and especially (CH₂)ₙCONR¹¹R¹².
   n is 0, 1 or 2, preferably 0 or 1 and
   r is 0, 1 or 2, preferably 0 or 1;
I.20) R¹⁰ is selected from the group consisting of H, alkyl comprising 1 to 4 carbon atoms, alkoxy comprising 1 to 4 carbon atoms, Hal, CH₂Hal, CH(Hal)₂, perhaloalkyl comprising 1 to 4 carbon atoms, NO₂, (CH₂)ₙCN, (CH₂)ₙNR¹¹R¹², (CH₂)ₙO(CH₂)ₖNR¹¹R¹², (CH₂)ₙNR¹¹(CH₂)ₖNR¹¹R¹², (CH₂)ₙO(CH₂)ₖOR¹¹, (CH₂)ₙNR¹¹(CH₂)ₖOR¹², (CH₂)ₙCOR¹³, (CH₂)ₙCOOR¹³, (CH₂)ₙCONR¹¹R¹², (CH₂)ₙSO₂NR¹¹R¹² and (CH₂)ₙS(O)ᵤR¹³, preferably alkyl comprising 1 to 4 carbon atoms, (CH₂)ₙNR¹¹R¹², (CH₂)ₙO(CH₂)ₖNR¹¹R¹², (CH₂)ₙCOR¹³, (CH₂)ₙCOOR¹³, (CH₂)ₙCONR¹¹R¹² and especially (CH₂)ₙCONR¹¹R¹² and
   r is 0, 1 or 2, preferably 0 or 1.

One preferred embodiment of the instant invention relates to compounds of formula I and preferably one or more of sub formulae I.1) to I.20), wherein p is 1, 2 or 3 and R⁸ is independently selected from the group consisting of methyl, ethyl, isopropyl, tert.-butyl, F, Cl, Br, CF₃, C(CF₃)₃, SO₂CF₃, methoxy, ethoxy, tert.-butoxy, perfluoro tert.-butoxy (OC(CF₃)₃), methyl sulfanyl (SCH₃), ethyl sulfanyl (SCH₂CH₃), acetyl (COCH₃), propionyl (COCH₂CH₃), butyryl (COCH₂CH₂CH₃). If p is 2 or 3, all substituents can be the same or different.

Another preferred embodiment of the instant invention relates to compounds of formula I and preferably one or more of sub formulae I.1) to I.20), wherein X is selected from the group consisting of S, N—R²¹, CH₂, CH₂CH₂, OCH₂ and CH₂O.

Another preferred embodiment of the instant invention relates to compounds of formula I and preferably one or more of sub formulae I.1) to I.20), wherein X is selected from the group consisting of S, CH₂.

Another even more preferred embodiment of the instant invention relates to compounds of formula I and preferably one or more of sub formulae I.1) to I.20), wherein X is O.

Another preferred embodiment of the instant invention relates to compounds of formula I and preferably one or more of sub formulae I.1) to I.20), wherein Y is selected from the group consisting of C(R²²)—NO₂, C(R²²)—CN and C(CN)₂.

Another more preferred embodiment of the instant invention relates to compounds of formula I and preferably one or more of sub formulae I.1) to I.20), wherein Y is selected from the group consisting of O, S and NR²¹.

Another even more preferred embodiment of the instant invention relates to compounds of formula I and preferably one or more of sub formulae I.1) to I.20), wherein Y is selected from the group consisting of O and S.

Another even more preferred embodiment of the instant invention relates to compounds of formula I and preferably one or more of sub formulae I.1) to I.20), wherein Y is O.

Another preferred embodiment of the instant invention relates to compounds of formula I and preferably one or more of sub formulae I.1) to I.20), wherein Ar² is pyridinyl.

Another preferred embodiment of the instant invention relates to compounds of formula I and preferably one or more of sub formulae I.1) to I.20), wherein r is either 0 or 1. If r is 1, R¹⁰ is preferably (CH₂)ₙCONR¹¹R¹² and especially (CH₂)ₙCONR¹¹R¹², wherein n in 0. In this embodiment, R¹¹ is preferably selected from the group consisting of H and A and more preferred from H and alkyl, and R¹² is preferably selected from the group consisting of H and A and more preferred from H and alkyl. Especially preferred as residue R¹⁰ are carbamoyl, more preferred alkyl carbamoyl or dialkyl carbamoyl, even more preferred methyl carbamoyl or dimethyl carbamoyl, ethyl carbamoyl or diethyl carbamoyl and especially preferred methyl carbamoyl (—CONHCH₃). This embodiment is especially preferred when Ar² is pyridinyl. When Ar² is pyridinyl, R¹⁰ is preferably bonded in a vicinal position to the nitrogen atom of the pyrindiyl residue, i.e. in 2- and/or 6-position of the pyridinyl residue.

Another preferred embodiment of the instant invention relates to compounds of formula I and preferably one or more of sub formulae I.1) to I.20), wherein Ar¹ is phenyl.

Another preferred embodiment of the instant invention relates to compounds of formula I and preferably one or more of sub formulae I.1) to I.20), wherein R⁷ is independently selected from a group consisting of Het, OHet, N(R¹¹)Het, (CR⁵R⁶)ₖHet, O(CR⁵R⁶)ₖHet, N(R¹¹)(CR⁵R⁶)ₖHet, (CR⁵R⁶)ₖNR¹¹R¹², (CR⁵R⁶)ₖOR¹³, O(CR⁵R⁶)ₖNR¹¹R¹², NR¹¹(CR⁵R⁶)ₖNR¹¹R¹², O(CR⁵R⁶)ₖR¹³, NR¹¹(CR⁵R⁶)ₖR¹³, O(CR⁵R⁶)ₖOR¹³, NR¹¹(CR⁵R⁶)ₖOR¹³, and more preferably from OHet, N(R¹¹)Het, (CR⁵R⁶)ₖHet, O(CR⁵R⁶)ₖHet, N(R¹¹)(CR⁵R⁶)ₖHet, O(CR⁵R⁶)ₖNR¹¹R¹², NR¹¹(CR⁵R⁶)ₖNR¹¹R¹², O(CR⁵R⁶)ₖOR¹³, NR¹¹(CR⁵R⁶)ₖOR¹³, and even more preferably O(CH₂)ₖR¹³, NR¹¹(CH₂)ₖR¹³, O(CH₂)ₖOR¹¹, NR¹¹(CH₂)ₖOR¹³, O(CH₂)ₖNR¹¹R¹², NR¹¹(CH₂)ₖNR¹¹R¹². In this embodiment, k is preferably 1, 2 or 3

Another preferred embodiment of the instant invention relates to compounds of formula I and preferably one or more of sub formulae I.1) to I.20)), wherein R⁷ comprises a group NR¹¹R¹² wherein R¹¹ and R¹² form, together with the N-atom they are bound to, a 5-, 6- or 7-membered heterocyclus which optionally contains 1 or 2 additional hetero atoms, selected from N, O and S, which optionally is substituted by one or more substituent, selected from A, R¹³, ═O, ═S and ═N—R¹⁴. In this embodiment, the heterocyclus is preferably selected from morpholine, piperazine, piperidine, pyrrolidine, especially from 1-piperidyl, 4-piperidyl, 1-methyl-piperidin-4-yl, 1-piperazyl, 1-(4-methyl)-piperazyl, 4-methylpiperazin-1-yl amine, 1-(4-(2-hydroxyethyl))-piperazyl, 4-morpholinyl, 1-pyrrolidinyl, 2-pyrrolidinyl, and/or oxomorpholine, oxopiperazine, oxopiperidine and oxopyrrolidine. More preferably, the oxo substituted heterocyclus is selected from 2-oxo-piperidin-1-yl, 2-oxo-piperidin-4-yl, 1-methyl-2-oxo-piperidin-4-yl, 2-oxo-piperazin-1-yl, 4-methyl-2-oxo-piperazin-1-yl, 4-methyl-2-oxo-piperazin-1-yl amine, 4-(2-hydroxyethyl)-2-oxo-piperazin-1-yl, 3-oxo-morpholin-4-yl, 2-oxo-pyrrolidin-1-yl, 2-oxo-pyrrolidin-5-yl and/or 3-oxo-piperidin-1-yl, 3-oxo-piperidin-4-yl, 1-methyl-3-oxo-piperidin-4-yl, 3-oxo-piperazin-1-yl, 4-methyl-3-oxo-piperazin-1-yl, 4-methyl-3-oxo-piperazin-1-yl amine, 4-(2-hydroxyethy)-3-oxo-piperazin-1-yl, 2-oxo-morpholin-4-yl, 3-oxo-pyrrolidin-1-yl, 4-oxo-pyrrolidin-3-yl.

Another preferred embodiment of the instant invention relates to compounds of formula I and preferably one or more of sub formulae I.1) to I.20), wherein R⁷ comprises a terminal group R¹¹, R¹², R¹³ or R¹⁴, preferably a group R¹³, that is selected from cycloalkyl and Het, more preferred from cycloalkyl and saturated heterocyclyl and especially from saturated heterocyclyl. In this embodiment, saturated heterocycle is preferably selected from 2-piperidyl, 3-piperidyl, 4-piperidyl, 1-methyl-piperidin-4-yl, 1-methyl-piperidin-3-yl, 1-methyl-piperidin-2-yl, 2-piperazyl, 3-piperazyl, 2-(4-methyl)-piperazyl, 3-(4-methyl)-piperazyl, 4-methylpiperazin-2-yl amine, 4-methylpiperazin-3-yl amine, 2-(4-(2-hydroxyethyl))-piperazyl, 3-(4-(2-hydroxyethyl))-piperazyl, 3-morpholinyl, 2-morpholinyl, 2-pyrrolidinyl, 3-pyrrolidinyl, and especially from

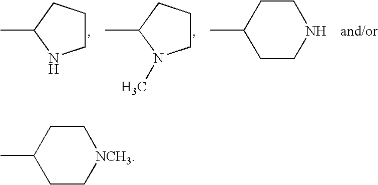

Another preferred embodiment of the instant invention relates to compounds of formula I and preferably one or more of sub formulae I.1) to I.20), wherein Ar¹ comprises one or more, preferably one substituent R⁷ that is selected from the group consisting of (CR⁵R⁶)ₖNR¹¹R¹², (CR⁵R⁶)ₖOR¹³, O(CR⁵R⁶)ₖNR¹¹R¹², NR¹¹(CR⁵R⁶)ₖNR¹¹R¹², NR¹¹(CR⁵R⁶)ₖR¹³, O(CR⁵R⁶)ₖOR¹³, NR¹¹(CR⁵R⁶)ₖOR¹³, wherein R¹¹, R¹² and R¹³ are defined as above and n is as defined above, preferably n is 1, 2 or 3 and especially is 1 or 2, and k is as defined above, preferably k is 1 to 4 and preferably 1, 2 or 3. In this embodiment R¹¹, R¹² and R¹³ are more preferably selected independently from each other from the group consisting of H, methyl and ethyl. In this embodiment, one or two substituents R⁷ and preferably one substituent R⁷ that is especially preferably selected from the group consisting of NHCH₂CH₂NH₂, OCH₂CH₂NH₂, NHCH₂C(CH₃)NH₂, OCH₂C(CH₃)NH₂, NHC(CH₃)CH₂NH₂, OC(CH₃)CH₂NH₂, N(CH₃)CH₂CH₂NH₂, N(CH₃)CH₂CH₂N(CH₃)₂, N(CH₃)CH₂CH₂N(CH₃)₂, N(CH₃)CH₂CH₂OCH₃, OCH₂CH₂N(CH₃)₂ and N(CH₃)CH₂CH₂OCH₃.

Another preferred embodiment of the instant invention relates to compounds of formula I and preferably one or more of sub formulae I.1) to I.20), wherein Ar¹ comprises one or more, preferably one substituent R⁷ that is selected from O(CR⁵R⁶)ₖAr³—NR¹¹R¹². In this embodiment, k is preferably 0, 1 or 2 and more preferably 0 or 1. In this embodiment, R⁵ and/or R⁶ are preferably H or A and more preferably H. In this embodiment, Ar³ is preferably substituted or unsubstituted phenyl and more preferably unsubstituted phenyl. In this embodiment, R¹¹ and/or R¹² are preferably selected from H, A and C(O)A and more preferably from H and C(O)A, wherein A is preferably C₁-C₆-alkyl. In this embodiment, O(CR⁵R⁶)ₖAr³—NR¹¹R¹² is preferably O-phenyl-NH—C(O)A, wherein A is preferably selected from C₁-C₆-alkyl and more preferably is CH₃ or CF₃.

Another preferred embodiment of the instant invention relates to compounds of formula I and preferably one or more of sub formulae I.1) to I.20), wherein Ar¹ comprises one or more, preferably one substituent R⁷ that comprises (CR⁵R⁶)ₙ and/or (CR⁵R⁶)ₖ groups, wherein one or more, preferably one of R⁵R⁶ represents an oxo group. In this embodiment, (CR⁵R⁶)ₙ and/or (CR⁵R⁶)ₖ groups are preferably selected from C(O), CR⁵R⁶—C(O), CR⁵R⁶—CR⁵R⁶—C(O), C(O)—CR⁵R⁶ and C(O)—CR⁵R⁶—CR⁵R⁶, wherein R⁵R⁶ preferably do not represent an oxo-group and more preferably are independently selected from H and A and even more preferably are H. In this embodiment, (CR⁵R⁶)ₙ and/or (CR⁵R⁶)ₖ groups are preferably selected from C(O), CH₂—C(O), CH₂—CH₂—C(O), C(O)—CH₂ and C(O)—CH₂CH₂ and more preferably from C(O), CH₂—C(O) and CH₂—CH₂—C(O).

Another preferred embodiment of the instant invention relates to compounds of formula I and preferably one or more of sub formulae I.1) to I.20), wherein Ar¹ comprises one or more, preferably one substituent R⁷ selected from C(O)NR¹¹R¹², CR⁵R⁶—C(O)—NR¹¹R¹² and CR⁵R⁶—CR⁵R⁶—C(O)—NR¹¹R¹², wherein R⁵R⁶ preferably do not represent an oxo-group and more preferably are independently selected from H and A and even more preferably are H. In this embodiment, NR¹¹ and/or R¹² are preferably independently selected from H, A CH₂)ₘAr³, and (CH₂)ₘHet. and more preferably from H and CH₂)ₘAr³, preferably H and CH₂)ₘAr³, wherein m is preferably 0 or 1 and/or wherein Ar³ is substituted or unsubstituted phenyl. In this embodiment, R⁷ is preferably selected from C(O)—NH₂, CH₂—C(O)—NH₂, C(O)—NH-phenyl and CH₂—C(O)—NH-phenyl.

Another preferred embodiment of the instant invention relates to compounds of formula I and preferably one or more of sub formulae I.1) to I.20), wherein Ar¹ comprises one or more, preferably one substituent R⁷ selected from (CR⁵R⁶)ₖNR¹¹R¹², wherein one or more, preferably one of R¹¹ and R¹² is selected from C(O)A, C(O)(CH₂)ₘAr³ and C(O)(CH₂)ₘHet. In this embodiment, NR¹¹R¹² is preferably selected from NH—C(O)A, NH—C(O)(CH₂)ₘAr³ and NH—C(O)(CH₂)ₘHet. In this embodiment, m is preferably 0, 1 or 2 and preferably is 0. In this embodiment, A is preferably selected from substituted or unsubstituted, preferably unsubstituted C₁-C₆-alkyl. Het is preferably selected from substituted or unsubstituted pyridinyl. Ar³ is preferably selected from substituted or unsubstituted phenyl. In this embodiment, R⁷ is preferably selected from NH—C(O)-A, CH₂—NH—C(O)-A, CH₂—CH₂—NH—C(O)-A, NH—C(O)—Ar³, CH₂—NH—C(O)—Ar³, CH₂—NH₂—NH—C(O)—Ar³, NH—C(O)—Ar³, CH₂—NH—C(O)—Ar³, CH₂—CH₂—NH—C(O)—Ar³, NH—C(O)-Het, CH₂—NH—C(O)-Het, CH₂—CH₂—NH—C(O)-Het and especially preferably from CH₂—NH—C(O)—CH₃, CH₂—CH₂—NH—C(O)—CH₃, NH—C(O)—C₅H₅N, CH₂—NH—C(O)—C₅H₅N, and CH₂—CH₂—NH—C(O)—C₅H₅N.

Another preferred embodiment of the instant invention relates to compounds of formula I and preferably one or more of sub formulae I.1) to I.20), wherein Ar¹ comprises one or more, preferably one substituent R⁷ selected from (CR⁵R⁶)ₖNR¹¹R¹², wherein one or more, preferably one of R¹¹ and R¹² is selected from S(O)ᵤA. In this embodiment, u is preferably 0 or 2 and more preferably 2. In this embodiment, A is preferably substituted or unsubstituted C₁-C₆-alkyl. In this embodiment, k is preferably 0, 1, 2 or 3, preferably 1, 2 or 3. More preferably, (CR⁵R⁶)ₖNR¹¹R¹² is selected from (CR⁵R⁶)NR¹¹SO₂A, (CR⁵R⁶)₂NR¹¹SO₂A, (CR⁵R⁶)₃NR¹¹SO₂A. More preferably, (CR⁵R⁶)ₖNR¹¹R¹² is selected from (CH₂)NR¹¹SO₂A, (CH₂)₂NR¹¹SO₂A and (CH₂)₃NR¹¹SO₂A and even more preferably from (CH₂)NHSO₂A, (CH₂)₂NHSO₂A and (CH₂)₃NHSO₂A. Especially preferably in this embodiment, (CR⁵R⁶)ₖNR¹¹R¹² is selected from (CH₂)NHSO₂CH₃, (CH₂)₂NHSO₂CH₃ and (CH₂)₃NHSO₂CH₃.

Another preferred embodiment of the instant invention relates to compounds of formula I and preferably one or more of sub formulae I.1) to I.20), wherein Ar¹ comprises one or more, preferably one substituent R⁷ selected from SO₂R¹³, SO₂(CR⁵R⁶)ₖOR¹³ and SO₂(CR⁵R⁶)ₖNR¹¹R¹². In this embodiment, R¹³ is preferably selected from H and A and especially from H and substituted or unsubstituted C₁-C₆-alkyl. In this embodiment, k is preferably 0, 1, 2 or 3, more preferably 0, 1 or 2 and especially 0 or 2. In this embodiment, R⁵ and/or R⁶ is preferably H or A and more preferably H. In this embodiment, R¹¹ and/or R¹² are preferably selected from H and A. More preferably in SO₂R¹³, R¹³ is selected from substituted or unsubstituted C₁-C₆-alkyl. Even more preferably, SO₂R¹³ is selected from SO₂CH₃, SO₂CHal₃ and especially from SO₂CH₃ and SO₂CF₃. More preferably in SO₂(CR⁵R⁶)ₖOR¹³, R¹³ is selected from H and substituted or unsubstituted C₁-C₆-alkyl. Even more preferably, SO₂(CR⁵R⁶)ₖOR¹³ is selected from SO₂(CR⁵R⁶)ₖOH, SO₂(CR⁵R⁶)ₖOCH₃ and SO₂(CR⁵R⁶)ₖOCF₃, and especially from SO₂OH, SO₂(CH₂)OH and SO₂(CH₂)₂OH.

Another preferred embodiment of the instant invention relates to compounds of formula I and preferably one or more of sub formulae I.1) to I.20), wherein Ar¹ comprises one or more, preferably one substituent R⁷ selected from divalent radicals of formula —SO₂—CR⁸═CR⁸—, wherein both valencies are bound vicinally to Ar¹. In this embodiment, R⁸ are preferably independently selected from the meanings given for R⁸, R⁹ and R¹⁰ and especially independently selected from H, A, R¹³, Hal, NO₂, CN, OR¹⁵, NR¹⁵R¹⁶, COOR¹⁵, CONR¹⁵R¹⁶, NR¹⁵COR¹⁶, NR¹⁵CONR¹⁵R¹⁶, NR¹⁶SO₂A, COR¹⁵, SO₂NR¹⁵R¹⁶, and S(O)ᵤA and more preferably from H, A, Hal and CN. In this embodiment, R⁷—Ar¹ is preferably selected from 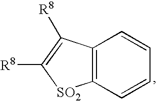
which is optionally additionally substituted by one or more residues R⁸. In this embodiment, (R⁸)ₚ—Ar¹—(R⁷)_{g} is more preferably selected from 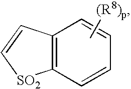
and especially from 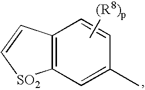
wherein R⁸ and q are as defined herein.

Another preferred embodiment of the instant invention relates to compounds of formula I and preferably one or more of sub formulae I.1) to I.20), wherein q is 0, i.e. the 6-membered aromatic, E, G, M, Q and U containing group bound to the urea moiety is unsubstituted.

Another preferred embodiment of the instant invention relates to compounds of formula I and preferably one or more of sub formulae I.1) to I.20), wherein q is 1, i.e. the 6-membered aromatic, E, G, M, Q and U containing group bound to the urea moiety is substituted by one substituent, preferably a substituent as defined above and more preferably a substituent selected from alkyl and hal, and especially selected from CH₃, CH₂CH₃ and hal.

Another preferred embodiment of the instant invention relates to compounds of formula I and preferably one or more of formulae I.1) to I.20), wherein (R⁸)ₚ—Ar¹ is selected from the group consisting of 3-acetyl-phenyl, 4-acetyl-phenyl, 2-bromo-phenyl, 3-bromo-phenyl, 4-bromo-phenyl, 4-bromo-2-chloro-phenyl, 4-bromo-3-methyl-phenyl, 4-bromo-3-trifluoromethyl-phenyl, 2-chloro-phenyl, 2-chloro-4-trifluoromethyl-phenyl, 2-chloro-5-trifluoromethyl-phenyl, 3-chloro-phenyl, 3-chloro-4-methyl-phenyl, 3-chloro-4-methoxy-phenyl, 3-chloro-4-methoxy-phenyl, 4-chloro-phenyl, 4-chloro-2-trifluoromethyl-phenyl, 4-chloro-3-trifluoromethyl-phenyl, 4-chloro-2-methyl-phenyl, 5-chloro-2-methyl-phenyl, 5-chloro-2-methoxy-phenyl, 2,3-dichloro-phenyl, 2,4-dichloro-phenyl, 2,5-dichloro-phenyl, 3,4-dichloro-phenyl, 3,5-dichloro-phenyl, 2,4,5-trichloro-phenyl, 4-fluoro-phenyl, 4-fluoro-3-trifluoromethyl-phenyl, 4-ethoxy-phenyl, 2-methoxy-phenyl, 2-methoxy-5-trifluoromethyl-phenyl, 4-methoxy-phenyl, 2,5-dimethoxy-phenyl, 2-trifluoromethyl-phenyl, 3-trifluoromethyl-phenyl, 3-trifluoromethoxy-phenyl, 4-trifluoromethyl-phenyl, 4-trifluoromethoxy-phenyl, 3,5-bis-trifluoromethyl-phenyl, 3-methoxy-phenyl, 3-methylsulfanyl-phenyl, 4-methylsulfanyl-phenyl, o-tolyl (2-methyl-phenyl), m-tolyl (3-methyl-phenyl), p-tolyl (4-methyl-phenyl), 2,3-dimethyl-phenyl, 2,3-di-methyl-phenyl, 2,5-dimethyl-phenyl, 3,4-dimethyl-phenyl, 3,5-dimethyl-phenyl, 2-ethyl-phenyl, 3-ethyl-phenyl, 4-ethyl-phenyl, 4-isopropyl-phenyl, 4-tert-butyl-phenyl and 5-tert-butyl-isoxazol-3-yl. Additionally preferred are compounds of formula I and preferably one or more of formulae I.1) to I.20), wherein (R⁸)ₚ—Ar¹ is selected from the residues given above and comprises one or two, preferably one substituent R⁷ and especially one or two, preferably one substituent R⁷ indicated herein as preferred, more preferred or especially preferred.

Another preferred embodiment of the instant invention relates to compounds of formula I and the subformulae related thereto and preferably one or more of formulae I.1) to I.20), wherein the residues (R⁸)ₚ—Ar¹—(R⁷)_{g} are selected from the following formulae: 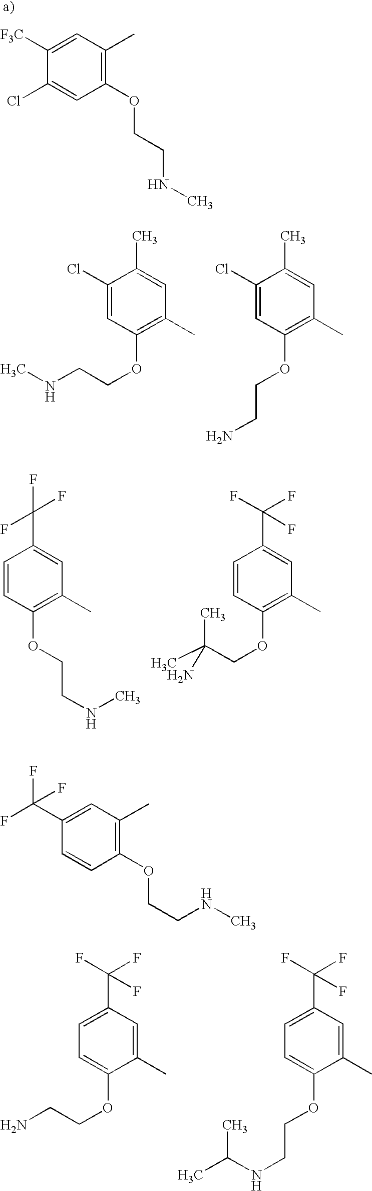 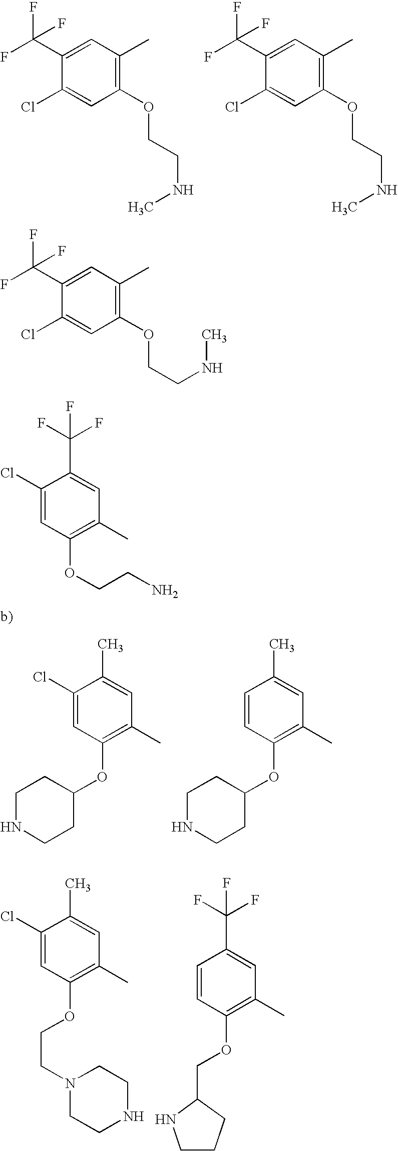 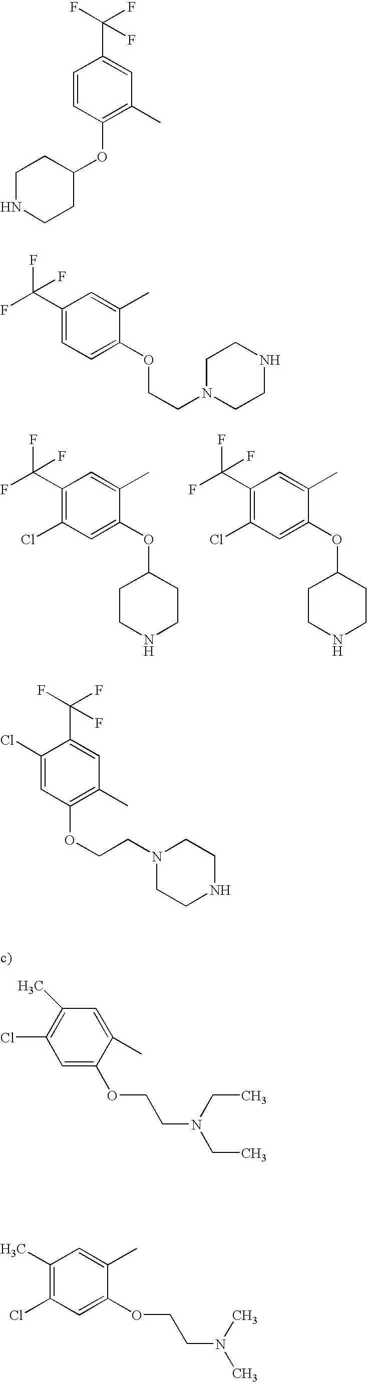 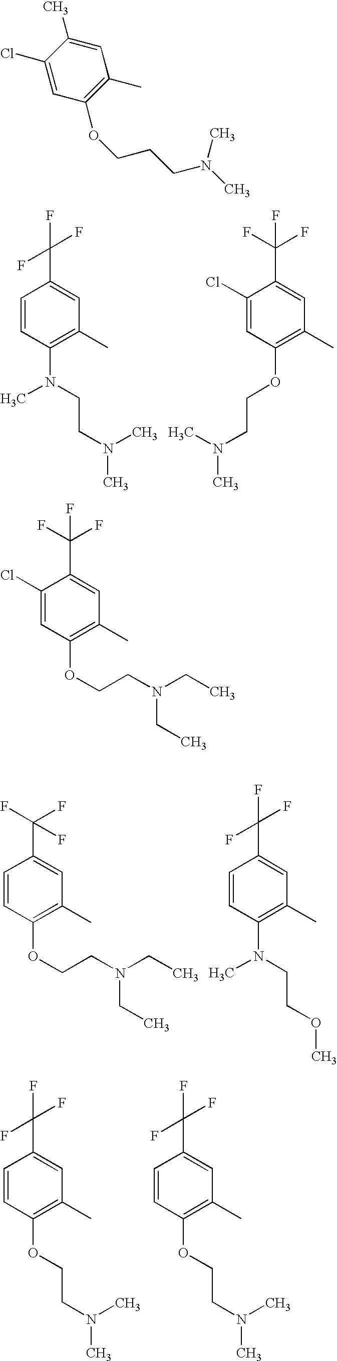 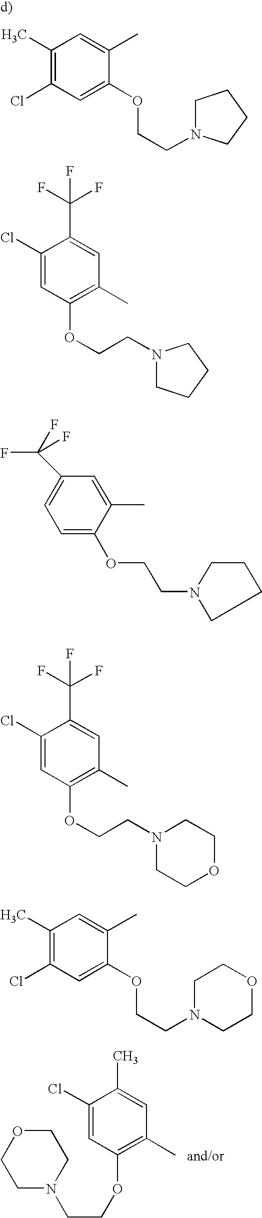 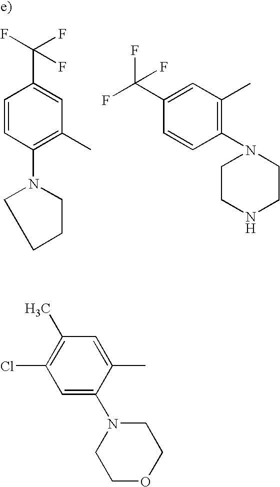
and/or residues of the structures given above that comprise one or two, preferably one additional substituent, independently selected from the meanings given for R⁷ and/or R⁸.

Another preferred embodiment of the instant invention relates to compounds of formula I and the subformulae related thereto and preferably one or more of formulae I.1) to I.20), wherein the residues (R⁸)ₚ—Ar¹—(R⁷)_{g} are selected from the following formulae: 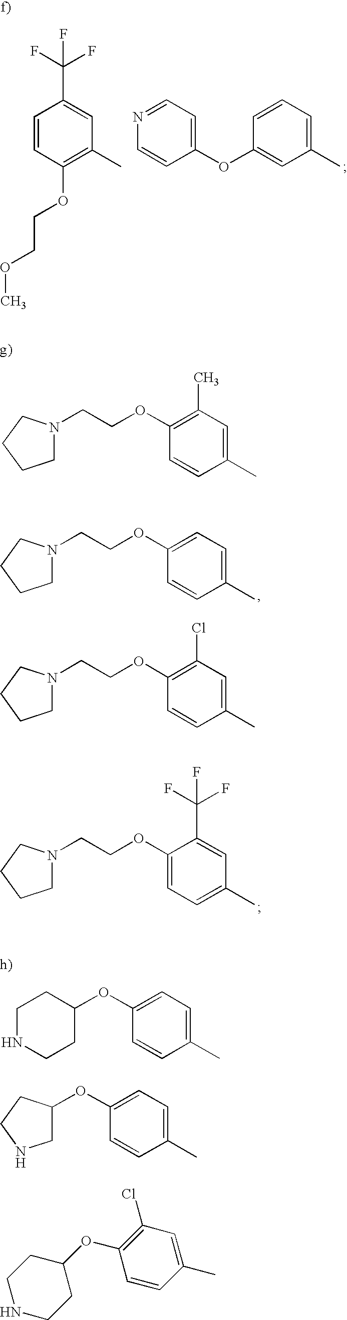 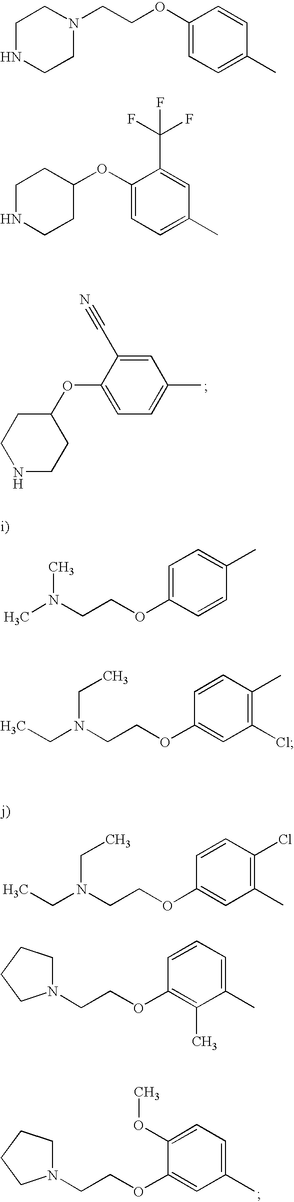 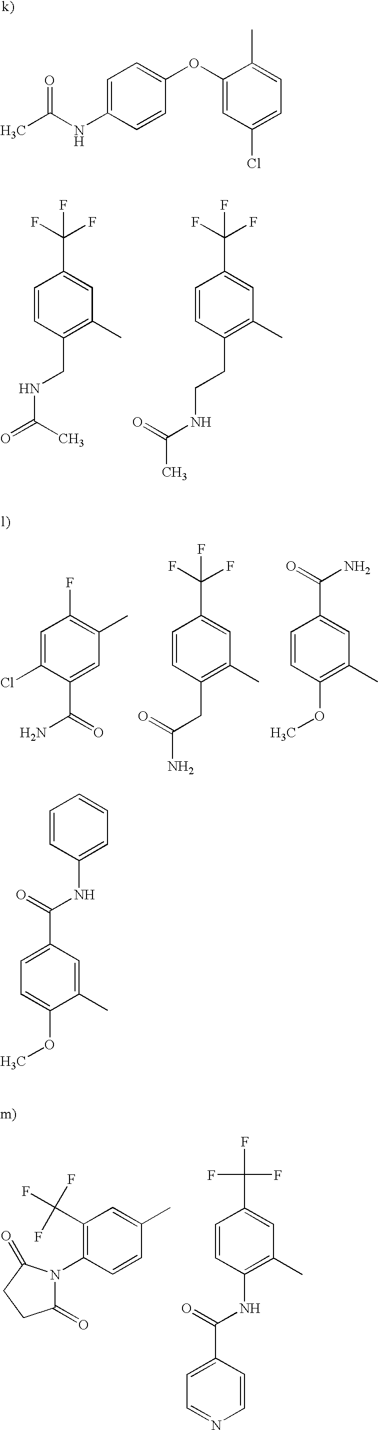 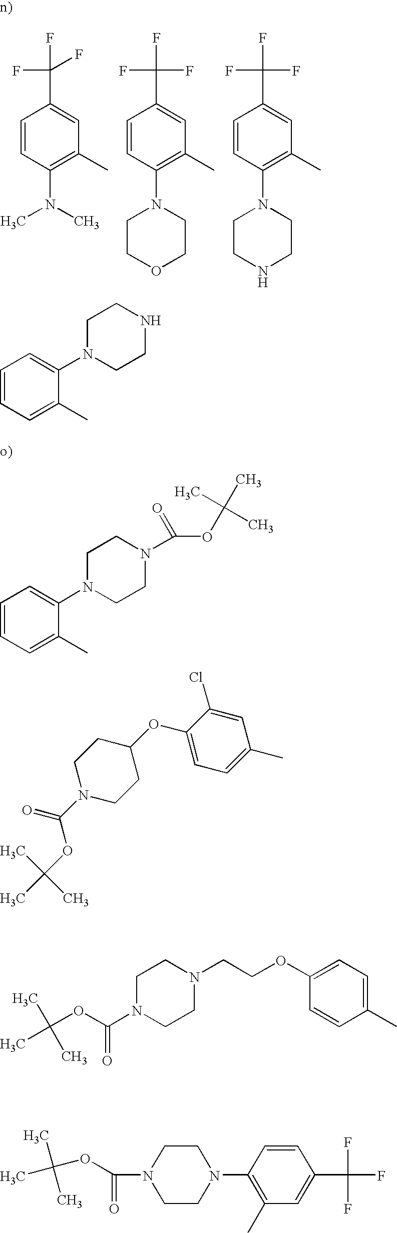 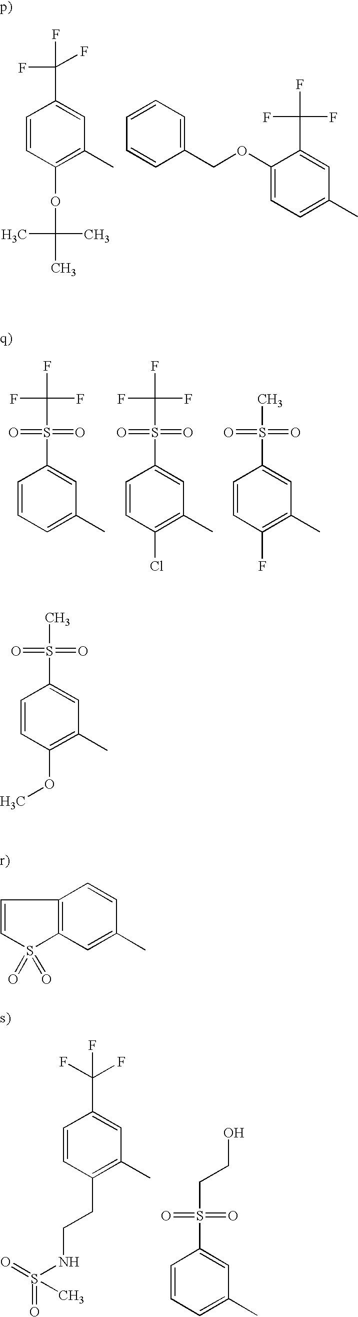 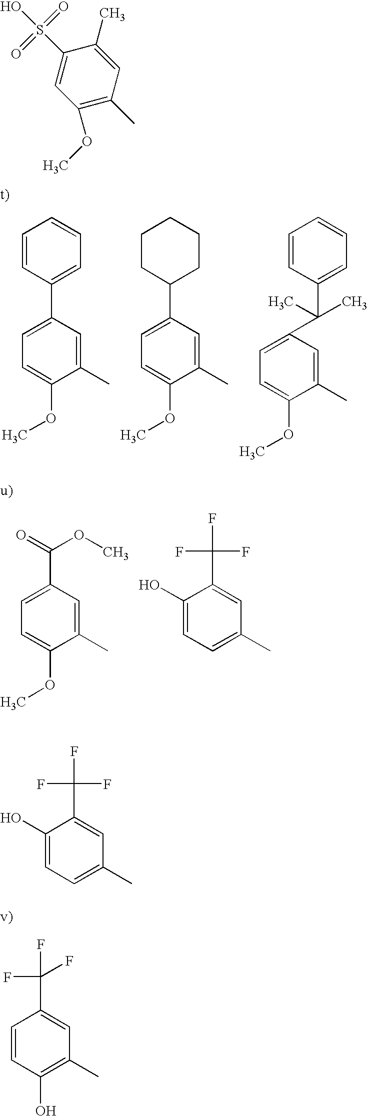
and/or residues of the structures given above that comprise one or two, preferably one additional substituent, independently selected from the meanings given for R⁷ and/or R⁸.

Another preferred embodiment of the instant invention relates to compounds of formula A-NH—CO—NH—B, wherein A is selected from the meanings of (R⁸)ₚ—Ar¹—(R⁷)_{g} as defined in the paragraph above, and B is selected from formulae

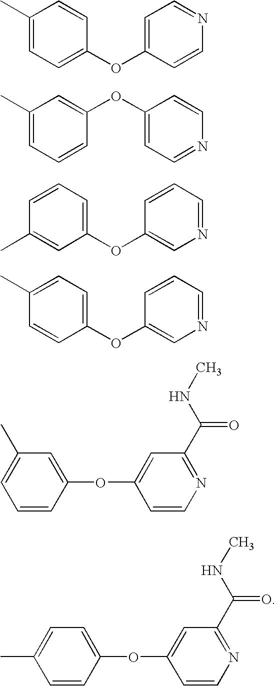

Another preferred embodiment of the instant invention relates to compounds of formula I and preferably one or more of sub formulae I.1) to I.20), wherein (R⁸)ₚ—Ar¹ is as defined above, but comprises one or more additional residues, preferably one additional residue. The additional residues are preferably selected from the meanings given for R⁷ and more preferably selected from the group consisting of O(CH₂)ₖR¹³, NR¹¹(CH₂)ₖR¹³, O(CH₂)ₖOR¹³, NR¹¹(CH₂)ₖOR¹³, O(CH₂)ₖNR¹¹R¹², NR¹¹(CH₂)ₖNR¹¹R¹², O(CH₂)ₙO(CH₂)ₖNR¹¹R¹², NR¹¹(CH₂)ₙO(CH₂)ₖNR¹¹R¹², O(CH₂)ₙNR¹²(CH₂)ₖNR¹¹R¹², NR¹¹(CH₂)ₙNR¹²(CH₂)ₖNR¹¹R¹², O(CH₂)ₙO(CH₂)ₖOR¹¹, NR¹¹(CH₂)ₙO(CH₂)ₖOR¹², O(CH₂)ₙNR¹¹(CH₂)ₖOR¹² and NR¹²(CH₂)ₙNR¹¹(CH₂)ₖOR¹², and even more preferably O(CH₂)ₖR¹³, NR¹¹(CH₂)ₖR¹³, O(CH₂)ₖOR¹³, NR¹¹(CH₂)ₖOR¹³, O(CH₂)ₖNR¹¹R¹², NR¹¹(CH₂)ₖNR¹¹R¹². In this embodiment, n is preferably 1 or 2. In this embodiment, k is preferably 1 or 2, and especially is 2.

Another preferred embodiment of the instant invention relates to compounds of formula I and the subformulae related thereto and preferably one or more of formulae I.1) to I.20), wherein the residues Ar²—(R¹⁰)ᵣ are selected from the group consisting of the following formulae: 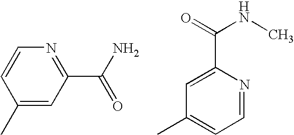
and/or residues of the structures given above that comprise one or two, preferably one additional substituent, independently selected from the meanings given for R¹⁰.

Another preferred embodiment of the instant invention relates to compounds of formula I and preferably one or more of sub formulae I.1) to I.20), wherein X is bonded in the para- (p-) or metha- (m-)position to the 6-membered aromatic, E, G, M, Q and U containing group that is bonded directly to the urea moiety.

Another preferred embodiment of the instant invention relates to compounds of formula I and preferably one or more of sub formulae I.1) to I.20), wherein Ar² is a pyridinyl residue and wherein said pyridinyl residue is bonded to X in the 3- or 4-position, preferably the 4-position, relative to the nitrogen atom of the pyridinyl residue.

Another preferred embodiment of the instant invention relates to compounds of formula I and preferably one or more of sub formulae I.1) to I.20), wherein Ar¹ comprises one or more substituents R⁸ as defined above/below; and one or two, preferably one substituent R⁷ that is selected from the group consisting of NHCH₂CH₂NH₂, N(CH₃)CH₂CH₂NH₂, N(CH₃)CH₂CH₂N(CH₃)₂, N(CH₃)CH₂CH₂N(CH₃)₂, N(CH₃)CH₂CH₂OCH₃, OCH₂CH₂N(CH₃)₂, OCH₂CH₂N(CH₂CH₃)₂, OCH₂CH₂NHCH₃ and/or the formulae aa):
aa) 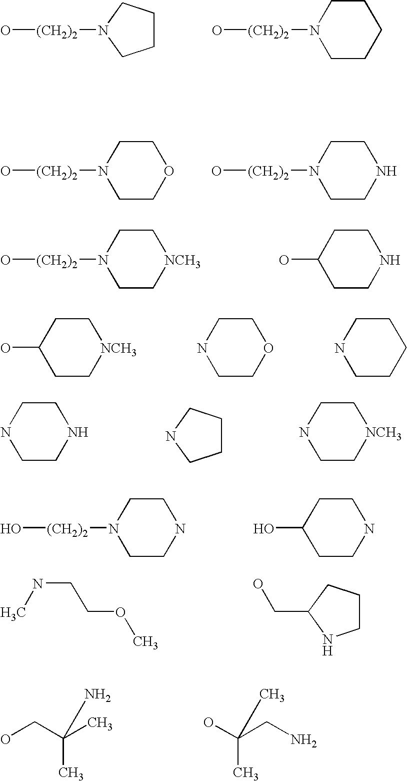
and/or bb):
bb) 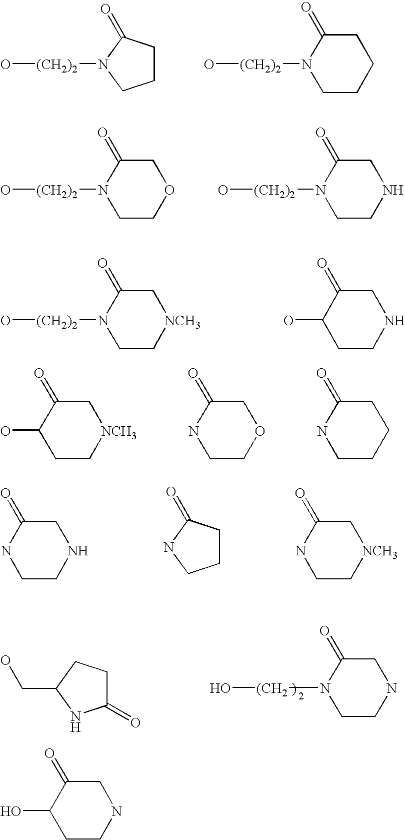
and/or cc):
cc) 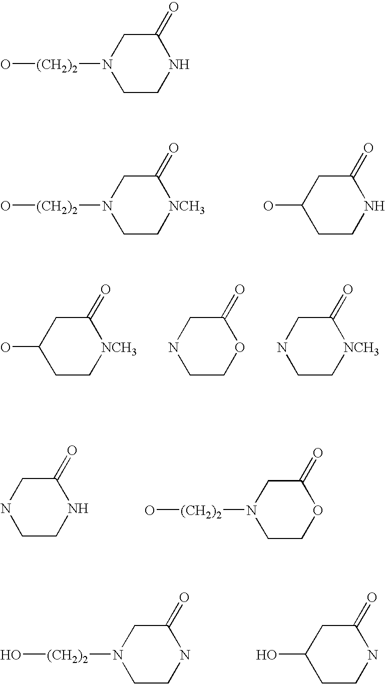
and/or Ar² comprises one or more substituents R¹⁰ and wherein one or two, preferably one substituent R¹⁰ is independently selected from the meanings given for R⁷ in this paragraph.

Another especially preferred embodiment of the instant invention relates to compounds of formula I and preferably one or more of sub formulae I.1) to I.20), wherein Ar¹ comprises one or two, preferably one substituent R⁷ that is selected from the group consisting of the formulae aa) and/or formulae bb) and/or formulae cc) as given above.

Another especially preferred embodiment of the instant invention relates to compounds of formula I and preferably one or more of sub formulae I.1) to I.20), wherein Ar¹ comprises one or two, preferably one substituent R⁷ that is selected from the group consisting of the formulae aa).

Another especially preferred embodiment of the instant invention relates to compounds of formula I and preferably one or more of sub formulae I.1) to I.20), wherein Ar¹ comprises one or two, preferably one substituent R⁷ that is selected from the group consisting of the formulae bb).

Another especially preferred embodiment of the instant invention relates to compounds of formula I and preferably one or more of sub formulae I.1) to I.20), wherein Ar¹ comprises one or more substituents R⁸ and one or two, preferably one substituent R⁷ that is selected from the group consisting of the formulae cc).

Another preferred embodiment of the instant invention relates to compounds of formula I and preferably one or more of sub formulae I.1) to I.20), wherein Ar¹ comprises one or more substituents R⁸ and wherein one or two, preferably one substituent R⁸ is selected from the group consisting of SO₂CH₃, SO₂CF₃, OSO₂CH₃, OSO₂CF₃, SO₂NH₂, SO₂NHCH(CH₃)₂, SO₂N(CH₃)₂, SO₂N(CH₂CH₃)₂ and 4-Morpholine-4-sulfonyl.

Another preferred embodiment of the instant invention relates to compounds of formula I and preferably one or more of sub formulae I.1) to I.20), wherein Ar² comprises one or more substituents R¹⁰ and wherein one or two, preferably one substituent R¹⁰ is selected from unsubstituted or substituted carbamoyl moieties. Substituted carbamoyl moieties are preferably selected from CONHR²³ or CONR²³R²⁴, preferably CONHR²³, wherein R²³ and R²⁴ are independently selected from the definitions given for R⁸, more preferably selected from alkyl, preferably methyl, ethyl, propyl and butyl, (CH₂)ₙNR¹¹R¹² and (CH₂)ₙOR¹², wherein R¹¹, R¹² and n are as defined above. In this embodiment, n is preferably not 0 and more preferred 1 to 3 and especially 1 or 2. Preferred examples for R²³ are selected from the group consisting of methyl, ethyl, CH₂CH₂NH₂, CH₂CH₂N(CH₃)₂, CH₂CH₂N(CH₂CH₃)₂, CH₂CH₂OH, CH₂CH₂OCH₃ and CH₂CH₂OCH₂CH₃.

Another preferred embodiment of the instant invention relates to compounds of formula I and preferably one or more of sub formulae I.1) to I.20), wherein Ar² comprises one or more substituents R¹⁰ and wherein one or two, preferably one substituent R¹⁰ is selected from substituted carbamoyl moieties. Substituted carbamoyl moieties are preferably selected from CONHR²³, wherein R²³ is preferably unsubstituted C₁-C₄-alkyl and especially methyl.

Another preferred embodiment of the instant invention relates to compounds of formula I and preferably one or more of sub formulae I.1) to I.20), wherein Ar² comprises one or more substituents R¹⁰ and wherein one or two, preferably one substituent R¹⁰ is selected from substituted carbamoyl moieties. Substituted carbamoyl moieties are preferably selected from CONHR²³, wherein R²³ is selected from (CH₂)ₙNR¹¹R¹² and (CH₂)ₙOR¹², wherein R¹¹, R¹² and n are as defined above. In this embodiment, n is preferably not 0 and more preferred 1 to 3 and especially 1 or 2. Preferred examples for R²³ are selected from the group consisting of CH₂CH₂NH₂, CH₂CH₂N(CH₃)₂, CH₂CH₂N(CH₂CH₃)₂, CH₂CH₂OH, CH₂CH₂OCH₃ and CH₂CH₂OCH₂CH₃.

Another preferred embodiment of the instant invention relates to compounds of formula I and preferably one or more of sub formulae I.1) to I.20), wherein —Ar²—(R¹⁰) is selected from the formulae 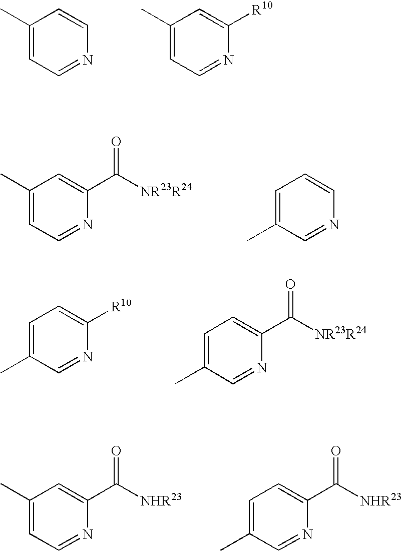
wherein R¹⁰, R²³ and R²⁴ are as defined above and below.

Another preferred embodiment of the instant invention relates to compounds of formula I and preferably the sub formulae related thereto, wherein R⁷ does not comprise OH, NH and/or NH₂ groups.

Another preferred embodiment of the instant invention relates to compounds of formula I and the sub formulae related thereto, wherein R⁸ does not comprise OH, NH and/or NH₂ groups.

Another preferred embodiment of the instant invention relates to compounds of formula I and the sub formulae related thereto, wherein R⁹ does not comprise OH, NH and/or NH₂ groups.

Another preferred embodiment of the instant invention relates to compounds of formula I and preferably the sub formulae related thereto, wherein Ar¹ and/or the 6-membered aromatic, E, G, M, Q and U containing group bound to the urea moiety, preferably Ar¹ and/or the phenyl group bound to the urea moiety, do not comprise a OH group in the ortho position to the urea moiety.

Another preferred embodiment of the instant invention relates to compounds of formula I and preferably the sub formulae related thereto, wherein Ar¹ and/or the 6-membered aromatic, E, G, M, Q and U containing group bound to the urea moiety, preferably Ar¹ and/or the phenyl group bound to the urea moiety, do not comprise a —NHSO₂— moiety in the ortho position to the urea moiety.

Another preferred embodiment of the instant invention relates to compounds of formula I and preferably the sub formulae related thereto, wherein Ar¹ and/or the 6-membered aromatic, E, G, M, Q and U containing group bound to the urea moiety, preferably Ar¹ and/or the phenyl group bound to the urea moiety, do not comprise a —NHSO₂— moiety in the ortho position to the urea moiety.

Another preferred embodiment of the instant invention relates to compounds of formula I and preferably the sub formulae related thereto, wherein Ar¹ and/or the 6-membered aromatic, E, G, M, Q and U containing group bound to the urea moiety, preferably Ar¹ and/or the phenyl group bound to the urea moiety, do not comprise a moiety in the ortho position to the urea moiety having an ionizable hydrogen and a pKa of 10 or less.

Another preferred embodiment of the instant invention relates to compounds of formula I and preferably the sub formulae related thereto, wherein both the aromatic groups bound directly to the urea moiety do not comprise a substituent in the ortho position to the urea moiety, selected from OH, substituents comprising a —NHSO₂— moiety, and substituents comprising moieties having an ionizable hydrogen and a pKa of 10 or less.

Another especially preferred embodiment of the instant invention relates to compounds of formula I, preferably the sub formulae related thereto and more preferably one or more of the sub formulae I.1) to I.20) and/or Ia to Iz and Iaa to Iuu, wherein one or more features of the above and below mentioned embodiments are combined in one compound.

Subject of the present invention are therefore preferably compounds of formula I according to one or both of the formulae Ia and Ib, 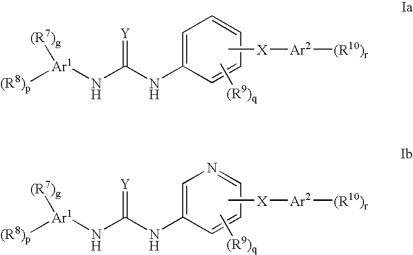
wherein Ar¹, R⁷, R⁸, p, g, Y, X, R⁹, q, Ar², R¹⁰ and r are as defined above and below, and preferably as defined in sub formulae I.1) to I.20) and/or the embodiments related thereto, and the pharmaceutically acceptable derivatives, solvates, salts and stereoisomers thereof, including mixtures thereof in all ratios, and more preferred the salts and/or solvates thereof, and especially preferred the physiologically acceptable salts and/or solvates thereof.

Subject of the present invention are therefore especially preferred compounds of formula I according to one or both of the formulae Ic and Id, 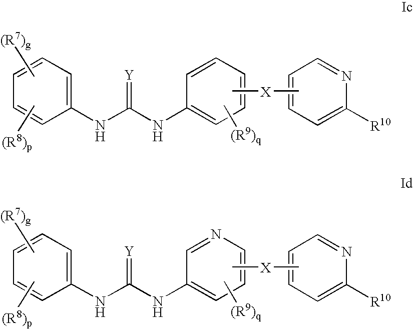
wherein R⁷, g, R⁸, p, Y, X, R⁹ and q are as defined above and below, R¹⁰ is H or as defined above/below, and preferably as defined in sub formulae I.1) to I.20) and/or the embodiments related thereto;
and/or compounds of formula I according to one or more of the formulae Ie to Iw, 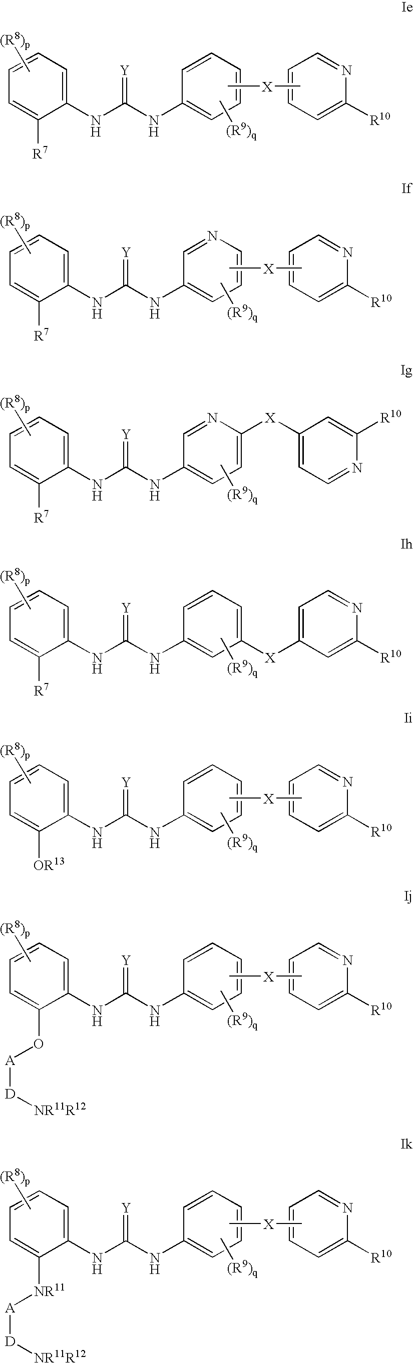 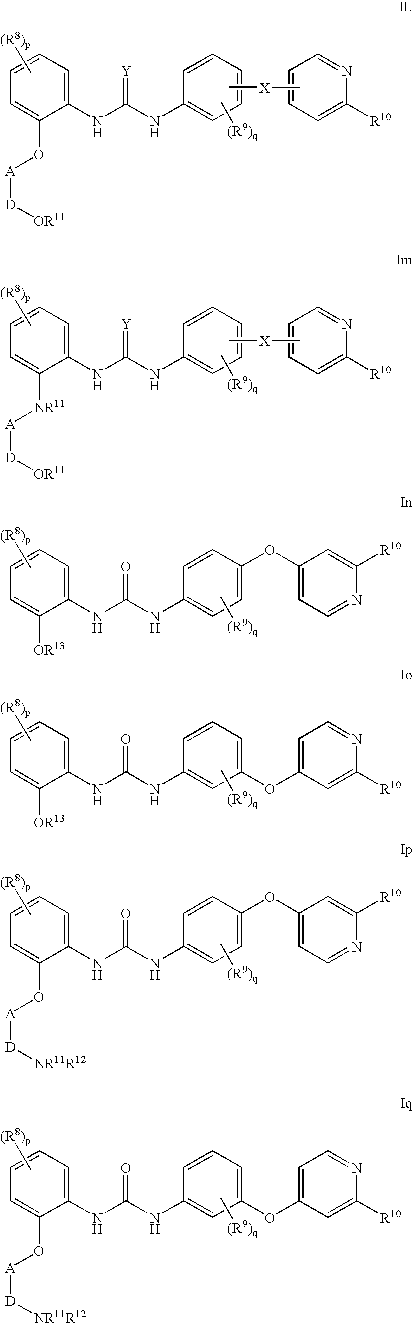 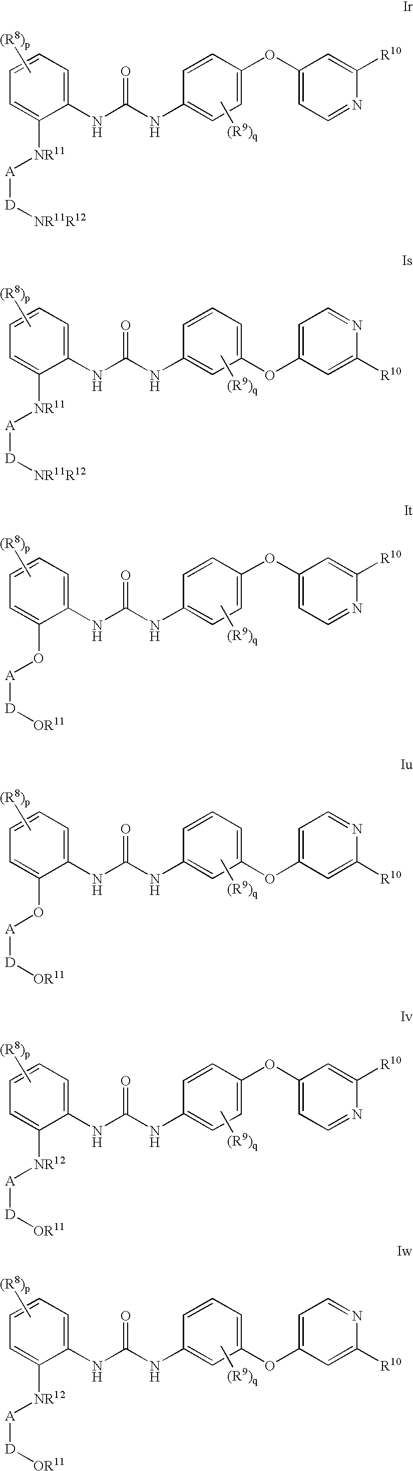
wherein R⁷, R⁸, R¹¹, R¹², R¹³, Y, X, R⁹, p and q are as defined above and below, R¹⁰ is H or as defined above/below, and preferably as defined in sub formulae I.1) to I.20) and/or the embodiments related thereto, and A and D are CR⁵R⁶, and the pharmaceutically acceptable derivatives, solvates, salts and stereoisomers thereof, including mixtures thereof in all ratios, and more preferred the salts and/or solvates thereof, and especially preferred the physiologically acceptable salts and/or solvates thereof.

Subject of the present invention are therefore especially preferred compounds of formula I according to one or more of the formulae Ix, Iy, Iz and Iaa to Iuu: 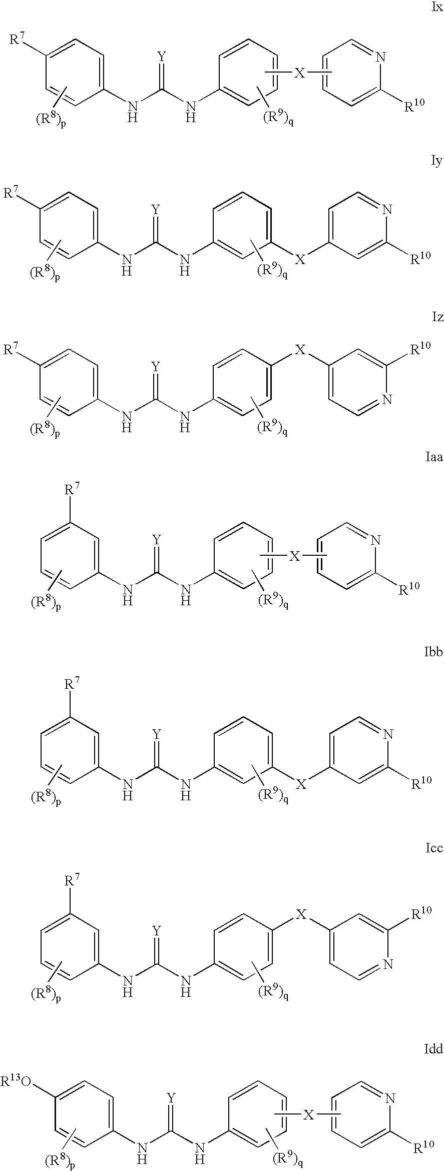 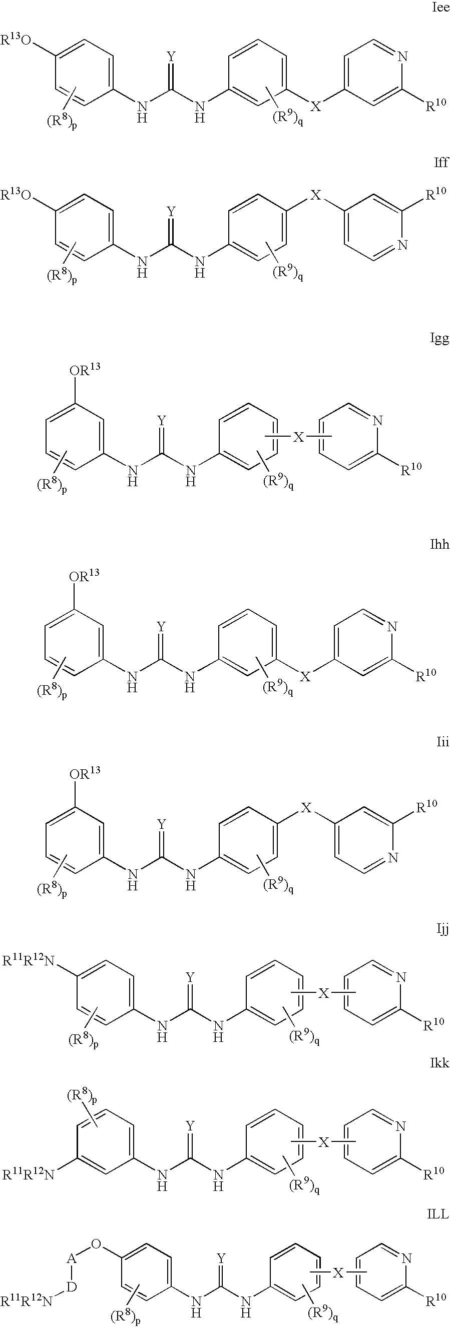 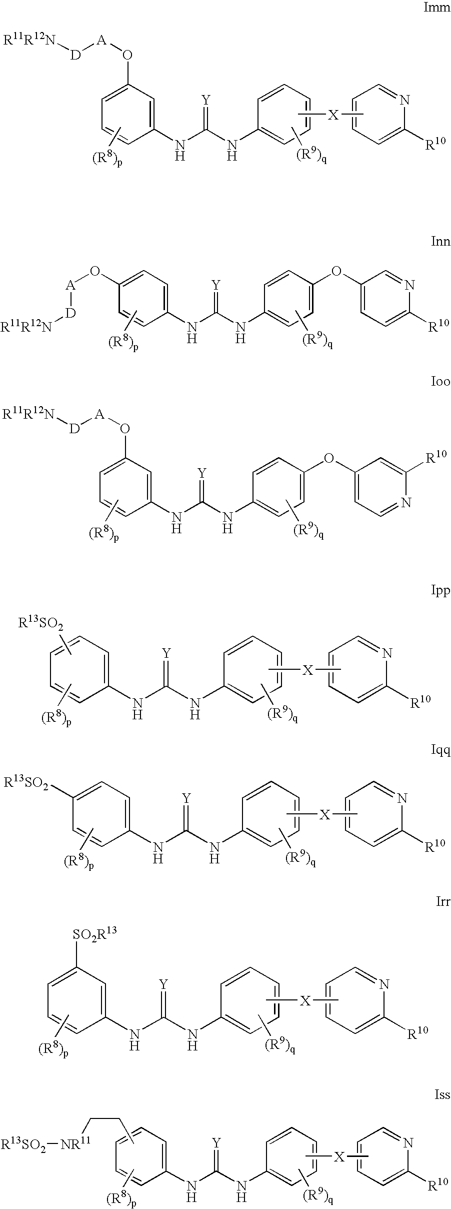 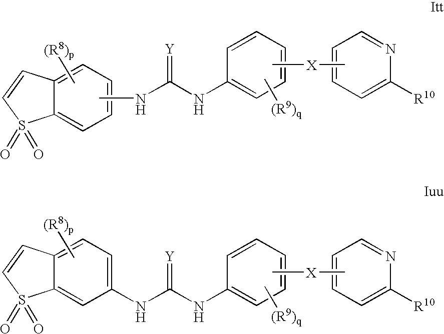
wherein R⁷, R⁸, R¹¹, R¹², R¹³, Y, X, R⁹, p and q are as defined above and below, R¹⁰ is H or as defined above/below, and preferably as defined in sub formulae I.1) to I.20) and/or the embodiments related thereto, and A and D are CR⁵R⁶, and the pharmaceutically acceptable derivatives, solvates, salts and stereoisomers thereof, including mixtures thereof in all ratios, and more preferred the salts and/or solvates thereof, and especially preferred the physiologically acceptable salts and/or solvates thereof.

Another preferred embodiment of the instant invention relates to compounds of formula I and preferably one or more of sub formulae I.1) to I.20) and Ia to Iw, Ix to Iz and/or Iaa to Iuu, wherein R¹⁰ is a substituted carbamoyl moiety CONHR²³ or CONR²³R²⁴, preferably CONHR²³, wherein R²³ and R²⁴ are independently selected from the definitions given for R⁸, more preferably selected from CH₃ and (CH₂)ₙNR¹¹R¹², wherein R¹¹, R¹² and n are as defined above. In this embodiment, n is preferably not 0 and more preferred 1 to 3 and especially 1 or 2. Preferred examples for R²³ are selected from the group consisting of CH₃, CH₂CH₂NH₂, CH₂CH₂N(CH₃)₂, CH₂CH₂N(CH₂CH₃)₂, CH₂CH₂OH, CH₂CH₂OCH₃ and CH₂CH₂OCH₂CH₃.

Another preferred embodiment of the instant invention relates to compounds of formula I and preferably one or more of sub formulae I.1) to I.20) and Ii and In, wherein R¹³ is (CH₂)ₘHet, wherein Het is preferably saturated heterocyclyl and wherein m is preferably 0, 1 or 2.

Another preferred embodiment of the instant invention relates to compounds of sub formulae Ij to Im and Ip to Iw, wherein A and D are independently selected from CH₂ and C(CH₃)₂.

It is understood that when a residue, for example R⁸, R⁹, R¹⁰ or R¹⁴ or R²³, is comprised twice or more times in one or more of the formulae I and the sub formulae corresponding thereto, it is in each case independently from one another selected from the meanings given for the respective residue. For example, R¹¹ and R¹² are defined to be independently selected from a group consisting of H, A, (CH₂)ₘAr³ and (CH₂)ₘHet. Then (CH₂)ₙNR¹¹(CH₂)ₘNR¹²R¹² can be (CH₂)ₙNA(CH₂)ₘNA₂ (if R¹¹=A, R¹²=A and R¹²═H) as well as (CH₂)ₙNA(CH₂)ₘNHA (if R¹¹=A, R¹²═H and R¹²=A or (CH₂)ₙNA(CH₂)ₘNH(CH₂ₘHet (if R¹¹=A, R¹²═H and R¹²=(CH₂)ₘHet). Accordingly, if a compound of formula I comprises one residue R⁸, R⁹ and R¹⁰, then for example R⁸, R⁹ and R¹⁰ can all be (CH₂)ₙCOOR¹³, wherein all residues R¹³ are the same (for example CH₂Hal, wherein Hal is Cl; then all residues R⁸, R⁹ and R¹⁰ are the same) or different (for example CH₂Hal, wherein in R⁸, Hal is Cl; in R⁹, Hal is F; and in R¹⁰, Hal is Br; then all residues R⁸, R⁹ and R¹⁰ are different); or for example R⁸ is (CH₂)ₙCOOR¹³, R⁹ is NO₂ and R¹⁰ is (CH₂)ₙSR¹¹, wherein R¹¹ and R¹³ can be the same (for example both can be H or both can be A which is methyl) of different (for example R¹¹ can be H and R¹³ can be A which is methyl).

If not stated otherwise, reference to compounds of formula I also includes the sub formulae related thereto, especially sub formulae I.1) to I.20) and Ia to Iw, Ix to Iz and/or Iaa to Iuu.

Subject of the instant invention are especially those compounds of formula I and preferably the sub formulae related thereto, in which at least one of the residues mentioned in said formulae has one of the preferred or especially preferred meanings given above and below.

Especially preferred as compounds according to the invention are the compounds given below: 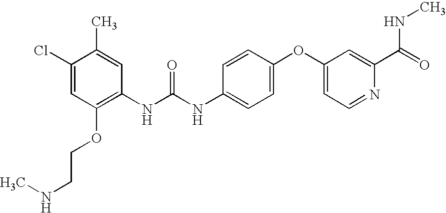
4-(4-{3-[4-Chloro-5-methyl-2-(2-methylamino-ethoxy)-phenyl]-ureido}-phenoxy)-pyridine-2-carboxylic acid methylamide (MW=483.95; Rt=2.08) 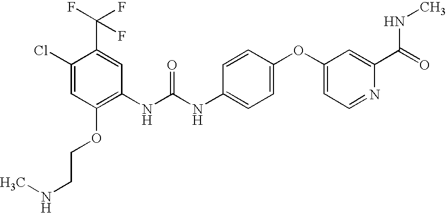
4-(4-{3-[Chloro-(2-methylamino-ethoxy)-trifluoromethyl-phenyl]-ureido}-phenoxy)-pyridine-2-carboxylic acid methylamide (MW=537.92; Rt=2.21) 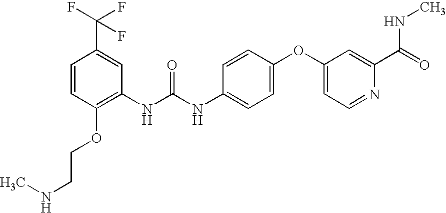
4-(4-{3-[(2-Methylamino-ethoxy)-trifluoromethyl-phenyl]-ureido}-phenoxy)-pyridine-2-carboxylic acidmethylamide (MW=503.48; Rt=2.09) 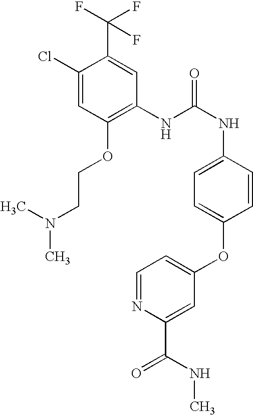
4-(4-{3-[Chloro-(2-dimethylamino-ethoxy)-trifluoromethyl-phenyl]-ureido}-phenoxy)-pyridine-2-carboxylic acid methylamide (MW=551.95; Rt=2.25) 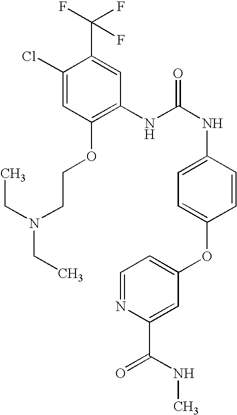
4-(4-{3-[Chloro-(2-diethylamino-ethoxy)-trifluoromethyl-phenyl]-ureido}-phenoxy)-pyridine-2-carboxylic acid methylamide (MW=580.00; Rt=2.29) 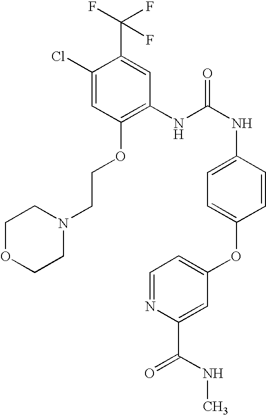
4-(4-{3-[Chloro-(2-morpholin-4-yl-ethoxy)-trifluoromethyl-phenyl]-ureido}-phenoxy)-pyridine-2-carboxylic acid methylamide (MW=593.99. Rt=2.26) 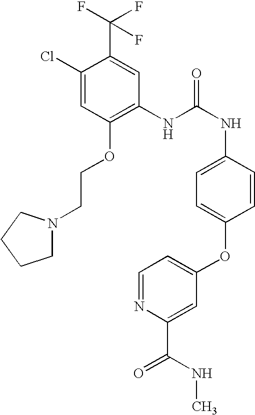
4-(4-{3-[Chloro-(2-pyrrolidin-1-yl-ethoxy)-trifluoromethyl-phenyl]-ureido}-phenoxy)-pyridine-2-carboxylic acid methylamide (MW=577.99; Rt=2.27) 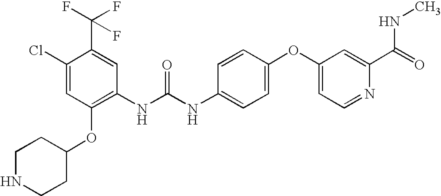
4-(4-{3-[Chloro-(piperidin-4-yloxy)-trifluoromethyl-phenyl]-ureido}-phenoxy)-pyridine-2-carboxylic acid methylamide (MW=563.96; Rt=2.29) 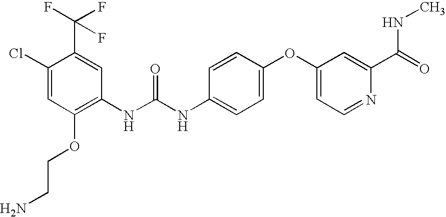
4-(4-{3-[(2-Amino-ethoxy)-chloro-trifluoromethyl-phenyl]-ureido}-phenoxy)-pyridine-2-carboxylic acidmethylamide (MW=523.90; Rt=2.21) 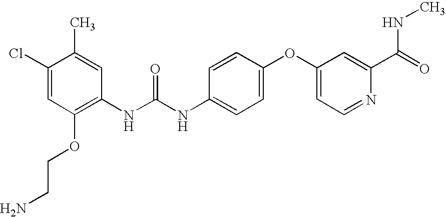
4-(4-{3-[2-(2-Amino-ethoxy)-4-chloro-5-methyl-phenyl]-ureido}-phenoxy)-pyridine-2-carboxylic acid methylamide (MW=469.93; Rt=2.03) 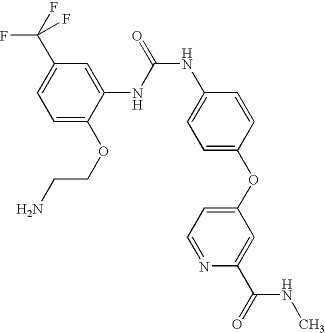
4-(4-{3-[(2-Amino-ethoxy)-trifluoromethyl-phenyl]-ureido}-phenoxy)-pyridine-2-carboxylic acid methylamide (MW=489.45; Rt=2.11); 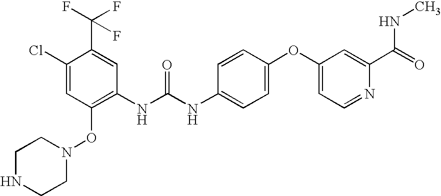
4-(4-{3-[Chloro-(2-piperazin-1-yl-ethoxy)-trifluoromethyl-phenyl]-ureido}-phenoxy)-pyridine-2-carboxylic acid methylamide (MW=593.00; Rt=2.24); 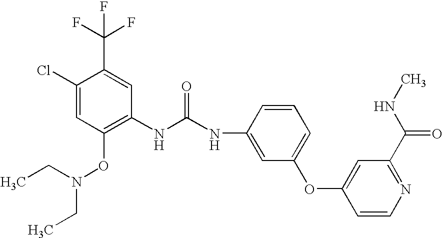
4-(3-{3-[Chloro-(2-diethylamino-ethoxy)-trifluoromethyl-phenyl]-ureido}-phenoxy)-pyridine-2-carboxylic acid methylamide (MW=580.00; Rt=2.29); 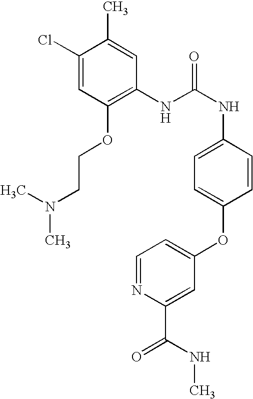
4-(4-[(3-{4-Chloro-2-(2-dimethylamino-ethoxy)-5-methyl-phenyl]-ureido}-phenoxy)-pyridine-2-carboxylic acid methylamide (MW=497.98; Rt=2.93^{a}); 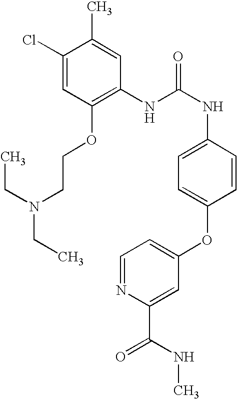
4-(4-{3-[4-Chloro-2-(2-diethylamino-ethoxy)-5-methyl-phenyl]-ureido}-phenoxy)-pyridine-2-carboxylic acid methylamide (MW=526.03; Rt=2.97^{a}); 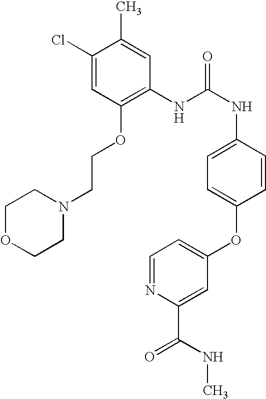
4-(4-{3-[4-Chloro-5-methyl-2-(2-morpholin-4-yl-ethoxy)-phenyl]-ureido}-phenoxy)-pyridine-2-carboxylic acid methylamide (MW=540.02; Rt=2.93^{a}); 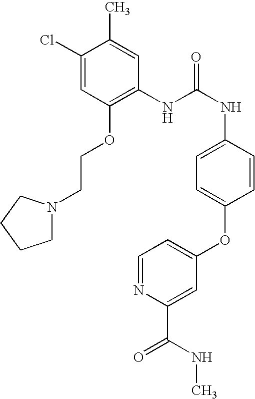
4-(4-{3-[4-Chloro-5-methyl-2-(2-pyrrolidin-1-yl-ethoxy)-phenyl]-ureido}-phenoxy)-pyridine-2-carboxylic acid methylamide (MW=524.02; Rt=2.99^{a}); 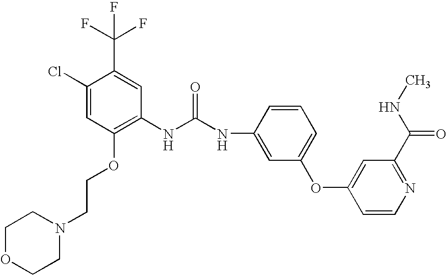
4-(3-{3-[Chloro-(2-morpholin-4-yl-ethoxy)-trifluoromethyl-phenyl]-ureido}-phenoxy)-pyridine-2-carboxylic acid methylamide (MW=593.99; Rt=2.27); 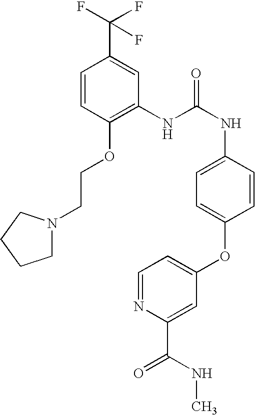
4-(4-{3-[(2-Pyrrolidin-1-yl-ethoxy)-trifluoromethyl-phenyl]-ureido}-phenoxy)-pyridine-2-carboxylic acid methylamide (MW=543.54; Rt=3.01^{a}); 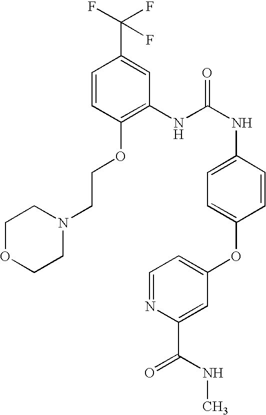
4-(4-{3-[(2-Morpholin-4-yl-ethoxy)-trifluoromethyl-phenyl]-ureido}-phenoxy)-pyridine-2-carboxylic acid methylamide (MW=559.54; Rt=2.98^{a}); 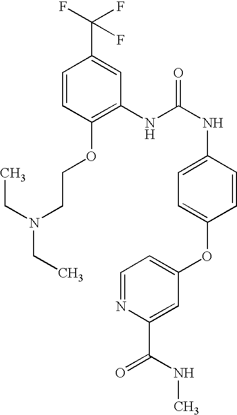
4-(4-{3-[(2-Diethylamino-ethoxy)-trifluoromethyl-phenyl]-ureido}-phenoxy)-pyridine-2-carboxylic acidmethylamide (MW=545.56; Rt=2.99^{a}); 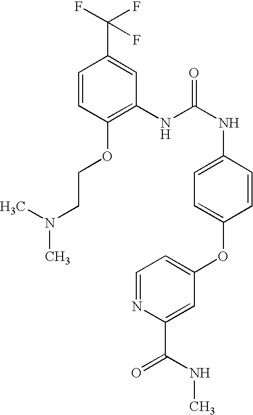
4-(4-{3-[(2-Dimethylamino-ethoxy)-trifluoromethyl-phenyl]-ureido}-phenoxy)-pyridine-2-carboxylic acid methylamide (MW=517.51; Rt=2.12); 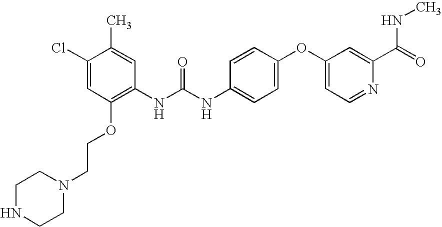
4-(4-{3-[4-Chloro-5-methyl-2-(2-piperazin-1-yl-ethoxy)-phenyl]-ureido}-phenoxy)-pyridine-2-carboxylic acid methylamide (MW=539.03; Rt=2.04); 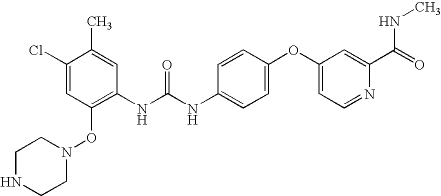
4-(4-{3-[4-Chloro-5-methyl-2-(piperidin-4-yloxy)-phenyl]-ureido}-phenoxy)-pyridine-2-carboxylic acidmethylamide (MW=509.99; Rt=2.14); 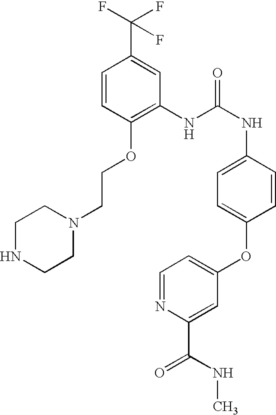
4-(4-{3-[(2-piperazin-1-yl-ethoxy)-trifluoromethyl-phenyl]-ureido}-phenoxy)-pyridine-2-carboxylic acid methylamide (MW=558.56; Rt=2.11); 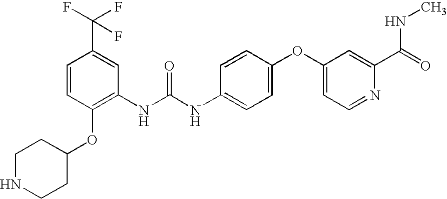
4-(4-{3-[(Piperidin-4-yloxy)-trifluoromethyl-phenyl]-ureido}-phenoxy)-pyridine-2-carboxylic acid methylamide (MW=529.52; Rt=2.17); 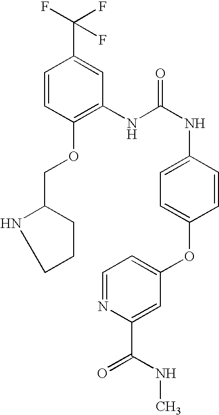
4-(4-{3-[(Pyrrolidin-2-ylmethoxy)-trifluoromethyl-phenyl]-ureido}-phenoxy)-pyridine-2-carboxylic acid methylamide (MW=529.52; Rt=2.14); 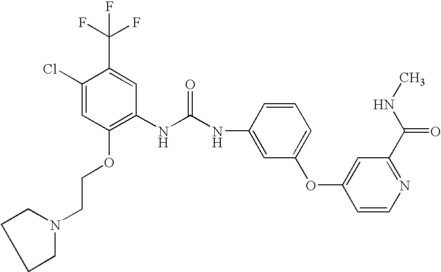
4-(3-{3-[Chloro-(2-pyrrolidin-1-yl-ethoxy)-trifluoromethyl-phenyl]-ureido}-phenoxy)-pyridine-2-carboxylic acid methylamide (MW=529.52; Rt=2.27); 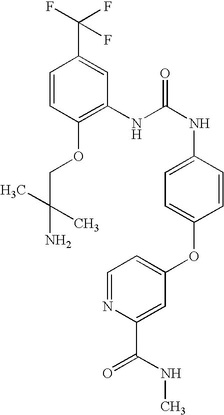
4-(4-{3-[(2-Amino-2-methyl-propoxy)-trifluoromethyl-phenyl]-ureido}-phenoxy)-pyridine-2-carboxylic acid methylamide (MW=517.51; Rt=2.13); 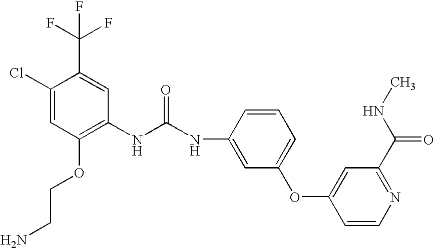
4-(3-{3-[(2-Amino-ethoxy)-chloro-trifluoromethyl-phenyl]-ureido}-phenoxy)-pyridine-2-carboxylic acidmethylamide (MW=523.90; Rt=2.23); 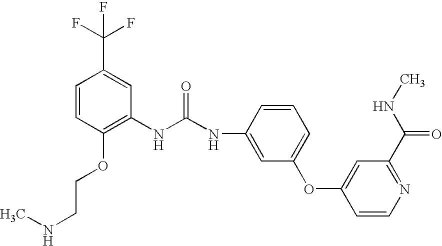
4-(3-{3-[(2-Methylamino-ethoxy)-trifluoromethyl-phenyl]-ureido}-phenoxy)-pyridine-2-carboxylic acidmethylamide (MW=503.48; Rt=2.14); 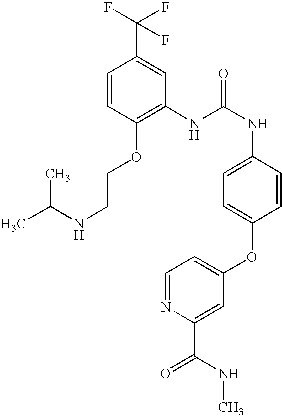
4-(4-{3-[(2-Isopropylamino-ethoxy)-trifluoromethyl-phenyl]-ureido}-phenoxy)-pyridine-2-carboxylic acid methylamide (MW=531.48; Rt=2.14); 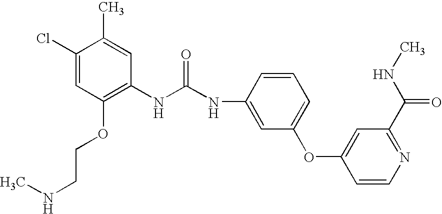
4-(3-{3-[4-Chloro-5-methyl-2-(2-methylamino-ethoxy)-phenyl]-ureido}-phenoxy)-pyridine-2-carboxylic acid methylamide (MW=483.95; Rt=2.11); 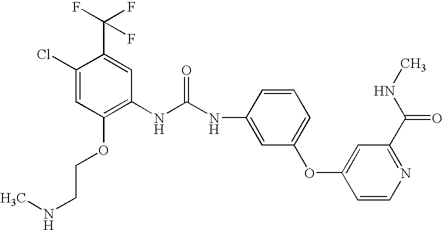
4-(3-{3-[Chloro-(2-methylamino-ethoxy)-trifluoromethyl-phenyl]-ureido}-phenoxy)-pyridine-2-carboxylic acid methylamide (MW=531.53; Rt=2.26); 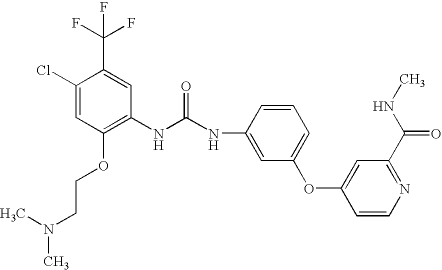
4-(3-{3-[Chloro-(2-dimethylamino-ethoxy)-trifluoromethyl-phenyl]-ureido}-phenoxy)-pyridine-2-carboxylic acid methylamide (MW=551.95; Rt=2.25); 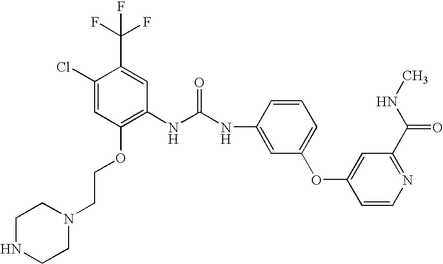
4-(3-{3-[Chloro-(2-piperazin-1-yl-ethoxy)-trifluoromethyl-phenyl]-ureido}-phenoxy)-pyridine-2-carboxylic acid methylamide (MW=593.00; Rt=2.2); 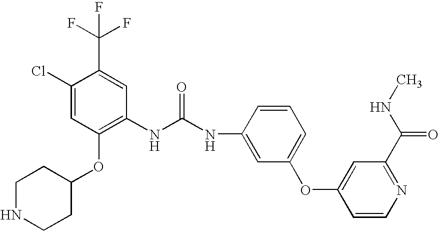
4-(3-{3-[Chloro-(piperidin-4-yloxy)-trifluoromethyl-phenyl]-ureido}-phenoxy)-pyridine-2-carboxylic acid methylamide (MW=563.96; Rt=2.31); 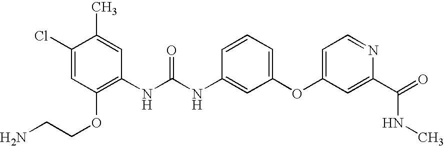
4-(3-{3-[2-(2-Amino-ethoxy)-4-chloro-5-methyl-phenyl]-ureido}-phenoxy)-pyridine-2-carboxylic acid methylamide (MW=469.93; Rt=2.07); 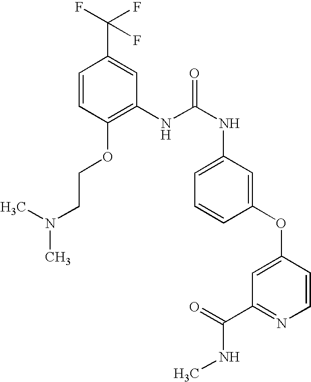
4-(3-{3-[(2-Dimethylamino-ethoxy)-trifluoromethyl-phenyl]-ureido}-phenoxy)-pyridine-2-carboxylic acid methylamide (MW=517.51; Rt=2.15); 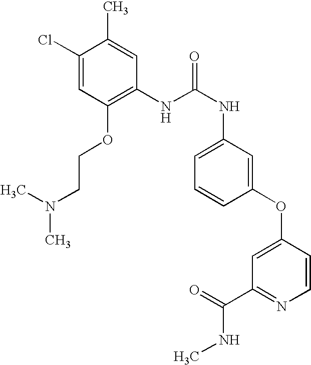
4-(3-{3-[(4-Chloro-2-(2-dimethylamino-ethoxy)-5-methyl-phenyl]-ureido}-phenoxy)-pyridine-2-carboxylic acid methylamide (MW=497.98; Rt=2.11); 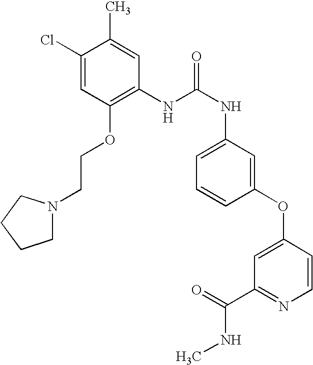
4-(3-{3-[4-Chloro-5-methyl-2-(2-pyrrolidin-1-yl-ethoxy)-phenyl]-ureido}-phenoxy)-pyridine-2-carboxylic acid methylamide (MW=524.02; Rt=2.21); 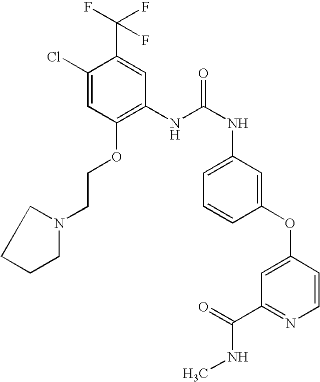
4-(3-{3-[(2-Pyrrolidin-1-yl-ethoxy)-trifluoromethyl-phenyl]-ureido}-phenoxy)-pyridine-2-carboxylic acid methylamide (MW=543.54; Rt=2.2); 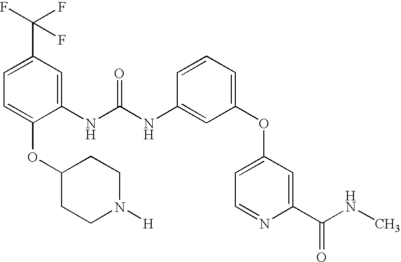
4-(3-{3-[(Piperidin-4-yloxy)-trifluoromethyl-phenyl]-ureido}-phenoxy)-pyridine-2-carboxylic acid methylamide (MW=529.52; Rt=2.2); 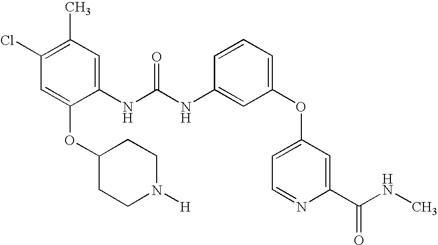
4-(3-{3-[4-Chloro-5-methyl-2-(piperidin-4-yloxy)-phenyl]-ureido}-phenoxy)-pyridine-2-carboxylic acidmethylamide (MW=509.99; Rt=2.17); 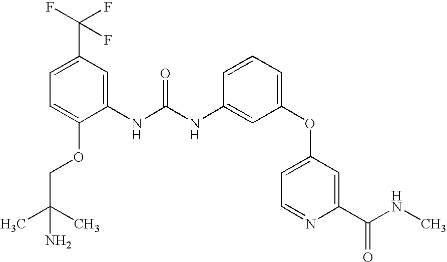
4-(3-{3-[(2-Amino-2-methyl-propoxy)-trifluoromethyl-phenyl]-ureido}-phenoxy)-pyridine-2-carboxylic acid methylamide (MW=517.51; Rt=2.17); 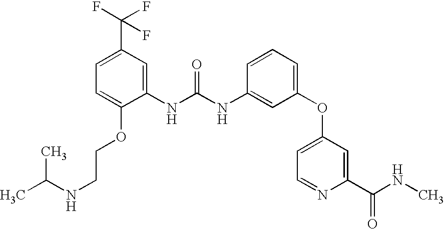
4-(3-{3-[(2-Isopropylamino-ethoxy)-trifluoromethyl-phenyl]-ureido}-phenoxy)-pyridine-2-carboxylic acid methylamide (MW=531.53; Rt=2.21); 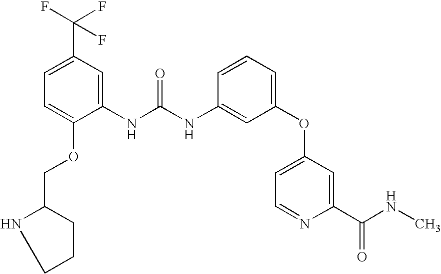
4-(3-{3-[(Pyrrolidin-2-ylmethoxy)-trifluoromethyl-phenyl]-ureido}-phenoxy)-pyridine-2-carboxylic acid methylamide (MW=529.52; Rt=2.22); 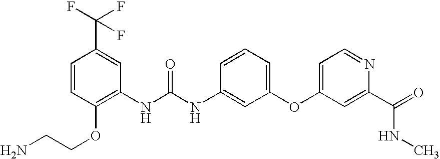
4-(3-{3-[(2-Amino-ethoxy)-trifluoromethyl-phenyl]-ureido}-phenoxy)-pyridine-2-carboxylic acid methylamide (MW=489.45; Rt=2.15);
   and the pharmaceutically acceptable derivatives, solvates, salts and stereoisomers thereof, including mixtures thereof in all ratios, and more preferred the salts and/or solvates thereof, and especially preferred the physiologically acceptable salts and/or solvates thereof.

Further especially preferred as compounds according to the invention are the compounds given below: 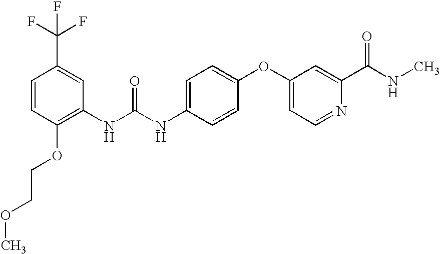
4-(4-{3-[2-(2-Methoxy-ethoxy)-5-trifluoromethyl-phenyl]-ureido}-phenoxy)-pyridine-2-carboxylic acid methylamide 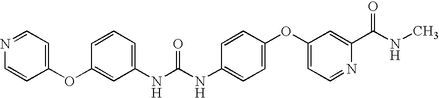
4-(4-{3-[3-(Pyridin-4-yloxy)-phenyl]-ureido}-phenoxy)-pyridine-2-carboxylic acid methylamide
   and the pharmaceutically acceptable derivatives, solvates, salts and stereoisomers thereof, including mixtures thereof in all ratios, and more preferred the salts and/or solvates thereof, and especially preferred the physiologically acceptable salts and/or solvates thereof.

Further especially preferred as compounds according to the invention are the compounds given below: 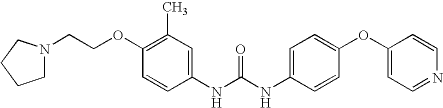
1-[3-Methyl-4-(2-pyrrolidin-1-yl-ethoxy)-phenyl]-3-[4-(pyridin-4-yloxy)-phenyl]-urea 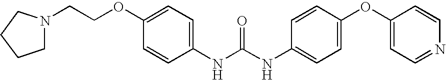
1-[4-(Pyridin-4-yloxy)-phenyl]-3-[4-(2-pyrrolidin-1-yl-ethoxy)-phenyl]-urea 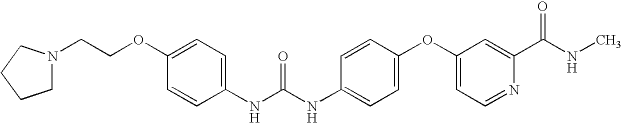
4-(4-{3-[4-(2-Pyrrolidin-1-yl-ethoxy)-phenyl]-ureido}-phenoxy)-pyridine-2-carboxylic acid methylamide 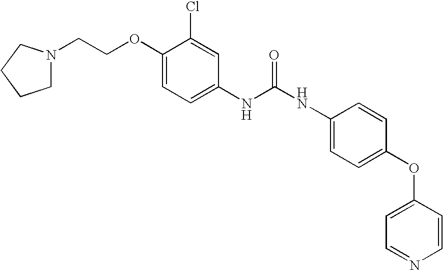
1-[3-Chloro-4-(2-pyrrolidin-1-yl-ethoxy)-phenyl]-3-[4-(pyridin-4-yloxy)-phenyl]-urea 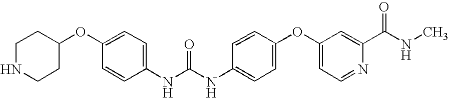
4-(4-{3-[4-(Piperidin-4-yloxy)-phenyl]-ureido}-phenoxy)-pyridine-2-carboxylic acid methylamide 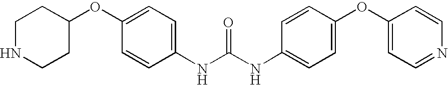
1-[4-(Piperidin-4-yloxy)-phenyl]-3-[4-(pyridin-4-yloxy)-phenyl]-urea 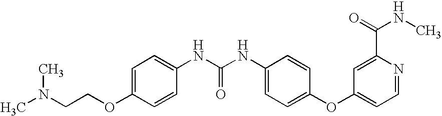
4-(4-{3-[4-(2-Dimethylamino-ethoxy)-phenyl]-ureido}-phenoxy)-pyridine-2-carboxylic acid methylamide 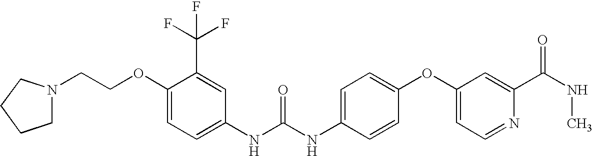
4-(4-{3-[4-(2-Pyrrolidin-1-yl-ethoxy)-3-trifluoromethyl-phenyl]-ureido}-phenoxy)-pyridine-2-carboxylic acid methylamide 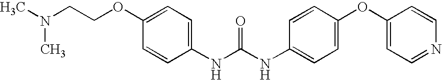
1-[4-(2-Dimethylamino-ethoxy)-phenyl]-3-[4-(pyridin-4-yloxy)-phenyl]-urea 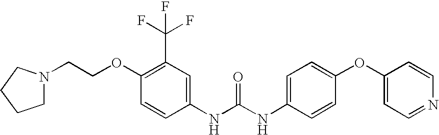
1-[4-(Pyridin-4-yloxy)-phenyl]-3-[4-(2-pyrrolidin-1-yl-ethoxy)-3-trifluoromethyl-phenyl]-urea 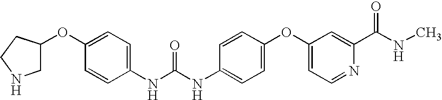
4-(4-{3-[4-(Pyrrolidin-3-yloxy)-phenyl]-ureido}-phenoxy)-pyridine-2-carboxylic acid methylamide 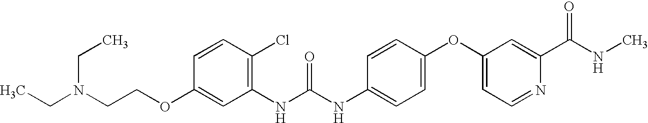
4-(4-{3-[2-Chloro-5-(2-diethylamino-ethoxy)-phenyl]-ureido}-phenoxy)-pyridine-2-carboxylic acid methylamide 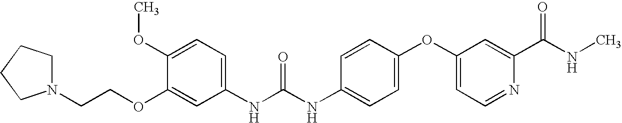
4-(4-{3-[4-Methoxy-3-(2-pyrrolidin-1-yl-ethoxy)-phenyl]-ureido}-phenoxy)-pyridine-2-carboxylic acid methylamide 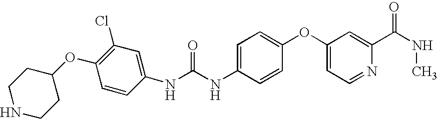
4-(4-{3-[3-Chloro-4-(piperidin-4-yloxy)-phenyl]-ureido}-phenoxy)-pyridine-2-carboxylic acid methylamide 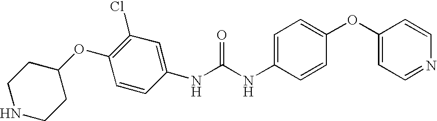
1-[3-Chloro-4-(piperidin-4-yloxy)-phenyl]-3-[4-(pyridin-4-yloxy)-phenyl]-urea 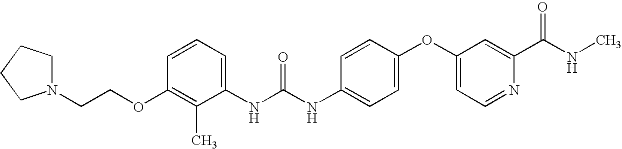
4-(4-{3-[2-Methyl-3-(2-pyrrolidin-1-yl-ethoxy)-phenyl]-ureido}-phenoxy)-pyridine-2-carboxylic acid methylamide 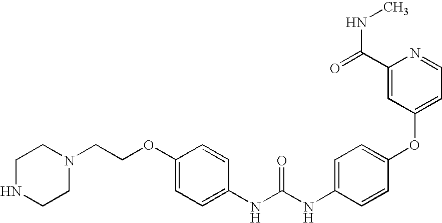
4-(4-{3-[4-(2-piperazin-1-yl-ethoxy)-phenyl]-ureido}-phenoxy)-pyridine-2-carboxylic acid methylamide 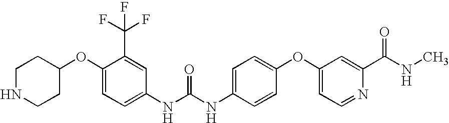
4-(4-{3-[4-(Piperidin-4-yloxy)-3-trifluoromethyl-phenyl]-ureido}-phenoxy)-pyridine-2-carboxylic acid methylamide 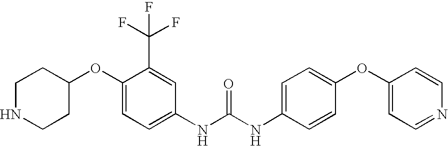
1-[4-(Piperidin-4-yloxy)-3-trifluoromethyl-phenyl]-3-[4-(pyridin-4-yloxy)-phenyl]-urea 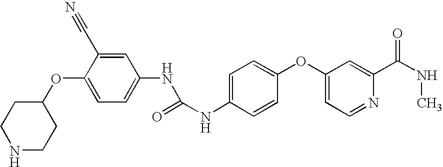
4-(4-{3-[3-Cyano-4-(piperidin-4-yloxy)-phenyl]-ureido}-phenoxy)-pyridine-2-carboxylic acid methylamide
   and the pharmaceutically acceptable derivatives, solvates, salts and stereoisomers thereof, including mixtures thereof in all ratios, and more preferred the salts and/or solvates thereof, and especially preferred the physiologically acceptable salts and/or solvates thereof.

Further especially preferred as compounds according to the invention are the compounds given below: 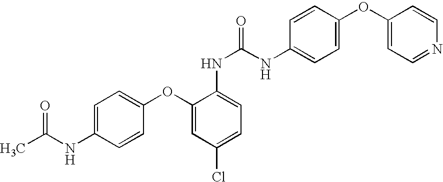
N-[4-(5-Chloro-2-{3-[4-(pyridin-4-yloxy)-phenyl]-ureido}-phenoxy)-phenyl]-acetamide 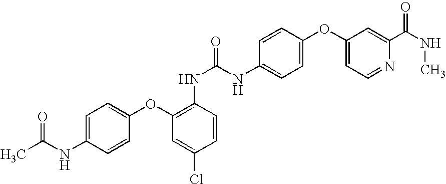
4-(4-{3-[2-(4-Acetylamino-phenoxy)-4-chloro-phenyl]-ureido}-phenoxy)-pyridine-2-carboxylic acid methylamide 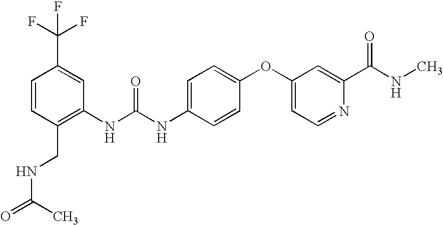
4-(4-{3-[2-(Acetylamino-methyl)-5-trifluoromethyl-phenyl]-ureido}-phenoxy)-pyridine-2-carboxylic acid methylamide 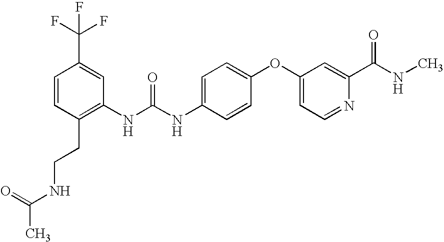
4-(4-{3-[2-(2-Acetylamino-ethyl)-5-trifluoromethyl-phenyl]-ureido}-phenoxy)-pyridine-2-carboxylic acid methylamide
   Amide: 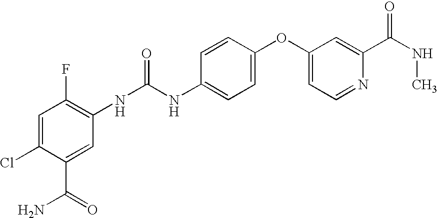
4-{4-[3-(5-Carbamoyl-4-chloro-2-fluoro-phenyl)-ureido]-phenoxy}-pyridine-2-carboxylic acid methylamide 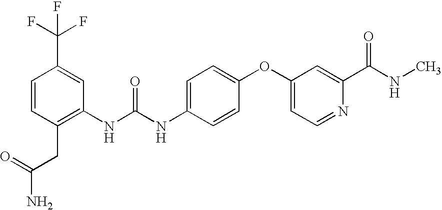
4-{4-[3-(2-Carbamoylmethyl-5-trifluoromethyl-phenyl)-ureido]-phenoxy}-pyridine-2-carboxylic acid methylamide 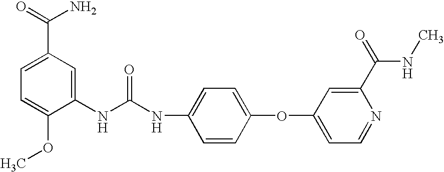
4-{4-[3-(5-Carbamoyl-2-methoxy-phenyl)-ureido]-phenoxy}-pyridine-2-carboxylic acid methylamide 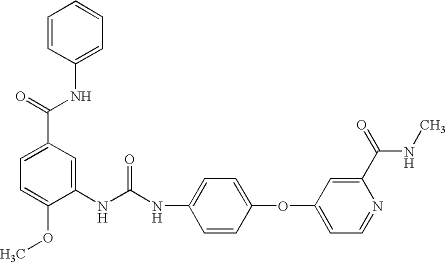
4-{4-[3-(2-Methoxy-5-phenylcarbamoyl-phenyl)-ureido]-phenoxy}-pyridine-2-carboxylic acid methylamide
   and the pharmaceutically acceptable derivatives, solvates, salts and stereoisomers thereof, including mixtures thereof in all ratios, and more preferred the salts and/or solvates thereof, and especially preferred the physiologically acceptable salts and/or solvates thereof.

Further especially preferred as compounds according to the invention are the compounds given below: 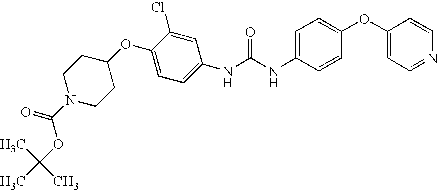
4-(2-Chloro-4-{3-[4-(pyridin-4-yloxy)-phenyl]-ureido}-phenoxy)-piperidine-1-carboxylic acid tert-butyl ester 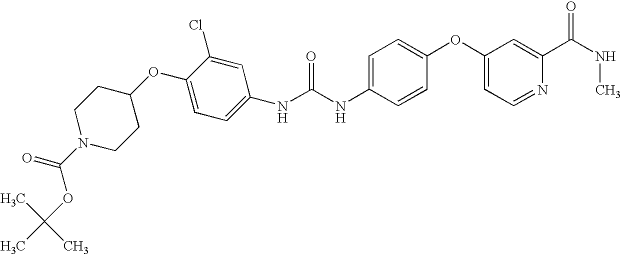
4-(2-Chloro-4-{3-[4-(2-methylcarbamoyl-pyridin-4-yloxy)-phenyl]-ureido}-phenoxy)-piperidine-1-carboxylic acid tert-butyl ester 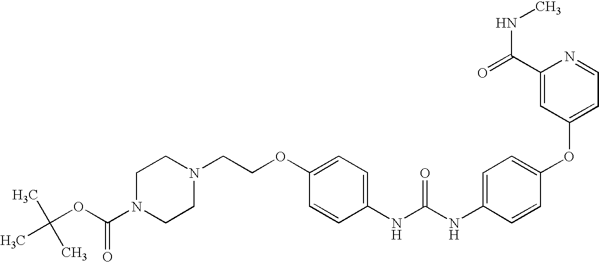
4-[2-(4-{3-[4-(2-Methylcarbamoyl-pyridin-4-yloxy)-phenyl]-ureido}-phenoxy)-ethyl]-piperazine-1-carboxylic acid tert-butyl ester 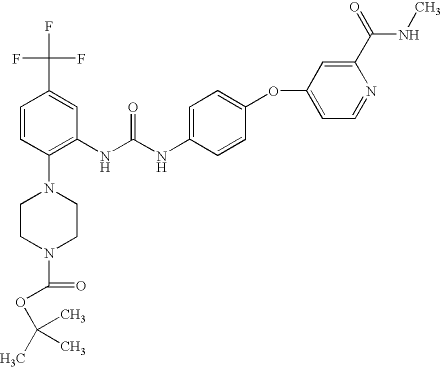
4-(2-{3-[4-(2-Methylcarbamoyl-pyridin-4-yloxy)-phenyl]-ureido}-4-trifluoromethyl-phenyl)-piperazine-1-carboxylic acid tert-butyl ester 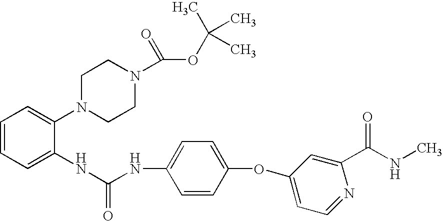
4-(2-{3-[4-(2-Methylcarbamoyl-pyridin-4-yloxy)-phenyl]-ureido}-phenyl)-piperazine-1-carboxylic acid tert-butyl ester
   and the pharmaceutically acceptable derivatives, solvates, salts and stereoisomers thereof, including mixtures thereof in all ratios, and more preferred the salts and/or solvates thereof, and especially preferred the physiologically acceptable salts and/or solvates thereof.

Further especially preferred as compounds according to the invention are the compounds given below: 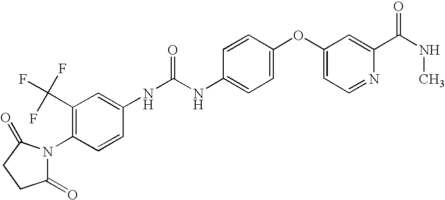
4-(4-{3-[4(2,5-Dioxo-pyrrolidin-1-yl)-3-trifluoromethyl-phenyl]-ureido}-phenoxy)-pyridine-2-carboxylic acid methylamide 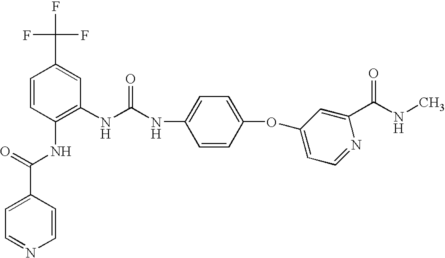
4-[4-(3-{2-[Pyridine-4-carbonyl)-amino]-5-trifluoromethyl-phenyl}-ureido)-phenoxy]-pyridine-2-carboxylic acid methylamide 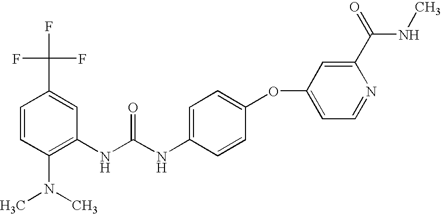
4-{4-[3-(2-Dimethylamino-5-trifluoromethyl-phenyl)-ureido]-phenoxy}-pyridine-2-carboxylic acid methylamide 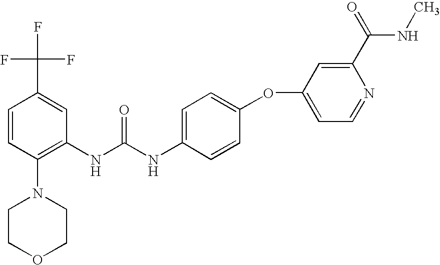
4-{4-[3-(2-Morpholin-4-yl-5-trifluoromethyl-phenyl)-ureido]-phenoxy}-pyridine-2-carboxylic acid methylamide 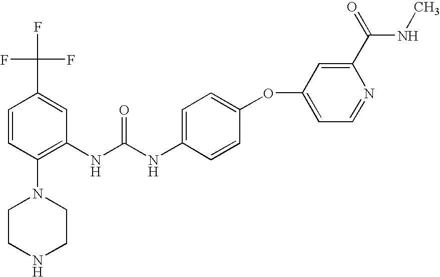
4-{4-[3-(2-piperazin-1-yl-5-trifluoromethyl-phenyl)-ureido]-phenoxy}-pyridine-2-carboxylic acid methylamide 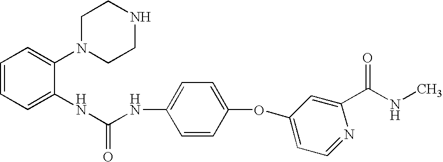
4-{4-[3-(2-piperazin-1-yl-phenyl)-ureido]-phenoxy}-pyridine-2-carboxylic acid methylamide
   and the pharmaceutically acceptable derivatives, solvates, salts and stereoisomers thereof, including mixtures thereof in all ratios, and more preferred the salts and/or solvates thereof, and especially preferred the physiologically acceptable salts and/or solvates thereof.

Further especially preferred as compounds according to the invention are the compounds given below: 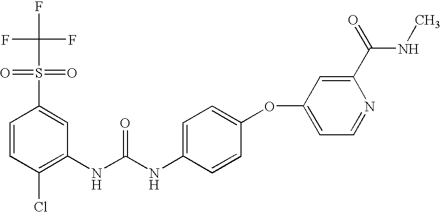
4-{4-[3-(2-Chloro-5-trifluoromethanesulfonyl-phenyl)-ureido]-phenoxy}-pyridine-2-carboxylic acid methylamide 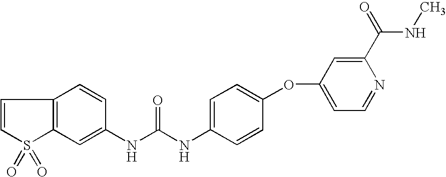
4-{4-[3-(1,2-Dioxo-1H-1I6-benzo[b]thiophen-6-yl)-ureido]-phenoxy}-pyridine-2-carboxylic acid methylamide 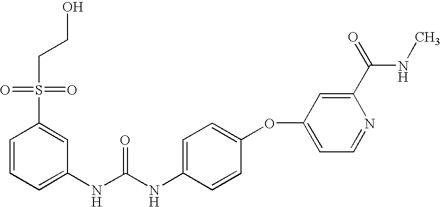
4-(4-{3-[3-(2-Hydroxy-ethanesulfonyl)-phenyl]-ureido}-phenoxy)-pyridine-2-carboxylic acid methylamide 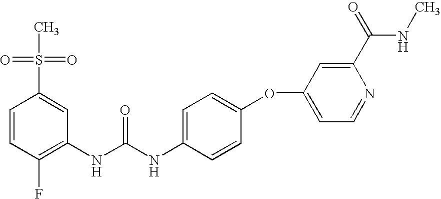
4-{4-[3-(2-Fluoro-5-methanesulfonyl-phenyl)-ureido]-phenoxy}-pyridine-2-carboxylic acid methylamide 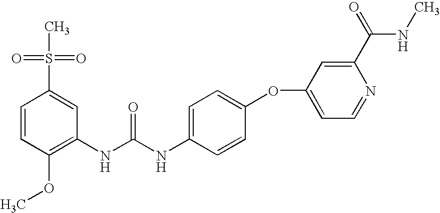
4-{4-[3-(5-Methanesulfonyl-2-methoxy-phenyl)-ureido]-phenoxy}-pyridine-2-carboxylic acid methylamide 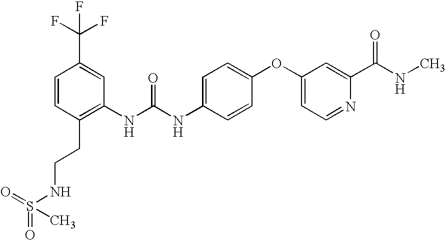
4-(4-{3-[2-(2-Methanesulfonylamino-ethyl)-5-trifluoromethyl-phenyl]-ureido}-phenoxy)-pyridine-2-carboxylic acid methylamide 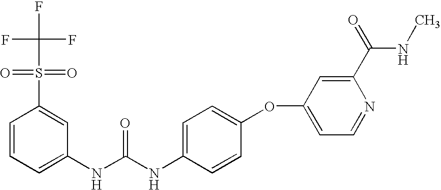
4-{4-[3-(3-Trifluoromethanesulfonyl-phenyl)-ureido]-phenoxy}-pyridine-2-carboxylic acid methylamide 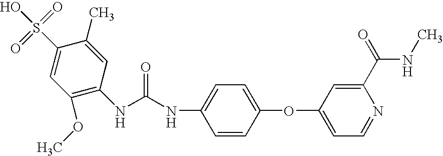
5-Methoxy-2-methyl-4-{3-[4-(2-methylcarbamoyl-pyridin-4-yloxy)-phenyl]-ureido}-benzenesulfonic acid
   and the pharmaceutically acceptable derivatives, solvates, salts and stereoisomers thereof, including mixtures thereof in all ratios, and more preferred the salts and/or solvates thereof, and especially preferred the physiologically acceptable salts and/or solvates thereof.

Further especially preferred as compounds according to the invention are the compounds given below: 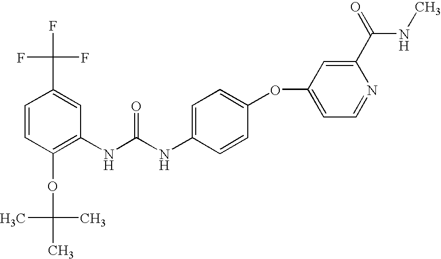
4-{4-[3-(2-tert-Butoxy-5-trifluoromethyl-phenyl)-ureido]-phenoxy}-pyridine-2-carboxylic acid methylamide 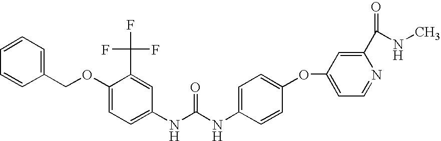
4-{4-[3-(4-Benzyloxy-3-trifluoromethyl-phenyl)-ureido]-phenoxy}-pyridine-2-carboxylic acid methylamide 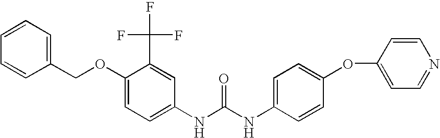
1-(4-Benzyloxy-3-trifluoromethyl-phenyl)-3-[4-(pyridin-4-yloxy)-phenyl]-urea
   and the pharmaceutically acceptable derivatives, solvates, salts and stereoisomers thereof, including mixtures thereof in all ratios, and more preferred the salts and/or solvates thereof, and especially preferred the physiologically acceptable salts and/or solvates thereof.

Further especially preferred as compounds according to the invention are the compounds given below: 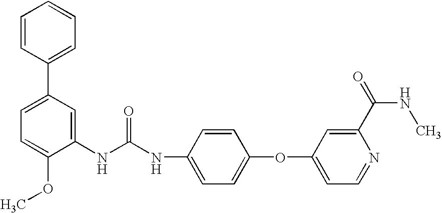
4-{4-[3-(4-Methoxy-biphenyl-3-yl)-ureido]-phenoxy}-pyridine-2-carboxylic acid methylamide 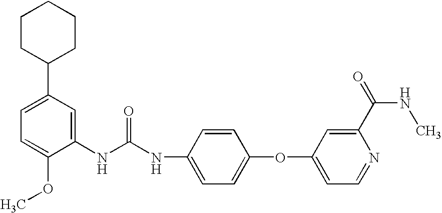
4-{4-[3-(5-Cyclohexyl-2-methoxy-phenyl)-ureido]-phenoxy}-pyridine-2-carboxylic acid methylamide 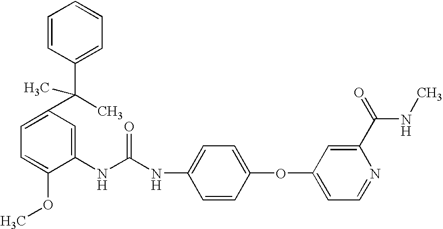
4-(4-{3-[2-Methoxy-5-(1-methyl-1-phenyl-ethyl)-phenyl]-ureido}-phenoxy)-pyridine-2-carboxylic acid methylamide 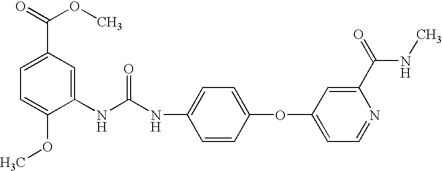
4-Methoxy-3-{3-[4-(2-methylcarbamoyl-pyridin-4-yloxy)-phenyl]-ureido}-benzoic acid methyl ester
   and the pharmaceutically acceptable derivatives, solvates, salts and stereoisomers thereof, including mixtures thereof in all ratios, and more preferred the salts and/or solvates thereof, and especially preferred the physiologically acceptable salts and/or solvates thereof.

Further especially preferred as compounds according to the invention are the compounds given below: 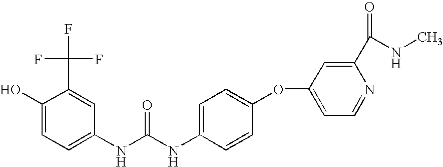
4-{4-[3-(4-Hydroxy-3-trifluoromethyl-phenyl)-ureido]-phenoxy}-pyridine-2-carboxylic acid methylamide 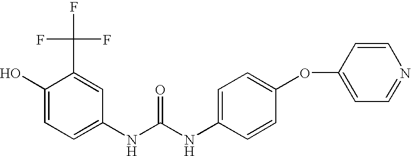
1-(4-Hydroxy-3-trifluoromethyl-phenyl)-3-[4-(pyridine-4-yloxy)-phenyl]-urea 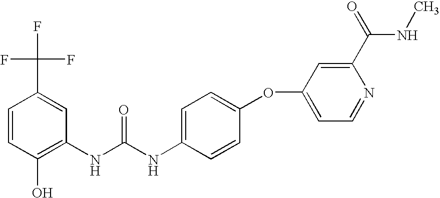
4-{4-[3-(2-Hydroxy-5-trifluoromethyl-phenyl)-ureido]-phenoxy}-pyridine-2-carboxylic acid methylamide
   and the pharmaceutically acceptable derivatives, solvates, salts and stereoisomers thereof, including mixtures thereof in all ratios, and more preferred the salts and/or solvates thereof, and especially preferred the physiologically acceptable salts and/or solvates thereof.

The nomenclature as used herein for defining compounds, especially the compounds according to the invention, is in general based on the rules of the IUPAC-organisation for chemical compounds and especially organic compounds.

Another aspect of the invention relates to a method for producing compounds of formula I, characterised in that
a) a compound of formula II, 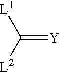
   wherein
   L¹ and L² either independently from one another represent a leaving group, or together represent a leaving group, and Y is as defined above/below,
   is reacted with
b) a compound of formula III 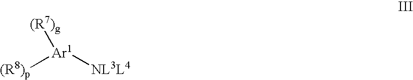
   wherein
   L³ and L⁴ are independently from one another H or a metal ion, and wherein R⁷, R⁸, g, p and Ar¹ are as defined above and below,
   and
c) a compound of formula IV, 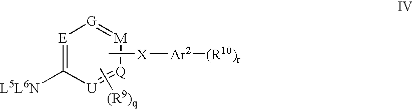
   wherein
   L⁵ and L⁶ are independently from one another H or a metal ion, and E, G, M, Q, U, R⁹, q, X, Ar², R¹⁰ and r are as defined above and below,
   and optionally
d) isolating and/or treating the compound of formula I obtained by said reaction within acid, to obtain the salt thereof.

The compounds of the formula I and also the starting materials for their preparation can be prepared by methods known per se, i.e. as described in the literature (for example in the standard works, such as Houben-Weyl, Methoden der organischen Chemie [Methods of Organic Chemistry], Georg-Thieme-Verlag, Stuttgart), to be precise under reaction conditions which are known and suitable for the said reactions. Use can also be made here of variants which are known per se, but are not mentioned here in greater detail.

If desired, the starting materials can also be formed in situ by not isolating them from the reaction mixture, but instead immediately converting them further into the compounds of the formula I. On the other hand, it is possible to carry out the reaction stepwise.

The compounds according to the invention can be manufactured or produced in an advantageous manner according to the methods of manufacture as described herein.

The reaction for the manufacture of compounds of formula I as described herein can be characterised as a carbonylation reaction of amines or the reaction of amines with carbon dioxide, carbon disulphide or derivatives or analogues thereof.

According to one aspect of the method according to the invention, in the compounds of formula II, L¹ and L² are preferably selected independently from one another from suitable leaving groups. Suitable leaving groups L¹ and L² for this type of reaction are known in the art, for example from the literature cited above. More preferably, L¹ and L² are independently selected from halogen, OR²⁵ and O—SO₂—R²⁵. The residue R²⁵ is preferably selected from substituted or unsubstituted alkyl groups and substituted or unsubstituted aryl groups, preferably substituted alkyl groups and substituted aryl groups. Preferred as alkyl groups in this respect are C1-C4-alkyl groups. Preferred as aryl group in this respect is phenyl. Suitable substituents for substituted alkyl groups are preferably selected from electronegative and/or electron withdrawing groups. Examples of electronegative and/or electron withdrawing groups for substituted alkyl groups include, but are not limited to halogen, especially Cl and/or F, cyano groups and nitro groups. Suitable substituents for substituted aryl groups are preferably selected from alkyl groups, preferably C₁-C₄ alkyl groups, and electronegative and/or electron withdrawing groups. Examples of electronegative and/or electron withdrawing groups for substituted aryl groups include, but are not limited to halogen, especially Cl and/or F, cyano groups and nitro groups. If R²⁵ is an unsubstituted alkyl group, it is preferably methyl. If R²⁵ his a substituted alkyl group, it is preferably CF₃ or CCl₃. If R²⁵ is an unsubstituted aryl group, it is preferably phenyl. If R²⁵ is a substituted aryl group, it is preferably selected from para-tolyl- (i.e. p-Me-C₆H₄) and para-Nitro-phenyl (i.e the p-O₂N—C₆H₄).

Even more preferably, the leaving groups OR²⁵ are selected from the para-Tosyl- (i.e. p-Me-C₆H₄—SO₃—) group, the para-Nitro-phenolate- (i.e the p-O₂N—C₆H₄—O—) group and the triflate- (i.e. the F₃C—SO₃—) group. Preferably, compounds of formula II, wherein L¹ and L² are selected independently from one another from suitable leaving groups, are selected from compounds IIa, IIb and IIc, 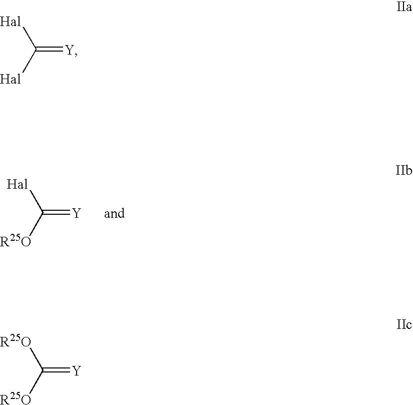
wherein Y, Hal and OR²⁵ are as described above/below.

According to another aspect of the method according to the invention, in the compounds of formula II, L¹ and L² together represent a leaving group. In this aspect, L¹ and L² together preferably represent Y as the leaving group, wherein the leaving group Y is as defined above/below and more preferably is O or S.

According to this aspect of the method according to the invention, the compound of formula II is a compound of formula II′,
Y═C═Y II′
wherein each Y is independently selected from the meaning given above/below, and especially is independently selected from O and S.

According to this aspect of the method according to the invention, the compound of formula II is preferably selected from compounds of formula IId, formula IIe and formula IIf, 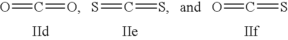
more preferably of compounds of formula IId and formula IIe. In this aspect, compounds of formula IIa are especially preferred.

In compounds of formula II, Y is preferably selected from O and S, and more preferably is O.

If compounds of formula II are desired wherein Y is other than 0, it can be advantageous however to carry out the reaction according to the invention selecting a compound of formula II wherein Y is O, and to modify or convert the corresponding C═O group (i.e. the C═Y group, wherein Y is O) in the compound of formula I into a C═NR²¹, C═C(R²²)—NO₂, C═C(R²²)—CN or C═C(CN)₂ group according to methods known in the art, for example from Houben-Weyl, Methods of Organic Chemistry.

In the method of manufacture according to the invention, the compound of formula II is even more preferably a compound of formula IIg, 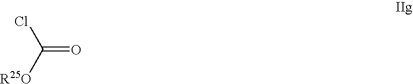
wherein R²⁵ is as defined above/below, and especially a compound of formula IIh,

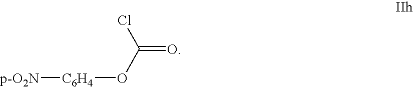

In the compounds of formula IV, L¹, L² and/or L³ is preferably H or a moiety which activates the amino group it is bonded to, for example a metal ion. Suitable metal ions are preferably selected from the group consisting of alkaline metal ions, alkaline-earth metal ions and aluminium ions. Especially preferred metal ions are alkaline metal ions, of which Li, Na K are especially preferred. In case of multi-valent metal ions, the metal ions and the compounds of formula IV form a complex containing one or more compounds of formula IV and one or more metal ions wherein the ratio between compounds of formula IV and metal ions is depending on the valency of the metal ion(s) according to the rules of stoichiometry and/or electroneutrality. Preferably, at least one of L¹, L² and L³, more preferred at least two of L¹, L² and L³ and even more preferred L¹, L² and L³ are hydrogen.

In detail, the reaction of the compounds of formula II, formula II and formula IV is carried out in the presence or absence of a preferably inert solvent at temperatures between about −20° C. and about 200° C., preferably between −10° C. and 150° C. and especially between 0° C. or room temperature (25°) and 120°. In many cases, it is advantageous to combine one compound of formula III with one compound of formula IV at the lower end of the given temperature range, preferably between −20° C. and 75° C., more preferred between 0° C. and 60° C. and especially between 10° C. and 40° C., for example at about room temperature, and heat the mixture up to a temperature at the upper end of the given temperature range, preferably between 65° C. and 180° C., more preferred between 75° C. and 150° C. and especially between 80° C. and 120° C., for example at about 80° C., at about 90° C. or at about 100° C. Proceeding in that manner can be advantageous in the case that pound of formula II is the compounds of formula II′. If the compound of formula II is not a compound of formula II′, the reaction can be regularly carried out without prolonged heating to higher temperatures. For example, it can preferably be carried out at a temperature between −10° C. and 60° C., more preferably between −5° C. and 40° C. and even more preferably at about 0° C. or at about room temperature. This given temperature range is especially advantageous, if the compound of formula II is selected from compounds of formula IIa, IIb, IIc and especially is a compound of formula IIg or IIh.

The method for manufacture according to the invention is preferably carried out in the presence of an acid binding means, for example one or more bases. This is especially advantageous, if the compound of formula II is selected from compounds of formula IIa-IIc an even preferred if the compound is selected from the compounds of formula IIg or formula IIh.

Suitable acid binding means are known in the art. Preferred as acid binding means are inorganic bases and especially organic bases. Examples for inorganic bases are alkaline or alkaline-earth hydroxides, alkaline or alkaline-earth carbonates and alkaline or alkaline-earth bicarbonates or other salts of a weak acid and alkaline or alkaline-earth metals, preferably of potassium, sodium, calcium or cesium. Examples for organic bases are triethyl amine, diisopropyl ethyl amine (DIPEA), diaza bicyclo undecen (DBU), dimethyl aniline, pyridine or chinoline. If an organic base is used, it is advantageous in general to use a base with a boiling point that is higher than the highest reaction temperature employed during the reaction. Especially preferred as organic bases are pyridine and DIPEA. In many cases it is advantageous to employ two different organic bases and especially to use pyridine and DIPEA.

Reaction times are generally in the range between some minutes and several days, depending on the reactivity of the respective compounds and the respective reaction conditions. Suitable reaction times are readily determinable by methods known in the art, for example reaction monitoring. Based on the reaction temperatures given above, suitable reaction times generally lie in the range 10 min and 36 hrs, preferably 30 min and 24 hrs and especially between 45 min and 18 hrs, for example about 1 h, about 2 hrs, about 4 hrs, about 6 or about 18 hrs.

Preferably, the reaction of the compounds of the formula III with the compounds of the formula IV is carried out in the presence of a suitable solvent, that is preferably inert under the respective reaction conditions. Examples of suitable solvents are hydrocarbons, such as hexane, petroleum ether, benzene, toluene or xylene; chlorinated hydrocarbons, such as trichlorethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether or ethylene glycol dimethyl ether (diglyme); ketones, such as acetone or butanone; amides, such as acetamide, dimethylacetamide, dimethylformamide (DMF) or N-methyl pyrrolidinone (NMP); nitriles, such as acetonitrile; sulfoxides, such as dimethyl sulfoxide (DMSO); nitro compounds, such as nitromethane or nitrobenzene; esters, such as ethyl acetate, or mixtures of the said solvents. Polar solvents are in general preferred. Examples for suitable polar solvents are chlorinated hydrocarbons, alcohols, glycol ethers, nitriles, amides and sulfoxides or mixtures thereof. More preferred are chlorinated hydrocarbons, especially dichloromethane, and amides, especially DMF.

In general, the compounds of formula III and/or formula IV are new. In any case, they can be prepared according to methods known in the art.

The compounds of formula III can be obtained according to methods known in the art. In an advantageous manner, they can be readily obtained by one or more of the reaction routes given below:

Compounds of formula III can be readily obtained from synthesis sequence as given below:

The reaction of derivatives of formula (A) 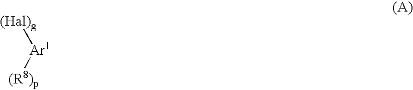
wherein Hal is Cl, Br or F and especially is F, and wherein g, R⁸, p and Ar¹ are as defined above/below, with compounds of formula (B)
L⁷-R⁷ (B)
wherein R⁷ is as defined above/below and L⁷ is preferably selected from H or a metal ion, if L⁷ is bound to an oxygen atom of R⁷ or to an nitrogen atom of R⁷, or selected from carbon atom activating groups, if L⁷ is bound to a carbon atom of R⁷, leads to compounds of formula (C).

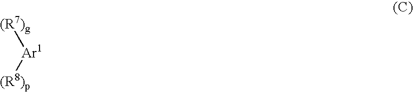

Suitable carbon atom activating groups for this type of reaction are known in the art. Suitable metal ions are preferably selected from the group consisting of alkaline metal ions, alkaline-earth metal ions and aluminium ions. Preferred metal ions are alkaline metal ions, of which Li, Na and/or K are especially preferred. Even more preferred as L⁷ is H.

Accordingly, preferred compounds of formula (B) for the method for manufacture according to the invention are compounds that comprise a hydroxy-group, a primary amino group or a secondary amino group. Thus, especially preferred are compounds of formula (B), that comprise an HO—, a H₂N-group, a HNR¹¹-group or a HNR¹²-group, and especially compounds that comprise a terminal HO—, a H₂N-group, a HNR¹¹-group or a HNR¹²-group, wherein R¹¹ and R¹² are as defined above/below.

This type of reaction is generally known as aromatic substitution. Suitable reaction conditions for the reaction of the compounds of formula (A) with the compounds of formula (B) are known in the art.

The compounds of formula (B) are preferably selected from HHet, HOHet, HN(R¹¹)Het, O(CR⁵R⁶)ₖHet, HN(R¹¹)(CR⁵R⁶)ₖHet, (CR⁵R⁶)ₖNR¹¹R¹², (CR⁵R⁶)ₖOR¹³, HO(CR⁵R⁶)ₖNR¹¹R¹², HNR¹¹(CR⁵R⁶)ₖNR¹¹R¹², HO(CR⁵R⁶)ₖR¹³, HNR¹¹(CR⁵R⁶)ₖR¹³, HO(CR⁵R⁶)ₖOR¹³, HNR¹¹(CR⁵R⁶)ₖOR¹³, HO(CR⁵R⁶)ₙO(CR⁵R⁶)ₖNR¹¹R¹², HNR¹¹(CR⁵R⁶)ₙO(CR⁵R⁶)ₖNR¹¹R¹², HO(CR⁵R⁶)ₙNR¹¹(CR⁵R⁶)ₖNR¹¹R¹², HNR¹¹(CR⁵R⁶)ₙNR¹¹(CR⁵R⁶)ₖNR¹¹R¹², HO(CR⁵R⁶)ₙO(CR⁵R⁶)ₖOR¹¹, HNR¹¹(CR⁵R⁶)ₙO(CR⁵R⁶)ₖOR¹², HO(CR⁵R⁶)ₙNR¹¹(CR⁵R⁶)ₖOR¹² and HNR¹²(CR⁵R⁶)ₙNR¹¹ (CR⁵R⁶)ₖOR¹², and the metal salts thereof.

If the compounds of formula (B) comprise more than one hydroxy group, primary amino group or secondary amino group (apart from the hydroxy group or amino group comprising L⁷), it is advantageous to proceed the reaction using derivatives of compounds of formula (B), wherein the additional hydroxy groups, primary amino groups or secondary amino groups are protected by so-called protecting groups, i.e. hydroxy protecting groups or amino protecting groups, respectively. Accordingly, if the compounds of formula I are to carry residues R⁷ comprising one or more of R¹¹, R¹², R¹³ and R¹⁴ that are H, it is advantageous to employ compounds of formula (B), wherein these H-atoms are replaced by suitable protecting groups.

Suitable protecting groups are known in the art. For example, primary amino groups can be advantageously protected as phthalimides, secondary amino groups can be advantageously protected with the BOC-protecting group. Suitable methods and reaction conditions for producing protected derivatives of compounds of formula (B) and methods and reaction conditions for removing such protection groups from the accordingly obtained protected products are known in the art.

The compound of formula (C) then can be transferred into the compound of formula III by methods known in the art.

Advantageously, the compound of formula (C) then can be transferred into a compound of formula (D), 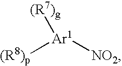
by a nitration reaction. Suitable methods and reaction conditions for nitration reactions are known in the art. Advantageously, the compounds of formula (D) can be obtained by reacting a compound of formula (C) with nitrating acid or a combination of concentrated sulfuric acid and potassium nitrate. If a combination of concentrated sulfuric acid and potassium nitrate is used, it can be advantageous to perform the reaction at a relatively low temperature, for example between −20° C. and +50° C., preferably between −10° C. and room temperature, more preferred between −5° C. and 0° C.

The compound of formula (D) then can be transferred into a compound of formula III, wherein L³ and L⁴ are hydrogen, preferably by a reduction reaction or hydrogenating reaction, preferably a hydrogenating reaction. Methods and reaction conditions for hydrogenating a NO₂-moiety into a NH₂-moiety are known in the art. In general, it is advantageous to carry out the hydrogenation reaction in a hydrogen atmosphere in the presence of a suitable catalyst, for example Pd/C or Raney-nickel, preferably Raney-nickel. In general, such hydrogenation reactions are carried out in a suitable solvent. Suitable solvents for hydrogenation reactions are known in the art. Suitable solvents, for example, are alcohols, especially methanol and ethanol and ethers, especially THF, and mixtures thereof. Preferred as solvent is a mixture of THF/methanol, preferably in about equal measures. In general, the hydrogenation reactions are carried out at about normal pressure or slightly elevated pressure, for example between normal pressure and 3 bar pressure (about 300 kPa). The hydrogenation reaction is usually carried out in the temperature range between −20° and 150°, preferably 0° and 50°. The obtained compound of formula III wherein L³ and L⁴ are hydrogen can optionally be isolated and/or purified and then optionally transferred into a compound of formula III wherein L³ and L⁴ are other than hydrogen, for example according to methods and reaction conditions as described herein.

Some of the starting materials of the formula V and/or the formula VI are known and preferably commercially available. If they are not known, they can be prepared by methods known per se.

Generally, the compounds of formula IV can be obtained according to methods known in the art.

If the compound of formula IV is a compound according to formula IVa, 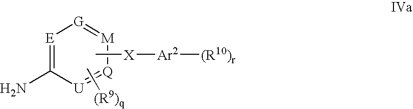
it can be readily obtained in an advantageous manner by reacting a compound of formula VIIa, 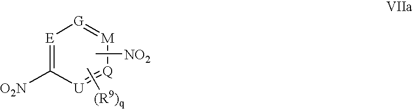
wherein R⁹ and q are as defined above/below,
with a compound of formula VIII,
L⁸-X—Ar²—(R¹⁰)ᵣ VIII
wherein L⁸ is H or a metal ion, preferably a metal ion selected from the group consisting of alkaline metal ions, alkaline-earth metal ions and aluminum ions, especially preferred alkaline metal ions, of which Li, Na and K are especially preferred, and even more preferred is H; and Ar², R¹⁰, r and X are as defined above/below, and especially wherein X is (CHR¹¹)ₕ-Q-(CHR¹²)ᵢ, wherein R¹¹, h and R¹² are defined above/below, i is 0 and Q is selected from a group consisting of O, S, N—R¹⁵, (CHR¹⁸—O)ⱼ, (CHR¹⁸CHR¹⁹—O)ⱼ, CH═N—O, CH═N—NR¹⁵, SO₂NR¹⁵, wherein R¹⁵, R¹⁸ and R¹⁹ are as defined above/below;
optionally isolating the reaction product,
and transferring the obtained reaction product of formula IX 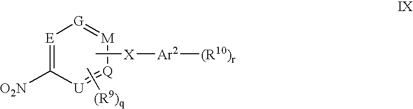
into a compound of formula IVa, preferably by hydrogenating the NO₂-moiety of the compound of formula IX into a NH₂-moiety. Methods and reaction conditions for hydrogenating said NO₂-moiety into a NH₂-moiety are known in the art. In general, it is advantageous to carry out the hydrogenation reaction in a hydrogen atmosphere in the presence of a suitable catalyst, preferably a Palladium catalyst, for example Pd/C. In general, such hydrogenation reactions are carried out in a suitable solvent. Suitable solvents for hydrogenation reactions are known in the art. Suitable solvents, for example, are alcohols, especially methanol and ethanol and ethers, especially THF, and mixtures thereof. In general, the hydrogenation reactions are carried out at about normal pressure or slightly elevated pressure, for example between normal pressure and 3 bar pressure (about 300 kPa). The hydrogenation reaction is usually carried out in the temperature range between −20° and 150°, preferably 0° and 50°.

Ar² is preferably pyridinyl. Accordingly, the compound of formula VIII is preferably selected from the group consisting of formulae VIIIa and VIIIb, 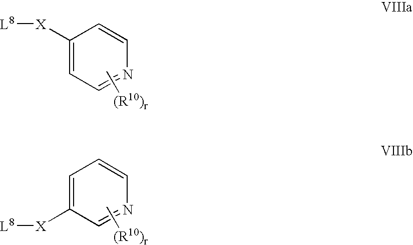
wherein L⁸, X, R¹⁰ and r are as defined above, and especially preferred from the group consisting of formulae VIIIc and VIIId, 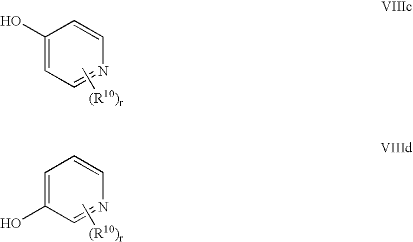
wherein R¹⁰ and r are as defined above, or the alkaline metal salts and especially the sodium or potassium salts thereof.

Accordingly, in formulae IVa, VIII, VIIIa, VIIIb and IX, the bridging group X is preferably O, S, OCH₂ and OCH₂CH₂ and especially is O.

In the formulae VIII, VIIIa and VIIIb, L⁸ is preferably H or selected from the group consisting of Na, K and Cs and especially preferred is H.

In general, this reaction is advantageous to produce compounds of formula IVaa, 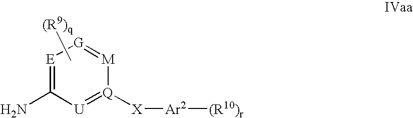
wherein R⁹, q, X, Ar², R¹⁰ and r are as defined above/below.

To obtain compounds of formula IVaa, it is reasonable to employ a compound of formula VII that is selected from the compounds of formula VIIIa, 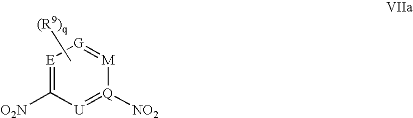
and proceed the reaction as described above/below.

Accordingly, by starting from a compound of formula VIIIa and a compound of formula VIIIa, the reaction preferably leads to compounds of formula IVaaa, 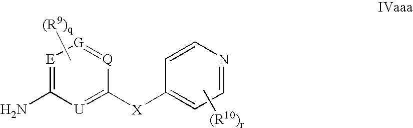
wherein R⁹, q, X, R¹⁰ and r are as defined above/below.

Accordingly, by starting from a compound of formula VIIIa and a compound of formula VIIIb, the reaction preferably leads to compounds of formula IVaab, 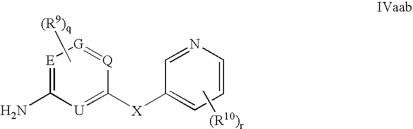
wherein R⁹, q, X, R¹⁰ and r are as defined above/below.

Accordingly, by starting from a compound of formula VIIIa and a compound of formula VIIIc, the reaction preferably leads to compounds of formula IVaac, 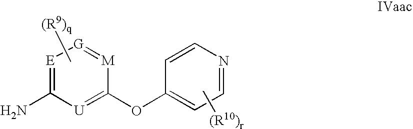
wherein R⁹, q, R¹⁰ and r are as defined above/below.

Accordingly, by starting from a compound of formula VIIa and a compound of formula VIIId, the reaction preferably leads to compounds of formula 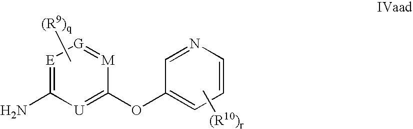
wherein R⁹, q, R¹⁰ and r are as defined above/below.

Some of the starting materials of the formula VII and/or the formula VIII are known and preferably commercially available. If they are not known, they can be prepared by methods known per se.

The reaction between the compound of formula VII and VIII is preferably carried out in the temperature range between 0° and 250°, more preferred room temperature and 200°, for example at about 120°, at about 150° or at about 180°. Reaction times depend on the respective reactants and the respective reaction temperature, but generally lie in the range between 30 min and 36 hrs, preferably 3 hrs and 24 hrs, more preferably 8 hrs and 20 hrs for example about 10 hrs, about 16 hrs or about 18 hrs.

The reaction can be carried out in the absence of solvent or preferably in the presence of an solvent, preferable a solvent that is inert under the respective reaction conditions. Suitable inert solvents for carrying out the reaction are known in the art. Examples for suitable solvents are high boiling aliphatic hydrocarbons, high boiling aromatic carbons, for example toluene, xylenes, high boiling chlorinated hydrocarbons, such as trichloroethylene, tetrachloroethanes, pentachloroethanes and hexachloroethanes; high boiling ethers, such as ethylene glycol and propylene glycols; glycol ethers, such as ethylene glycol monomethyl or monoethyl ether or ethylene glycol dimethyl ether (diglyme); amides, such as acetamide, dimethylacetamide, dimethylformamide (DMF) or N-methylpyrrolidinone (NMP); sulfoxides, such as dimethyl sulfoxide (DMSO); or mixtures of the said solvents. Preferred are amides, especially dimethylformamide (DMF).

Preferably, the reaction is carried out in the presence of a base. Suitable bases are known in the art. Preferred bases are organic bases and especially inorganic bases. Examples for inorganic bases are alkaline or alkaline-earth hydroxides, alkaline or alkaline-earth carbonates and alkaline or alkaline-earth bicarbonates or other salts of a weak acid and alkaline or alkaline-earth metals, preferably of potassium, sodium, calcium or cesium. Preferred inorganic bases are K₂CO₃, Na₂CO₃, MgCO₃, CaCO₃, NaOH and KOH, especially preferred is K₂CO₃. Examples for organic bases are triethyl amine, diisopropyl ethyl amine (DIPEA), dimethyl aniline, pyridine or chinoline. If an organic base is used, it is advantageous in general to use a base with a boiling point that is higher than the highest reaction temperature employed during the reaction.

Alternatively, if the compound of formula IV is a compound according to formula IVb, 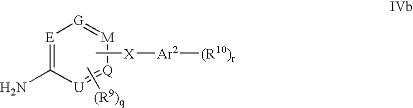
it can be readily obtained in an advantageous manner by reacting a compound of formula VIIb, 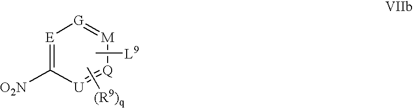
wherein R⁹ and q are as defined above/below and wherein L⁹ is selected independently from the meanings given for L¹. Preferably, L⁹ is halogen. More preferred, L⁹ is selected from the group consisting of Cl, Br and I. Especially preferred, L⁹ is Cl.
with a compound of formula VIIIb,
L¹⁰-X—Ar²—(R¹⁰)ᵣ VIIIb
wherein L¹⁰ is H or a metal ion, preferably a metal ion, more preferred a metal ion selected from the group consisting of alkaline metal ions, alkaline-earth metal ions and aluminium ions, especially preferred alkaline metal ions, of which Li, Na and K are especially preferred; and Ar², R¹⁰, r and X are as defined above/below, and especially wherein X is (CHR¹¹)ₕ-Q-(CHR¹²)ᵢ, CH═N—O, CH═N—NR¹⁵, SO₂NR¹⁵, wherein R¹⁵, R¹⁸ and R¹⁹ are as defined above/below;
optionally isolating the reaction product,
and transferring the obtained reaction product of formula IXb 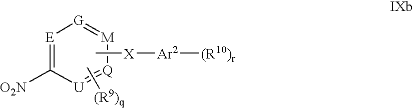
into a compound of formula IVa, preferably by hydrogenating the NO₂-moiety of the compound of formula IX into a NH₂-moiety. Methods and reaction conditions for hydrogenating said NO₂-moiety into a NH₂-moiety are known in the art. In general, it is advantageous to carry out the hydrogenation reaction in a hydrogen atmosphere in the presence of a suitable catalyst, preferably a Palladium catalyst, for example Pd/C. In general, such hydrogenation reactions are carried out in a suitable solvent. Suitable solvents for hydrogenation reactions are known in the art. Suitable solvents, for example, are alcohols, especially methanol and ethanol, ethers, especially THF, and mixtures thereof. In general, the hydrogenation reactions are carried out at about normal pressure or slightly elevated pressure, for example between normal pressure or slightly elevated pressure, for example between normal pressure and 3 bar pressure (about 300 kPa). The hydrogenation reaction is usually carried out in the temperature range between −20° and 150°, preferably 0° and 50°.

Ar² is preferably pyridinyl. Accordingly, the compound of formula VIIIb is preferably selected from the group consisting of formulae VIIIe and VIIIf, 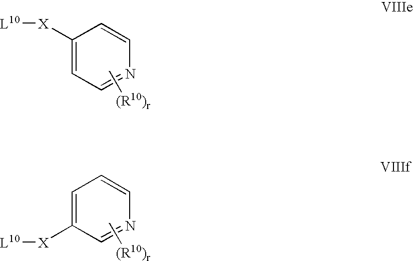
wherein L¹⁰, X, R¹⁰ and r are as defined above, and especially preferred from the group consisting of formulae VIIIg and VIIIh, 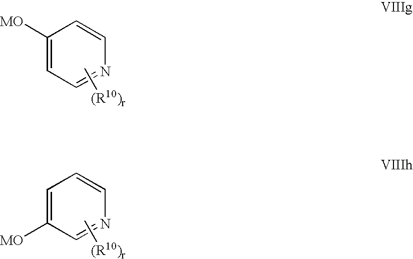
wherein R¹⁰ and r are as defined above, and wherein M is an alkaline metal ion and especially sodium or potassium, or the corresponding alcohols thereof.

Accordingly, in formulae IVb, VIIIb, VIIIe, VIIIf and IXb, the bridging group X is preferably O, S, OCH₂ and OCH₂CH₂ and especially is O.

In general, this alternative reaction is advantageous to produce compounds of formula IVbb, 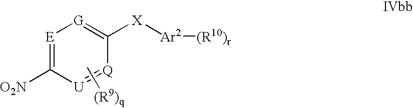
wherein R⁹, q, X, Ar², R¹⁰ and r are as defined above/below.

To obtain compounds of formula IVbb, it is reasonable to employ a compound of formula VIIb that is selected from the compounds of formula VIIbb, 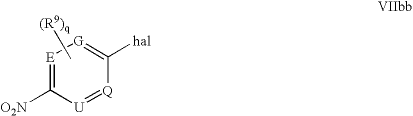
wherein hal is as defined above/below and especially is Cl, and proceed the alternative reaction as described above/below.

Accordingly, by starting from a compound a formula VIIbb and a compound of formula VIIe, the reaction preferably leads to compounds of formula IVbbe, 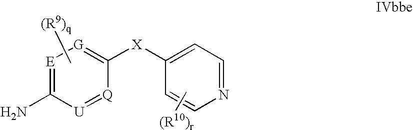
wherein R⁵⁹, q, X, R¹⁰ and r are as defined above/below.

Accordingly, by starting from a compound of formula VIIbb and a compound of formula VIIIf, the reaction preferably leads to compounds of formula IVbbf, 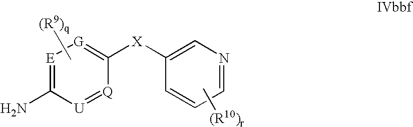
wherein R¹⁹, q, X, R¹⁰ and r are as defined above/below.

Accordingly, by starting from a compound of formula VIIbb and a compound of formula VIIIg, the reaction preferably leads to compounds of formula IVbbg, 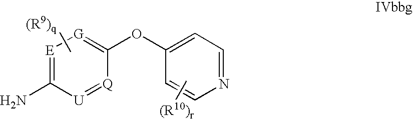
wherein R⁹, q, R¹⁰ and r are as defined above/below.

Accordingly, by starting from a compound of formula VIIb and a compound of formula VIIIh, the reaction preferably leads to compounds of formula IVbbh, 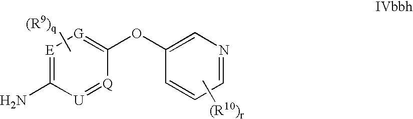
wherein R⁹, q, R¹⁰ and r are as defined above/below.

Some of the starting materials of the formula VIIb and/or the formula VIIIb are known and preferably commercially available. If they are not known, they can be prepared by methods known per se.

The reaction between the compound of formula VIIb and VIIIb is preferably carried out in the temperature range between 0° and 250°, more preferred 50° and 220°, for example at about 90°, at about 120°, at about 160°, at about 180° or at about 200°. Reaction times depend on the respective reactants and the respective reaction temperature, but generally lie in the range between 10 min and 24 hrs, preferably 30 min and 12 hrs, more preferably 1 h and 6 hrs for example about 1.5 hrs, about 3 hrs, about 4 hrs or about 5 hrs.

The reaction can be carried out in the absence or the presence of a solvent, preferable a solvent that is inert under the respective reaction conditions. Suitable inert solvents for carrying out the reaction are known in the art. Examples for suitable solvents are high boiling aliphatic hydrocarbons, aromatic carbons, for example toluene and xylenes, high boiling chlorinated hydrocarbons, such as dichloromethane, trichloromethane trichloroethylene, tetrachloroethanes, pentachloroethanes and hexachloroethanes; ethers, such as diethylether, tert.-butyl methyl ether, ethylene glycol and propylene glycols; glycol ethers, such as ethylene glycol monomethyl or monoethyl ether or ethylene glycol dimethyl ether (diglyme); nitriles, such as acetonitrile, amides such as acetamide, dimethyacetamide, dimethylformamide (DMF) or N-methylpyrrolidinone (NMP); sulfoxides, such as dimethyl sulfoxide (DMSO); or mixtures of the said solvents.

Preferably, the reaction is carried out in the presence of a catalyst. Suitable catalysts are known in the art. Preferred are catalytic active metals and especially copper.

Preferably, the reaction is carried out by heating up a reaction mixture comprising one compound of formula VIIb and one compound of formula VIIIb to a suitable reaction temperature, which preferably lies at the upper end of the given temperature ranges and more preferred is in the range between 150° and 200°, for example at about 180°, preferably in the presence of the suitable catalyst and especially in the presence of copper. Reaction times at this temperature are preferably as given above and especially in the range between 1 h and 5 hrs, for example about 3 hrs. Preferably, the reaction mixture is then allowed to cool down to a temperature in the lower range of the given temperature, more preferred to a temperature in the range between 50° and 150°, for example to about 90°. Preferably, a suitable solvent, preferably tert.-butyl methyl ether, is then added and the reaction mixture is preferably kept at about the same temperature for some more time, preferably for 30 min to 2 hrs and more preferred for about one hour.

If the compound IV is a compound according to formula IVc, 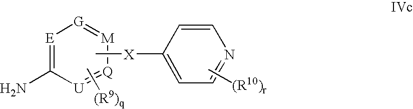
it can be readily obtained in an advantageous manner by reacting a compound of formula XI 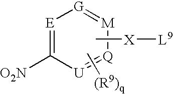
wherein L⁹ is H or a metal ion, preferably a metal ion selected from the group consisting of alkaline metal ions, alkaline-earth metal ions and aluminium ions, especially preferred alkaline metal ions, of which Li, Na and K are especially preferred, and even more preferred is H; and R⁹, q and X are as defined above/below, and especially wherein X is (CHR¹¹)ₕ-Q-(CHR¹²)ᵢ, wherein R¹¹, h and R¹² are defined above/below, i is O and Q is selected from a group consisting of O, S, N—R¹⁵, (CHR¹⁸—O)ⱼ, (CHR¹⁸CHR¹⁹—O)ⱼ, CH═N—O, CH═N—NR¹⁵, SO₂NR¹⁵, wherein R¹⁵, R¹⁸ and R¹⁹ are as defined above/below;
with a compound of formula XII, 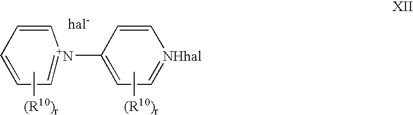
wherein hal is independently select selected from the group consisting of Cl, Br and I, the residues R¹⁰ are the same or different and have the meanings given above/below and preferably have both the same meaning, and the indices r are the same or different and have the meanings given above/below and preferably are the same,
optionally isolating the reaction product, and transferring the obtained reaction product of formula XIII 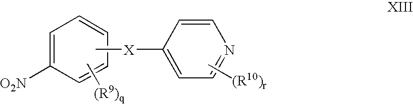
into a compound of formula IVc, preferably by hydrogenating the NO₂-moiety of the compound of formula XIII into a NH₂-moiety, for example as described above for the compound of formula IX.

In the compounds IVc, XII and XIII, r is preferably in each case identical and even more preferred in each case 0.

In formulae IVc, XI and XIII, the bridging group X is preferably O, S, OCH₂ and OCH₂CH₂ and especially is O.

In the formula XI, L⁹ is preferably H or selected from the group consisting of Na and K, and especially preferred is H.

The reaction between the compound of formula XI and XII is preferably carried out in the temperature range between 0° and 250°, more preferred room temperature and 200°, for example at about 120°, at about 150° or at about 180°. Reaction times depend on the respective reactants and the respective reaction temperature, but generally lie in the range between 30 min and 24 hrs, preferably one hour and 12 hrs, for example about 2 hrs, about 3 hrs or about 6 hrs. The reaction can be carried out in the absence of solvent or in the presence of an solvent, preferable a solvent that is inert under the respective reaction conditions. Suitable inert solvents for carrying out the reaction are known in the art.

Some of the starting materials of the formula XI and/or the formula XIII are known and preferably commercially available. If they are not known, they can be prepared by methods known per se.

Independently of the chosen reaction route, it is in many cases possible or even feasible to introduce residues R⁷, R⁸, R⁹ and/or R¹⁰ into one or more of the compounds described above, or, if the compound already comprises one or more residues R⁷, R⁸, R⁹ and/or R¹⁰, to introduce additional residues R⁷, R⁸, R⁹ and/or R¹⁰ into said compound. The introduction of additional residues can be readily performed by methods known in the art and especially by aromatic substitution, for example nucleophilic aromatic substitution or electrophilic aromatic substitution. For example, in compounds comprising Ar¹, wherein Ar¹ comprises one or more halogen and preferably fluorine substituents, one or more of the halogen/fluorine substituents can be easily substituted by hydroxy, thio and/or amino substituted hydrocarbons, preferably selected from the group consisting of HO(CH₂)ₖNR¹¹R¹², HO(CH₂)ₖR¹³, HO(CH₂)ₖOR¹¹, HO(CH₂)ₙO(CH₂)ₖNR¹¹R¹², HO(CH₂)ₙNR¹¹(CH₂)ₖOR¹², HO(CH₂)ₙNR¹¹(CH₂)ₖNR¹¹R¹², HO(CH₂)ₙCOOR¹³, HO(CH₂)ₙS(O)R¹³, HNR¹¹(CH₂)ₖNR¹¹R¹², HNR¹¹(CH₂)ₖOR¹¹, HNR¹¹(CH₂)ₙO(CH₂)ₖNR¹¹R¹², HNR¹¹(CH₂)ₙNR¹¹(CH₂)ₖOR¹², HNR¹¹(CH₂)ₙNR¹¹(CH₂)ₖNR¹¹R¹², HNR¹¹(CH₂)ₙCOOR¹² and HNR¹¹(CH₂)ₙS(O)ᵤR¹³, and the metal salts thereof, wherein R¹¹, R¹² and R¹³ are defined as above and n is as defined above, preferably n is 0, 1 or 2 and especially is 0, k is 1 to 4 and preferably 1 or 2, and u is preferably 2. Even more preferred, the hydroxy, thio and/or amino substituted hydrocarbons are selected from the group consisting of NH₃, HN(CH₃)₂, NH₂CH₃, HN(C₂H₅)₂, H₂NCH₂CH₂NH₂, HOCH₂CH₂NH₂, HOCH₂CH₂NHCH₃, HN(CH₃)CH₂CH₂NH₂, HN(CH₃)CH₂CH₂N(CH₃)₂, HN(CH₃)CH₂CH₂N(CH₃)₂, HN(CH₃)CH₂CH₂OCH₃, HOCH₂CH₂N(CH₃)₂, HOCH₂CH₂N(CH₂CH₃)₂, HSCH₃, HSC₂H₅, and compounds of the formulae 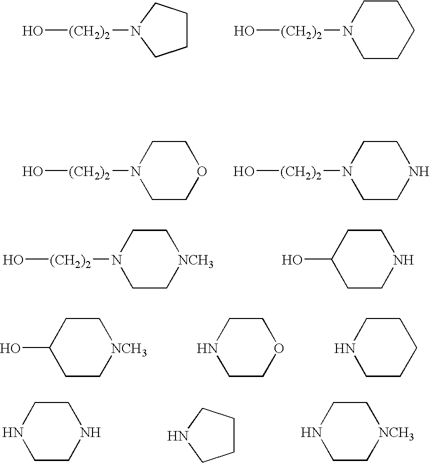
or salts and especially metal salts thereof.

On the other hand, it is in many cases possible or even feasible to modify or derivatize one or more of the residues R⁷, R⁸, R⁹ and/or R¹⁰ into residues R⁷, R⁸, R⁹ and/or R¹⁰ other than the ones originally present. For example, CH₃-groups can be oxidized into aldehyde groups or carboxylic acid groups, thio atom containing groups, for example S-alkyl or S-aryl groups, can be oxidized into SO₂-alkyl or SO₂-aryl groups, respectively, carboxylic acid groups can be derivatized to carboxylic acid ester groups or carboxylic acid amide groups and carboxylic acid ester groups or carboxylic acid amide groups can be hydrolysed into the corresponding carboxylic acid groups. Methods for performing such modifications or derivatizations are known in the art, for example from Houben-Weyl, Methods of Organic Chemistry.

Every reaction step described herein can optionally be followed by one or more working up procedures and/or isolating procedures. Suitable such procedures are known in the art, for example from standard works, such as Houben-Weyl, Methoden der organischen Chemie [Methods of Organic Chemistry], Georg-Thieme-Verlag, Stuttgart). Examples for such procedures include, but are not limited to evaporating a solvent, distilling, crystallization, fractionised crystallization, extraction procedures, washing procedures, digesting procedures, filtration procedures, chromatography, chromatography by HPLC and drying procedures, especially drying procedures in vacuo and/or elevated temperature.

A base of the formula I can be converted into the associated acid-addition salt using an acid, for example by reaction of equivalent amounts of the base and the acid in a preferably inert solvent, such as ethanol, followed by evaporation. Suitable acids for this reaction are, in particular, those which give physiologically acceptable salts. Thus, it is possible to use inorganic acids, for example sulfuric acid, sulfurous acid, dithionic acid, nitric acid, hydrohalic acids, such as hydrochloric acid or hydrobromic acid, phosphoric acids, such as, for example, orthophosphoric acid, sulfamic acid, furthermore organic acids, in particular aliphatic, alicyclic, araliphatic, aromatic or heterocyclic monobasic or polybasic carboxylic, sulfonic or sulfuric acids, for example formic acid, acetic acid, propionic acid, hexanoic acid, octanoic acid, decanoic acid, hexadecanoic acid, octadecanoic acid, pivalic acid, diethylacetic acid, malonic acid, succinic acid pimelic acid, fumaric acid, maleic acid, lactic acid, tartaric acid, malic acid, citric acid, gluconic acid, ascorbic acid, nicotinic acid, isonicotinic acid, methane- or ethanesulfonic acid, ethanedisulfonic acid, 2-hydroxyethanesulfonic acid, benzenesulfonic acid, trimethoxybenzoic acid, adamantanecarboxylic acid, p-toluenesulfonic acid, glycolic acid, embonic acid, chlorophenoxyacetic acid, aspartic acid, glutamic acid, proline, glyoxylic acid, palmitic acid, parachlorophenoxyisobutyric acid, cyclohexanecarboxylic acid, glucose 1-phosphate, naphthalenemono- and -disulfonic acids or laurylsulfuric acid. Salts with physiologically unacceptable acids, for example picrates, can be used to isolate and/or purify the compounds of the formula I. On the other hand, compounds of the formula I can be converted into the corresponding metal salts, in particular alkali metal salts or alkaline earth metal salts, or into the corresponding ammonium salts, using bases (for example sodium hydroxide, potassium hydroxide, sodium carbonate or potassium carbonate). Suitable salts are furthermore substituted ammonium salts, for example the dimethyl-, diethyl- and diisopropylammonium salts, monoethanol-, diethanol- and diisopropanolammonium salts, cyclohexyl- and dicyclohexylammonium salts, dibenzylethylenediammonium salts, furthermore, for example, salts with arginine or lysine.

On the other hand, if desired, the free bases of the formula I can be liberated from their salts using bases (for example sodium hydroxide, potassium hydroxide, sodium carbonate or potassium carbonate).

The invention relates to compounds of the formula I and physiologically acceptable salts and solvates thereof as medicaments.

The invention also relates to the compounds for the formula I and physiologically acceptable salts and solvates thereof as kinase inhibitors.

The invention furthermore relates to the use of the compounds of the formula I and/or physiologically acceptable salts and/or solvates thereof for the preparation of pharmaceutical compositions and/or pharmaceutical preparations, in particular by non-chemical methods. In this cases, one or more compounds according to the invention can be converted into a suitable dosage form together with at least one solid, liquid and/or semi-liquid excipient or adjuvant and, if desired, in combination with one or more further active ingredients.

The invention further relates to the use of one or more of the compounds according to the invention, selected from the group consisting of compounds of the formula I as free bases, solvates of compounds of the formula I, salts of compounds of formula I, for the production of pharmaceutical compositions and/or pharmaceutical preparations, in particular by a non-chemical route. In general, non-chemical routes for the production of pharmaceutical compositions and/or pharmaceutical preparations comprise processing steps on suitable mechanical means known in the art that transfer one or more compounds according to the invention into a dosage form suitable for administration to a patient in need of such a treatment. Usually, the transfer of one or more compounds according to the invention into such a dosage form comprises the addition of one or more compounds, selected from the group consisting of carriers, excipients, auxiliaries and pharmaceutical active ingredients other than the compounds according to the invention. Suitable processing steps include, but are not limited to combining, milling, mixing, granulating, dissolving, dispersing, homogenizing, casting and/or compressing the respective active and non-active ingredients. In this respect, active ingredients are preferably at least one compound according to this invention and one or more additional compounds other than the compounds according to the invention, which show valuable pharmaceutical properties, preferably those pharmaceutical active agents other than the compounds according to invention which are disclosed herein.

The process for preparing pharmaceutical compositions and/or pharmaceutical preparations preferably comprises one or more processing steps, selected from the group consisting of combining, milling, mixing, granulating, dissolving, dispersing, homogenizing and compressing. The one or more processing steps are preferably performed on one or more of the ingredients which are to form the pharmaceutical composition and/or pharmaceutical preparation preferably according to invention. Even more preferred, said processing steps are performed on two or more of the ingredients which are to form the pharmaceutical composition and/or pharmaceutical preparation, said ingredients comprising one or more compounds according to the invention and, additionally, one or more compounds, preferably selected from the group consisting of active ingredients other than the compounds according to the invention, excipients, auxiliaries, adjuvants and carriers. Mechanical means for performing said processing steps are known in the art, for example from Ullmann's Encyclopedia of Industrial Chemistry, 5th Edition.

Preferably, one or more compounds according to the invention are converted into a suitable dosage form together with at least one compound selected from the group consisting of excipients, auxiliaries, adjuvants and carriers, especially solid, liquid and/or semi-liquid excipients, auxiliaries, adjuvants and carriers, and, if desired, in combination with one or more further active ingredients.

Suitable dosage forms include, but are not limited to tablets, capsules, semi-solids, suppositories, aerosols, which can be produced according to methods known in the art, for example as described below:

| | |
|---|---|
| tablets | mixing of active ingredient/s and auxiliaries, |
| | compression of said mixture into tablets (direct |
| | compression), optionally granulation of part of |
| | mixture before compression |
| capsules | mixing of active ingredient/s and auxiliaries to |
| | obtain a flowable powder, optionally |
| | granulating powder, filling powders/granulate |
| | into opened capsules, capping of capsules |
| semi-solids (ointments, | dissolving/dispersing active |
| gels, creams) | ingredient/s in an aqueous or fatty carrier; |
| | subsequent mixing of aqueous/fatty phase |
| | with complementary fatty resp. aqueous |
| | phase, homogenisation (creams only) |
| suppositories (rectal | dissolving/dispersing active |
| and vaginal) | ingredient/s in carrier material liquified by heat |
| | (rectal: carrier material normally a wax; |
| | vaginal: carrier normally a heated solution of a |
| | gelling agent), casting said mixture into |
| | suppository forms, annealing and withdrawal |
| | suppositories from the forms |
| aerosols: | dispersing/dissolving active agent/s in a |
| | propellant, bottling said mixture into an |
| | atomizer |

The invention thus relates to pharmaceutical compositions and/or pharmaceutical preparations comprising at least one compound of the formula I and/or one of its physiologically acceptable salts and/or solvates.

Preferably, the pharmaceutical compositions and/or pharmaceutical preparations according to the invention contain a therapeutic effective amount of one or more compounds according to the invention. Said therapeutic effective amount of one or more of the compounds according to the invention is known to the skilled artisan or can be easily determined by standard methods known in the art. For example, the compounds according to the invention can be administered to a patient in an analogous manner to other compounds that are effective as raf-kinase inhibitors, especially in an analogous manner to the compounds described in WO 00/42012 (Bayer). Usually, suitable doses that are therapeutically effective lie in the range between 0.0005 mg and 1000 mg, preferably between 0.005 mg and 500 mg and especially between 0.5 and 100 mg per dose unit. The daily dose comprises preferably more than 0.001 mg, more preferred more than 0.01 milligram, even more preferred more than 0.1 mg and especially more than 1.0 mg, for example more than 2.0 mg, more than 5 mg, more than 10 mg, more than 20 mg, more than 50 mg or more than 100 mg, and preferably less than 1500 mg, more preferred less than 750 mg, even more preferred less than 500 mg, for example less than 400 mg, less than 250 mg, less than 150 mg, less than 100 mg, less than 50 mg or less than 10 mg.

The specific dose for the individual patient depends, however, on the multitude of factors, for example on the efficacy of the specific compounds employed, on the age, body weight, general state of health, the sex, the kind of diet, on the time and route of administration, on the excretion rate, the kind of administration and the dosage form to be administered, the pharmaceutical combination and severity of the particular disorder to which the therapy relates. The specific therapeutic effective dose for the individual patient can readily be determined by routine experimentation, for example by the doctor or physician which advises or attends the therapeutic treatment.

However, the specific dose for each patient depends on a wide variety of factors, for example on the efficacy of the specific compound employed, on the age, body weight, general state of health, sex, on the diet, on the time and method of administration, on the rate of excretion, medicament combination and severity of the particular illness to which the therapy applies. Parenteral administration is preferred. Oral administration is especially preferred.

These compositions and/or preparations can be used as medicaments in human or veterinary medicine. Suitable excipients are organic or inorganic substances which are suitable for enteral (for example oral), parenteral or topical administration and do not react with the novel compounds, for example water, vegetable oils, benzyl alcohols, alkylene glycols, polyethylene glycols, glycerol triacetate, gelatine, carbohydrates, such as lactose or starch, magnesium stearate, talc or vaseline. Examples for suitable dosage forms, which are especially suitable for oral administration are, in particular, tablets, pills, coated tablets, capsulees, powders, granules, syrups, juices or drops. Further examples for suitable dosage forms, which are especially suitable for rectal administration are suppositories, further examples for suitable dosage forms, which are especially suitable for parenteral administration are solutions, preferably oil-based or aqueous solutions, furthermore suspensions, emulsions or implants, and suitable for topical application are ointments, creams or powders. The novel compounds may also be lyophilised and the resultant lyophilisates used, for example, for the preparation of injection preparations. The compositions and/or preparations indicated may be sterilized and/or comprise assistants, such as lubricants, preservatives, stabilizers and/or wetting agents, emulsifiers, salts for modifying the osmotic pressure, buffer substances, dyes and flavors and/or one or more further active ingredients, for example one or more vitamins.

For administration as an inhalation spray, it is possible to use sprays in which the active ingredient is either dissolved or suspended in a propellant gas or propellant gas mixture (for example CO₂ or chlorofluorocarbons). The active ingredient is advantageously used here in micronized form, in which case one or more additional physiologically acceptable solvents may be present, for example ethanol. Inhalation solutions can be administered with the aid of conventional inhalers.

The compounds of the formula I and their physiologically acceptable salts and solvates can be employed for combating one or more diseases, for example allergic diseases, psoriasis and other skin diseases, especially melanoma, autoimmune diseases, such as, for example, rheumatoid arthritis, multiple sclerosis, Crohn's disease, diabetes mellitus or ulcerative colitis.

In General, the substances according to the invention are preferably administered in doses corresponding to the compound rolipram of between 1 and 500 mg, in particular between 5 and 100 mg per dosage unit. The daily dose is preferably between about 0.02 and 10 mg/kg of body weight. However, the specific dose for each patient depends on a wide variety of factors, for example on the efficacy of the specific compound employed, on the age, body weight, general state of health, sex, on the diet, on the time and method of administration, on the excretion rate, medicament combination and severity of the particular illness to which the therapy applies. Oral administration is preferred.

The compounds of the formula I according to claim 1 and/or their physiologically acceptable salts are also used in pathological processes which are maintained or propagated by angiogenesis, in particular in tumors, restenoses, diabetic retinopathy, macular degenerative disease or rheumatois arthritis.

Those of skill will readily appreciate that dose levels can vary as a function of the specific compound, the severity of the symptoms and the susceptibility of the subject to side effects. Some of the specific compounds are more potent than others. Preferred dosages for a given compound are readily determinable by those of skill in the art by a variety of means. A preferred means is to measure the physiological potency of a given compound.

For use in the subject methods, the subject compounds may be formulated with pharmaceutically active agents other than the compounds according to the invention, particularly other anti-metastatic, antitumor or anti-angiogenic agents. Angiostatic compounds of interest include angiostatin, enclostatin, carboxy terminal peptides of collagen alpha (XV), etc. Cytotoxic and cytostatic agents of interest include adriamycin, aleran, Ara-C, BICNU, busulfan, CNNU, cisplatinum, cytoxan, daunorubicin, DTIC, 5-FU, hydrea, ifosfamicle, methotrexate, mithramycin, mitomycin, mitoxantrone, nitrogen mustard, velban, vincristine, vinblastine, VP-16, carboplatinum, fludarabine, gemcitabine, idarubicin, irinotecan, leustatin, navelbine, taxol, taxotere, topotecan, etc.

The compounds of the invention have been shown to have antiproliferative effect in an in vivo xenograft tumor model. The subject compounds are administered to a subject having a hyperproliferative disorders, e.g., to inhibit tumor growth, to decrease inflammation associated with a lymphoproliferative disorder, to inhibit graft rejection, or neurological damage due to tissue repair, etc. The present compounds are useful for prophylactic or therapeutic purposes. As used herein, the term “treating” is preferably also used to refer to both prevention of disease, and treatment of pre-existing conditions. The prevention of proliferation is accomplished by administration of the subject compounds prior to development of overt disease, e.g., to prevent the regrowth of tumors, prevent metastatic growth, diminish restenosis associated with cardiovascular surgery, etc. Alternatively the compounds are used to treat ongoing disease, by stabilizing or improving the clinical symptoms of the patient.

The host, or patient, may be from any mammalian species, e.g., primate sp., particularly human; rodents, including mice, rats and hamsters; rabbits; equines, bovines, canines, felines; etc. Animal models are of interest for experimental investigations, providing a model for treatment of human disease.

The susceptibility of a particular cell to treatment with the subject compounds may be determined by in vitro testing. Typically a culture of the cell is combined with a subject compound at varying concentrations for a period of time sufficient to allow the active agents to induce cell death or inhibit migration, usually between about one hour and one week. For in vitro testing, cultured cells from a biopsy sample may be used. The viable cells left after treatment are then counted.

The dose will vary depending on the specific compound utilized, specific disorder, patient status, etc. Typically a therapeutic dose will be sufficient to substantially decrease the undesirable cell population in the targeted tissue, while maintaining patient viability. Treatment will generally be continued until there is a substantial reduction, e.g., at least about 50%, decrease in the cell burden, and may be continued until there are essentially none of the undesirable cells detected in the body.

The compounds according to the invention are preferably administered to human or nonhuman animals, more preferred to mammalian animals and especially to humans.

The compounds also find use in the specific inhibition of a signaling pathway mediated by protein kinases. Protein kinases are involved in signaling pathways for such important cellular activities as responses to extracellular signals and cell cycle checkpoints. Inhibition of specific protein kinases provided a means of intervening in these signaling pathways, for example to block the effect of an extracellular signal, to release a cell from cell cycle checkpoint, etc. Defects in the activity of protein kinases are associated with a variety of pathological or clinical conditions, where there is a defect in the signaling mediated by protein kinases. Such conditions include those associated with defects in cell cycle regulation or in response to extracellular signals, e.g., immunological disorders, autoimmune and immunodeficiency diseases; hyperproliferative disorders, which may include psoriasis, arthritis, inflammation, endometriosis, scarring, cancer, etc. The compounds of the present invention are active in inhibiting purified kinase proteins preferably raf kinases, e.g., there is a decrease in the phosphorylation of a specific substrate in the presence of the compound. The compounds of the invention may also be useful as reagents for studying signal transduction or any of the clinical disorders listed throughout this application.

There are many disorders associated with a dysregulation of cellular proliferation. The conditions of interest include, but are not limited to, the following conditions. The subject compounds are useful in the treatment of a variety of conditions where there is proliferation and/or migration of smooth muscle cells, and/or inflammatory cells into the intimal layer of a vessel, resulting in restricted blood flow through that vessel, e.g., neointimal occlusive lesions. Occlusive vascular conditions of interest include atherosclerosis, graft coronary vascular disease after transplantation, vein graft stenosis, peri-anastomatic prothetic graft stenosis, restenosis after angioplasty or stent placement, and the like.

Diseases where there is hyperproliferation and tissue remodelling or repair or reproductive tissue, e.g., uterine, testicular and ovarian carcinomas, endometriosis, squamous and glandular epithelial carcinomas of the cervix, etc. are reduced in cell number by administration of the subject compounds. The growth and proliferation of neural cells is also of interest.

Tumor cells are characterized by uncontrolled growth, invasion to surrounding tissues, and metastatic spread to distant sites. Growth and expansion requires an ability not only to proliferate, but also to down-modulate cell death (apoptosis) and activate angiogenesis to product a tumor neovasculature.

Tumors of interest for treatment include carcinomas, e.g., colon, duodenal, prostate, breast, melanoma, ductal, hepatic, pancreatic, renal, endometrial, stomach, dysplastic oral mucosa, polyposis, invasive oral cancer, non-small cell lung carcinoma, transitional and squamous cell urinary carcinoma etc.; neurological malignancies; e.g. neuroplastoma, gliomas, etc.; hematological malignancies, e.g., childhood acute leukaemia, non-Hodgkin's lymphomas, chronic lymphocytic leukaemia, malignant cutaneous T-cells, mycosis fungoides, non-MF cutaneous T-cell-lymphoma, lymphomatoid papulosis, T-cell rich cutaneous lymphoid hyperplasia, bullous pemphigoid, discoid lupus erythematosus, lichen planus, etc.; and the like.

Tumors of neural tissue are of particular interest, e.g., gliomas, neuromas, etc. Some cancers of particular interest include breast cancers, which are primarily adenocarcinoma subtypes. Ductal carcinoma in situ is the most common type of noninvasive breast cancer. In DCIS, the malignant cells have not metastasized through the walls of the ducts into the fatty tissue of the breast. Infiltration (or invasive) ductal carcinoma (IDC) has metastasized through the wall of the duct and invaded the fatty tissue of the breast. Infiltrating (or invasive) lobular carcinoma (ILC) is similar to IDC, in that it has the potential to metastasize elsewhere in the body. About 10% to 15% of invasive breast cancers are invasive lobular carcinomas.

Also of interest is non-small cell lung carcinoma. Non-small cell lung cancer (NSCLC) is made up of three general subtypes of lung cancer. Epidermoid carcinoma (also called squamos cell carcinoma) usually starts in one of the larger bronchial tubes and grows relatively slowly. The size of these tumors can range from very small to quite large. Adenocarcinoma starts growing near the outside surface of the lung and may vary in both size and growth rate. Some slowly growing adenocarcinomas are described as alveolar cell cancer. Large cell carcinoma starts near the surface of the lung, grows rapidly, and the growth is usually fairly large when diagnosed. Other less common forms of lung cancer are carcinoid, cylindroma, mucoepidermoid, and malignant mesothelioma.

Melanoma is a malignant tumor of melanocytes. Although most melanomas arise in the skin, they also may arise from mucosal surfaces or at other sites to which neural crest cells migrate. Melanoma occurs predominantly in adults, and more than half of the cases arise in apparently normal areas of the skin. Prognosis is affected by clinical and histological factors and by anatomic location of the lesion. Thickness and/or level of invasion of the melanoma, mitotic index, tumor infiltrating lymphocytes, and ulceration or bleeding at the primary site affect the prognosis. Clinical staging is based on whether the tumor has spread to regional lymph nodes or distant sites. For disease clinically confined to the primary site, the greater the thickness and depth of local invasion of the melanoma, the higher the chance of lymph node metastases and the worse the prognosis. Melanoma can spread by local extension (through lymphatics) and/or by hematogenous routes to distant sites. Any organ may be involved by metastases, but lungs and liver are common sites.

Other hyperproliferative diseases of interest relate to epidermal hyperproliferation, tissue, remodeling and repair. For example, the chronic skin inflammation of psoriasis is associated with hyperplastic epidermal keratinocyctes as well as infiltrating mononuclear cells, including CD4+ memory T cells, neutrophils and macrophages.

The proliferation of immune cells is associated with a number of autoimmune and lymphoproliferative disorders. Diseases of interest include multiple sclerosis, rheumatoid arthritis and insulin dependent diabetes mellitus. Evidence suggests that abnormalities in apoptosis play a part in the pathogenesis of systemic lupus erythematosus (SLE). Other lymphoproliferative conditions the inherited disorder of lymphocyte apoptosis, which is an autoimmune lymphoproliferative syndrome, as well as a number of leukemia's and lymphomas. Symptoms of allergies to environmental and food agents, as well as inflammatory bowel disease, may also be alleviated by the compounds of the invention.

Surprisingly, it has been found that bisarylurea derivatives according to invention are able to interact with signaling pathways, especially the signaling pathways described herein and preferably the raf-kinase signaling pathway. Bisarylurea derivatives according to the invention preferably show advantageous biological activity which can easily be demonstrated according to methods known in the art, for example by enzyme based assays. Suitable assays are known in the art, for example from the literature cited herein and the references cited in the literature, or can be developed and/or performed in an analogous manner thereof. In such enzyme based assays, bisarylurea derivatives according to invention show an effect, preferably a modulating and especially an inhibiting effect which is usually documented by IC₅₀ values in a suitable range, preferably in the micromolar range and more preferred in the nanomolar range.

In general, compounds according to the invention are to be regarded as suitable kinase-modulators and especially suitable kinase-inhibitors according to the invention if they show an effect or an activity to one or more kinases, preferably to one or more raf-kinases that preferably lies, determined as IC₅₀-value, in the range of 100 μmol or below, preferably 10 μmol or below, more preferably in the range of 3 μmol or below, even more preferably in the range of 1 μmol or below and most preferably in the nanomolar range. Especially preferred for use according to the invention are kinase-inhibitors as defined above/below, that show an activity, determined as IC₅₀-value, to one or more raf-kinases, preferably including A-raf, B-raf and c-raf1 or consisting of A-raf, B-raf and c-raf1 and more preferred including c-raf1 or consisting of c-raf1, in the range of 0.5 μmol or below and especially in the range of 0.1 μmol or below. In many cases an IC₅₀-value at the lower end of the given ranges is advantageous and in some cases it is highly desirable that the IC₅₀-value is as small as possible or the IC₅₀-values are as small as possible, but in general IC₅₀-values that lie between the above given upper limits and a lower limit in the range of 0.0001 μmol, 0.001 μmol, 0.01 μmol or even above 0.1 μmol are sufficient to indicate the desired pharmaceutical activity. However, the activities measured can vary depending on the respective testing system or assay chosen.

Alternatively, the advantageous biological activity of the compounds according to the invention can easily be demonstrated in in vitro assays, such as in vitro proliferation assays or in vitro growth assays. Suitable in vitro assays are known in the art, for example from the literature cited herein and the references cited in the literature or can be performed as described below, or can be developed and/or performed in an analogous manner thereof.

As an example for an in vitro growth assay, human tumor cell lines, for example HCT116, DLD-1 or MiaPaCa, containing mutated K-ras genes can be used in standard proliferation assays, for example for anchorage dependent growth on plastic or anchorage independent growth in soft agar.

Human tumor cell lines are commercially available, for example from ATCC (Rockville Md.), and can be cultured according to methods known in the art, for example in RPMI with 10% heat inactivated fetal bovine serum and 200 mM glutamine. Cell culture media, fetal bovine serum and additives are commercially available, for example from Invitrogen/Gibco/BRL (Karlsruhe, Germany) and/or QRH Biosciences (Lenexa, Kans.). In a standard proliferation assay for anchorage dependent growth, 3×10³ cells can be seeded into 96-well tissue culture plates and allowed to attach, for example overnight at 37° C. in a 5% CO₂ incubator. Compounds can be titrated in media in dilution series and added to 96 well cell cultures. Cells are allowed to grow, for example for 1 to 5 days, typically with a feeding of fresh compound containing media at about half of the time of the growing period, for example on day 3, if the cells are allowed to grow 5 days. Proliferation can be monitored by methods known in the art, such as measuring metabolic activity, for example with standard XTT calorimetric assay (Boehringer Mannheim) measured by standard ELISA plate reader at OD 490/560, by measuring ³H-thymidine incorporation into DNA following an 8 h culture with 1 μCu ³H-thymidine, harvesting the cells onto glass fiber mats using a cell harvester and measuring ³H-thymidine incorporation by liquid scintillation counting, or by staining techniques, such as crystal violet staining. Other suitable cellular assay systems are known in the art.

Alternatively, for anchorage independent cell growth, cells can be plated at 1×10³ to 3×10³ in 0.4% Seaplaque agarose in RPMI complete media, overlaying a bottom layer containing only 0.64% agar in RPMI complete media, for example in 24-well tissue culture plates. Complete media plus dilution series of compounds can be added to wells and incubated, for example at 37° C. in a 5% CO₂ incubator for a sufficient time, for example 10-14 days, preferably with repeated feedings of fresh media containing compound, typically at 3-4 day intervals. Colony formation and total cell mass can be monitored, average colony size and number of colonies can be quantitated according to methods known in the art, for example using image capture technology and image analysis software. Image capture technology and image analysis software, such as Image Pro Plus or media Cybernetics.

As discussed herein, these signaling pathways are relevant for various disorders. Accordingly, by interacting with one or more of said signaling pathways, bisarylurea derivatives are useful in the prevention and/or the treatment of disorders that are dependent from said signaling pathways.

The compounds according to the invention are preferably kinase modulators and more preferably kinase inhibitors. According to the invention, kinases include, but are not limited to one or more Raf-kinases, one or more Tie-kinases, one or more VEGFR-kinases, one or more PDGFR-kinases, p38-kinase and/or SAPK2alpha.

Raf-kinases in this respect are respect preferably include or consist of A-Raf, B-Raf and c-Raf1.

Tie-kinases in this respect preferably include or consist of Tie-2 kinase.

VEGFR-kinases in this respect preferably include or consist of VEGFR-2 kinase.

The compounds according to the invention are more preferably modulators and especially inhibitors of kinases, preferably kinases selected from the group consisting of serine/threonine kinases and receptor tyrosine kinases.

According to the invention, receptor tyrosine kinases are preferably selected from Tie-kinases, VEGFR-kinases, PDGFR-kinases, SAPK-kinases and p38-kinases.

According to the invention, serine/threonine kinases are preferably selected from raf-kinases.

Accordingly, the compounds according to the invention are preferably modulators and more preferably inhibitors of one or more kinases, selected from the group consisting of A-Raf, B-Raf, c-Raf1, Tie-1, Tie-2, Tie-3, VEGFR-1, VEGFR-2, VEGFR-3, p38-kinase and Ltk-kinase.

Due to the kinase modulating or inhibiting properties of the compounds according to the invention, the compounds according to the invention preferably interact with one or more signalling pathways which are preferably cell signalling pathways, preferably by downregulating or inhibiting said signaling pathways. Examples for such signalling pathways include, but are not limited to the raf-kinase pathway, the Tie-kinase pathway, the VEGFR-kinase pathway, the PDGFR-kinase pathway, the p38-kinase pathway, the SAPK2alpha pathway and/or the Ras-pathway.

Modulation of the raf-kinase pathway plays an important role in various cancerous and noncancerous disorders, preferably cancerous disorders, such as dermatological tumors, haematological tumors, sarcomas, squamous cell cancer, gastric cancer, head cancer, neck cancer, oesophageal cancer, lymphoma, ovary cancer, uterine cancer and/or prostate cancer. Modulation of the raf-kinase pathway plays a even more important role in various cancer types which show a constitutive activation of the raf-kinase dependent signalling pathway, such as melanoma, colorectal cancer, lung cancer, brain cancer, pancreatic cancer, breast cancer, gynaecological cancer, ovarian cancar, thyroid cancer, chronic leukaemia and acute leukaemia, bladder cancer, hepatic cancer and/or renal cancer. Modulation of the raf-kinase pathway plays also an important role in infection diseases, preferably the infection diseases as mentioned above/below and especially in Helicobacter pylori infections, such as Helicobacter pylori infection during peptic ulcer disease.

One or more of the signalling pathways mentioned above/below and especially the VEGFR-kinase pathway plays an important role in angiogenesis. Accordingly, due to the kinase modulating or inhibiting properties of the compounds according to the invention, the compounds according to the invention are suitable for the prophylaxis and/or treatment of pathological processes or disorders caused, mediated and/or propagated by angiogenesis, for example by inducing anti-angiogenesis. Pathological processes or disorders caused, mediated and/or propagated by angiogenesis include, but are not limited to tumors, especially solid tumors, arthritis, especially heumatic or rheumatoid arthritis, diabetic retinopathy, psoriasis, restenosis; fibrotic disorders; mesangial cell proliferative disorders, diabetic nephropathy, malignant nephrosclerosis, thrombotic microangiopathy syndromes, organ transplant rejection, glomerulopathies, metabolic disorders, inflammation and neurodegenerative diseases, and especially solid tumors, rheumatic arthritis, diabetic retinopathy and psoriasis.

Modulation of the p38-signalling pathway plays an important role in various cancerous and although in various noncancerous disorders, such as fibrosis, atherosclerosis, restenosis, vascular disease, cardiovascular disease, inflammation, renal disease and/or angiogenesis, and especially noncancerous disorders such as rheumatoid arthritis, inflammation, autoimmune disease, chronic obstructive pulmonary disease, asthma and/or inflammatory bowel disease.

Modulation of the PDGF-signalling pathway plays an important role in various cancerous and although in various noncancerous disorders, such as rheumatoid arthritis, inflammation, autoimmune disease, chronic obstructive pulmonary disease, asthma and/or inflammatory bowel disease, and especially noncancerous disorders such as fibrosis, atherosclerosis, restenosis, vascular disease, cardiovascular disease, inflammation, renal disease and/or angiogenesis.

Subject of the present invention are therefore bisarylurea derivatives according to the invention as promoters or inhibitors, preferably as inhibitors, of the signaling pathways described herein. Preferred subject of the invention are therefore bisarylurea derivatives according to the invention as promoters or inhibitors, preferably as inhibitors of the raf-kinase pathway. More preferred subject of the invention are therefore bisarylurea derivatives according to the invention as promoters or inhibitors, preferably as inhibitors of the raf-kinase. Even more preferred subject of the invention are bisarylurea derivatives according to invention as promoters or inhibitors, preferably as inhibitors of one or more raf-kinases, selected from the group consisting of A-raf, B-raf and c-raf1. Especially preferred subject of the invention are bisarylurea derivatives according to the invention as promoters or inhibitors, preferably as inhibitors of c-raf1.

Thus, subject of the present invention are bisarylurea derivatives according to the invention as medicaments. Subject of the present invention are bisarylurea derivatives according to the invention as medicament active ingredients. Further subject of the present invention is the use of one or more bisarylurea derivatives according to the invention as a pharmaceutical. Further subject of the present invention is the use of one or more bisarylurea derivatives according to the invention in the treatment and/or the prophylaxis of disorders, preferably the disorders described herein, more preferred disorders that are caused, mediated and/or propagated by signalling pathways discussed herein, even more preferred disorders that are caused, mediated and/or propagated by raf-kinases and especially disorders that are caused, mediated and/or propagated by raf-kinases, selected from the group consisting of A-raf, B-raf and c-raf1. Usually, the disorders discussed herein are divided into two groups, hyperproliferative and non hyperproliferative disorders. In this context, psioarsis, arthritis, inflammation, endometriosis, scarring, begnin prostatic hyperplasia, immunological diseases, autoimmune diseases and immunodeficiency diseases are to be regarded as noncancerous disorders, of which arthritis, inflammation, immunological diseases, autoimmune diseases and immunodeficiency diseases are usually regarded as non hyperproliferative disorders. In this context, brain cancer, lung cancer, squamous cell cancer, bladder cancer, gastric cancer, pancreatic cancer, hepatic cancer, renal cancer, colorectal cancer, breast cancer, head cancer, neck cancer, oesophageal cancer, gynaecological cancer, thyroid cancer, lymphoma, chronic leukaemia and acute leukaemia are to be regarded as cancerous disorders, all of which are usually regarded as hyperproliferative disorders. Especially cancerous cell growth and especially cancerous cell growth mediated by raf-kinase is a disorder which is a target of the present invention. Subject of the present invention therefore are bisarylurea derivatives according to the invention as medicaments and/or medicament active ingredients in the treatment and/or the prophylaxis of said disorders and the use of bisarylurea derivatives according to the invention for the manufacture of a pharmaceutical for the treatment and/or the prophylaxis of said disorders as well as a method of treatment of said disorders, comprising administering one or more bisarylurea derivatives according to the invention to a patient in need of such an administration. Subject of the present invention therefore are bisarylurea derivatives according to the invention as medicaments and/or medicament active ingredients in the treatment and/or the prophylaxis said disorders and the use of bisarylurea derivatives according to the invention for the manufacture of a pharmaceutical for the treatment and/or the prophylaxis of said disorders as well as a method of treatment of said disorders, comprising administering one or more bisarylurea derivatives according to the invention to a patient in need of such an administration.

Accordingly, subject of the present invention are pharmaceutical compositions that contain one or more bisarylurea derivatives according to the invention. Subject of the present invention are especially pharmaceutical compositions that contain one or more bisarylurea derivatives according to the invention and one or more additional compounds (other than the compounds of the instant invention), preferably selected from the group consisting of physiologically acceptable excipients, auxiliaries, adjuvants, carriers and pharmaceutically active ingredients other than the compounds according to the invention.

Accordingly, subject of the present invention is a process for the manufacture of a pharmaceutical composition, wherein one or more bisarylurea derivatives according to the invention and one or more compounds (other than the compounds of the instant invention), preferably selected from the group consisting of carriers, excipients, auxiliaries, adjuvants and pharmaceutically active ingredients other than the compounds according to the invention.

Accordingly, the use of the compounds according to the invention in the treatment of Hyperproliferative disorders is a subject of the instant invention.

Accordingly, the use of the compounds according to the invention for producing a medicament for the treatment of hyperproliferative disorders is a subject of the instant invention.

Above and below, all temperatures are given in ° C. In the examples below, “conventional work-up” means that the organic phase is washed with saturated NaHCO₃ solution, if desired with water and saturated NaCl solution, the phases are separated, the organic phase is dried over sodium sulfate and evaporated, and the product is purified by chromatography on silica gel, by preparative HPLC and/or by crystallization.

The present invention relates to bisarylurea derivatives of formula I, the use of the compounds of formula I as inhibitors of raf-kinase, the use of the compounds of formula I for the manufacture of a pharmaceutical composition and a method of treatment, comprising administering said pharmaceutical composition to a patient.

### EXAMPLES

i) Synthesis of the Pyridine Units 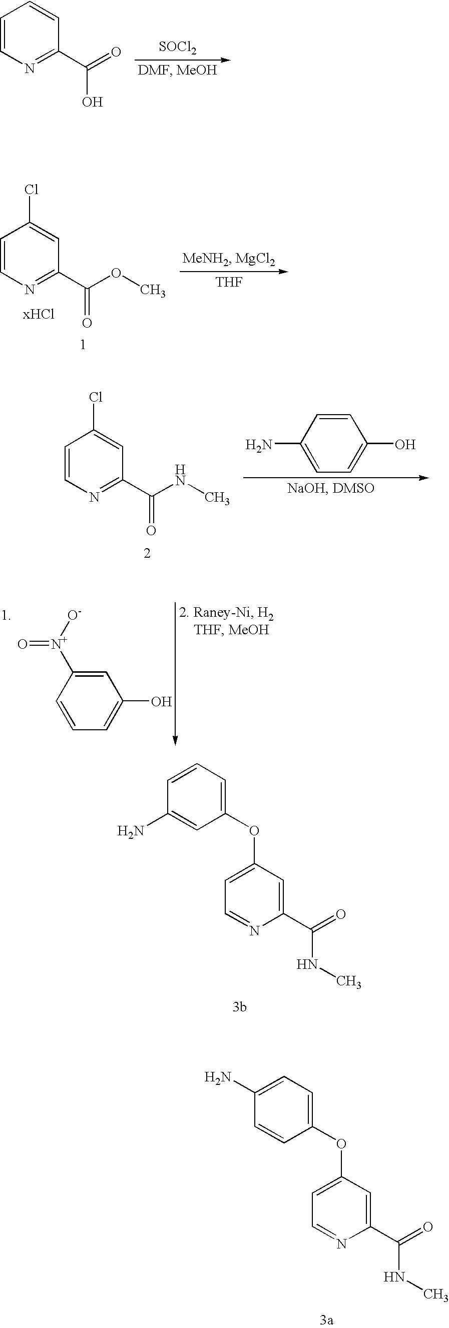
1) 750 ml of thionyl chloride are heated to 45° C. under an N₂ atmosphere, and 23 ml of DMF are added dropwise. 250 g (2.031 mol) of pyridine-2-carboxylic acid are subsequently added in portions, and the reaction mixture is stirred at 45° C. for a further 15 minutes and at 80° C. for 24 hours. The yellow suspension is evaporated, and the residue is entrained a number of times with toluene. The oily residue is dissolved in 180 ml of toluene, the solution is cooled to 0° C., and 110 ml of methanol are added dropwise. The suspension is stirred for a further hour, and the precipitated solid is filtered off with suction and rinsed with toluene. The resultant crude product is recrystallised a number of times from acetone and dried in a vacuum drying cabinet.

Yield: 140 g (33%) of 1, pale crystals

2) 140 g (0.673 mol) of 1 are stirred with 32 g (0.336 mol) of magnesium chloride and 2 l of THF at room temperature. After 5 minutes, 1.36 l (2.369 mol) of methylamine are added dropwise over the course of 20 minutes. The suspension is stirred at room temperature for a further 16 hours. 1.3 l of water and 680 ml of 1N HCl solution are added to the reaction mixture, and the mixture is extracted with ethyl acetate (3×1 l). The combined organic phases are washed with a saturated NaCl solution, dried using sodium sulfate, filtered and evaporated. The crude product is taken up in 300 ml of ethyl acetate and extracted with 200 ml of 1N HCl solution. The aqueous phase is adjusted to pH 9 using a 25% NH₄OH solution and extracted with ethyl acetate (2×400 ml). The organic phase is dried using sodium sulfate, filtered and evaporated.

Yield: 93 g (81%) of 2, brown oil

3a) 50 g (0.293 mol) of 2 and 32.6 g (0.293 mol) of 4-aminophenol are dissolved in DMSO, and 29.3 g (0.733 mol) of sodium hydroxide are slowly added. The solution is then heated at 100° C. overnight. After a further 29.3 g (0.733 mol) of sodium hydroxide had been added, the reaction mixture is again stirred at 100° C. overnight. The reaction mixture is cooled to room temperature, ice-water is added, and the mixture is extracted a number of times with diethyl ether. The combined organic phases are dried using sodium sulfate, filtered and evaporated.

Yield: 36 g (51%) of 3a, brown oil

3b) 2.8 g (16.41 mmol) 2 and 4.6 g (32.83 mmol) 3-nitrophenol are stirred at 150° C. overnight. The reaction mixture is cooled to room temperature, treated with ethyl acetate and 2N NaOH-solution. The organic phase is separated and the water phase is extracted 2× with ethylacetate. The combined organic phases are washed 2× with brine, dried over sodium sulfate, filtered and evaporated. The residue is put on silica gel and purified by column chromatography (eluent: n-heptane/ethylacetate).

Yield: 2.88 g (62%), pale yellow crystals

The accordingly obtained product is hydrogenated with H₂/Raney-Ni in THF/methanol at room temperature. The reaction mixture is filtered through a Seitz-filtre and rinsed with MeOH. The filtrate is concentrated, taken up in dichloromethane, dried over sodium sulfate, filtered and evaporated.

Yield: 2.29 g (92%) 3b, brown oil

### 4-(4-Pyridinyloxy)-phenylamin

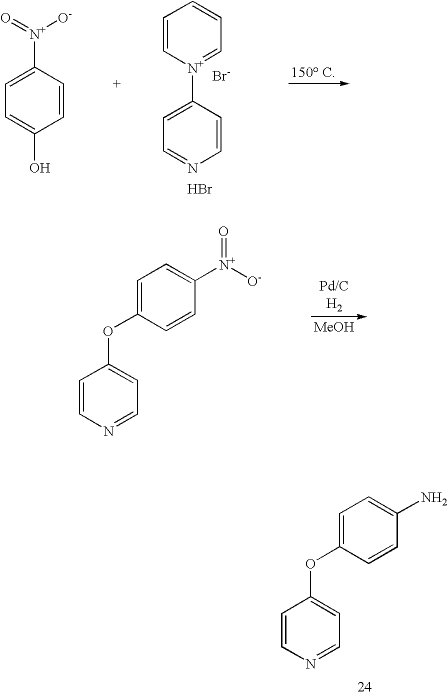 a) 195 g (1.4 mol) 4-Nitrophenol and 445.2 g (1.4 mol) Bipyridine are mixed and heated slowly to 150° C. After 3 h of stirring at 150° C., the still hot reaction mixture is poured onto 5 l ice/water. The reaction mixture is made acidic with hydrochloric acid and the water phase is washed 2× with 3 l methyl-tert.butylether. The water phase is made alkaline (pH 12) with concentrated NaOH solution and extracted 2× with 3 l methyl-tert.butylether. The combined organic phases are washed 4× with 1 l water, dried using Na₂SO₄, filtered and evaporated. The residue is crystallised from ether/petroleum ether added crystals are dried in vacuo.

Yield: 75 g (25%) brown crystals

b) The thus obtained nitro-compound is hydrogenated using Pd/C in MeOH at room temperature. The reaction mixture is filtered and the filtrate is evaporated. The residue is digested with diethyl ether: petroleum ether=2:1, filtered by suction, rinsed with petroleum ether and dried in vacuo

Yield: 50.94 g (76%) 24, brown crystals

### 3-(4-Pyridinyloxy)-phenylamin

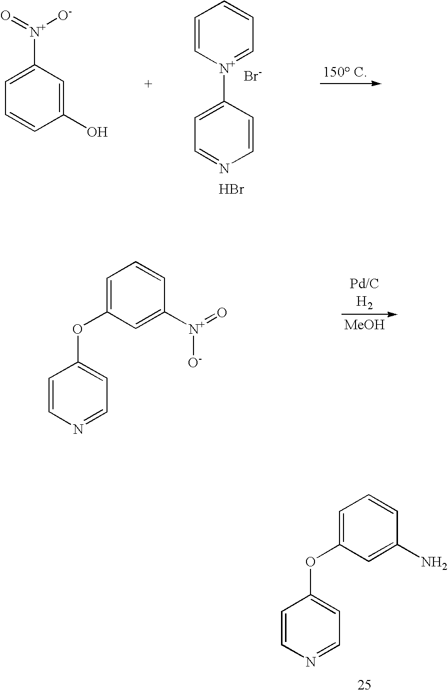 a) 200 g (1.44 mol) 3-Nitrophenol and 457.93 g (1.44 mol) Bipyridine are mixed and heated to 150° C. After 3 h of stirring at 150° C. the still hot reaction mixture is poured onto 5 l ice/water. The reaction mixture is made acidic with hydrochloric acid and the water phase is washed 2× with 3 l methyl-tert.butylether. The water phase is made alkaline (pH 12) with concentrated NaOH solution and extracted 2× with 3 l methyl-tert.butylether. The combined organic phases are washed 4× with 1 l water, dried using Na₂SO₄, filtered and evaporated. The residue is dissolved in 2 l diethylether, treated with 20 g charcoal, stirred for 1 h and filtered. The filtrate is concentrated to 200 ml and the product is crystallised by adding 500 ml petroleum ether in an ice bath. The crystals are separated and dried in vacuo.

Yield: 131 g (42%) beige crystals

b) The thus obtained nitro-compound is hydrogenated using Pd/C in MeOH at room temperature. The reaction mixture is filtered and the filtrate is evaporated. The residue is digested with diethyl ether, filtered by suction, rinsed with diethyl ether and dried in vacuo.

Yield: 98.08 g (87%) 25, pale brown crystals
ii) Synthesis of the Anilines

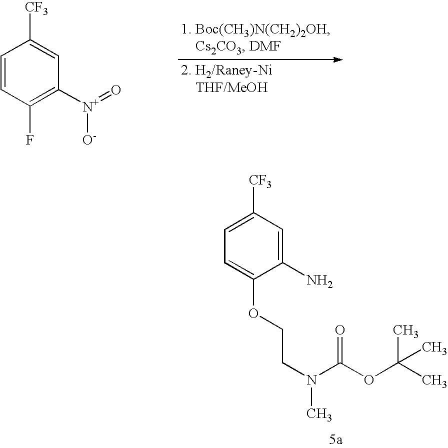

3 ml (21 mmol) 4-Fluoro-3-nitrobenzotrifluoride are dissolved in dimethylformamide (DMF), treated with 4.4 g (25 mmol) N-Boc-N-methylaminoethanol and 20.7 g (63 mmol) cesium carbonate and stirred at 55° C. overnight. The reaction mixture is filtered by suction and the filtrate is evaporated. The residue is taken up in ethyl acetate and washed several times with water. The organic phase is dried over Na₂SO₄, filtered and evaporated to dryness.

Yield: 6.9 g (90%), brown oil which crystallises upon standing

The accordingly obtained nitro compound is hydrogenated with H₂/Raney-Ni in THF/methanol—1/1 at room temperature within 1 h. The catalyst is removed by filtration and the filtrate is evaporated to dryness. The crystalline residue is digested with petrol ether and filtered by suction.

Yield: 4.66 g (72%) 5a, pale grey crystals

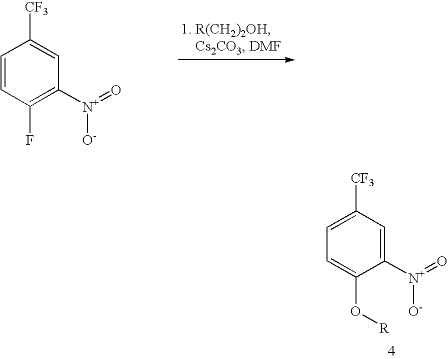

5 mmol 4-Fluoro-3-nitrobenzotrifluoride, 5-7.5 mmol substituted 2-amino ethanol (R(CH₂)₂OH) and 11.5-12.5 mmol cesium carbonate are dissolved in DMF and stirred at room temperature or 50-80° C. until a full conversion is achieved. Depending from the reaction route chosen, the reaction mixture is worked up according the following variants:

Variant A: the reaction mixture is filtered and the residue rinsed with ethyl acetate. The filtrate is diluted with ethyl acetate, washed 3× with water and 1× with brine, dried over Na₂SO₄, filtered and evaporated. The residue is purified by column chromatography (silica gel, eluent: DCM/MeOH 0-5% in 45 min).

Variant B: the reaction mixture is filtered by suction and rinsed with little DMF. The filtrate is evaporated. The oily residue is taken up in 100 ml water and extracted 3× with acyl acetate. The combined organic phases are washed 2× with water and 1× with brine, dried over Na₂SO₄, and evaporated. The residue is purified by column chromatography (silica gel, eluent: DCM/MeOH 0-5% in 45 min).

Variant C: the reaction mixture is filtered by suction and rinsed with little DMF. The filtrate is evaporated. The all the residue is taken up in 100 ml water and extracted 3× with acyl acetate. The combined organic phases are washed 2× with water and 1× with brine, dried over Na₂SO₄, and evaporated.

Substituents, reaction conditions and yields:

4b: R═(CH₂)₂N(CH₂)₄; 50° C., over night, working up procedure: A, 71%, yellow oil

4c: R═(CH₂)₂N(CH₃)₂; room temperature, over night, working up procedure: A, 85%, yellow oil

4d: R═(CH₂)₂N(C₂H₅)₂; 70° C., 2 h, working up procedure: A, 90%, yellow oil

4e: R═(CH₂)₂N(CH₂)₂O(CH₂)₂; 50° C., over night, working up procedure: B, 74%, red-brown oil

4f: R═(CH₂)₂N(CH₂)₂NBoc(CH₂)₂; 50° C., over night, working up procedure: A, 84%, yellow oil

4g: R═(CH₂)₂NBocCH(CH₃)₂; 80° C., 4 h, working up procedure: C, 65%, yellow oil

4h: R═CH₂C(CH₃)₂NBoc; 50° C., 2.5 h, working up procedure: B, 78%, yellow crystals

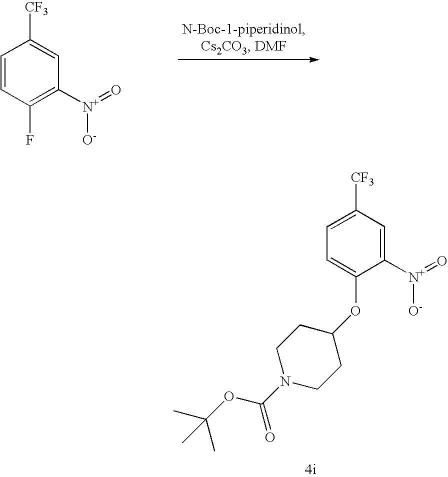

Manufacture according to the general working procedure for the compounds 4b-4h:
4i: 80° C., 5 h, working up procedure: A, 62%, yellow oil

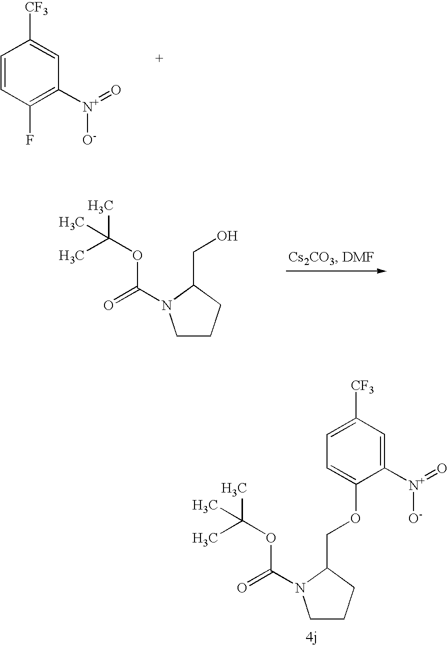

Manufacture ac cording to the general working procedure for the compounds 4b-4h.
4j: 50° C., 3 h, working up procedure: C, 92%, yellow oil

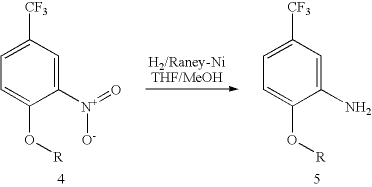

The thus obtained nitro compounds 4b-j are hydrogenated in THF with H₂ and Pd/C (5%) or THF/Methanol—1/1 with H₂ and Raney-Ni (5%) at room temperature until a full conversion is achieved. The catalyst is removed by filtration and the filtrate is evaporated to dryness.

Substituents, reaction conditions and yields:

5b: R═(CH₂)₂N(CH₂)₄; Pd/C, 18 h, 99.5%, yellow oil, crystallises upon standing

5c: R═(CH₂)₂N(CH₃)₂; Pd/C, 23 h, 98%, yellow crystals

5d: R═(CH₂)₂N(C₂H₅)₂; Pd/C, 21 h, 77%, brown oil

5e: R═(CH₂)₂N(CH₂)₂O(CH₂)₂; Pd/C, 21 h, 99%, beige solid

5f: R═(CH₂)₂N(CH₂)₂NBoc(CH₂)₂; Raney-Ni, 21 h, 92%, brown oil

5g: R═(CH₂)₂NBocCH(CH₃)₂; Pd/C, 16 h, 98%, brown oil

5h: R═CH₂C(CH₃)₂NBoc; Pd/C, 42 h, 99%, beige crystals

5i: from 4i, Raney-Ni, 23 h, 95%, grey solid

5j: from 4j, Pd/C, 18 h, 97%, colourless oil

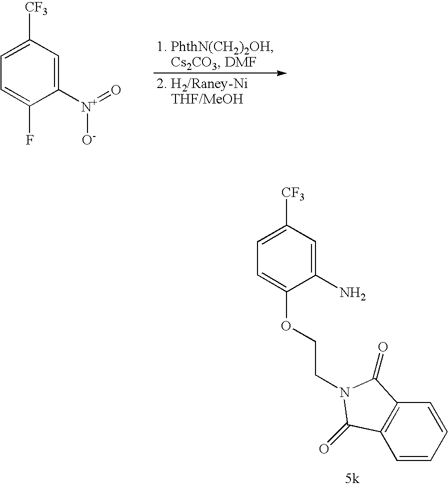

0.67 ml (4.6 mmol) 4-Fluoro-3-nitrobenzotrifluoride are dissolved in DMF, treated with 1.08 g (5.6 mmol) N-(2-Hydroxyethyl)phthalimide and 3.82 g (11.6 mmol) cesium carbonate and stirred for 5.5 h at 50° C. The reaction mixture is filtered by suction and the filtrate is evaporated to dryness. The residue is taken up in ethyl acetate and washed several times with water. The organic phase is dried over Na₂SO₄, filtered and evaporated to dryness.

Yield: 1.15 g (61%) 4k, yellow solid

1.1 g (2.7 mmol) of the accordingly obtained nitro compound is hydrogenated with H₂/Raney-Ni in THF/methanol—1/1 at room temperature overnight. The catalyst is removed by filtration and the filtrate is evaporated to dryness. The crystalline residue is digested with methanol and filtered by suction.

Yield: 1.04 g (93%) 5k, yellow solid

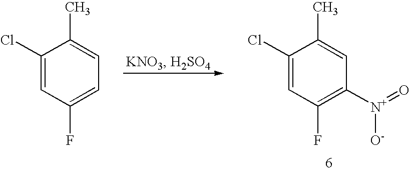

55 g (380 mmol) 2-Chloro-4-fluoro toluene are dissolved in 500 ml concentrated sulfuric acid and cooled to −5-0° C. in an ice bath. To this solution, 50.6 g (500 mmol) potassium nitrate are added in several portions within 1 h. The reaction mixture is warmed up to room temperature overnight and then poured onto ice. The yellow suspension is extracted 3× with 1 l tert.-Butyl-methylether each time and the combined organic phases are washed neutral with NaHCO₃-solution. The organic phase is stirred with Na₂SO₄ and 10 g charcoal, filtered and the filtrate is evaporated to dryness.

Yield: 60 g (81%) 6, yellow oil, which crystallises in the refrigerator

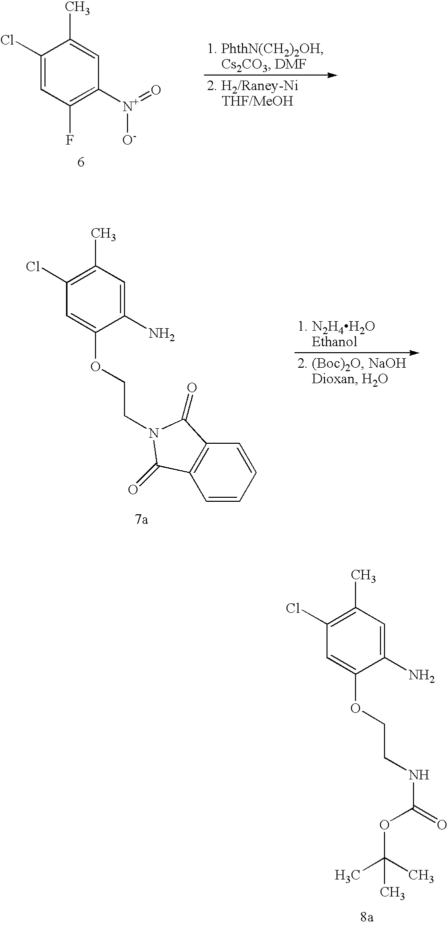

8 g (42.2 mmol) 2-Chloro-4-fluoro-5-nitrotoluene are dissolved in DMF, treated with 8.07 g (42.2 mmol) N-(2-Hydroxyethyl)phthalimide and 27.5 g (84.4 mmol) cesium carbonate and stirred at 80° C. for 5.5 h. The reaction mixture is cooled to room temperature, filtered by suction and washed with DMF. The filtrate is evaporated to dryness. The residue is taken up in ethyl acetate, washed 3× with water and 1× with brine, dried over Na₂SO₄, filtered and evaporated. The residue is digested with diethylether/MTB-ether (1:1), filtered by suction, washed with ethyl acetate/MTB-ether (1:1) and dried in vacuo.

Yield: 3.65 g (24%), pale yellow solid

3.65 g (10.1 mmol) of the accordingly obtained nitro compound is hydrogenated with H₂/Raney-Ni in THF/methanol—1/1 at room temperature overnight. The catalyst is removed by filtration and the filtrate is evaporated to dryness.

Yield: 3.09 g (92%) 7a, pale grey solid

0.7 g (2.05 mmol) 7a are suspended in 30 ml ethanol under stirring, treated with 114 μl (2.36 mmol) hydrazine hydrate and the reaction mixture is then heated 2 days to reflux. The formed precipitate is filtered off by suction and washed with ethanol. The combined filtrates are evaporated to dryness, the residue is taken up in ethyl acetate and extracted 2× with 1N HCl-solution. The combined water phases are made alkaline with 2N NaOH-solution and extracted 3× with ethyl acetate. The combined organic phases are washed 2× with water and 1× with brine, dried over Na₂SO₄ filtered and evaporated.

Yield: 0.42 g (95%), pale brown oil

0.34 g (1.58 mmol) of the accordingly obtained amine are dissolved in 3.5 ml dioxane, 1.7 ml 1N NaOH and 1.7 ml water by stirring at room temperature. The solution is cooled to 0° C. and at this temperature treated slowly with a solution of 379 mg (1.74 mmol) di-tert.-butyl dicarbonate in 1 ml dioxane. The reaction mixture is slowly warmed to room temperature, stirred for 18 h and then evaporated. The residue is taken up in 20 ml ethyl acetate, washed 2× with 15 ml water each time and 1× with brine, dried over Na₂SO₄, filtered and evaporated.

Yield: 0.47 g (99%) 8a, beige solid

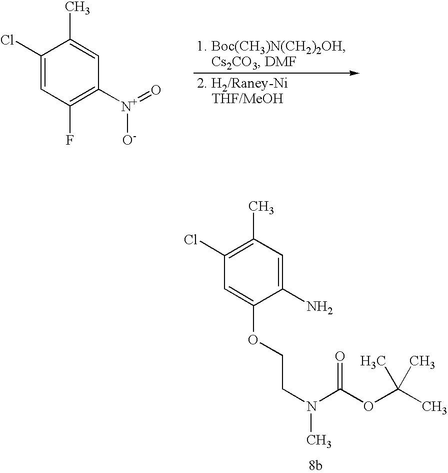

0.55 g (2.81 mmol) 2-Chloro-4-fluoro-5-nitrotoluene are dissolved in DMF, treated with 0.59 g (3.38 mmol) N-Boc-N-methylaminoethanol and 2.11 g (6.47 mmol) cesium carbonate and stirred at 50° C. overnight. The reaction mixture is filtered by suction and the filtrate is evaporated. The residue is taken up in ethyl acetate, washed several times with water, dried over Na₂SO₄, filtered and then evaporated to dryness.

Yield: 0.94 g (97%) 7b, brown oil

The accordingly obtained nitro compound is hydrogenated with H₂/Raney-Ni in THF at room temperature. The catalyst is removed by filtration and the filtrate is evaporated to dryness.

Yield: 0.83 g (96%) 8b, brown oil

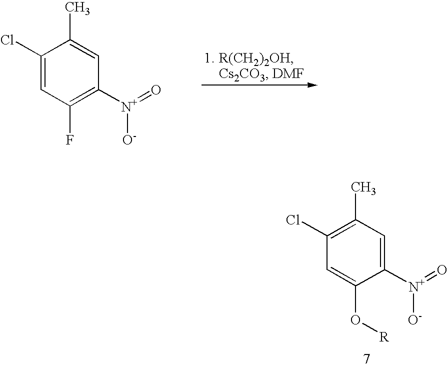

5 mmol 2-Chloro-4-fluoro-5-nitrotoluene, 5-7.5 mmol substituted 2-amino ethanol (R(CH₂)₂OH) and 11.5-12.5 mmol cesium carbonate are dissolved in DMF and stirred at room temperature or 50-80° C. until a full conversion is achieved. Depending from the reaction route chosen, the reaction mixture is worked up according the following variants:

Variant A: the reaction mixture is filtered and the residue rinsed with ethyl acetate. The filtrate is diluted with ethyl acetate, washed 3× with water and 1× with brine, dried over Na₂SO₄, filtered and evaporated. The residue is purified by column chromatography (silica gel, eluent: DCM/MeOH 0-5% in 45 min).

Variant B: the reaction mixture is filtered by suction and rinsed with little DMF. The filtrate is evaporated. The oily residue is taken up in 100 ml water and extracted 3× with acyl acetate. The combined organic phases are washed 2× with water and 1× with brine, dried over Na₂SO₄, and evaporated. The residue is purified by column chromatography (silica gel, eluent: DCM/MeOH 0-5% in 45 min).

Variant C: the reaction mixture is filtered by suction and rinsed with little DMF. The filtrate is evaporated. The oily residue is taken up in 100 ml water and extracted 3× with acyl acetate. The combined organic phases are washed 2× with water and 1× with brine, dried over Na₂SO₄, and evaporated.

Substituents, reaction conditions and yields:

7c: R═(CH₂)₂N(CH₂)₄; 50° C., over night, working up procedure: C, 87%, red-brown solid

7d: R═(CH₂)₂N(CH₃)₂; 50° C., overnight, working up procedure: C, 93%, brown oil

7e: R═(CH₂)₂N(C₂H₅)₂; 50° C., overnight, working up procedure: B, 72%, yellow oil

7f: R═(CH₂)₂N(CH₂)₂O(CH₂)₂; 50° C., overnight, working up procedure: B, 71%, brown crystals

7g: R═(CH₂)₂N(CH₂)₂NBoc(CH₂)₂; 50° C., overnight, working up procedure: C, 90%, brown oil

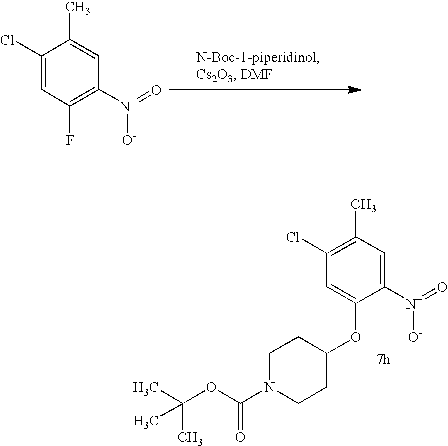

Manufacture according to the general working procedure for the compounds 7c-7g.
7h: 50° C., overnight, working up procedure: C, 99%, brown oil

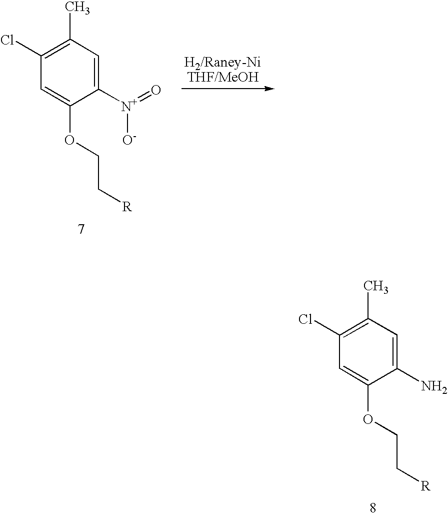

The accordingly obtained nitro compounds 7c-h are hydrogenated with H₂/Raney-Ni in THF at room temperature until a full conversion is achieved. The catalyst is removed by filtration and the filtrate is evaporated to dryness. The crystalline residue is digested with petrol ether and filtered by suction.

Substituents, reaction conditions and yields:

8c: R═N(CH₂)₄; 23 h, 95%, brown crystals

8d: R═N(CH₃)₂; 17 h, 79%, brown crystals

8e: R═N(C₂H₅)₂; 18.5 h, 99%, brown oil

8f: R═N(CH₂)₂O(CH₂)₂; 23 h, 80%, yellow solid

8g: R═N(CH₂)₂NBoc(CH₂)₂; 17 h, 99%, brown crystals

8h: from 7h, 45 h, 99%, brown oil

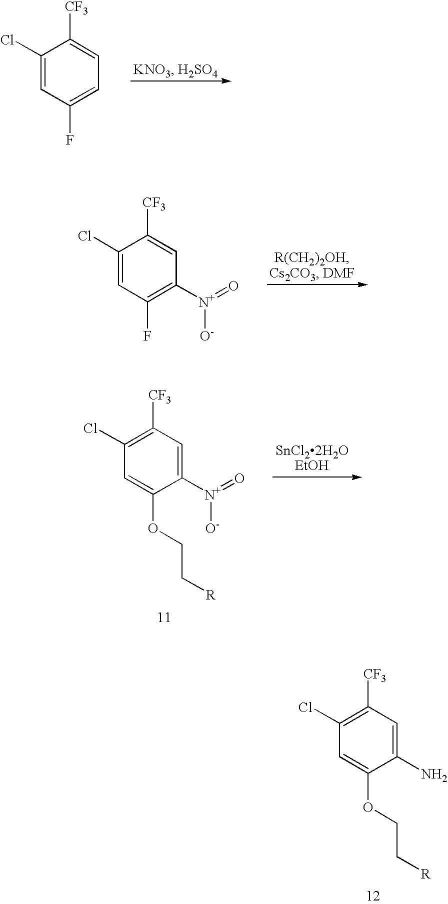

46 g (227 mmol) 2-Chloro-4-fluoro-benzotrifluoride are dissolved in 460 ml concentrated sulfuric acid and cooled to −5-0° C. in an ice bath. To this solution, 27.55 g (272.5 mmol) potassium nitrate are added in several portions within 1 h. After 30 min the reaction mixture is warmed to room temperature and stirred for 22 h. The reaction mixture is poured onto ice and extracted 3× with ethyl acetate. The combined organic phases are washed 1× with saturated NaHCO₃-solution and 1× with brine, dried over, Na₂SO₄, filtered and evaporated. The residue crystallises upon standing overnight. The crystals were digested with little petrol ether, filtered by suction and dried in vacuo.

Yield: 48.8 g (88%) 10, pale yellow crystals

10 mmol 5-Chloro-4-fluoro-3-nitrobenzotrifluoride are dissolved in DMF together with 12-20 mmol substituted 2-aminoethanol (R(CH₂)₂OH) and 23-25 mmol cesium carbonate in DMF and stirred until a full conversion is achieved. Depending on the course of the reaction the reaction mixture is worked up according to the following variants:

Variant A: the reaction mixture is filtered and the residue washed with dichloromethane. The filtrate is diluted with dichloromethane, washed 3× with water and 1× with brine, dried over Na₂SO₄, filtered and evaporated. The residue is purified by column chromatography (120 g silica gel, eluent: DCM/MeOH 0-5% in 45 min). The accordingly isolated product is taken up again in dichloromethane, washed 1× with 1N NaOH, 2× with water and 1× with brine, dried over Na₂SO₄, filtered and evaporated.

Variant B: the reaction mixture is filtered by suction and washed with DMF. The filtrate is evaporated. The oily residue is taken up in 40 ml water and extracted 4× with ethyl acetate. The combined organic phases are washed 2× with 1N NaOH and with water, dried over Na₂SO₄, filtered and evaporated.

Variant C: the reaction mixture is filtered by suction and washed with DMF. The filtrate is evaporated. The oily residue is taken up in 100 ml water and extracted 3× with ethyl acetate. The combined organic phases are washed 2× with water and 1× with brine, dried over Na₂SO₄, filtered and evaporated. The accordingly obtained pale brown solid is digested with dichloromethane and the filtrate is concentrated.

Variant D: the reaction mixture is filtered by suction, the filtrate diluted with ethyl acetate and extracted 2× with water. The organic phases dried over Na₂SO₄, filtered and evaporated. The oily residue is taken up in dichloromethane, washed 1× with 1N NaOH and then 1× with water, dried over Na₂SO₄, filtered and evaporated.

Variant E: the reaction mixture is filtered by suction and the filtrate is evaporated. The oily residue is digested with dichloromethane. The solid is filtered off by suction and washed with dichloromethane. The filtrate is washed 1× with 1N NaOH and 2× with water, dried over Na₂SO₄, filtered and evaporated.

Substituents, reaction conditions and yields:

11a: R═N(CH₃)₂; room temperature, 3 h, working up procedure: A, 54%, yellow oil, crystallises upon standing

11b: R═N(C₂H₅)₂; 50° C., 2 h, working up procedure: B, 67%, pale brown oil

11c: R═N(CH₂)₄; 50° C., 1 h, working up procedure: C, 62%, yellow solid

11d: R═N(CH₂)₂O(CH₂)₂; 50° C., 1 h, working up procedure: C, 62%, yellow solid

11e: R═N(CH₂)₂NBoc(CH₂)₂; room temperature, 15 h, working up procedure: D, 92%, brown oil

11f: R═N(CH₃)Boc; 50° C., 2.5 h, working up procedure: E, 93%, yellow oil

10 mmol of the nitro compounds 11a-f are stirred in Ethanol mit 40-50 mmol tin(II)chloride-dihydrate at room temperature or at 70° C. until a full conversion is achieved. The reaction mixture is made alkaline with NaHCO₃-solution. The formed precipitate is filtered off by suction over kieselguhr and the precipitate is washed with ethanol and ethyl acetate. The filtrate is concentrated with a Rotavapor until a water phase is obtained, which is extracted 3× with ethyl acetate. The combined organic phases are washed 1× with brine, dried over Na₂SO₄, filtered and evaporated.

Substituents, reaction conditions and yields:

12a: R═N(CH₃)₂; 70° C., 3 h, 84%, yellow oil, crystallises upon standing

12b: R═N(C₂H₅)₂; 70° C., 1.5 h, 75%, pale brown oil

12c: R═N(CH₂)₄; 70° C., 1 h, 58%, brown oil

12d: R═N(CH₂)₂O(CH₂)₂; 70° C., 1.5 h, 56%, pale brown oil

12e: R═N(CH₂)₂NBoc(CH₂)₂; room temperature, 1.5 h, 41%, brown oil

12f: R═N(CH₃)Boc; room temperature, 1.5 h, 53%, brown oil

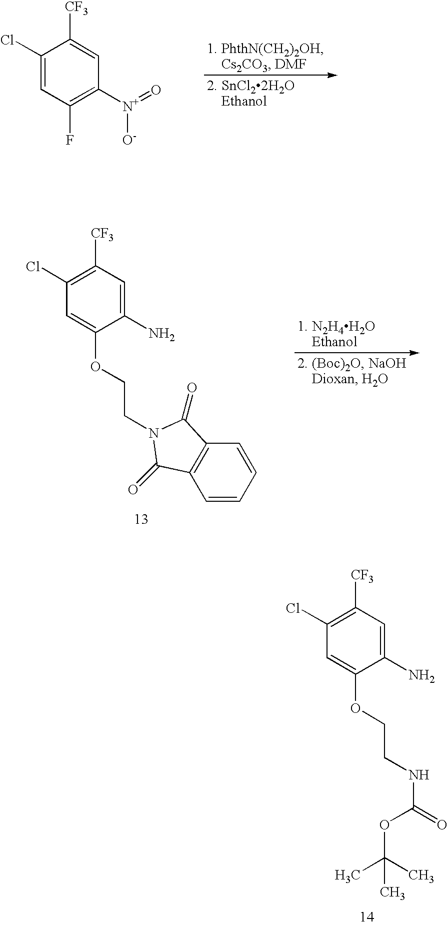

8 g (32.85 mmol) 5-Chloro-4-fluoro-3-nitrobenzotrifluoride are dissolved in DMF, treated with 9.68 g (50.64 mmol) N-(2-Hydroxyethyl)phthalimide and 34.37 g (105.5 mmol) cesium carbonate and stirred for 30 min at 80° C. The reaction mixture is cooled to room temperature, filtered by suction and washed with DMF. The filtrate is evaporated to dryness. The residue is taken up in ethyl acetate, washed 3× with water and 1× with brine, dried over Na₂SO₄, filtered and concentrated to ca. 30% of its volume. The formed precipitate is filtered by suction, washed with ethyl acetate and diethylether and dried in vacuo. The mother liquor is evaporated, the solid residue digested with ethyl acetate/diethylether (8:2), filtered by suction, washed with diethylether and dried in vacuo.

From the mother liquor, additional product is obtained by chromatography (150 g silica gel, eluent: dichloromethane/MeOH—98/2).

Yield: 10.73 g (77%), pale yellow solid

150 mg (0.36 mmol) of the nitro compounds and 404 mg (1.79 mmol) tin(II)chloride-dihydrate in THF are stirred for 1.5 h at room temperature. The reaction mixture is made alkaline with saturated NaHCO₃-solution. The formed precipitate is filtered off by suction over kieselguhr and washed with ethanol and ethyl acetate. The filtrate is concentrated with a Rotavapor until a water phase is obtained, which is extracted 3× with ethyl acetate. The combined organic phases are washed 1× with brine, dried over Na₂SO₄, filtered and evaporated.

Yield: 109 mg (79%) 13, pale yellow solid

17.7 g (39.56 mmol) 13 are suspended in 30 ml ethanol under stirring, treated with 4.81 ml (98.91 mmol) hydrazine hydrate and the reaction mixture is then heated 15 h to reflux. The formed precipitate is filtered off by suction and washed with ethanol. The combined filtrates are evaporated to dryness, the residue is taken up in ethyl acetate and extracted 2× with 1N HCl-solution. The combined water phases are made alkaline with 2N NaOH-solution and extracted 3× with ethyl acetate. The combined organic phases are washed 2× with water and 1× with brine, dried over Na₂SO₄ filtered and evaporated.

Yield: 9.4 g (93%), brown oil

8.69 g (34.1 mmol) of the accordingly obtained amine are dissolved in 50 ml dioxane, 40 ml 1N NaOH and 40 ml water by stirring at room temperature. The solution is cooled to 0° C. and at this temperature treated slowly with a solution of 8.16 g (36.8 mmol) di-tert.-butyl dicarbonate in 30 ml dioxane. The reaction mixture is slowly warmed to room temperature, stirred for 1 h. The formed precipitate is filtered off by suction, washed with water, taken up in ethyl acetate, dried over Na₂SO₄, filtered and evaporated.

Yield: 10.6 g (87%) 14, brown oil

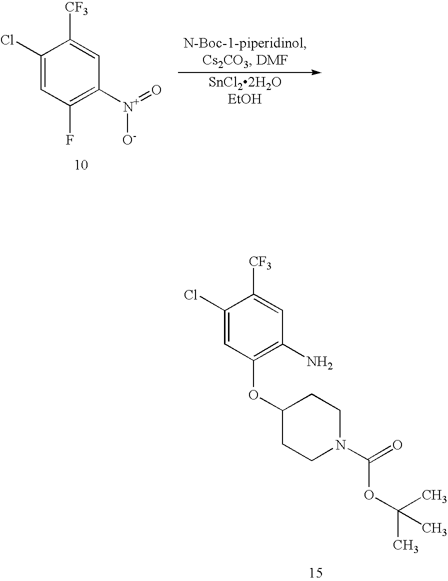

6.5 g (26.69 mmol) 5-Chloro-4-fluoro-3-nitrobenzotrifluoride, 6.45 g (32.03 mmol) N-Boc-1-piperidinol and 21.75 g (66.72 mmol) cesium carbonate are dissolved in DMF and stirred overnight at 50° C. The reaction mixture is filtered by suction and washed with little DMF. The filtrate is evaporated. The oily residue is taken up in ethyl acetate, washed, 2× with water, dried over Na₂SO₄, filtered and evaporated. The thus obtained crude product is purified by column chromatography (700 g silica gel, eluent: ethyl acetate/petrol ether—1/1).

Yield: 3.9 g (34%), yellow oil

3.9 g (9.18 mmol) of the nitro compounds and 10.36 g (45.91 mmol) tin(II)chloride-dihydrate in ethanol are stirred for 2 h at room temperature. The reaction mixture is made alkaline with saturated NaHCO₃-solution. The formed precipitate is filtered off by suction over kieselguhr and washed with ethanol and ethyl acetate. The filtrate is concentrated with a Rotavapor until a water phase is obtained, which is extracted 3× with ethyl acetate. The combined organic phases are washed 1× with brine, dried over Na₂SO₄, filtered and evaporated.

Yield: 3.6 g (97.5%) 15, brown solid
Synthesis of the Ureas

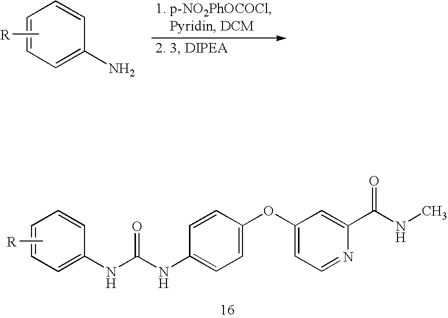

200 mmol of the anilines 5a-k, 8a-h, 12a-f, 14 and 15 are dissolved in dichloromethane together with 220 μmol p-nitrophenyl chloroformate, treated with 220 μmol pyridine at room temperature and stirred for 20-35 min. After the reaction is completed, 200 μmol 3 and 400 μmol DIPEA are added and the reaction mixture is stirred at room temperature until a full conversion is achieved (30 min-17 h). The reaction mixture is diluted with dichloromethane, successively extracted 2× with 1N NaOH, 1× with water and 1× with brine, dried over Na₂SO₄, filtered and evaporated. The accordingly obtained crude product is purified according to the following variants:

Variant A: the residue is purified by column chromatography (12 g silica gel, eluent: DCM/MeOH 3% in 45-55 min).

Variant B: the oily residue is crystallised in ethyl acetate by addition of little dichloromethane and MeOH, filtered off by suction and dried.

Starting materials, reaction conditions and yields:

16a: from 5a, 17 h, working up procedure: A, 87%, yellow oil

16b: from 5b, 1 h, working up procedure: A, 56%, colourless crystals

16c: from 5c, 1 h, working up procedure: A, 55%, colourless crystals

16d: from 5d, 1 h, working up procedure: A, 63%, colourless crystals

16e: from 5e, 1 h, working up procedure: A, 67%, colourless crystals

16f: from 5f, 1 h, working up procedure: A, 47%, colourless crystals

16g: from 5g, 30 min, working up procedure: A, 72%, pale yellow crystals

16h: from 5h, 45 min, working up procedure: A, 95%, yellow oil

16i: from 5i, 2 h, working up procedure: A, 77%, yellow oil

16j: from 5j, overnight, working up procedure: A, 76%, colourless crystals

16k: from 5k, 45 min, working up procedure: B, 59%, colourless crystals

17a: from 8a, 1 h, working up procedure: A, 96%, yellow oil

17b: from 8b, 17 h, working up procedure: A, 67.5%, yellow oil

17c: from 8c, 1 h, working up procedure: A, 52%, colourless crystals

17d: from 8d, 1 h, working up procedure: A, 55.5%, colourless crystals

17e: from 8e, 1 h, working up procedure: A, 54%, colourless crystals

17f: from 8f, 1 h, working up procedure: A, 73%, colourless crystals

17g: from 8g, 2 h, working up procedure: A, 71%, colourless crystals

17h: from 8h, 2 h, working up procedure: A, 54.5%, colourless oil

18a: from 12a, 30 min, working up procedure: A, 73%, colourless crystals

18b: from 12b, 30 min, working up procedure: A, 62%, colourless crystals

18c: from 12c, 1 h, working up procedure: A, 49%, yellow crystals

18d: from 12d, 30 min, working up procedure: A, 71%, colourless crystals

18e: from 12e, 1 h, working up procedure: A, 66%, beige solid

18f: from 12f, 1 h, working up procedure: A, 65%, yellow oil

18g: from 14, 1 h, working up procedure: A, 75%, yellow oil

18h: from 15, 1 h, working up procedure: A, 65.5%, yellow oil

Removal of the Protective Groups

A) BOC-Protective Group:

16a, 16f, 16g, 16h, 16i, 16j, 17a, 17b, 17g, 17h, 18e, 18f, 18g, 18h are treated with dichloromethane/trifluoro acetic acid—1/1 at room temperature and stirred for 10 min. The reaction mixture is diluted with dichloromethane, successively extracted 2× with saturated NaHCO₃ solution, 2× with water, dried over Na₂SO₄, filtered and evaporated. The residue is taken up in ethyl acetate, frozen and freeze-dried overnight.

Starting materials and yields:

19a: from 16a, 86%, yellow solid

19b: from 16f, 88%, yellow solid

19c: from 16g, 99%, colourless solid

19d: from 16h, 94%, colourless solid

19e: from 16i, 77.5%, yellow solid

19f: from 16j, 95%, yellow solid

19h: from 17a, 79%, yellow solid

19i: from 17b, 94%, yellow solid

19j: from 17g, 90%, colourless solid

19k: from 17h, 78%, colourless solid

19l: from 18e, 92.5%, yellow solid

19m: from 18f, 92%, yellow solid

19n: from 18g, extraction with ethylacetate, 99%, yellow solid

19o: from 18h, 79%, yellow solid

B) Phthalimide-Protecting Group:

16k and hydrazine hydrate (1.2 equ.) in ethanol are heated to reflux for 1 h. The reaction mixture is cooled down, the formed precipitate is separated by filtration by suction and rinsed with cold ethanol. The filtrate is evaporated to dryness, the residue taken up in ethyl acetate, the formed precipitate is removed by filtration by suction and rinsed with ethylacetate. The filtrate is evaporated to dryness.

Yield: 85%, 17g, colourless crystals

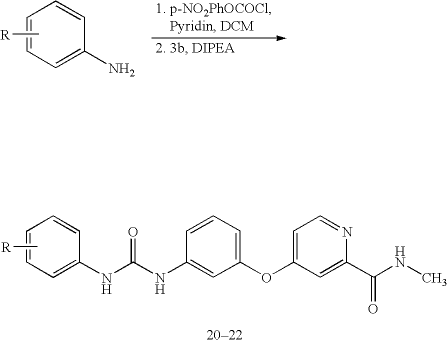

200 μmol of the respective aniline 5a, 5b, 5c, 5g, 5h, 5i, 5j, 5k, 8a, 8b, 8c, 8d, 8h, 12a, 12b, 12c, 12d, 12e, 12f, 14, 15 are dissolved together with 200-220 μmol p-nitrophenyl chloroformate in dichloromethane, treated with 220 μmol pyridine at room temperature and stirred for 20-35 min gerührt. After the reaction is finished, 200 μmol 3a and 400 μmol DIPEA are added and the reaction mixture is stirred at room temperature until the full conversion is achieved. The reaction mixture is diluted with dichloromethane, extracted consecutively 1× with water, 2× with 1N NaOH, 1× with water and 1× with brine, dried over Na₂SO₄, filtered and evaporated. The accordingly obtained crude product is purified according to the following variants:

Variant A: The residue is purified by column chromatography (12 g silica gel, eluent: DCM/acetone 10%).

Variant B: The residue is purified by column chromatography (12 g silica gel, eluent: DCM/MeOH 3%).

Variant C: The residue is recrystallised from methanol, filtered by suction, rinsed with little methanol and dried.

Variant D: The residue is purified by column chromatography (12 g silica gel, eluent: Petrolether/ethylacetate—7/3).

Variant E: The residue is recrystallised from ethylacetate, filtered by suction, rinsed with little ethyl acetate and dried.

Variant F: The residue is recrystallised from ethylacetate/petrol ether, filtered by suction, rinsed with petrol ether and dried.

Variant G: The residue is recrystallised from dichloromethane/petrol ether, filtered by suction, rinsed with little petrol ether and dried.

Variant H: The crystalline residue is digested with diethyl ether/petrol ether—1/4 and filtered by suction. From the mother liquor, additional product is obtained by crystallization.

Starting materials, reaction conditions and yields:

20a: from 5a, overnight, working up procedure: A, 41%, colourless solid

20b: from 5b, overnight, working up procedure: B, 31%, colourless crystals

20c: from 5c, overnight, working up procedure: B, 41.5%, colourless crystals

20g: from 5g, overnight, working up procedure: C, 64.5%, colourless crystals

20h: from 5h, overnight, working up procedure: D, 93%, colourless crystals

20i: from 5l, overnight, working up procedure: E, 75%, colourless crystals

20j: from 5j, overnight, working up procedure: B, 76%, colourless crystals

20k: from 5k, overnight, working up procedure: E, 82%, colourless crystals

21a: from 8a, overnight, working up procedure: F, 61%, beige solid

21b: from 8b, overnight, working up procedure: A, 34%, orange-brown solid

21c: from 8c, overnight, working up procedure: B, 32%, colourless crystals

21d: from 8d, overnight, working up procedure: B, 42%, colourless crystals

21h: from 8h, overnight, working up procedure: C, 39%, beige crystals

22a: from 12a, overnight, no working up, 81%, yellow solid

22b: from 12b, overnight, working up procedure: E, 30%, colourless solid

22c: from 12c, 3 h, working up procedure: G, 44%, beige crystals

22d: from 12d, 1 h, working up procedure: H, 50%, pale yellow crystals

22e: from 12e, overnight, working up procedure: F, 60%, colourless solid

22f: from 12f, overnight, working up procedure: A, 28%, colourless solid

22g: from 14, 4 h, working up procedure: G, 46%, beige solid

22h: from 15, overnight, working up procedure: F, 30%, colourless solid

Removing the Protecting Groups:

a) BOC-Protecting Group:

20a, 20g, 20h, 20i, 20j, 21a, 21b, 21h, 22e, 22f, 22g and 22h are treated with dichloromethane/trifluoro acetic acid—1/1 at room temperature and stirred for 20-30 min. The reaction mixture is diluted with dichloromethane, washed 1× with saturated NaHCO₃-solution and 2× with water, dried over Na₂SO₄, filtered and evaporated. The residue is taken up in acetonitrile/water, frozen and freeze-dried overnight.

Starting materials and yields:

23a: from 20a, 49%, colourless solid

23b: from 20g, 100%, colourless crystals

23c: from 20h, 100%, colourless crystals

23d: from 20i, 94%, colourless crystals

23e: from 20j, 95%, colourless solid

23g: from 21a, 72.5%, beige solid

23h: from 21b, 66%, colourless solid

23i: from 21h, 98%, beige solid

23j: from 22e, 94.5%, colourless solid

23k: from 22f, 76%, colourless solid

23l: from 22g, 73%, colourless solid

23m: from 22h, 100%, colourless solid

B) Phthalimide-Protecting Group:

20k and hydrazine hydrate (1.1 equ.) in ethanol are heated 2.5 h to reflux. The reaction mixture is cooled down, the formed precipitate is separated by filtration by suction and rinsed with cold ethanol. The filtrate is evaporated to dryness, the residue taken up in ethyl acetate and extracted with 4n HCl-solution. The water phase is made alkaline with NaOH and extracted several times with ethyl acetate. The combined organic phases are washed 1× with brine, dried over Na₂SO₄, filtered and evaporated.

Yield: 40%, 23f, colourless crystals

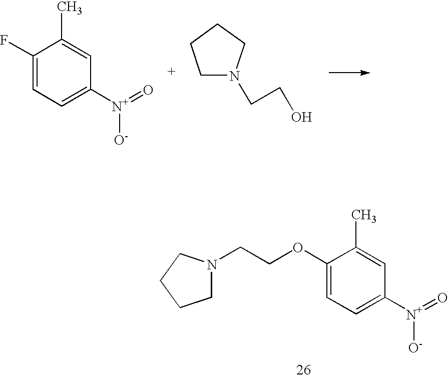

3.2 g (20.2 mmol) 2-Fluoro-5-nitro toluene in 20 ml DMF are treated consecutively with 14 g (43 mmol) Cs₂CO₃ and 3.2 g (26.4 mmol) N-(2-Hydroxyethyl)pyrrolidine and stirred at 100° C. overnight. The reaction mixture is evaporated, the residue dissolved in water and extracted with dichloromethane. The combined organic phases are washed with water, dried using Na₂SO₄, filtered and evaporated.

Yield: 2.3 g (43%) 26, brown-yellow crystals

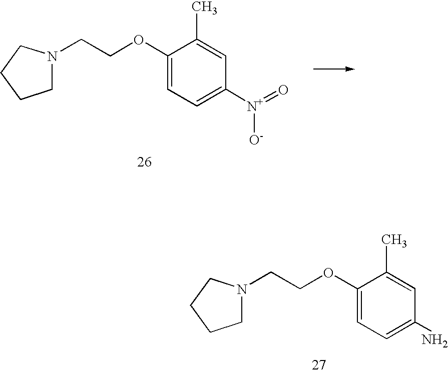

2.3 g (8.64 mmol) 26 are hydrogenated in methanol using H₂ and Pd/C (5%) at room temperature. The catalyst is removed by filtration and the filtrate is evaporated.

Yield: 1.89 g (99%) 27, brown oil

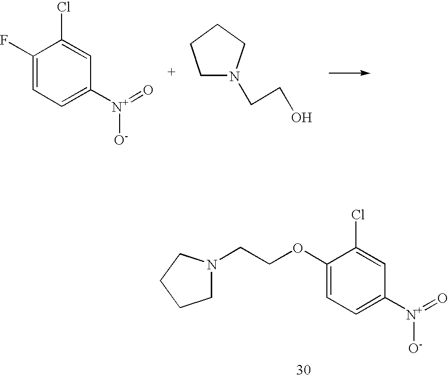

5 g (28 mmol) 3-Chloro-4-fluoronitrobenene in 25 ml DMF are treated consecutively with 19.5 g (60 mmol) Cs₂CO₃ and 4.5 g (37 mmol) N-(2-Hydroxyethyl)pyrrolidine and stirred at 100° C. overnight. The reaction mixture is evaporated, the residue dissolved in water and extracted with dichloromethane. The combined organic phases are washed with water, dried using Na₂SO₄, filtered and evaporated. The oily residue crystallises upon standing. The crystals either digested with petroleum ether/MTB ether, filtered and dried.

Yield: 3.27 g (43%) 30, brown crystals

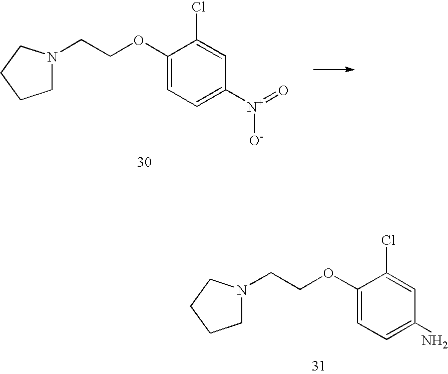

3.22 g (12 mmol) 30 are hydrogenated at room temperature using Methanol, H₂ and Raney-Ni. The catalyst is removed by filtration and the filtrate is evaporated to dryness.

Yield: 2.85 g (99%) 31, brown oil

In an analogous manner, the following compounds are obtained:

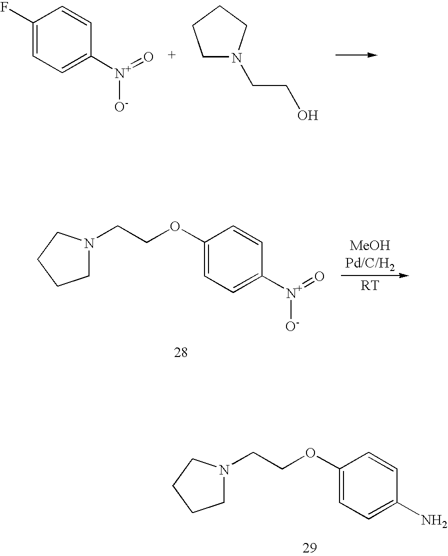

Yields: 70% 28, yellow oil; 98% 29, pale brown oil

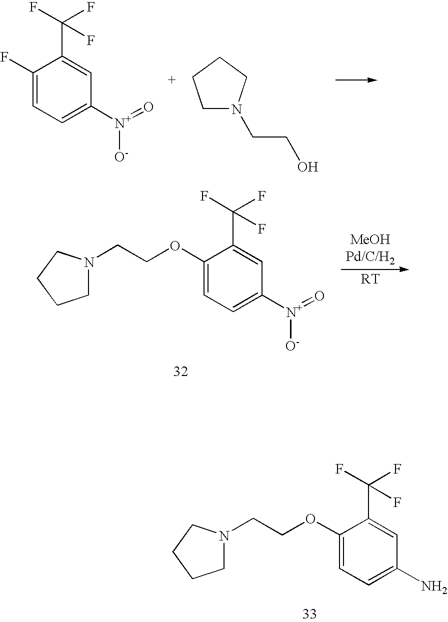

Yields: 53% 32, brown crystals; 97% 33, brown oil

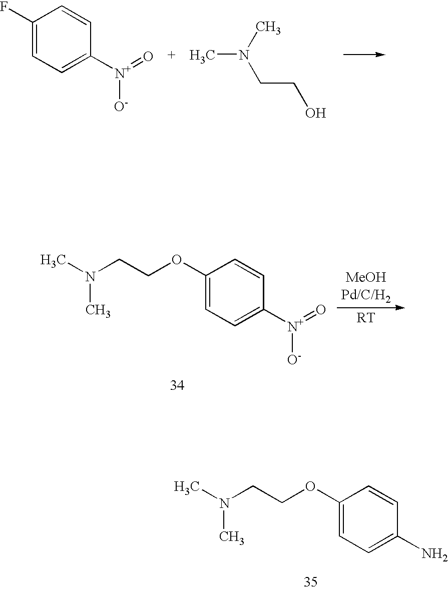

Yields: 82% 34, yellow crystals; 91% 35, brown oil

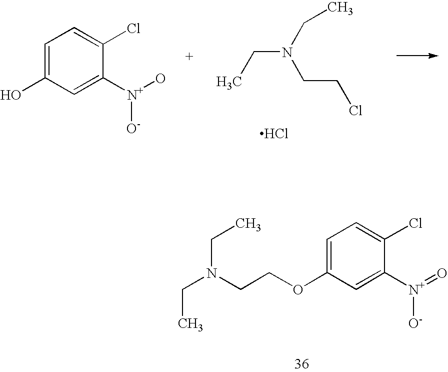

1 g (5.76 mmol) 4-Chloro-3-nitrophenol in 20 ml DMF are treated with 2 g (14.47 mmol) K₂CO₃ and 992 mg (5.76 mmol) N-(2-Chloroethyl)-N,N-diethyl ammonium chloride in 20 ml DMF. The reaction mixture is stirred at 80° C. overnight. The reaction mixture is evaporated, the residue is dissolved in water and extracted with EtOAc. The combined organic phases are washed several times with water, dried using Na₂SO₄, filtered and evaporated.

Yield: 1.39 g (88%) 36, dark oil

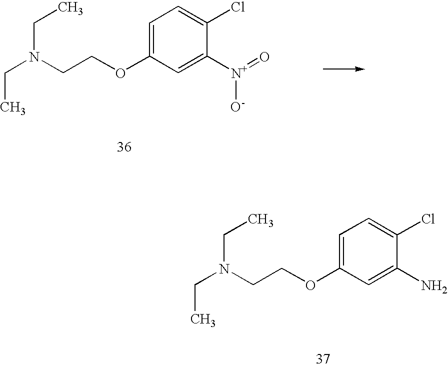

1.39 g (4.84 mmol) 36 in 30 ml EtOAc/Ethanol—2/1 are treated with 6.66 g (29.5 mmol tin (II)chloride-dihydrate and stirred for 2 h at 70° C. the reaction mixture is cooled down, poured onto 100 ml water and neutralised with 70 ml concentrated NaHCO₃ solution. The reaction mixture is filtered using kieselguhr, rinsed with ethylacetate. The organic phase is separated and the water phase is extracted with ethylacetate. The organic phases are dried using Na₂SO₄, filtered and evaporated.

Yield: 0.98 g (83%) 37, dark oil

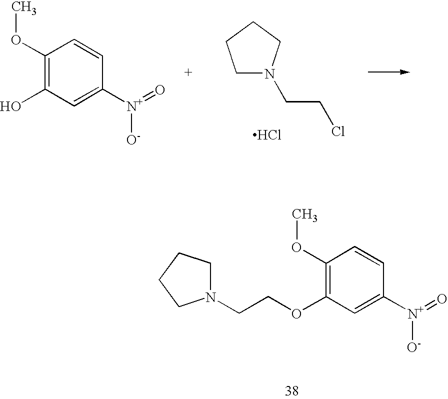

2 g (11.59 mmol) 2-Methoxy-5-nitrophenol in 30 ml DMF are treated with 4 g (28.94 mmol) K₂CO₃. 2.4 g (13.83 mmol) N-(2-Chloroethyl)-pyrrolidine hydrochloride are added and the reaction mixture is stirred at 100° C. overnight. The reaction mixture is evaporated, the residue is dissolved in water and extracted with EtOAc. The combined organic phases are extracted 2N-HCl, the water phase made alkaline with solid K₂CO₃ and extracted with EtOAc. The combined organic phases are dried using Na₂SO₄, filtered and evaporated.

Yield: 0.9 g (27%) 38, dark oil

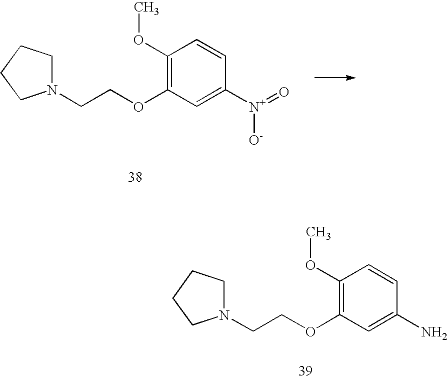

0.9 g (3.08 mmol) 38 are hydrogenated at room temperature in Methanol using H₂ and Pd/C (5%). The catalyst is removed by filtration and the filtrate is evaporated to dryness.

Yield: 0.8 g (94%) 39, dark oil

In an analogous manner, the following compounds are obtained:

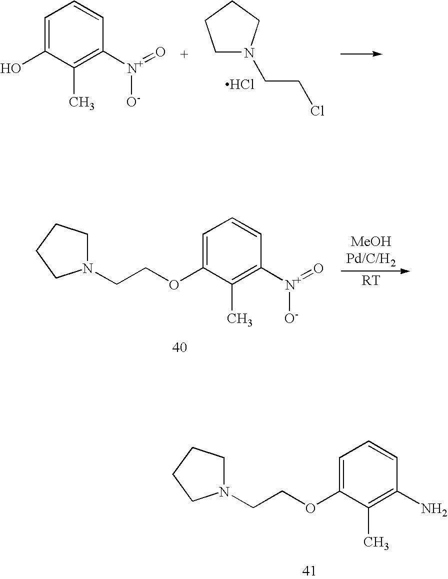

Yields: 66% 40, dark oil; 84% 41, yellow oil

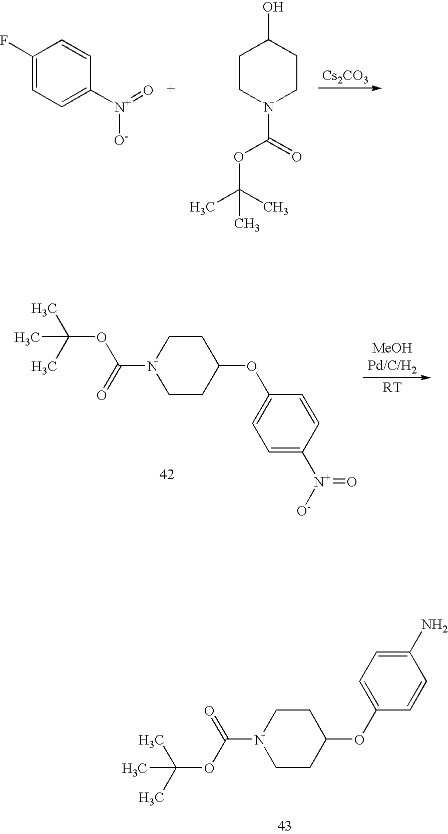

Yields: 99% 42, yellow oil; 99% 43

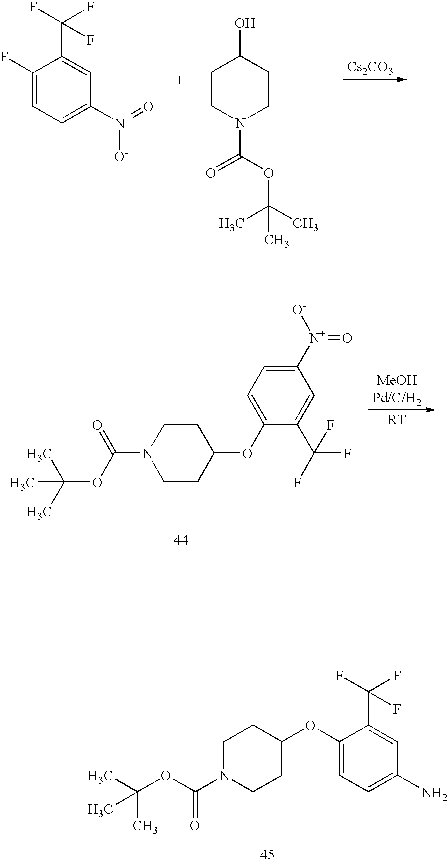

Yields: 96% 44, yellow oil; 97% 45 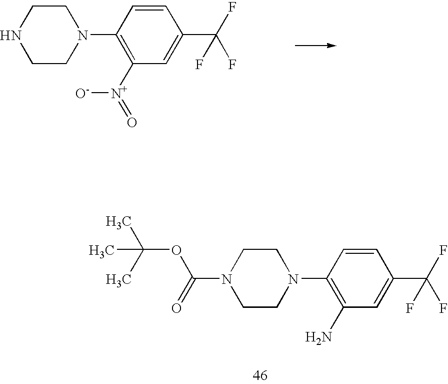
a) 2 g (7.05 mmol) 1-(2-Nitro-4-trifluoro methylphenyl)piperazine in 5 ml dichloromethane are treated consecutively with 1.08 ml (7.75 mmol) triethyl amine and 1.66 ml (7.75 mmol) Di-tert.-butyldicarbonate and stirred for 3 h at room temperature. The reaction mixture is diluted dichloromethane, washed with 1M NaOH-solution, 0.5 M HCl and with water, dried using Na₂SO₄, filtered and evaporated.

Yield: 2.65 g (99%), yellow crystals

b) 2.65 g (6.99 mmol) of the nitro compound are hydrogenated in methanol using H₂ and Raney-Ni at room temperature. The catalyst is removed by filtration and the filtrate is evaporated to dryness.

Yield: 2.46 g (100%) 46

As described herein or an analogous manner thereof, the following compounds are obtained:

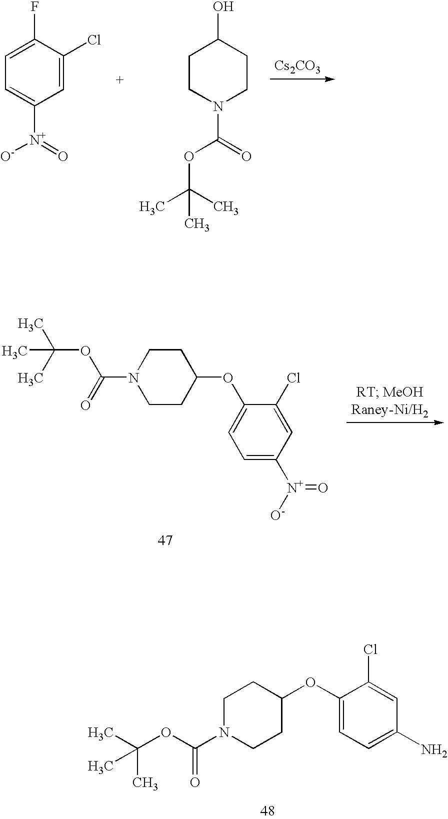

Yields: 70% 47, yellow crystals; 99% 48

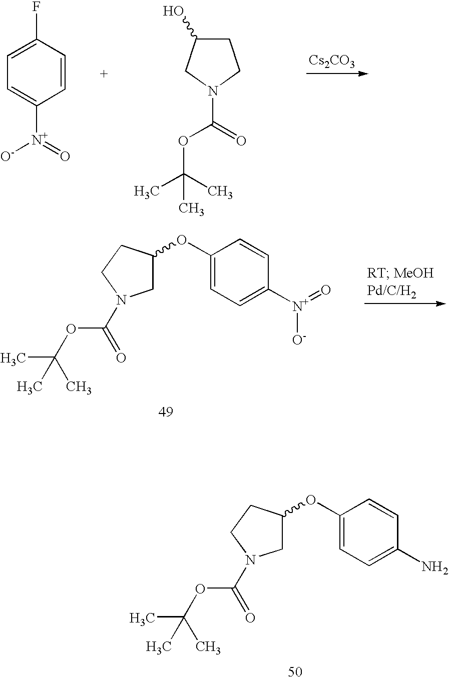

Yields: 2.9 g (100%) 49, yellow oil; 80% 50

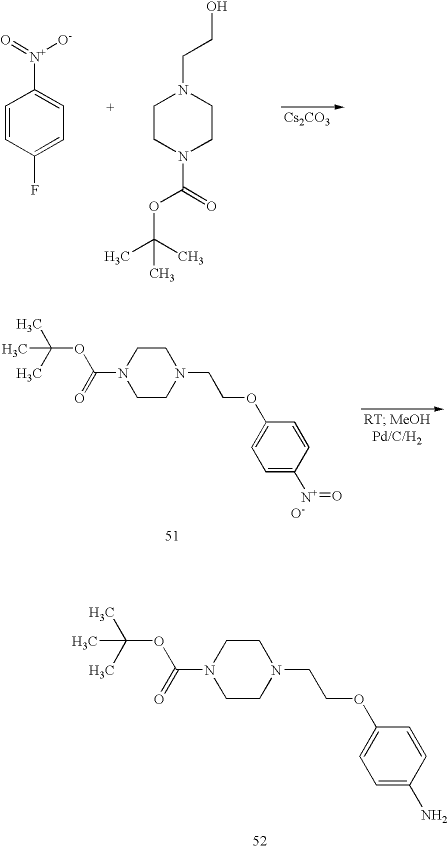

Yield: 6.33 g (100%) 51, yellow crystals; yield: 4.4 g (78%) 52, pale brown crystals

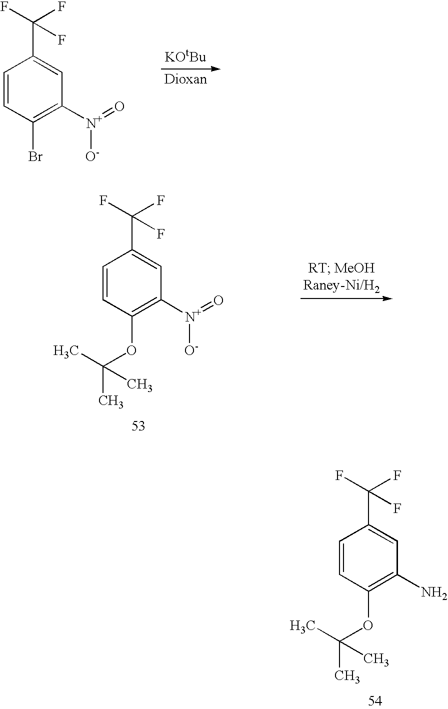

Yield: 1.84 g (9%) 53, yellow oil; yield: 902 mg (64%) 54, colourless oil

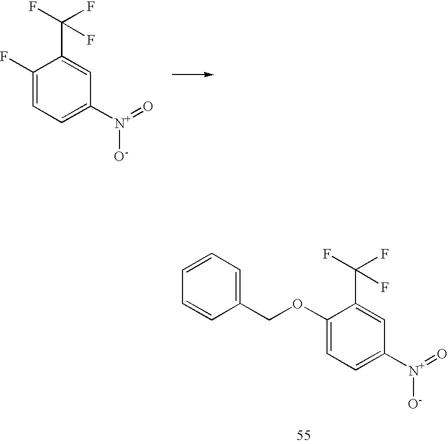

2.15 g (71.67 mmol) sodium hydride in 50 ml DMF are treated under cooling with a solution of 5.5 ml (53.4 mmol) benzyl alcohol in 25 ml DMF. After stirring for 30 min at room temperature a solution of 4-Fluoro-3-trifluoromethyl nitro benzene in 25 ml DMF are added and the reaction mixture is stirred for another 16 hours at room temperature. The product is crystallised by addition of 500 ml H₂O and the precipitated crystals are removed by filtration, rinsed with water and dried.

Yield: 6.59 g (63%) 55, yellow crystals

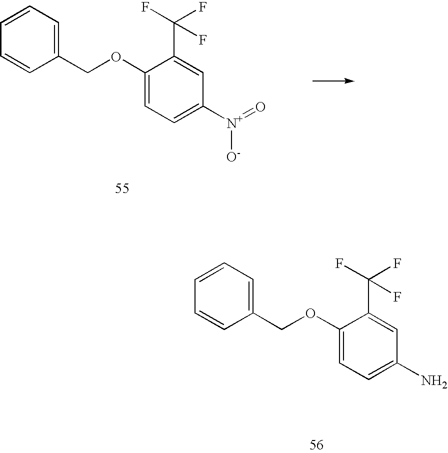

6.58 g (22.14 mmol) 55 are hydrogenated at room temperature in THF using H₂ and Pt/C (5%). The catalyst is removed by filtration and the filtrate is evaporated to dryness.

Yield: 4.79 g (81%) 56, brown oil

As described herein or an analogous manner thereof, the following compounds are obtained:

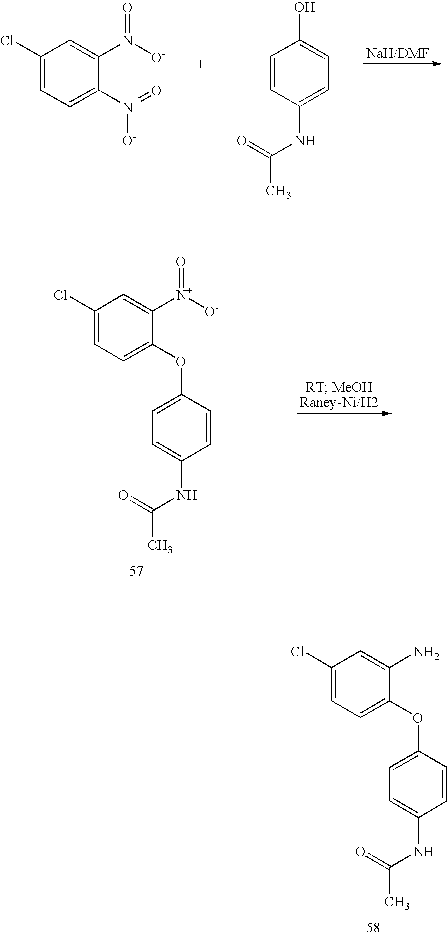

Yield: 36 g (98%) 57; yield: 3.4 g (65%) 58, grey crystals 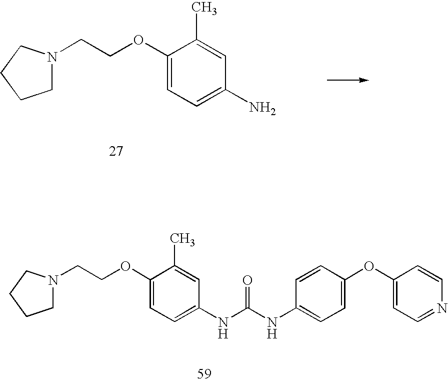
a) 300 mg (1.36 mmol) 27 in 10 ml THF are cooled to 0° C. and treated with 133 mg (0.45 mmol) bis(trichloromethyl)-carbonate and then with 350 μl (2.52 mmol) triethyl amine which is added within 5 min at 0° C. dropwise. Stirring is continued for another 5 min.
b) The thus obtained solution is treated with 250 mg (1.34 mmol) 24 in 10 ml THF at 10° C. and stirred overnight at room temperature. The reaction mixture is treated with water extracted with ethylacetate. The organic phase is washed with water, dried using Na₂SO₄, filtered and evaporated. The residue is purified by preparative thin layer-chromatography (eluent: DCM/15% MeOH/1% NH₄OH).

Yield: 100 mg (18%) 59 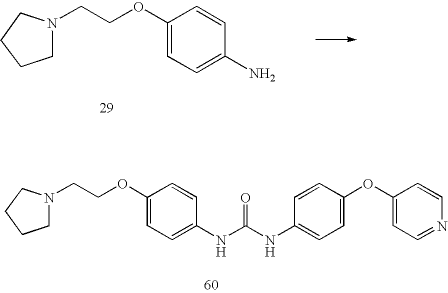
a) 250 mg (1.21 mmol) 29 in 10 ml THF are cooled to 0° C. and treated with 133 mg (0.45 mmol) bis(trichloromethyl)-carbonate and then with 350 μl (2.52 mmol) triethyl amine which is added within 5 min at 0° C. dropwise. Stirring is continued for another 5 min.
b) The thus obtained solution is treated with 250 mg (1.34 mmol) 24 in 10 ml THF at 10° C. and stirred overnight at room temperature. The reaction mixture is treated with water extracted with ethylacetate. The organic phase is washed with water, dried using Na₂SO₄, filtered and evaporated. The residue is purified by chromatography (silica gel, eluent: DCM/2-10% MeOH/0-0.4% NH₄OH).

Yield: 200 mg (41%) 60 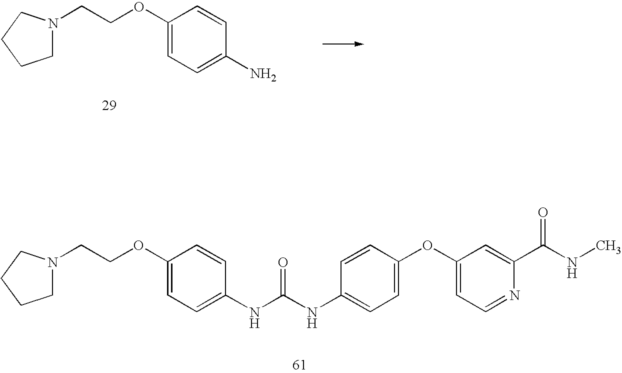
a) 250 mg (1.21 mmol) 29 in 10 ml THF are cooled to 0° C. and treated with 133 mg (0.45 mmol) bis(trichloromethyl)-carbonate and then with 350 μl (2.52 mmol) triethyl amine which is added within 5 min at 0° C. dropwise. Stirring is continued for another 5 min.
b) The thus obtained solution is treated with 250 mg (1.03 mmol) 3a in 10 ml THF at 10° C. and stirred overnight at room temperature. The reaction mixture is treated with water extracted with ethylacetate. The organic phase is washed with water, dried using Na₂SO₄, filtered and evaporated. The residue is purified by chromatography (silica gel, eluent: DCM/5-20% MeOH/0-1% NH₄OH).

Yield: 277 mg (49%) 61

In an analogous manner, compound 62 is obtained in 49% yield from 31 and 24:

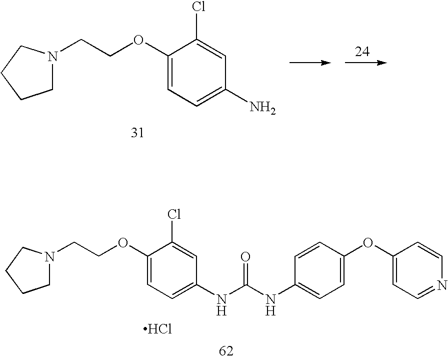

In an analogous manner, compound 63 is obtained in 33% yield from 33 and 3a:

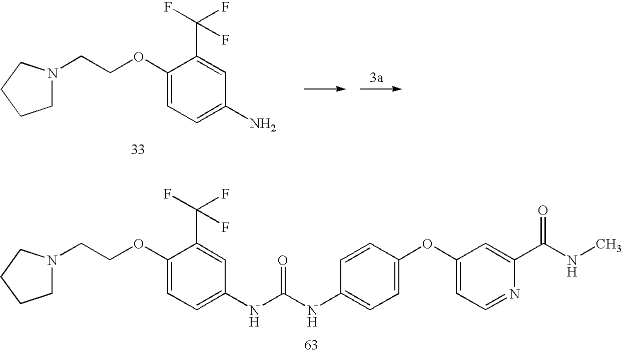

In an analogous manner, compound 64 is obtained in 5% yield from 33 and 24:

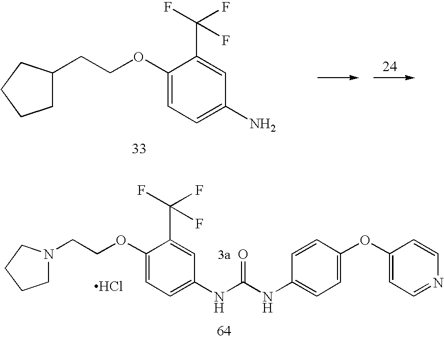

In an analogous manner, compound 65 is obtained in 84% yield from 35 and 24:

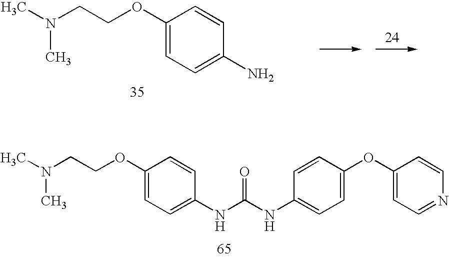

In an analogous manner, compound 66 is obtained in 24% yield from 35 and 3a:

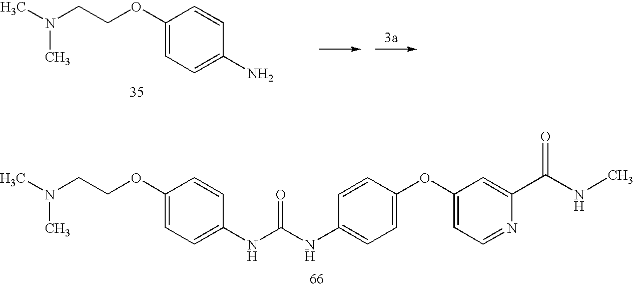

In an analogous manner, compound 67 is obtained in 76% yield from 37 and 3a:

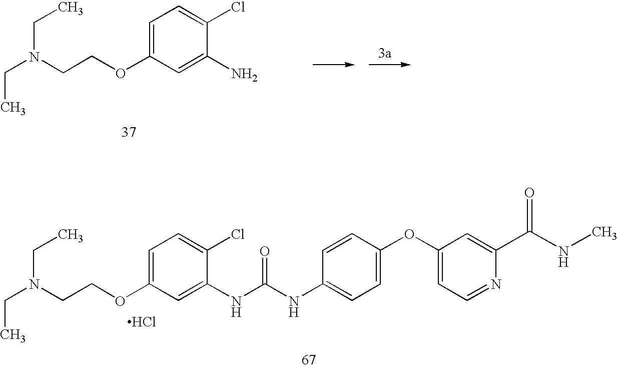

In an analogous manner, compound 68 is obtained from 39 and 3a and purified by RP-chromatography (yield: 12%):

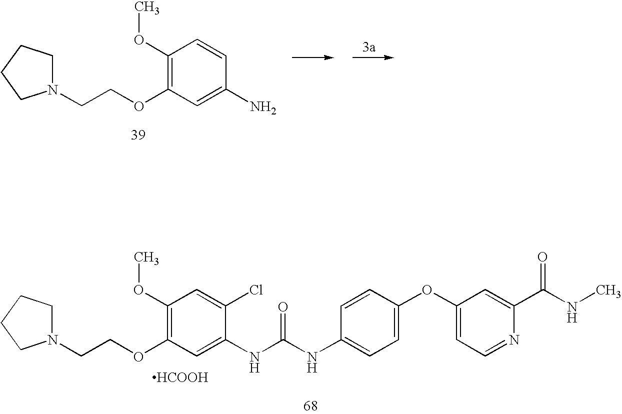

In an analogous manner, compound 69 is obtained from 41 and 3a and purified by RP-chromatography (yield: 6%): 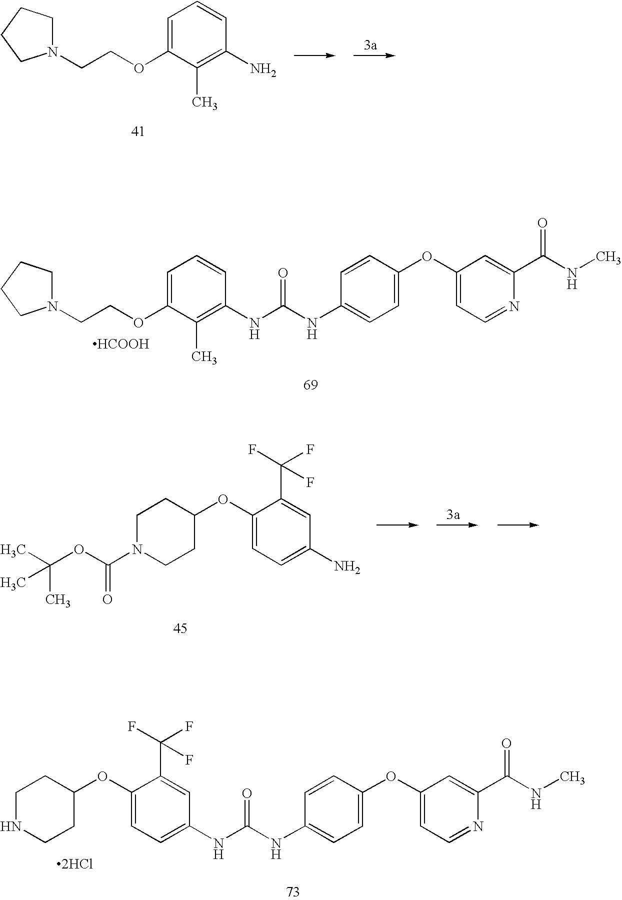
a) 770 mg (1.5 mmol) 45 in 25 ml THF are cooled to 0° C. and treated with 150 mg (0.51 mmol) Bis(trichloromethyl)-carbonate and then with 500 μl (2.94 mmol) N-Ethyldiisopropyl amine which is added within 5 min at 0° C. dropwise. Stirring is continued for another 15 min.
b) The thus obtained solution is treated with 350 mg (1.44 mmol) 3a in 1 ml THF at 10° C. and stirred overnight at room temperature. The reaction mixture is treated with water and extracted with ethylacetate. The organic phase is separated, washed with water, dried using Na₂SO₄, filtered and evaporated. The residue is purified by chromatography on silica gel.

Yield: 750 mg (80%)

c) 750 mg (1.19 mmol) of the Boc-protected product in 10 ml MeOH are treated with 10 ml 4N-HCl in dioxane and stirred overnight. The reaction mixture is evaporated, the residue digested with acetone and the crystals are separated by filtration and dried.

Yield: 500 mg (68%) 73

In an analogous manner as described for compound 73, compound 70 is obtained in 73% yield from 43 and 24:

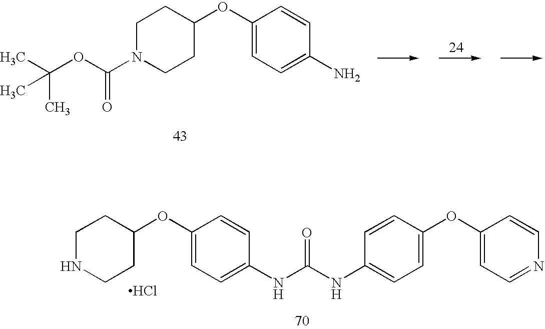

In an analogous manner as described for compound 73, compound 71 is obtained in 40% yield from 43 and 3a:

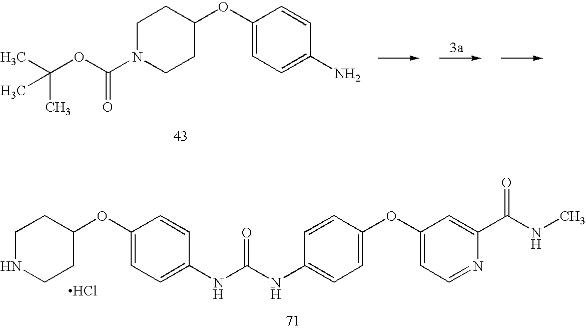

In an analogous manner as described for compound 73, compound 72 is obtained in 66% yield from 43 and 24: 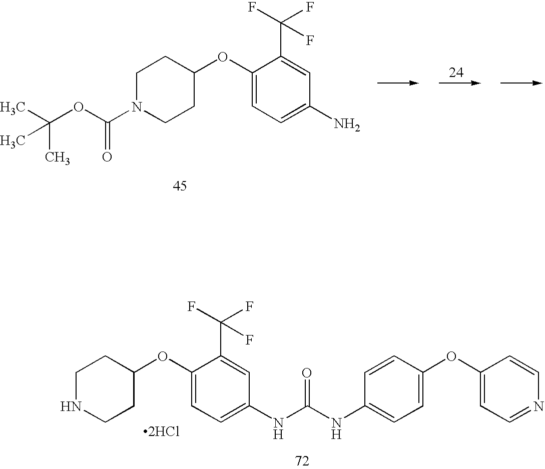 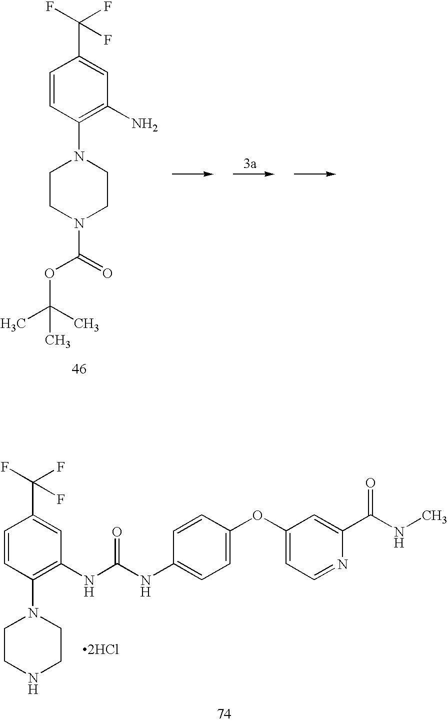
a) 261 mg (0.88 mmol) Triphosgen in 25 ml abs. THF are cooled to −70° C. and treated with 648 mg (2.66 mmol) 3a and 906 μl triethylamine in THF within 15 min in a nitrogen atmosphere. After 10 min of stirring at this temperature, the reaction mixture is treated with a mixture of 920 mg (2.66 mmol) 46 and 453 μl triethylamine within 10 min. Stirring is continued for 1 h at this temperature and for 20 h at room temperature. The reaction mixture is evaporated, the residue taken up in EtOAc, washed 3× with 5% KHSO₄-solution and 1× with 5% NaHCO₃-solution. The organic phase is separated, dried using Na₂SO₄, filtered and evaporated. The residue is purified by chromatography (40 g silica gel, eluent: DCM/MeOH (0-8%).

Yield: 907 mg (55%)

b) 675 mg (1.1 mmol) of the Boc-protected product are treated with 2 ml 2-Propanol and 10 ml 4N-HCl in Dioxan, stirred for 30 min and evaporated. The residue is digested with diethyl ether, filtered and dried.

Yield: 595 mg (92%) 74, pale yellow solid

In an analogous manner, compound 75 is obtained in 73% yield from 48 and 24:

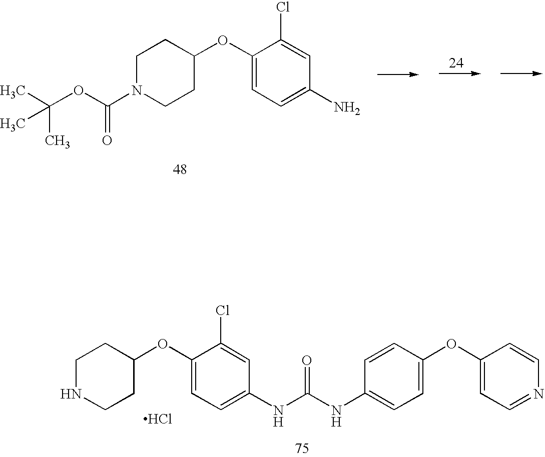

In an analogous manner, compound 76 is obtained in 85% yield from 48 and 3a:

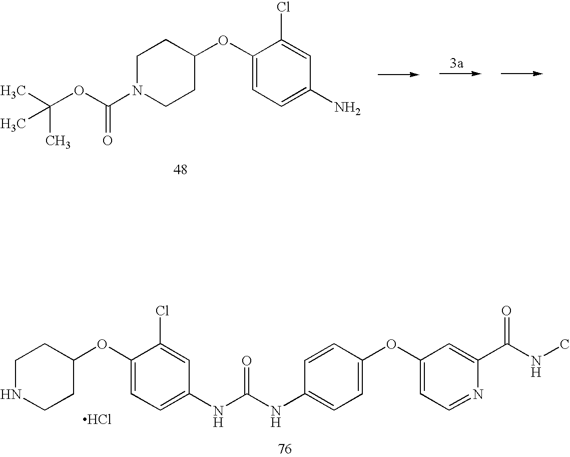

In an analogous manner, compound 77 is obtained in 13% yield from 50 and 3a:

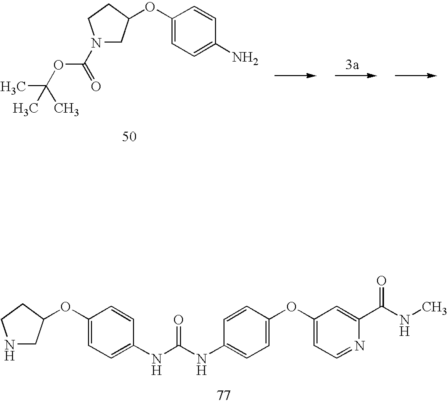

In an analogous manner, compound 78 is obtained in 64% yield from 52 and 3a: 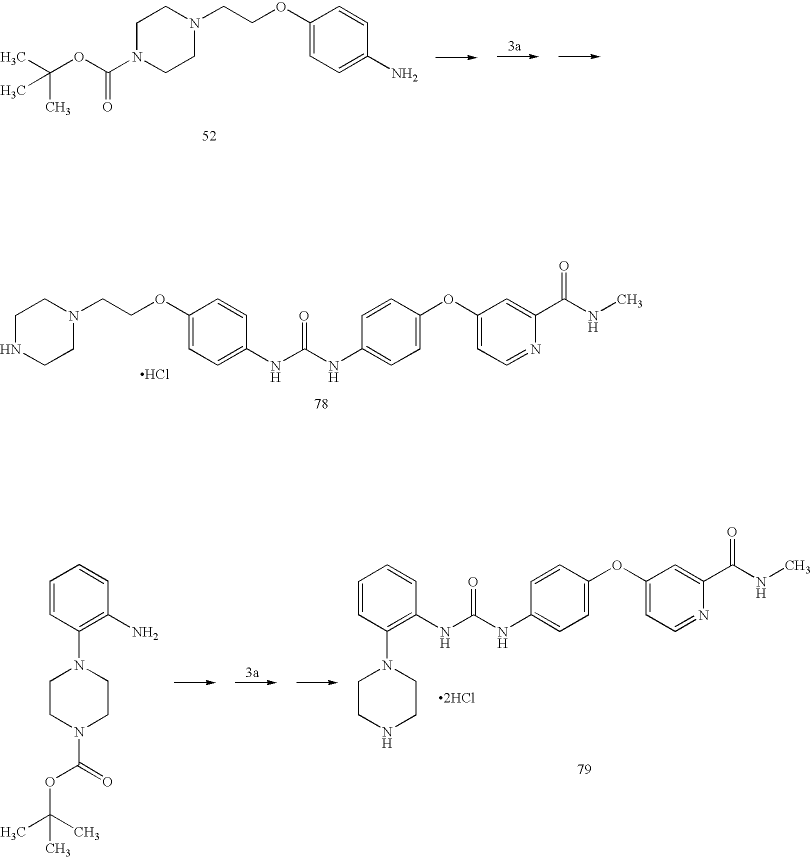
a) 153.5 mg (0.52 mmol) Triphosgen in 20 ml abs. THF is cooled to −60° C. and treated with 435 mg (1.57 mmol) 4-(2-Aminophenyl)-piperazine-1-carboxylic acid tert.-butylester and 666 μl triethylamine in THF within 10 min in a nitrogen atmosphere. Stirring is continued for 30 min at this temperature and then a mixture of 495.5 (1.57 mmol) 3a*2HCl and 666 μl triethylamine is added within 10 min. Stirring is continued for 1 h at this temperature and 20 h at room temperature. Then the reaction mixture is evaporated, the residue taken up in EtOAc and washed 3× with 5% KHSO₄-solution and 1× with 5% NaHCO₃-solution. The organic phase is dried using Na₂SO₄, filtered and evaporated. The residue is purified by chromatography (40 g silica gel, eluent: DCM/MeOH (0-8%).

Yield: 468 mg (55%)

b) 428 mg (0.78 mmol) of the Boc-protected product are treated with 10 ml Methanol and 15 ml 4N-HCl in Dioxan, stirred for 1 h and evaporated. The residue is digested with diethyl ether, filtered and dried.

Yield: 225 mg (55%) 79 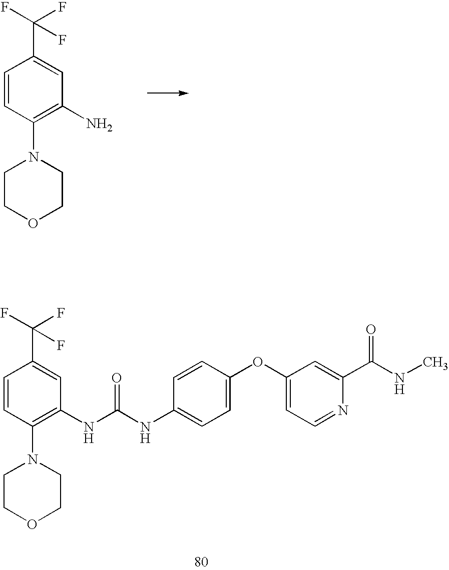
a) 130 mg (0.44 mmol) Triphosgen in 20 ml abs. THF wurde auf −70° C. is cooled to −70° C. and treated with 328 mg (1.33 mmol) 2-Morpholino-5-trifluoromethyl aniline and 453 μl triethylaminin THF within 15 min in a nitrogen atmosphere. Stirring is continued for 10 min at this temperature, then a mixture of 324 (1.33 mmol) 3a and 227 μl triethylamine is added within 10 min. Stirring is continued for 1 h at this temperature and then for 18 h at room temperature. The reaction mixture is evaporated and the residue is taken up in EtOAc, washed 3× with 5% KHSO₄-solution and 1× with 5% NaHCO₃-solution. The organic phase is dried using Na₂SO₄, filtered and evaporated. The residue is purified by chromatography (12 g silica gel, eluent: DCM/MeOH (0-10%). The residue is digested with dichloromethane, filtered and dried.

Yield: 248 mg (36%) 80, colourless solid

The compound 81 is obtained in 71% yield in an analogous manner to the procedures described for compounds 59, 60 and 61:

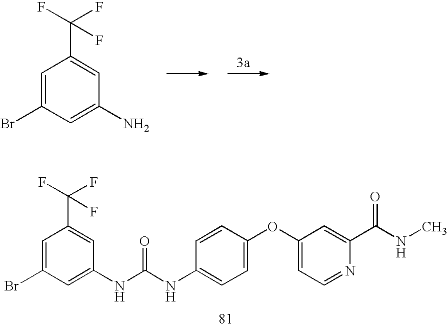

The compound 82 is obtained in 59% yield in an analogous manner to the procedures described for compounds 59, 60, 61 and 81:

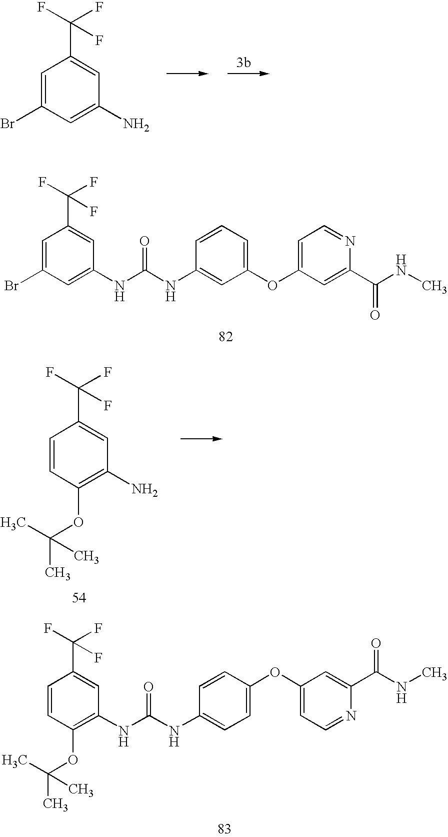

93.2 mg (0.31 mmol) Triphosgen in THF are cooled to −70° C. in a N₂ atmosphere and treated with 220 mg (0.94 mmol) 54 and 200 μl triethylamine in THF within 15 min. After a few minutes, the cooling bath is removed and the reaction mixture is allowed to warm up to room temperature. Then, the reaction mixture is treated with 229.4 (0.94 mmol) 3a and 130 μl triethylamine within 5 min and stirring is continued for another 3 h. The reaction mixture is evaporated, the residue is taken up in EtOAc, washed 2× with 5% KHSO₄-solution and 1× with 5% NaHCO₃-solution. The organic phase is dried using Na₂SO₄, filtered, evaporated and the residue is purified by chromatography (12 g silica gel, eluent: DCM/MeOH (0-10%).

Yield: 136 mg (29%) 83

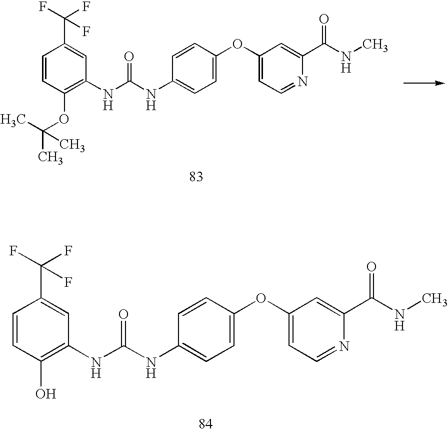

90.6 mg (0.18 mmol) 83 are stirred with 8 ml 4N-HCl in dioxane for 2 h at room temperature Then the reaction mixture is evaporated.

Yield: 69 mg (79%) 84, pale brown solid

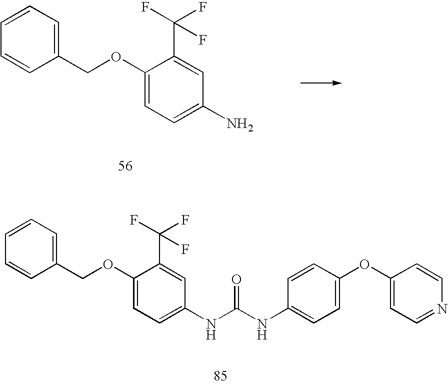

1 g (3.74 mmol) 56, 697 mg (3.74 mmol) 24 and 3.18 ml (18.7 mmol) N-Ethyldiisopropylamine in 40 ml THF are cooled to 0° C., treated with 740 mg (2.49 mmol) Bis(trichloromethyl)-Carbonate in 10 ml THF and stirred for 2 h at room temperature. Then, the reaction mixture is filtered and the filtrate is evaporated. The residue is purified by chromatography (silica gel, eluent: petroleum ether/EtOAc) and/or recrystallisation from diethylether.

Yield: 305 mg (17%) 85, colourless crystals

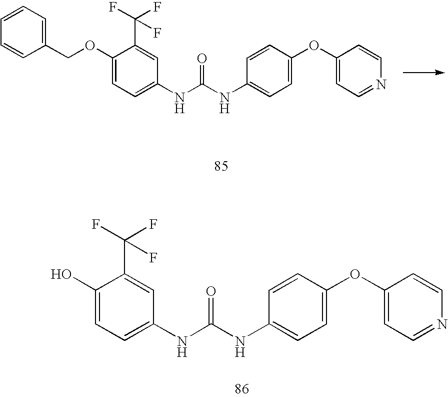

290 mg (0.61 mmol) 85 are hydrogenated in THF at room temperature using H₂ and Pd/C (5%). The catalyst is removed by filtration and the filtrate is evaporated.

Yield: 205 mg (87%) 86, colourless crystals

The compound 87 is obtained in 10% yield in an analogous manner to the procedure described for compound 85:

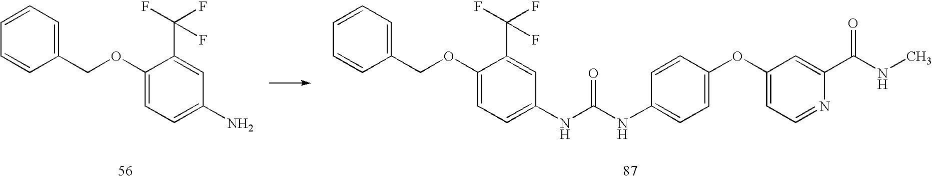

The compound 88 is obtained in 41% yield in an analogous manner to the procedure described for compound 86: 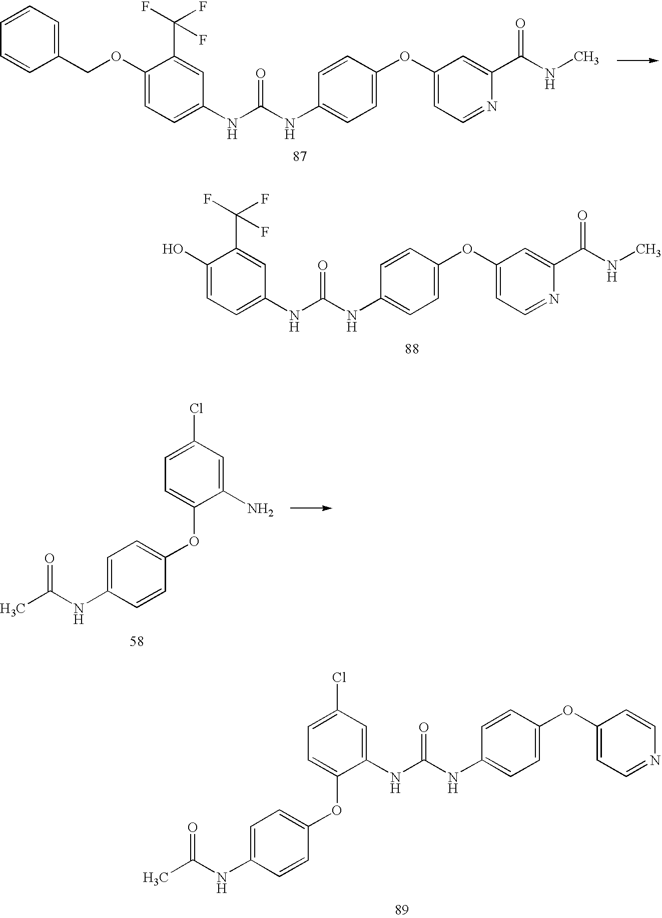
a) 400 mg (1.23 mmol) 58 in 10 ml THF are cooled to 5° C., treated with 133 mg (0.45 mmol) Bis(trichloromethyl)-Carbonate, then with 350 μl (2.52 mmol) triethylamine which is added within 5 min at 0° C., and stirring is continued for 10 min.
b) The thus obtained solution is treated with 250 mg (1.34 mmol) 24 in 10 ml THF at 10° C. and stirring is continued overnight at room temperature. The reaction mixture is treated with ethylacetate and water, the organic phase is separated, washed with water, dried using Na₂SO₄, filtered and evaporated. The residue is crystallised from dichloromethane by addition of methanol.

Yield: 52 mg (8%) 89 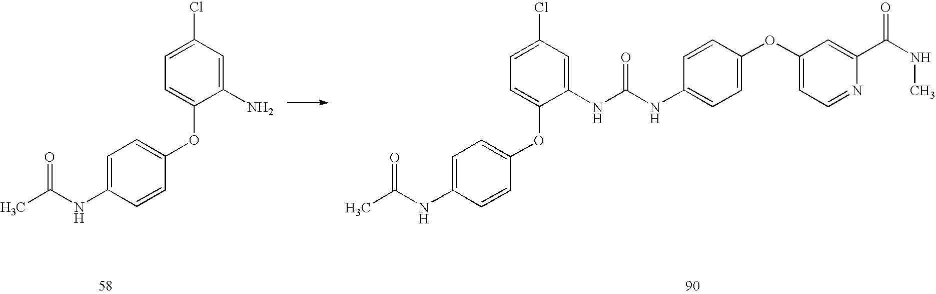
a) 300 mg (0.92 mmol) 58 in 10 ml THF are cooled to 10° C., treated with 100 mg (0.34 mmol) Bis(trichloromethyl)-Carbonate and then with 250 μl (1.8 mmol) triethylamine within 5 min at 10° C., and stirring is continued for 10 min.
b) The thus obtained solution is treated at 10° C. with 250 mg (1.03 mmol) 3a in 4 ml THF and stirring is continued overnight at room temperature. The reaction mixture is treated with ethylacetate and water, the organic phase is separated, washed with water, dried using Na₂SO₄, filtered and evaporated. The residue is purified by chromatography (silica gel, eluent: MTB-ether/methanol (0-10%).

Yield: 78 mg (14%) 90

The compound 91 is obtained in 57% yield in an analogous manner to the procedure described for compound 90, with the exception that compound 90 is purified by RP-chromatography:

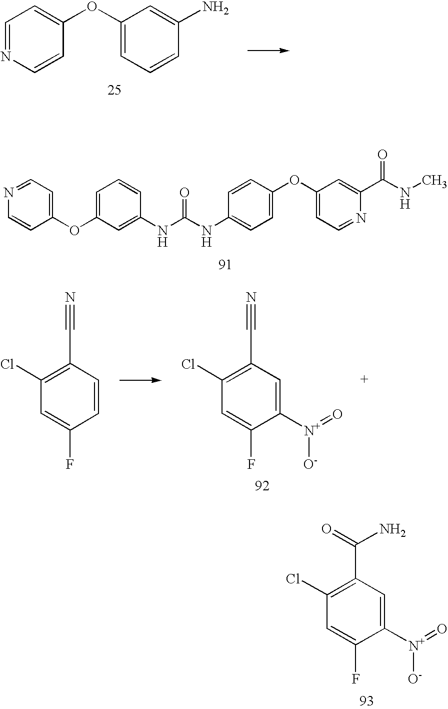

2 g (12.86 mmol) 2-Chloro-4-fluorobenzonitrile in 20 ml H₂SO₄ are treated with 1.7 g (16.81 mmol) KNO₃ in small portions at 5-10° C. The reaction mixture is allowed to warm up to room temperature overnight, then poured onto ice. The obtained precipitate is removed by filtration and rinsed neutral with water. The solid is digested with dichloromethane, filtered and dried.

Yield: 780 mg (26%) 93, colourless solid

The filtrate is evaporated and the residue is purified by chromatography (110 g silica gel, n-heptane/DCM (50-100%)).

Yield: 680 mg (26%) 92, colourless solid

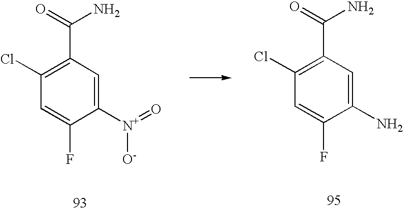

780 mg (3.28 mmol) 93 and 3.8 g (16.84 mmol) SnCl₂.2H₂O in 10 ml EtOAc and 5 ml Ethanol are stirred 2 h at 70° C. The reaction mixture is neutralised (pH 7) using Na₂CO₃-solution, the formed precipitate is removed by filtration and the filtrate is diluted with water. The organic phase is separated, washed with water, dried using Na₂SO₄, filtered and evaporated.

Yield: 570 mg (78%) 95, pale yellow solid 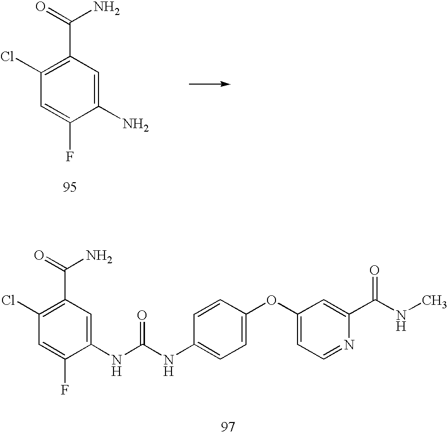
a) 200 mg (0.9 mmol) 95 in 10 ml THF are cooled to 10° C., treated with 100 mg (0.34 mmol) Bis(trichloromethyl)-Carbonate, then with 250 μl (1.8 mmol) triethylamine within 5 min at 10° C., and stirring is continued for 10 min.
b) The thus obtained solution is treated with 237 mg (0.97 mmol) 3a in 4 ml THF at 10° C. and stirring is continued overnight at room temperature. The reaction mixture is diluted with water, the formed precipitate is removed by filtration and dried.

Yield in this: 239 mg (54%) 97

a) 10 g (48.51 mmol) 4-Nitro-2-(trifluoromethyl)-aniline, 5.73 g (48.51 mmol) succinic acid and 60 g polyphosphoric acid are combined and heated to 85° C. under stirring for 20 h. The reaction mixture is stirred into water (500 ml), the formed precipitate is removed by filtration, rinsed with water and dried.

Yield: 13.57 g (97%)

b) 13.4 g (46.5 mmol) of the obtained nitro compound are hydrogenated at room temperature in methanol/THF—3/1 using H₂ and Raney-Ni. The catalyst is removed by filtration and the filtrate is evaporated. The residue is purified by chromatography (silica gel, DCM/MTB-ether—9/1).

Yield: 7.11 g (59%) 98

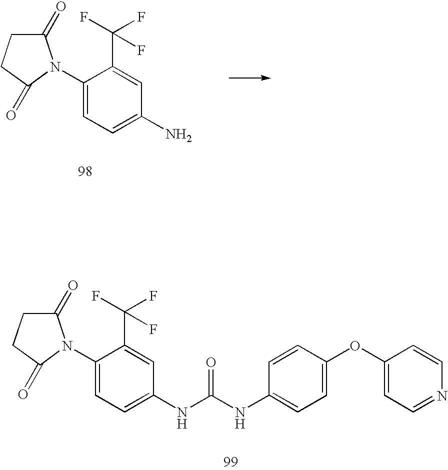

Compound 98 is reacted, purified and isolated in an analogous manner to compound 59.

Yield: 42%, 99 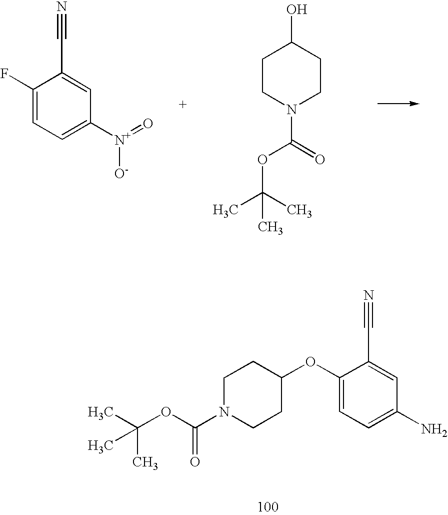
a) 1.3 g (7.67 mmol) 2-Fluoro-5-nitrobenzonitrile, 5.5 g (16.89 mmol) Cs₂CO₃ and 2.2 g (10.6 mmol) 4-Hydroxypiperidine-N-carboxylic acid, tert.-butylester in 15 ml DMF are reacted and isolated according to the procedure as described for compound 42.

Yield: 0.98 g (37%)

b) 0.96 g (2.76 mmol) of the nitro compound are hydrogenated at room temperature in methanol using H₂ and Pd—C (5%). The catalyst is removed by filtration and the filtrate is evaporated to dryness.

Yield: 0.81 g (85%) 100

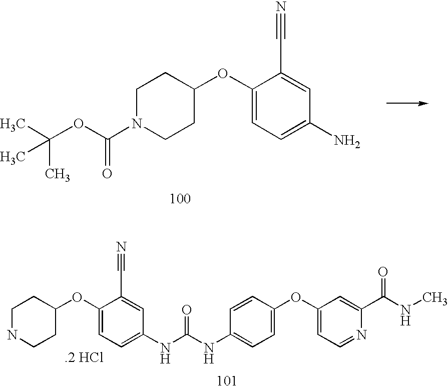

Compound 100 is reacted and isolated in an analogous manner as described for compound 73. Yield: 22% 101

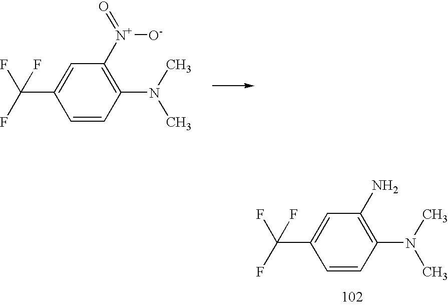

2 g (8.54 mmol) 4-(N,N-Dimethylamino)-3-nitrobenzotrifluoride are hydrogenated at room temperature in THF using H₂ and Pd—C (5%). The catalyst is removed and the filtrate is evaporated to dryness. Yield: 1.69 g (97%) 102

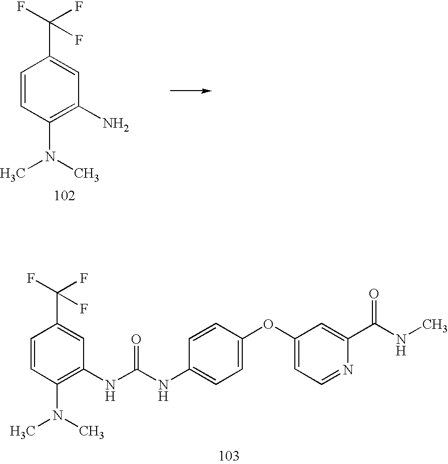

Compound 102 is reacted and isolated in an analogous manner as described for compound 80. Yield: 43%, 103

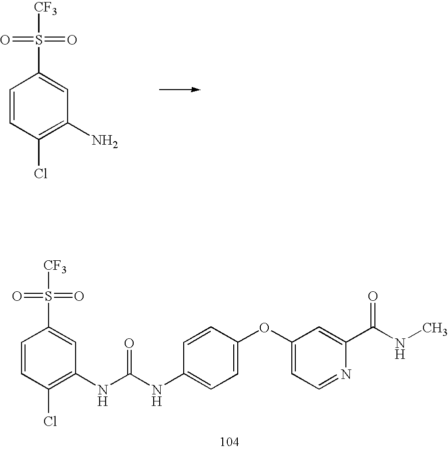

2-Chloro-5-(trifluoromethylsulfonyl)-aniline is reacted and isolated in an analogous manner as described for 80. Yield: 5%, 104

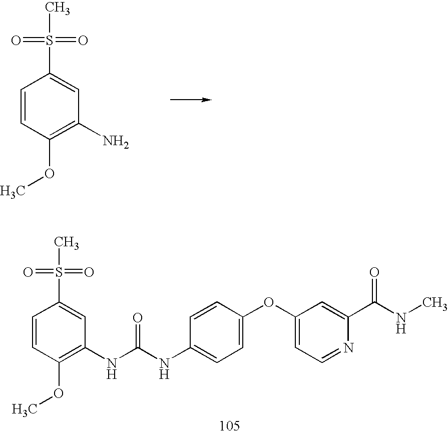

2-Methoxy-5-(methylsulfonyl)-anilines reacted and isolated in an analogous manner as described for 80. Yield: 25%, 105

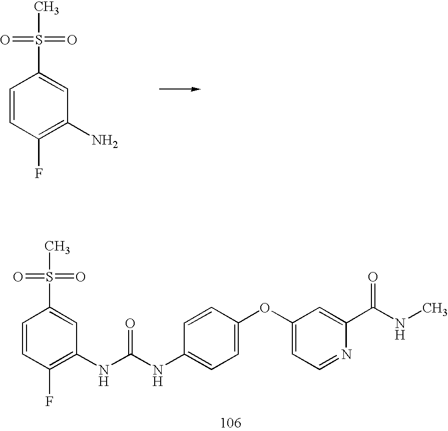

2-Fluoro-5-(methylsulfonyl)-aniline is reacted and isolated in an analogous manner as described for 80. Yield: 7%, 106

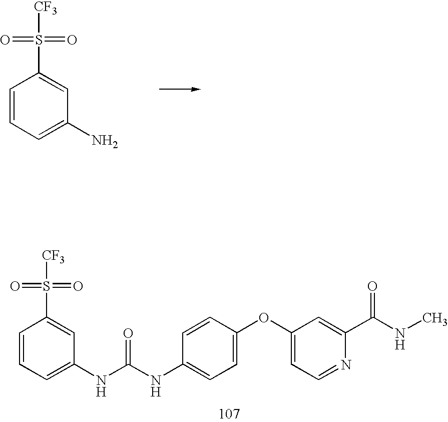

5-(Trifluoromethylsulfonyl)-aniline is reacted and isolated in an analogous manner as described for 80. Yield: 32%, 107

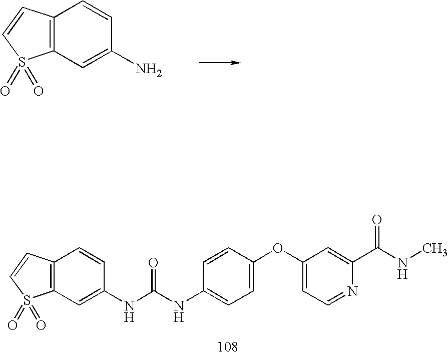

1,1-Dioxo-1H-benzo[b]thiophenyl-6-amine is reacted and isolated in an analogous manner as described for 80. Yield: 30%, 108

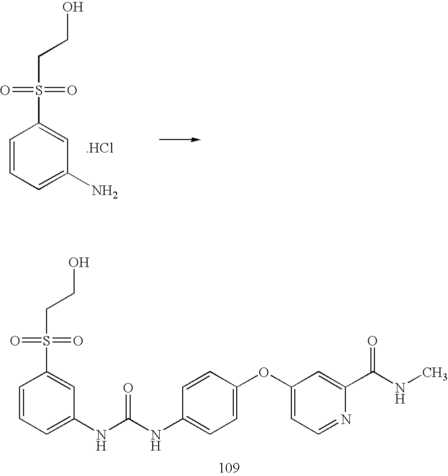

2-(3-Aminobenzenesulfonyl)ethanol hydrochloride is reacted and isolated in an analogous manner as described for 80. Yield: 20%, 109

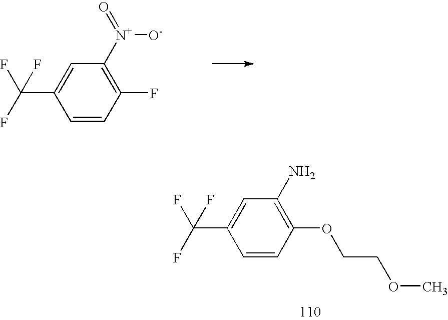

Compound 110 is synthesised according to the general working procedure as described for compounds 4 and 5, respectively. Yield: 90%

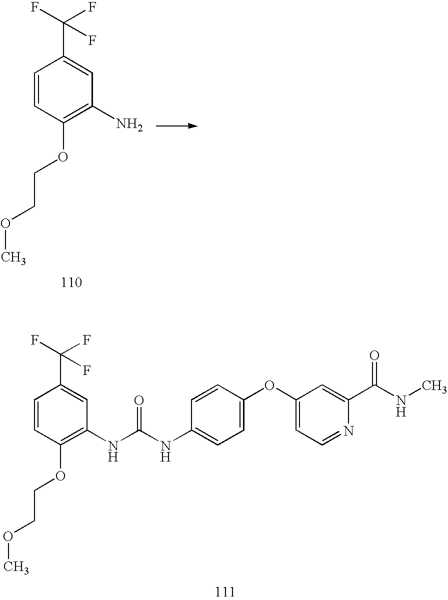

Compound 110 is reacted and isolated in an analogous manner as described for compound 16. Yield: 27%, 111

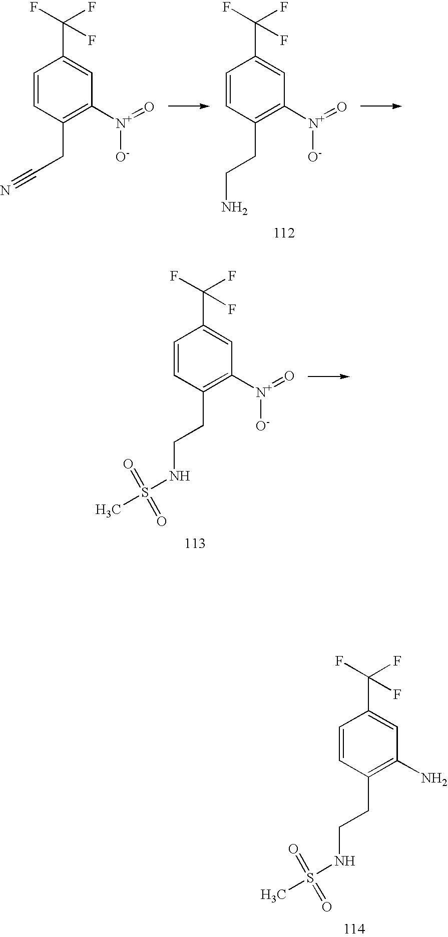

1.75 g (7.6 mmol) (2-Nitro-4-trifluoromethylphenyl)-acetonitrile in 20 ml THF are cooled to 0° C. in a nitrogen atmosphere and treated with 60 ml BH₃/THF-complex (1M in THF). The reaction mixture is allowed to slowly warm up to room temperature overnight. After 72 h the reaction solution is slowly given to 50 ml 5 N HCl gegeben and then 1 h heated to reflux. The reaction mixture is evaporated to dryness, the residue made alkaline (pH 12-14) with 25% NaOH solution and extracted 2× with 100 ml ethylacetate. The combined organic phases are washed with brine, dried using Na₂SO₄, filtered and evaporated.

Yield: 1.8 g (77%) 112, brown oil

500 mg (2.14 mmol) 112 in 5 ml Dichloromethane are treated with 360 μl (4.46 mmol) pyridine, cooled to 0° C., then treated under stirring with 119 μl (2.45 mmol) Methansulfonylchloride and stirred overnight at room temperature. After dilution with DCM, 1N HCl is added, the organic phase is separated, washed 1× with brine, dried using Na₂SO₄, filtered and evaporated.

Yield: 640 mg (74%) 113, brown oil

compound 113 is hydrogenated in THF at room temperature using H₂ and Pd—C (5%). The catalyst is removed by filtration and the filtrate is evaporated.

Yield: 99%, 114

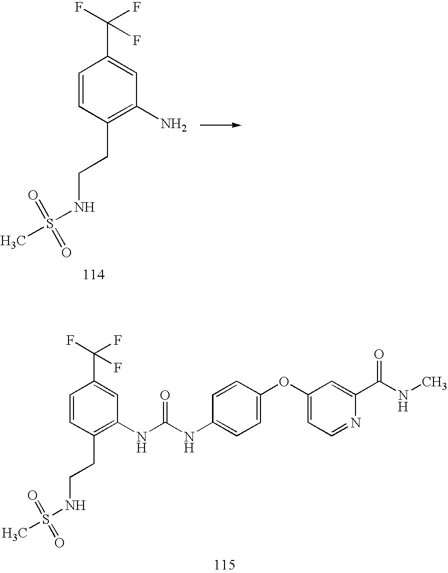

Compound 114 is reacted and isolated in an analogous manner as described for compound 16. Yield: 6%, 115

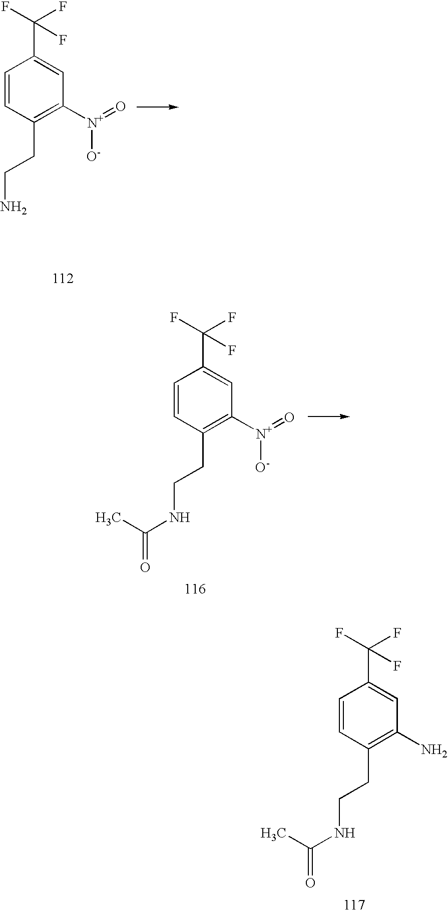

Compound 112 is reacted with acetyl chloride and isolated in an analogous manner as described for compound 113. Yield: 60%, 116 Compound 116 is hydrogenated at room temperature in THF using H₂ and Raney-Ni. The catalyst is removed by filtration and the filtrate is evaporated to dryness. Yield: 77%, 117

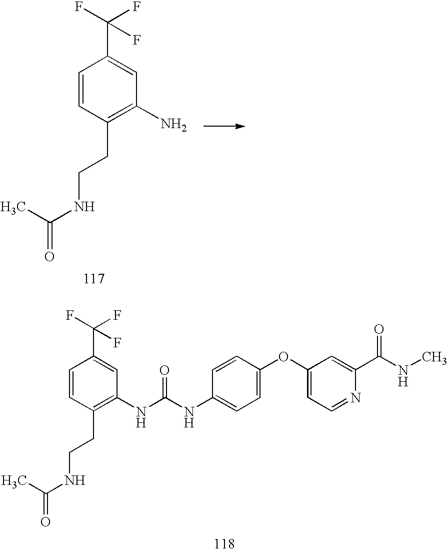

Compound 117 is reacted and isolated in an analogous manner as described for compound 16. Yield: 50%, 118

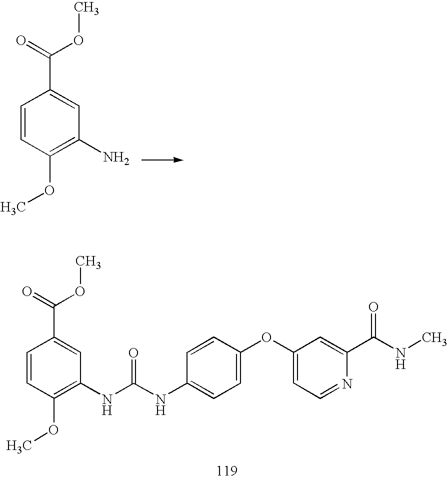

3-Amino-4-methoxy-benzoic acid methylester is reacted and isolated in an analogous manner as described for compound 80. Yield: 19%, 119

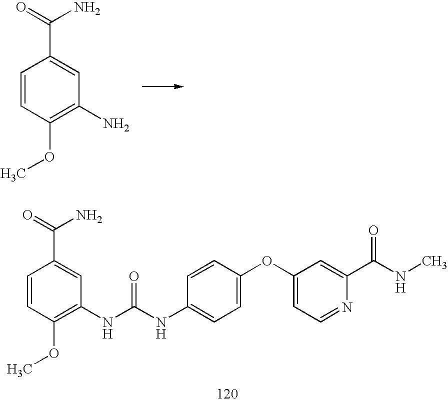

3-Amino-4-methoxy-benzoic acid amide is reacted and isolated in an analogous manner as described for compound 80. Yield: 4%, 120

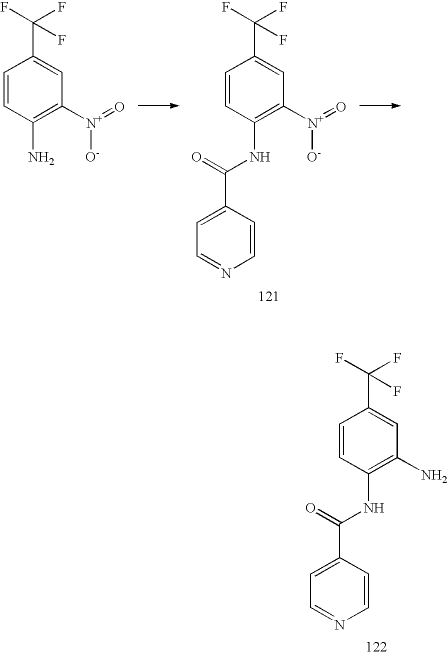

10.31 g (50 mmol) 2-Nitro-4-(trifluoromethyl)-aniline in 100 ml Pyridin are treated with 10.31 g (55 mmol) Isonicotinic acid chloride-hydrochloride and heated for 4 h or the steam bath. The reaction mixture is diluted with water and the formed precipitate is removed by filtration, rinsed with water and dried. Yield: 15.17 g (98%) 121 Compound 121 is hydrogenated at room temperature in methanol using H₂ and Raney-Ni. The catalyst is removed by filtration and the filtrate is evaporated to dryness. Yield: 77%, 122

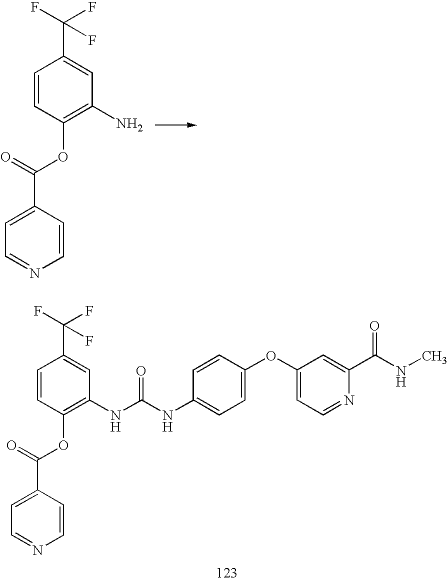

Compound 123 is reacted and isolated as described for compound 80. Yield: 13%, 123

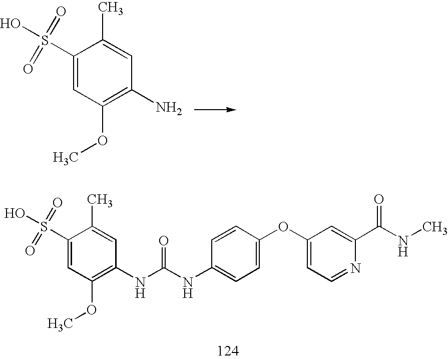

4-Amino-5-methoxy-2-methylbenzenesulfonic acid is reacted in an analogous manner as described for compound 80. Yield: 63%, 124

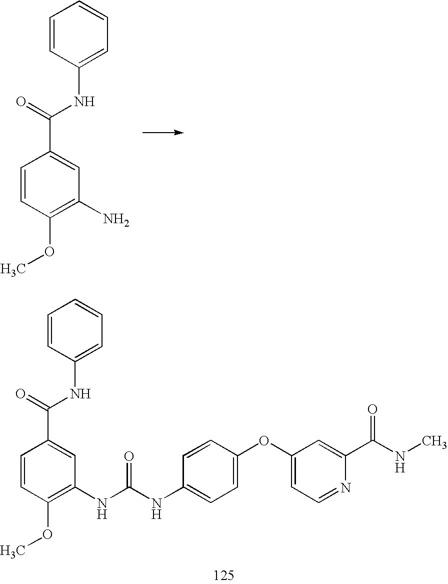

3-Amino-4-methoxy-N-phenyl-benzoic acid amide is reacted and isolated in an analogous manner as described for compound 80. Yield: 70%, 125

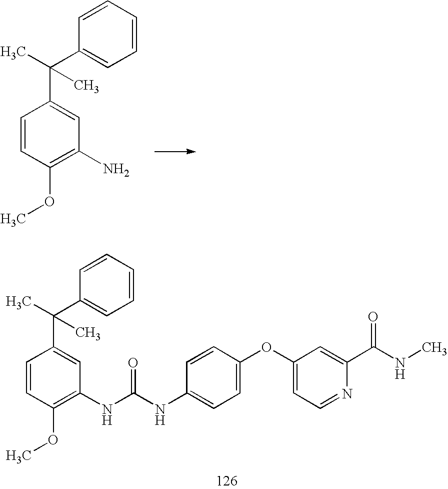

2-Methoxy-5-(1-methyl-1-phenylethyl)-aniline is reacted and isolated in an analogous manner as described for compound 80. Yield: 33%, 126

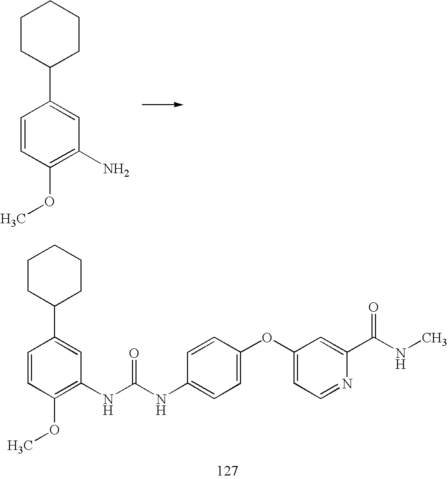

5-Cyclohexyl-2-methoxy-aniline is reacted and isolated in an analogous manner as described for compound 80. Yield: 38%, 127

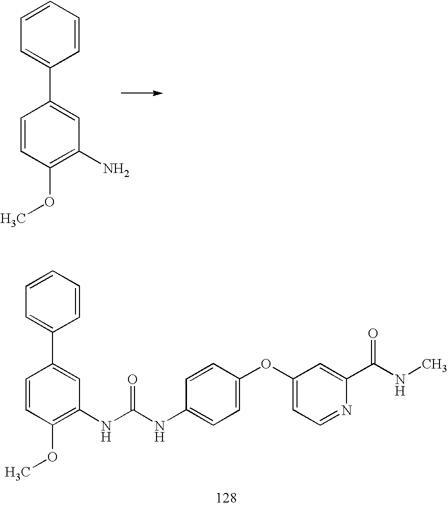

2-Methoxy-5-phenyl-aniline is reacted and isolated in an analogous manner as described for compound 80. Yield: 12%, 128

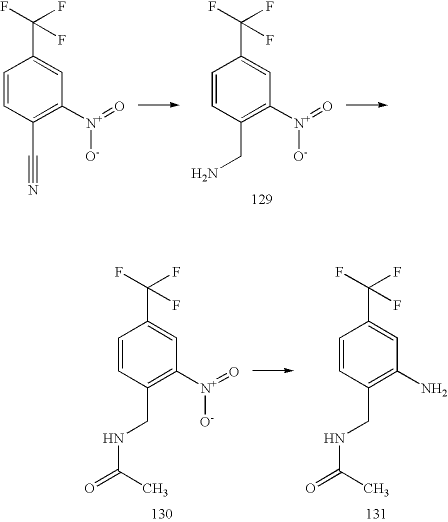

2-Nitro-4-trifluoromethyl-benzonitrile is reacted with the BH₃/THF-complex (1M in THF) and isolated in an analogous manner as described for compound 112. Yield: 62%, 129 Compound 129 is reacted with acetylchloride and isolated in an analogous manner as described for compound 116. Yield: 92%, 130 Compound 130 is hydrogenated at room temperature in THF using H₂ and Raney-Ni. The catalyst is removed by filtration and the filtrate is evaporated to dryness. Yield: 87%, 131

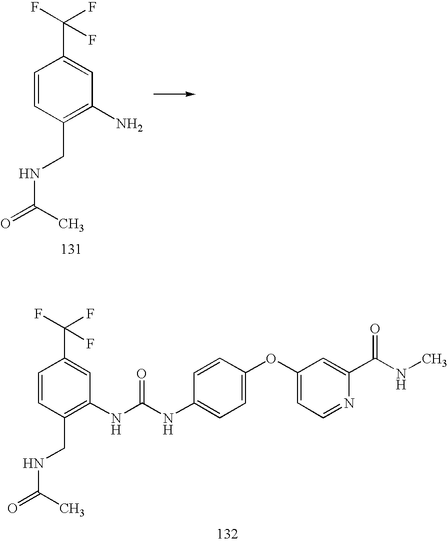

Compound 131 is reacted and isolated in an analogous manner as described for compound 16. Yield: 33%, 132

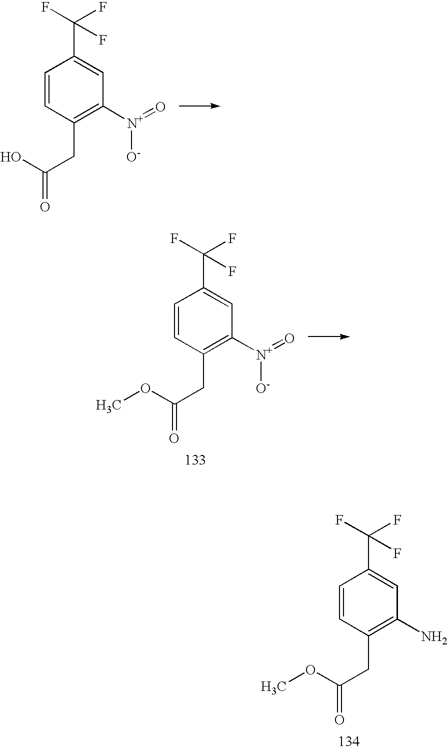

2.2 g (8.83 mmol) (2-Nitro-4-trifluoromethyl-phenyl)acetic acid in 50 ml methanol are treated with 1 ml sulfuric acid and stirred for 90 min at 65° C. After cooling down to room temperature, the reaction mixture is evaporated, treated with 100 ml ethylacetate, washed with 2×50 ml NaHCO₃ solution. The organic phase is dried using Na₂SO₄, filtered and evaporated. Yield: 1.99 g (85%) 133, yellow solid

Compound 133 is hydrogenated in THF at room temperature using H₂ and Pd—C (5%). The catalyst is removed by filtration and the filtrate is evaporated to dryness. Yield: 99%, 134

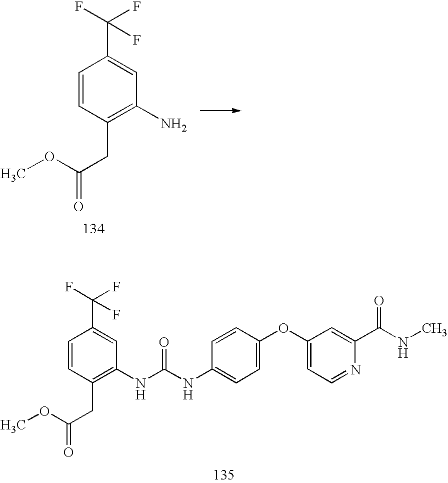

Compound 134 is reacted and isolated in an analogous manner as described for compound 16. Yield: 10%, 135

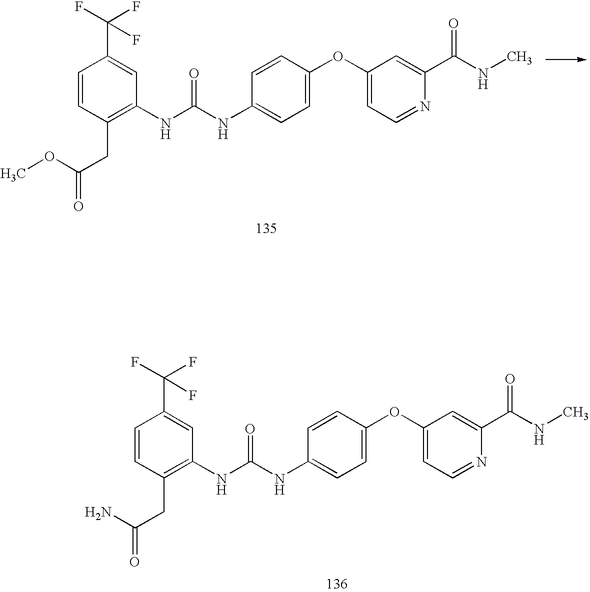

24 mg (0.48 mmol) 135 in 1 ml THF are treated with 0.5 ml NH₃-solution (25%). After addition of 0.5 ml MeOH stirring is continued overnight. The reaction mixture was evaporated, the residue taken up in ethylacetate, washed with water, dried using Na₂SO₄, filtered and evaporated. The residue is purified using RP-Chromatography.

Yield: 3 mg (13%) 136, colourless crystals

Retention times (Rt) as disclosed herein are, if not indicated otherwise, HPLC retention times, obtained according the following methods:

General Method:

Gradient: 5.5 min; flow rate: 2.75 ml/min from 90:10 to 0:100 H₂O/ACN Water+TFA (0.01% by vol.); acetonitrile+TFA (0.01% by vol.)

Column: Chromolith SpeedROD RP 18e 50-4.6

Wavelength: 220 nm.

Method a:

Gradient: 5.5 min; flow rate: 2.75 ml/min from 99:1 to 0:100 H₂O/ACN Water+TFA (0.01% by vol.); acetonitrile+TFA (0.01% by vol.)

Column: Chromolith SpeedROD RP 18e 50-4.6

Wavelength: 220 nm.

The compounds disclosed herein can preferably be produced according to the procedures described herein or in an analogous manner thereof.

### Example A

### Injection Vials

A solution of 100 g of an active compound of the formula I and 5 g of disodium hydrogenphosphate is adjusted to pH 6.5 in 3 l of double-distilled water using 2N hydrochloric acid, sterile-filtered, dispensed into injection vials, lyophilized under sterile conditions and aseptically sealed. Each injection vial contains 5 mg of active compound.

### Example B

### Suppositories

A mixture of 20 g of an active compound of the formula I is fused with 100 g of soya lecithin and 1400 g of cocoa butter, poured into moulds and allowed to cool. Each suppository contains 20 mg of active compound.

### Example C

### Solution

A solution of 1 g of an active compound of the formula I, 9.38 g of NaH₂PO₄.2H₂O, 28.48 g of Na₂HPO₄.12H₂O and 0.1 g of benzalkonium chloride in 940 ml of double-distilled water is prepared. It is adjusted to pH 6.8, made up to 1 l and sterilized by irradiation. This solution can be used in the form of eye drops.

### Example D

### Ointment

500 mg of an active compound of the formula I is mixed with 99.5 g of petroleum jelly under aseptic conditions.

### Example E

### Tablets

A mixture of 1 kg of active compound of the formula I, 4 kg of lactose, 1.2 kg of potato starch, 0.2 kg of talc and 0.1 kg of magnesium stearate is compressed to give tablets in a customary manner such that each tablet contains 10 mg of active compound.

### Example F

### Coated Tablets

Analogously to Example E, tablets are pressed and are then coated in a customary manner using a coating of sucrose, potato starch, talc, tragacanth and colourant.

### Example G

### Capsules

2 kg of active compound of the formula I are dispensed into hard gelatin capsules in a customary manner such that each capsule contains 20 mg of the active compound.

### Example H

### Ampoules

A solution of 1 kg of active compound of the formula I in 60 l of double-distilled water is sterile-filtered, dispensed into ampoules, lyophilized under sterile conditions and aseptically sealed. Each ampoule contains 10 mg of active compound.

1. Bisarylurea derivatives of formula I 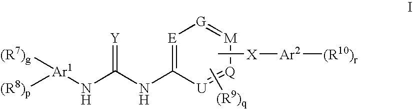
wherein
Ar¹, Ar² are selected independently from one another from aromatic hydrocarbons containing 6 to 14 carbon atoms and ethylenical unsaturated or aromatic heterocyclic residues containing 3 to 10 carbon atoms and one or two heteroatoms, independently selected from N, O and S,
E, G, M, Q and U are selected, independently from one another, from carbon atoms and nitrogen atoms, with the proviso that one or more of E, G, M, Q and U are carbon atoms and that X is bonded to a carbon atom,
R⁷ is independently selected from a group consisting of Het, OHet, N(R¹¹)Het, (CR⁵R⁶)ₖHet, O(CR⁵R⁶)ₖHet, N(R¹¹)(CR⁵R⁶)ₖHet, (CR⁵R⁶)ₖNR¹¹R¹², (CR⁵R⁶)ₖOR¹³, O(CR⁵R⁶)ₖNR¹¹R¹², NR¹¹(CR⁵R⁶)ₖNR¹¹R¹², O(CR⁵R⁶)ₖR¹³, NR¹¹(CR⁵R⁶)ₖR¹³, O(CR⁵R⁶)ₖOR¹³, NR¹¹(CR⁵R⁶)ₖOR¹³, (CR⁵R⁶)ₙO(CR⁵R⁶)ₖNR¹¹R¹², O(CR⁵R⁶)ₙO(CR⁵R⁶)ₖNR¹¹R¹², NR¹¹(CR⁵R⁶)ₙO(CR⁵R⁶)ₖNR¹¹R¹², (CR⁵R⁶)ₙNR¹¹(CR⁵R⁶)ₖNR¹¹R¹², O(CR⁵R⁶)ₙNR¹¹(CR⁵R⁶)ₖNR¹¹R¹², NR¹¹(CR⁵R⁶)ₙNR¹²(CR⁵R⁶)ₖNR¹¹R¹², (CR⁵R⁶)ₙO(CR⁵R⁶)ₖOR¹¹, O(CR⁵R⁶)ₙO(CR⁵R⁶)ₖOR¹¹, NR¹¹(CR⁵R⁶)ₙO(CR⁵R⁶)ₖOR¹², (CR⁵R⁶)ₙNR¹¹(CR⁵R⁶)ₖOR¹², O(CR⁵R⁶)ₙNR¹¹(CR⁵R⁶)ₖOR¹², NR¹²(CR⁵R⁶)ₙNR¹¹(CR⁵R⁶)ₖOR¹², O(CR⁵R⁶)ₖAr³—NR¹¹R¹², SO₂R¹³, SO₂(CR⁵R⁶)ₖOR¹³ and SO₂(CR⁵R⁶)ₖNR¹¹R¹², wherein
R⁵, R⁶ are in each case independently from one another selected from H and A; or R⁵ and R⁶ together optionally represent an oxo-group; or
R⁷ is selected from divalent radicals of formula —SO₂—CR⁸═CR⁸—, wherein both valencies are bound vicinally to Ar¹,
n and/or k independently are 0, 1, 2, 3 or 4, preferably 1, 2, 3 or 4, and even more preferred is 2 or 3;
R⁸, R⁹ and R¹⁰ are independently selected from a group consisting of H, A, cycloalkyl comprising 3 to 7 carbon atoms, Hal, CH₂Hal, CH(Hal)₂, C(Hal)₃, NO₂, (CH₂)ₙCN, (CH₂)ₙNR¹¹R¹², (CH₂)ₙO(CH₂)ₖNR¹¹R¹², (CH₂)ₙNR¹¹(CH₂)ₖNR¹¹R¹², (CH₂)ₙO(CH₂)ₖOR¹¹, (CH₂)ₙNR¹¹(CH₂)ₖOR¹², (CH₂)ₙCOOR¹³, (CH₂)ₙCOR¹³, (CH₂)ₙCONR¹¹R¹², (CH₂)ₙNR¹¹COR¹³, (CH₂)ₙNR¹¹CONR¹¹R¹², (CH₂)ₙNR¹¹SO₂A, (CH₂)ₙSO₂NR¹¹R¹², (CH₂)ₙS(O)ᵤR¹³, (CH₂)ₙOC(O)R¹³, (CH₂)ₙCOR¹³, (CH₂)ₙSR¹¹, CH═N—OA, CH₂CH═N—OA, (CH₂)ₙNHOA, (CH₂)ₙCH═N—R¹¹, (CH₂)ₙOC(O)NR¹¹R¹², (CH₂)ₙNR¹¹COOR¹³, (CH₂)ₙN(R¹¹)CH₂CH₂OR¹³, (CH₂)ₙN(R¹¹)CH₂CH₂OCF₃, (CH₂)ₙN(R¹¹)C(R¹³)HCOOR¹², (CH₂)ₙN(R¹¹)C(R¹³)HCOR¹¹, (CH₂)ₙN(R¹¹)CH₂CH₂N(R¹²)CH₂COOR¹¹, (CH₂)ₙN(R¹¹)CH₂CH₂NR¹¹R¹², CH═CHCOOR¹³, CH═CHCH₂NR¹¹R¹², CH═CHCH₂NR¹¹R¹², CH═CHCH₂OR¹³, (CH₂)ₙN(COOR¹³)COOR¹⁴, (CH₂)ₙN(CONH₂)COOR¹³, (CH₂)ₙN(CONH₂)CONH₂, (CH₂)ₙN(CH₂COOR¹³)COOR¹⁴, (CH₂)ₙN(CH₂CONH₂)COOR¹³, (CH₂)ₙN(CH₂CONH₂)CONH₂, (CH₂)ₙCHR¹³COR¹⁴, (CH₂)ₙCHR¹³COOR¹⁴, (CH₂)ₙCHR¹³CH₂OR¹⁴, (CH₂)ₙOCN and (CH₂)ₙNCO, wherein
R¹¹, R¹² are independently selected from a group consisting of H, A, C(O)A, (CH₂)ₘAr³, C(O)(CH₂)ₘAr³, (CH₂)ₘHet, C(O)(CH₂)ₘHet and S(O)ᵤA, or in NR¹¹R¹², R¹¹ and R¹² form, together with the N-atom they are bound to, a 5-, 6- or 7-membered heterocyclus which optionally contains 1 or 2 additional hetero atoms, selected from N, O and S, which optionally is substituted by one or more substituent, selected from A, R¹³, ═O, ═S and ═N—R¹⁴,
R¹³, R¹⁴ are independently selected from a group consisting of H, Hal, A, (CH₂)ₘAr⁴ and (CH₂)ₘHet,
A is selected from the group consisting of alkyl, alkenyl, cycloalkyl, alkylenecycloalkyl, alkoxy, alkoxyalkyl and saturated heterocyclyl, preferably from the group consisting of alkyl, alkenyl, cycloalkyl, alkylenecycloalkyl, alkoxy and alkoxyalkyl,
Ar³, Ar⁴ are independently from one another aromatic hydrocarbon residues comprising 5 to 12 and preferably 5 to 10 carbon atoms which are optionally substituted by one or more substituents, selected from a group consisting of A, Hal, NO₂, CN, OR¹⁵, NR¹⁵R¹⁶, COOR¹⁵, CONR¹⁵R¹⁶, NR¹⁵COR¹⁶, NR¹⁵CONR¹⁵R¹⁶, NR¹⁶SO₂A, COR¹⁵, SO₂NR¹⁵R¹⁶, S(O)ᵤA and OOCR¹⁵,
Het is a saturated, unsaturated or aromatic heterocyclic residue which is optionally substituted by one or more substituents, selected from a group consisting of A, C(O)A, R¹³, ═O, ═S, ═N—R¹⁴, Hal, NO₂, CN, OR¹⁵, NR¹⁵R¹⁶, COR¹⁵, CONR¹⁵R¹⁶, NR¹⁵COR¹⁶, NR¹⁵CONR¹⁵R¹⁶, NR¹⁶SO₂A, COR¹⁵, SO₂NR¹⁵R¹⁶, S(O)ᵤA and OOCR¹⁵,
R¹⁵, R¹⁶ are independently selected from a group consisting of H, A, and (CH₂)ₘAr⁶, wherein
Ar⁶ is a 5- or 6-membered aromatic hydrocarbon which is optionally substituted by one or more substituents selected from a group consisting of methyl, ethyl, propyl, 2-propyl, tert.-butyl, Hal, CN, OH, NH₂ and CF₃,
k, n and m are independently of one another 0, 1, 2, 3, 4, or 5,
X represents a bond or is (CR¹¹R¹²)ₕ, or (CHR¹¹)ₕ-Q-(CHR¹²), wherein
Q is selected from a group consisting of O, S, N—R¹⁵, (CHal₂)ⱼ, (O—CHR¹⁸)ⱼ, (CHR¹⁸—O)ⱼ, CR¹⁸═CR¹⁹, (O—CHR¹⁸CHR¹⁹)ⱼ, (CHR¹⁸CHR¹⁹—O)ⱼ, C═O, C═S, C═NR¹⁵, CH(OR¹⁵), C(OR¹⁵)(OR²⁰), C(═O)O, OC(═O), OC(═O)O, C(═O)N(R¹⁵), N(R¹⁵)C(═O), OC(═O)N(R¹⁵), N(R¹⁵)C(═O)O, CH═N—O, CH═N—NR¹⁵, OC(O)NR¹⁵, NR¹⁵C(O)O, S═O, SO₂, SO₂NR¹⁵ and NR¹⁵SO₂, wherein
h, i are independently from each other 0, 1, 2, 3, 4, 5, or 6, and
j is 1, 2, 3, 4, 5, or 6,
Y is selected from O, S, NR²¹, C(R²²)—NO₂, C(R²²)—CN and C(CN)₂, wherein
R²¹ is independently selected from the meanings given for R¹³, R¹⁴ and
R²² is independently selected from the meanings given for R¹¹, R¹²,
g is 1, 2 or 3, preferably 1 or 2,
p, r are independently from one another 0, 1, 2, 3, 4 or 5,
q is 0, 1, 2, 3 or 4, preferably 0, 1 or 2,
u is 0, 1, 2 or 3, preferably 0, 1 or 2,
and
Hal is independently selected from a group consisting of F, Cl, Br and I;
and the pharmaceutically acceptable derivatives, salts and solvates thereof.

2. Bisarylurea derivatives according to claim 1,
wherein
Ar¹, Ar² are selected independently from one another from aromatic hydrocarbons containing 6 to 10 and especially 6 carbon atoms and ethylenical unsaturated or aromatic heterocyclic residues containing 3 to 8 and especially 4 to 6 carbon atoms and one or two heteroatoms, independently selected from N, O and S and especially selected from N and O,
R⁷ is independently selected from a group consisting of Het, OHet, N(R¹¹)Het, (CR⁵R⁶)ₖHet, O(CR⁵R⁶)ₖHet, N(R¹¹)(CR⁵R⁶)ₖHet, (CR⁵R⁶)ₖNR¹¹R¹², (CR⁵R⁶)ₖOR¹³, O(CR⁵R⁶)ₖNR¹¹R¹², NR¹¹(CR⁵R⁶)ₖNR¹¹R¹², O(CR⁵R⁶)ₖR¹³, NR¹¹(CR⁵R⁶)ₖR¹³, O(CR⁵R⁶)ₖOR¹³, NR¹¹(CR⁵R⁶)ₖOR¹³, O(CR⁵R⁶)ₙO(CR⁵R⁶)ₖNR¹¹R¹², NR¹¹(CR⁵R⁶)ₙO(CR⁵R⁶)ₖNR¹¹R¹², O(CR⁵R⁶)ₙNR¹¹(CR⁵R⁶)ₖNR¹¹R¹², NR¹¹(CR⁵R⁶)ₙNR¹²(CR⁵R⁶)ₖNR¹¹R¹², O(CR⁵R⁶)ₙO(CR⁵R⁶)ₖOR¹¹, NR¹¹(CR⁵R⁶)ₙO(CR⁵R⁶)ₖOR¹², O(CR⁵R⁶)ₙNR¹¹(CR⁵R⁶)ₖOR¹² and NR¹²(CR⁵R⁶)ₙNR¹¹(CR⁵R⁶)ₖOR¹², O(CR⁵R⁶)ₖAr³—NR¹¹R¹², SO₂R¹³, SO₂(CR⁵R⁶)ₖOR¹³ and SO₂(CR⁵R⁶)ₖNR¹¹R¹², wherein
R⁵, R⁶ are in each case independently from one another selected from H and A; or R⁵ and R⁶ together optionally represent an oxo-group; or
R⁷ is selected from divalent radicals of formula —SO₂—CR⁸═CR⁸═CR⁸—, wherein both valencies are bound vicinally to Ar¹, and
n and/or k independently are 0, 1, 2, 3 or 4, preferably 1, 2, 3 or 4, and even more preferred are 2 or 3;
R⁸, R⁹ and R¹⁰ are independently selected from a group consisting of H, A, cycloalkyl comprising 3 to 7 carbon atoms, Hal, CH₂Hal, CH(Hal)₂, C(Hal)₃, NO₂, (CH₂)ₙCN, (CH₂)ₙNR¹¹R¹², (CH₂)ₙO(CH₂)ₖNR¹¹R¹², (CH₂)ₙNR¹¹(CH₂)ₖNR¹¹R¹², (CH₂)ₙO(CH₂)ₖOR¹¹, (CH₂)ₙNR¹¹(CH₂)ₖOR¹², (CH₂)ₙCOR¹³, (CH₂)ₙCOOR¹³, (CH₂)ₙCONR¹¹R¹², (CH₂)ₙNR¹¹COR¹³, (CH₂)ₙNR¹¹CONR¹¹R¹², (CH₂)ₙNR¹¹SO₂A, (CH₂)ₙSO₂NR¹¹R¹², (CH₂)ₙS(O)ᵤR¹³, (CH₂)ₙOC(O)R¹³, (CH₂)ₙCOR¹³, (CH₂)ₙSR¹¹, (CH₂)ₙNHOA, (CH₂)ₙNR¹¹COOR¹³, (CH₂)ₙN(R¹¹)CH₂CH₂OR¹³, (CH₂)ₙN(R¹¹)CH₂CH₂OCF₃, (CH₂)ₙN(R¹¹)C(R¹³)HCOOR¹², (CH₂)ₙN(R¹¹)C(R¹³)HCOR¹¹, (CH₂)ₙN(COOR¹³)COOR¹⁴, (CH₂)ₙN(CONH₂)COOR¹³, (CH₂)ₙN(CONH₂)CONH₂, (CH₂)ₙN(CH₂COOR¹³)COOR¹⁴, (CH₂)ₙN(CH₂CONH₂)COOR¹³, (CH₂)ₙN(CH₂CONH₂)CONH₂, (CH₂)ₙCHR¹³COR¹⁴, (CH₂)ₙCHR¹³COOR¹⁴ and (CH₂)ₙCHR¹³CH₂OR¹⁴, wherein
n and/or k independently are 0, 1, 2, 3 or 4, preferably 0, 1, 2 or 3, and even more preferred are 0 or 2;
X represents a bond or is (CR¹¹R¹²)ₕ, or (CHR¹¹)ₕ-Q-(CHR¹²)ᵢ, wherein
Q is selected from a group consisting of O, S, N—R¹⁵, (CHal₂)ⱼ, (O—CHR¹⁸)ⱼ, (CHR¹⁸—O), CR¹⁸═CR¹⁹, (O—CHR¹⁸CHR¹⁹)ⱼ, (CHR¹⁸CHR¹⁹—O)ⱼ, C═O, C═NR¹⁵, CH(OR¹⁵), C(OR¹⁵)(OR²⁰), C(═O)N(R¹⁵), N(R¹⁵)C(═O), CH═N—NR¹⁵, S═O, SO₂, SO₂NR¹⁵ and NR¹⁵SO₂, wherein
h, i are independently from each other 0, 1, 2, 3, 4, 5 or 6, preferably 0, 1, 2 or 3 and
j is 1, 2, 3, 4, 5 or 6, preferably 1, 2, 3 or 4,
g is 1 or 2, preferably 1,
p is 1, 2 or 3, preferably 1 or 2, and
r is 0, 1, 2, or 3, preferably 0, 1 or 2;
and the pharmaceutically acceptable derivatives, solvates, salts and stereoisomers thereof

3. Bisarylurea derivatives according to claim 1 or 2,
wherein
R⁷ is independently selected from a group consisting of Het, OHet, N(R¹¹)Het, (CR⁵R⁶)ₖHet, O(CR⁵R⁶)ₖHet, N(R¹¹)(CR⁵R⁶)ₖHet, (CR⁵R⁶)ₖNR¹¹R¹², (CR⁵R⁶)ₖOR¹³, O(CR⁵R⁶)ₖNR¹¹R¹², NR¹¹(CR⁵R⁶)ₖNR¹¹R¹², O(CR⁵R⁶)ₖR¹³, NR¹¹(CR⁵R⁶)ₖR¹³, O(CR⁵R⁶)ₖOR¹³, NR¹¹(CR⁵R⁶)ₖOR¹³, O(CR⁵R⁶)ₖAr³—NR¹¹R¹², SO₂R¹³, SO₂(CR⁵R⁶)ₖOR¹³, SO₂(CR⁵R⁶)ₖNR¹¹R¹² and divalent radicals of formula —SO₂—CR⁸═CR⁸—, wherein both valencies are bound vicinally to Ar¹; and more preferably from OHet, N(R¹¹)Het, (CR⁵R⁶)ₖHet, O(CR⁵R⁶)ₖHet, N(R¹¹)(CR⁵R⁶)ₖHet, O(CR⁵R⁶)ₖNR¹¹R¹², NR¹¹(CR⁵R⁶)ₖNR¹¹R¹², O(CR⁵R⁶)ₖOR¹³ and NR¹¹(CR⁵R⁶)ₖOR¹³, O(CR⁵R⁶)ₖAr³—NR¹¹R¹², SO₂R¹³, SO₂(CR⁵R⁶)ₖOR¹³, SO₂(CR⁵R⁶)ₖNR¹¹R¹² and divalent radicals of formula —SO₂—CR⁸═CR⁸—, wherein both valencies are bound vicinally to Ar¹, and
n and k are independently from one another 0, 1, 2, 3 or 4.

4. Bisarylurea derivative according to one of the claims 1 to 3, selected from the compounds of formula Ia, Ib, Ic, Id, Ie, If, Ig, Ih, Ii, Ij, Ik, IL, Im, In, Io, Ip, Iq, Ir, Is, It, Iu, Iv, Iw, Ix, Iy, Iz and Iaa to Iuu, 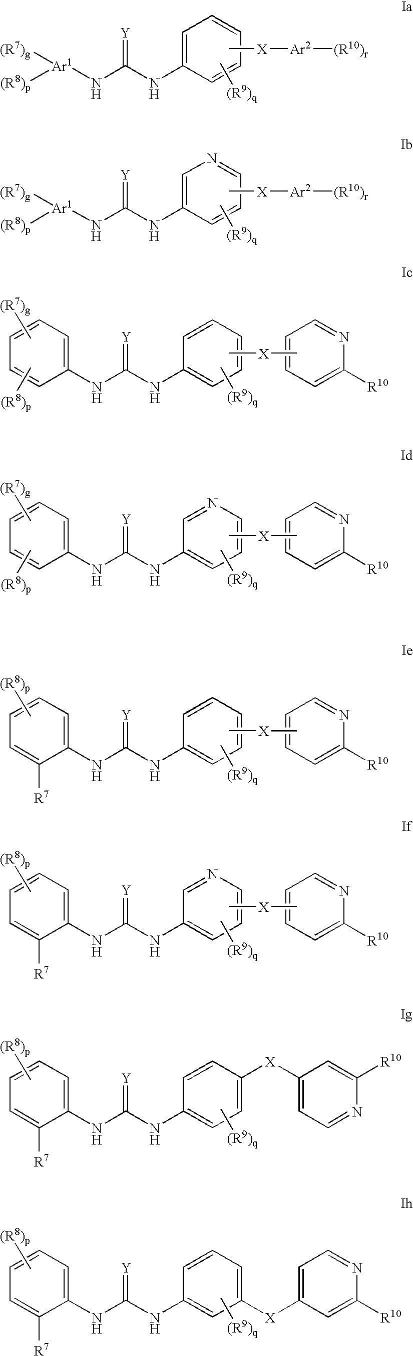 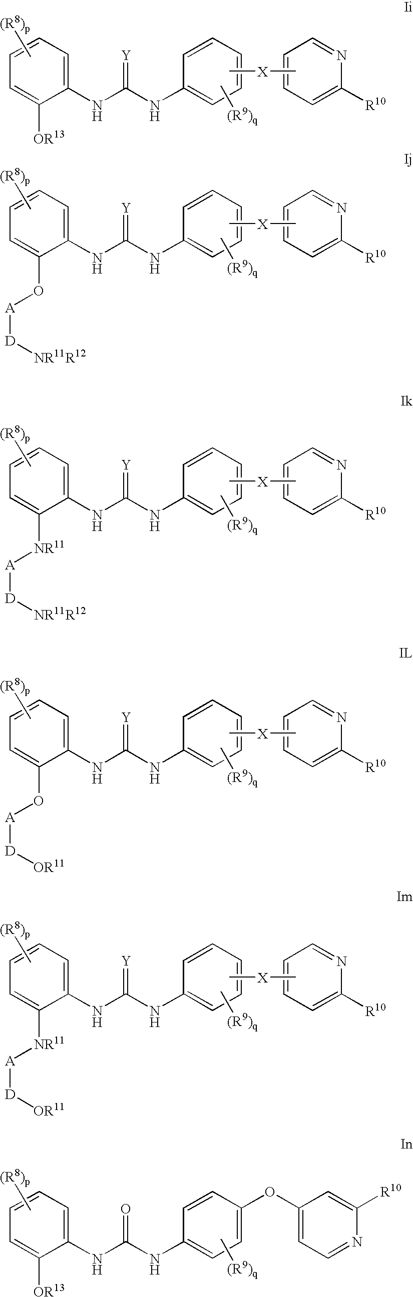 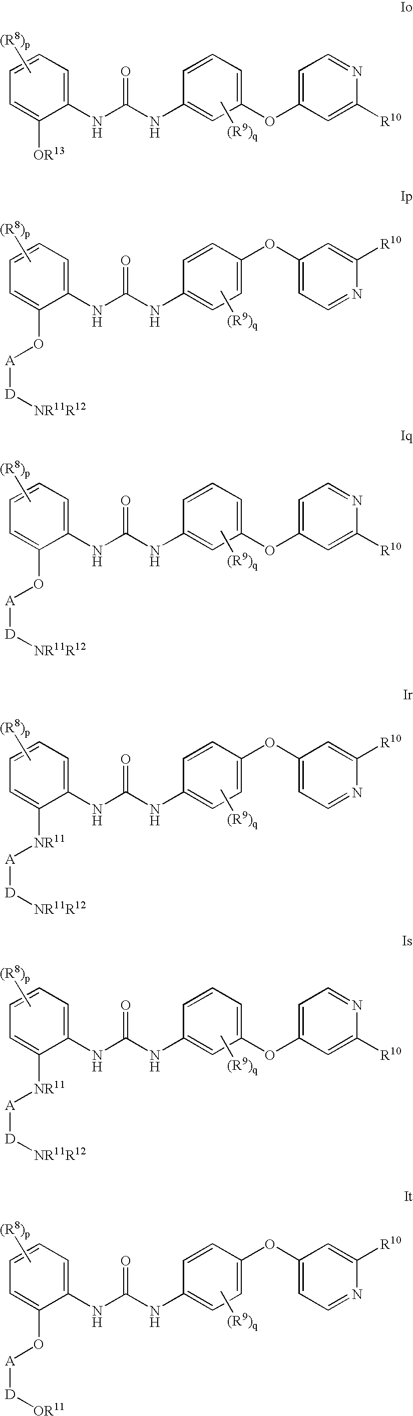 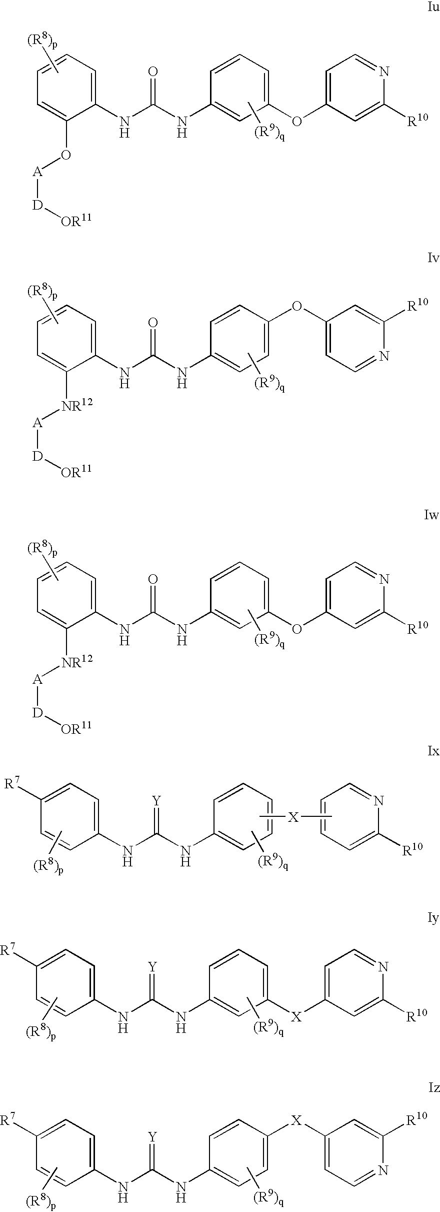 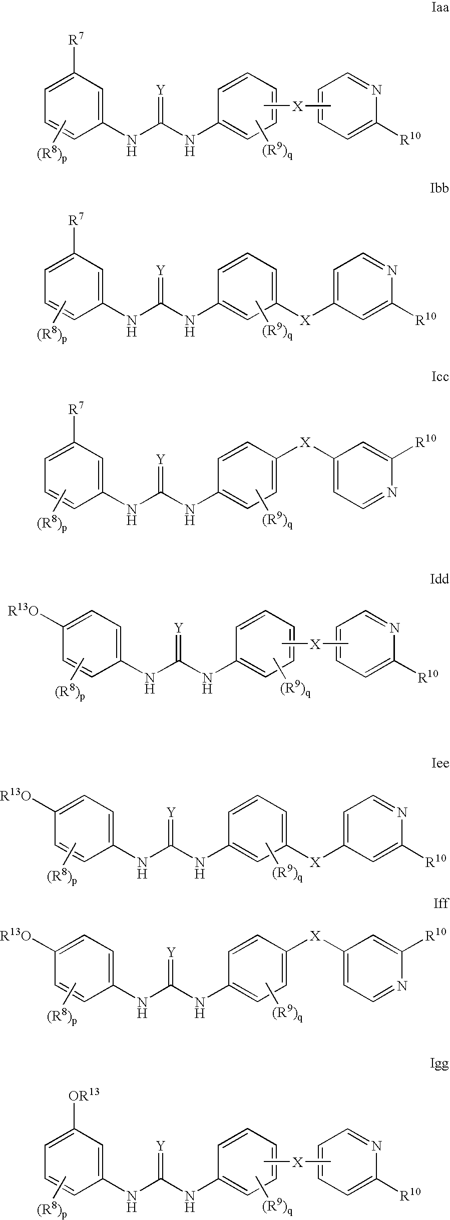 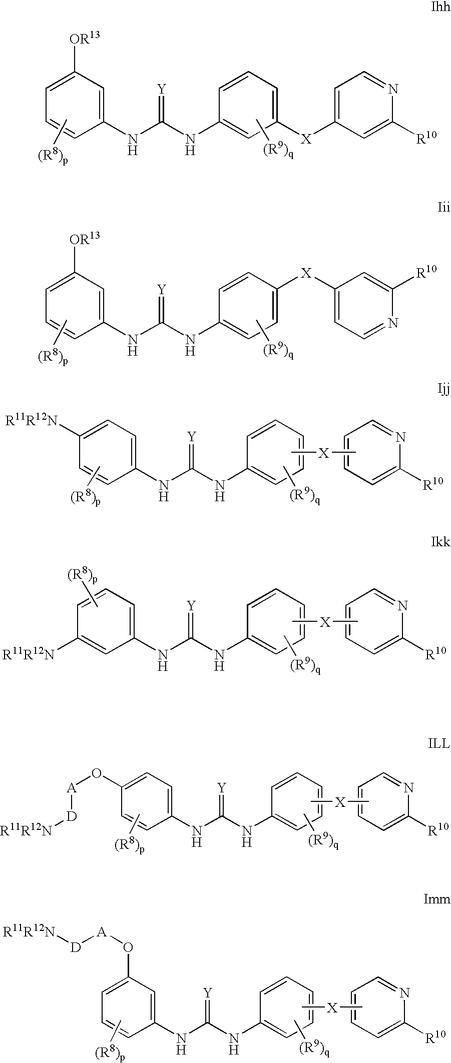 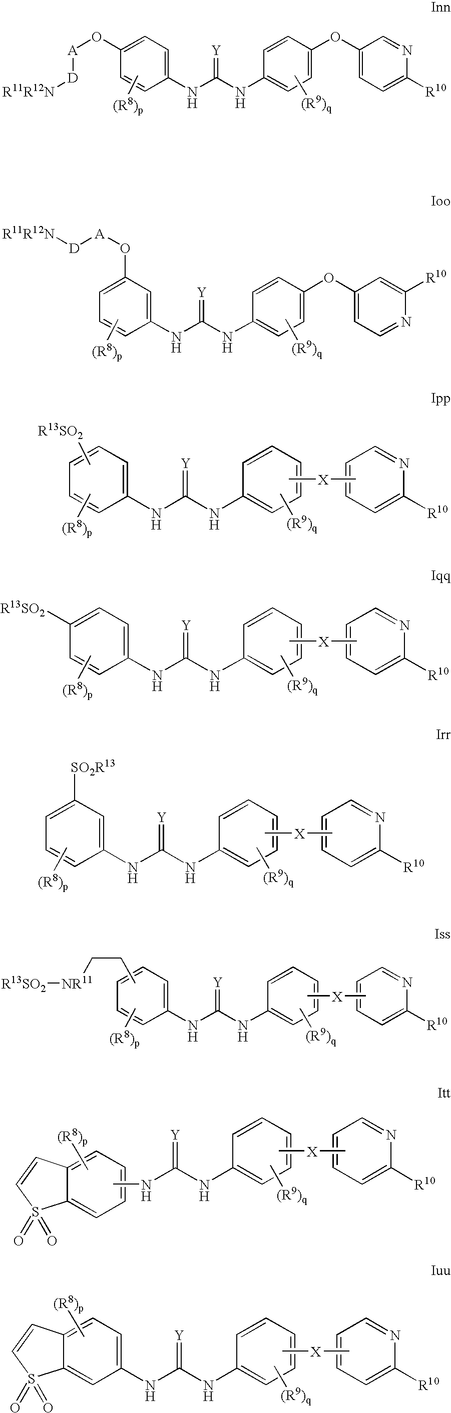
wherein R⁷, R⁸, R¹¹, R¹², R¹³, Y, X, R⁹, A, D, g, p and q are as defined in one of the claims 1 to 3, R¹⁰ is H or as defined in one of the claims 1 to 3; and A and D are CR⁵R⁶, wherein R⁵ and R⁶ are as defined in claim 1, and the pharmaceutically acceptable derivatives, salts and solvates thereof.

5. Bisarylurea derivative according to claim one of the claims 1 to 4, selected from 4-(4-{3-[4-Chloro-5-methyl-2-(2-methylamino-ethoxy)-phenyl]-ureido}-phenoxy)-pyridine-2-carboxylic acid methylamide;
4-(4-{3-[Chloro-(2-methylamino-ethoxy)-trifluoromethyl-phenyl]-ureido}-phenoxy)-pyridine-2-carboxylic acid methylamide;
4-(4-{3-[(2-Methylamino-ethoxy)-trifluoromethyl-phenyl]-ureido}-phenoxy)-pyridine-2-carboxylic acidmethylamide;
4-(4-{3-[Chloro-(2-dimethylamino-ethoxy)-trifluoromethyl-phenyl]-ureido}-phenoxy)-pyridine-2-carboxylic acid methylamide;
4-(4-{3-[Chloro-(2-diethylamino-ethoxy)-trifluoromethyl-phenyl]-ureido}-phenoxy)-pyridine-2-carboxylic acid methylamide;
4-(4-{3-[Chloro-(2-morpholin-4-yl-ethoxy)-trifluoromethyl-phenyl]-ureido}-phenoxy)-pyridine-2-carboxylic acid methylamide;
4-(4-{3-[Chloro-(2-pyrrolidin-1-yl-ethoxy)-trifluoromethyl-phenyl]-ureido}-phenoxy)-pyridine-2-carboxylic acid methylamide;
4-(4-{3-[Chloro-(piperidin-4-yloxy)-trifluoromethyl-phenyl]-ureido}-phenoxy)-pyridine-2-carboxylic acid methylamide;
4-(4-{3-[(2-Amino-ethoxy)-chloro-trifluoromethyl-phenyl]-ureido}-phenoxy)-pyridine-2-carboxylic acidmethylamide;
4-(4-{3-[2-(2-Amino-ethoxy)-4-chloro-5-methyl-phenyl]-ureido}-phenoxy)-pyridine-2-carboxylic acid methylamide;
4-(4-{3-[(2-Amino-ethoxy)-trifluoromethyl-phenyl]-ureido}-phenoxy)-pyridine-2-carboxylic acid methylamide;
4-(4-{3-[Chloro-(2-piperazin-1-yl-ethoxy)-trifluoromethyl-phenyl]-ureido}-phenoxy)-pyridine-2-carboxylic acid methylamide;
4-(3-{3-[Chloro-(2-diethylamino-ethoxy)-trifluoromethyl-phenyl]-ureido}-phenoxy)-pyridine-2-carboxylic acid methylamide;
4-(4-{3-[4-Chloro-2-(2-dimethylamino-ethoxy)-5-methyl-phenyl]-ureido}-phenoxy)-pyridine-2-carboxylic acid methylamide;
4-(4-{3-[4-Chloro-2-(2-diethylamino-ethoxy)-5-methyl-phenyl]-ureido}-phenoxy)-pyridine-2-carboxylic acid methylamide;
4-(4-{3-[4-Chloro-5-methyl-2-(2-morpholin-4-yl-ethoxy)-phenyl]-ureido}-phenoxy)-pyridine-2-carboxylic acid methylamide;
4-(4-{3-[4-Chloro-5-methyl-2-(2-pyrrolidin-1-yl-ethoxy)-phenyl]-ureido}-phenoxy)-pyridine-2-carboxylic acid methylamide;
4-(3-{3-[Chloro-(2-morpholin-4-yl-ethoxy)-trifluoromethyl-phenyl]-ureido}-phenoxy)-pyridine-2-carboxylic acid methylamide;
4-(4-{3-[(2-Pyrrolidin-1-yl-ethoxy)-trifluoromethyl-phenyl]-ureido}-phenoxy)-pyridine-2-carboxylic acid methylamide;
4-(4-{3-[(2-Morpholin-4-yl-ethoxy)-trifluoromethyl-phenyl]-ureido}-phenoxy)-pyridine-2-carboxylic acid methylamide;
4-(4-{3-[(2-Diethylamino-ethoxy)-trifluoromethyl-phenyl]-ureido}-phenoxy)-pyridine-2-carboxylic acidmethylamide;
4-(4-{3-[(2-Dimethylamino-ethoxy)-trifluoromethyl-phenyl]-ureido}-phenoxy)-pyridine-2-carboxylic acid methylamide;
4-(4-{3-[4-Chloro-5-methyl-2-(2-piperazin-1-yl-ethoxy)-phenyl]-ureido}-phenoxy)-pyridine-2-carboxylic acid methylamide;
4-(4-{3-[4-Chloro-5-methyl-2-(piperidin-4-yloxy)-phenyl]-ureido}-phenoxy)-pyridine-2-carboxylic acidmethylamide;
4-(4-{3-[(2-piperazin-1-yl-ethoxy)-trifluoromethyl-phenyl]-ureido}-phenoxy)-pyridine-2-carboxylic acid methylamide;
4-(4-{3-[(Piperidin-4-yloxy)-trifluoromethyl-phenyl]-ureido}-phenoxy)-pyridine-2-carboxylic acid methylamide;
4-(4-{3-[(Pyrrolidin-2-ylmethoxy)-trifluoromethyl-phenyl]-ureido}-phenoxy)-pyridine-2-carboxylic acid methylamide;
4-(3-{3-[Chloro-(2-pyrrolidin-1-yl-ethoxy)-trifluoromethyl-phenyl]-ureido}-phenoxy)-pyridine-2-carboxylic acid methylamide;
4-(4-{3-[(2-Amino-2-methyl-propoxy)-trifluoromethyl-phenyl]-ureido}-phenoxy)-pyridine-2-carboxylic acid methylamide;
4-(3-{3-[(2-Amino-ethoxy)-chloro-trifluoromethyl-phenyl]-ureido}-phenoxy)-pyridine-2-carboxylic acidmethylamide;
4-(3-{3-[(2-Methylamino-ethoxy)-trifluoromethyl-phenyl]-ureido}-phenoxy)-pyridine-2-carboxylic acidmethylamide;
4-(4-{3-[(2-Isopropylamino-ethoxy)-trifluoromethyl-phenyl]-ureido}-phenoxy)-pyridine-2-carboxylic acid methylamide;
4-(3-{3-[4-Chloro-5-methyl-2-(2-methylamino-ethoxy)-phenyl]-ureido}-phenoxy)-pyridine-2-carboxylic acid methylamide;
4-(3-{3-[Chloro-(2-methylamino-ethoxy)-trifluoromethyl-phenyl]-ureido}-phenoxy)-pyridine-2-carboxylic acid methylamide;
4-(3-{3-[Chloro-(2-dimethylamino-ethoxy)-trifluoromethyl-phenyl]-ureido}-phenoxy)-pyridine-2-carboxylic acid methylamide;
4-(3-{3-[Chloro-(2-piperazin-1-yl-ethoxy)-trifluoromethyl-phenyl]-ureido}-phenoxy)-pyridine-2-carboxylic acid methylamide;
4-(3-{3-[Chloro-(piperidin-4-yloxy)-trifluoromethyl-phenyl]-ureido}-phenoxy)-pyridine-2-carboxylic acid methylamide;
4-(3-{3-[2-(2-Amino-ethoxy)-4-chloro-5-methyl-phenyl]-ureido}-phenoxy)-pyridine-2-carboxylic acid methylamide;
4-(3-{3-[(2-Dimethylamino-ethoxy)-trifluoromethyl-phenyl]-ureido}-phenoxy)-pyridine-2-carboxylic acid methylamide;
4-(3-{3-[4-Chloro-2-(2-dimethylamino-ethoxy)-5-methyl-phenyl]-ureido}-phenoxy)-pyridine-2-carboxylic acid methylamide;
4-(3-{3-[4-Chloro-5-methyl-2-(2-pyrrolidin-1-yl-ethoxy)-phenyl]-ureido}-phenoxy)-pyridine-2-carboxylic acid methylamide;
4-(3-{3-[(2-Pyrrolidin-1-yl-ethoxy)-trifluoromethyl-phenyl]-ureido}-phenoxy)-pyridine-2-carboxylic acid methylamide;
4-(3-{3-[(Piperidin-4-yloxy)-trifluoromethyl-phenyl]-ureido}-phenoxy)-pyridine-2-carboxylic acid methylamide;
4-(3-{3-[4-Chloro-5-methyl-2-(piperidin-4-yloxy)-phenyl]-ureido}-phenoxy)-pyridine-2-carboxylic acidmethylamide;
4-(3-{3-[(2-Amino-2-methyl-propoxy)-trifluoromethyl-phenyl]-ureido}-phenoxy)-pyridine-2-carboxylic acid methylamide;
4-(3-{3-[(2-Isopropylamino-ethoxy)-trifluoromethyl-phenyl]-ureido}-phenoxy)-pyridine-2-carboxylic acid methylamide;
4-(3-{3-[(Pyrrolidin-2-ylmethoxy)-trifluoromethyl-phenyl]-ureido}-phenoxy)-pyridine-2-carboxylic acid methylamide;
4-(3-{3-[(2-Amino-ethoxy)-trifluoromethyl-phenyl]-ureido}-phenoxy)-pyridine-2-carboxylic acid methylamide;
1-[3-Methyl-4-(2-pyrrolidin-1-yl-ethoxy)-phenyl]-3-[4-(pyridin-4-yloxy)-phenyl]-urea;
1-[4-(Pyridin-4-yloxy)-phenyl]-3-[4-(2-pyrrolidin-1-yl-ethoxy)-phenyl]-urea;
4-(4-{3-[4-(2-Pyrrolidin-1-yl-ethoxy)-phenyl]-ureido}-phenoxy)-pyridine-2-carboxylic acid methylamide;
1-[3-Chloro-4-(2-pyrrolidin-1-yl-ethoxy)-phenyl]-3-[4-(pyridin-4-yloxy)-phenyl]-urea;
4-(4-{3-[4-(Piperidin-4-yloxy)-phenyl]-ureido}-phenoxy)-pyridine-2-carboxylic acid methylamide;
1-[4-(Piperidin-4-yloxy)-phenyl]-3-[4-(pyridin-4-yloxy)-phenyl]-urea;
4-(4-{3-[4-(2-Pyrrolidin-1-yl-ethoxy)-3-trifluoromethyl-phenyl]-ureido}-phenoxy)-pyridine-2-carboxylic acid methylamide;
1-[4-(2-Dimethylamino-ethoxy)-phenyl]-3-[4-(pyridin-4-yloxy)-phenyl]-urea;
1-[4-(Pyridin-4-yloxy)-phenyl]-3-[4-(2-pyrrolidin-1-yl-ethoxy)-3-trifluoromethyl-phenyl]-urea;
4-(4-{3-[4-(Pyrrolidin-3-yloxy)-phenyl]-ureido}-phenoxy)-pyridine-2-carboxylic acid methylamide;
N-[4-(5-Chloro-2-{3-[4-(pyridin-4-yloxy)-phenyl]-ureido}-phenoxy)-phenyl]-acetamide;
4-(2-Chloro-4-{3-[4-(pyridin-4-yloxy)-phenyl]-ureido}-phenoxy)-piperidine-1-carboxylic acid tert-butyl ester;
4-(2-Chloro-4-{3-[4-(2-methylcarbamoyl-pyridin-4-yloxy)-phenyl]-ureido}-phenoxy)-piperidine-1-carboxylic acid tert-butyl ester;
4-(4-{3-[4-(2-Dimethylamino-ethoxy)-phenyl]-ureido}-phenoxy)-pyridine-2-carboxylic acid methylamide;
4-(4-{3-[2-Chloro-5-(2-diethylamino-ethoxy)-phenyl]-ureido}-phenoxy)-pyridine-2-carboxylic acid methylamide;
4-(4-{3-[4-Methoxy-3-(2-pyrrolidin-1-yl-ethoxy)-phenyl]-ureido}-phenoxy)-pyridine-2-carboxylic acid methylamide;
4-(4-{3-[3-Chloro-4-(piperidin-4-yloxy)-phenyl]-ureido}-phenoxy)-pyridine-2-carboxylic acid methylamide;
1-[3-Chloro-4-(piperidin-4-yloxy)-phenyl]-3-[4-(pyridin-4-yloxy)-phenyl]-urea;
4-(4-{3-[2-Methyl-3-(2-pyrrolidin-1-yl-ethoxy)-phenyl]-ureido}-phenoxy)-pyridine-2-carboxylic acid methylamide;
(4-{3-[3-(Pyridin-4-yloxy)-phenyl]-ureido}-phenoxy)-pyridine-2-carboxylic acid methylamide;
4-{4-[3-(5-Carbamoyl-4-chloro-2-fluoro-phenyl)-ureido]-phenoxy}-pyridine-2-carboxylic acid methylamide;
4-[2-(4-{3-[4-(2-Methylcarbamoyl-pyridin-4-yloxy)-phenyl]-ureido}-phenoxy)-ethyl]-piperazine-1-carboxylic acid tert-butyl ester;
4-(4-{3-[4-(2-piperazin-1-yl-ethoxy)-phenyl]-ureido}-phenoxy)-pyridine-2-carboxylic acid methylamide;
4-(4-{3-[4-(2,5-Dioxo-pyrrolidin-1-yl)-3-trifluoromethyl-phenyl]-ureido}-phenoxy)-pyridine-2-carboxylic acid methylamide;
4-(4-{3-[2-(4-Acetylamino-phenoxy)-4-chloro-phenyl]-ureido}-phenoxy)-pyridine-2-carboxylic acid methylamide;
4-{4-[3-(2-tert-Butoxy-5-trifluoromethyl-phenyl)-ureido]-phenoxy}-pyridine-2-carboxylic acid methylamide;
4-(4-{3-[4-(Piperidin-4-yloxy)-3-trifluoromethyl-phenyl]-ureido}-phenoxy)-pyridine-2-carboxylic acid methylamide;
1-[4-(Piperidin-4-yloxy)-3-trifluoromethyl-phenyl]-3-[4-(pyridin-4-yloxy)-phenyl]-urea;
4-{4-[3-(2-Hydroxy-5-trifluoromethyl-phenyl)-ureido]-phenoxy}-pyridine-2-carboxylic acid methylamide;
4-(4-{3-[3-Cyano-4-(piperidin-4-yloxy)-phenyl]-ureido}-phenoxy)-pyridine-2-carboxylic acid methylamide;
4-{4-[3-(2-Dimethylamino-5-trifluoromethyl-phenyl)-ureido]-phenoxy}-pyridine-2-carboxylic acid methylamide;
4-{4-[3-(2-Chloro-5-trifluoromethanesulfonyl-phenyl)-ureido]-phenoxy}-pyridine-2-carboxylic acid methylamide;
4-{4-[3-(1,1-Dioxo-1H-1I6-benzo[b]thiophen-6-yl)-ureido]-phenoxy}-pyridine-2-carboxylic acid methylamide;
4-(4-{3-[3-(2-Hydroxy-ethanesulfonyl)-phenyl]-ureido}-phenoxy)-pyridine-2-carboxylic acid methylamide;
4-{4-[3-(2-Fluoro-5-methanesulfonyl-phenyl)-ureido]-phenoxy}-pyridine-2-carboxylic acid methylamide;
4-{4-[3-(5-Methanesulfonyl-2-methoxy-phenyl)-ureido]-phenoxy}-pyridine-2-carboxylic acid methylamide;
4-(4-{3-[2-(2-Methoxy-ethoxy)-5-trifluoromethyl-phenyl]-ureido}-phenoxy)-pyridine-2-carboxylic acid methylamide;
4-(4-{3-[2-(2-Methanesulfonylamino-ethyl)-5-trifluoromethyl-phenyl]-ureido}-phenoxy)-pyridine-2-carboxylic acid methylamide;
4-{4-[3-(3-Trifluoromethanesulfonyl-phenyl)-ureido]-phenoxy}-pyridine-2-carboxylic acid methylamide;
4-{4-[3-(2-Carbamoylmethyl-5-trifluoromethyl-phenyl)-ureido]-phenoxy}-pyridine-2-carboxylic acid methylamide;
4-(2-{3-[4-(2-Methylcarbamoyl-pyridin-4-yloxy)-phenyl]-ureido}-4-trifluoromethyl-phenyl)-piperazine-1-carboxylic acid tert-butyl ester;
4-{4-[3-(2-Morpholin-4-yl-5-trifluoromethyl-phenyl)-ureido]-phenoxy}-pyridine-2-carboxylic acid methylamide;
4-{4-[3-(2-piperazin-1-yl-5-trifluoromethyl-phenyl)-ureido]-phenoxy}-pyridine-2-carboxylic acid methylamide;
4-(4-{3-[2-(Acetylamino-methyl)-5-trifluoromethyl-phenyl]-ureido}-phenoxy)-pyridine-2-carboxylic acid methylamide;
4-(4-{3-[2-(2-Acetylamino-ethyl)-5-trifluoromethyl-phenyl]-ureido}-phenoxy)-pyridine-2-carboxylic acid methylamide;
4-{4-[3-(4-Methoxy-biphenyl-3-yl)-ureido]-phenoxy}-pyridine-2-carboxylic acid methylamide;
4-{4-[3-(5-Cyclohexyl-2-methoxy-phenyl)-ureido]-phenoxy}-pyridine-2-carboxylic acid methylamide;
4-(4-{3-[2-Methoxy-5-(1-methyl-1-phenyl-ethyl)-phenyl]-ureido}-phenoxy)-pyridine-2-carboxylic acid methylamide;
4-{4-[3-(2-Methoxy-5-phenylcarbamoyl-phenyl)-ureido]-phenoxy}-pyridine-2-carboxylic acid methylamide;
4-Methoxy-3-{3-[4-(2-methylcarbamoyl-pyridin-4-yloxy)-phenyl]-ureido}-benzoic acid methyl ester;
5-Methoxy-2-methyl-4-{3-[4-(2-methylcarbamoyl-pyridin-4-yloxy)-phenyl]-ureido}-benzenesulfonic acid;
4-{4-[3-(4-Benzyloxy-3-trifluoromethyl-phenyl)-ureido]-phenoxy}-pyridine-2-carboxylic acid methylamide;
1-(4-Benzyloxy-3-trifluoromethyl-phenyl)-3-[4-(pyridin-4-yloxy)-phenyl]-urea;
4-{4-[3-(4-Hydroxy-3-trifluoromethyl-phenyl)-ureido]-phenoxy}-pyridine-2-carboxylic acid methylamide;
4-{4-[3-(5-Carbamoyl-2-methoxy-phenyl)-ureido]-phenoxy}-pyridine-2-carboxylic acid methylamide;
1-(4-Hydroxy-3-trifluoromethyl-phenyl)-3-[4-(pyridin-4-yloxy)-phenyl]-urea;
4-(2-{3-[4-(2-Methylcarbamoyl-pyridin-4-yloxy)-phenyl]-ureido}-phenyl)-piperazine-1-carboxylic acid tert-butyl ester;
4-{4-[3-(2-piperazin-1-yl-phenyl)-ureido]-phenoxy}-pyridine-2-carboxylic acid methylamide;
4-[4-(3-{2-[(Pyridine-4-carbonyl)-amino]-5-trifluoromethyl-phenyl}-ureido)-phenoxy]-pyridine-2-carboxylic acid methylamide;
and the pharmaceutically acceptable derivatives, salts and solvates thereof.

6. Bisarylurea derivative according to one of the claims 1 to 5 as a medicament.

7. Bisarylurea derivative according to one of the claims 1 to 5 as a kinase inhibitor.

8. Bisarylurea derivative according to claim 7, characterized in that the kinases are selected from raf-kinases, Tie-kinases, PDGFR-kinases and VEGFR-kinases.

9. Pharmaceutical composition, characterised in that it contains one or more compounds according to one of the claims 1 to 5.

10. Pharmaceutical composition according to claim 9, characterised in that it contains one or more additional compounds, selected from the group consisting of physiologically acceptable excipients, auxiliaries, adjuvants, carriers and pharmaceutical active ingredients other than the compounds according to one of the claims 1 to 5.

11. Process for the manufacture of a pharmaceutical composition, characterised in that one or more compounds according to one of the claims 1 to 5 and one or more compounds, selected from the group consisting of carriers, excipients, auxiliaries and pharmaceutical active ingredients other than the compounds according to one of the claims 1 to 5, is processed by mechanical means into a pharmaceutical composition that is suitable as dosage form for application and/or administration to a patient.

12. Use of a compound according to one of the claims 1 to 5 as a pharmaceutical.

13. Use of a compound according to one of the claims 1 to 5 in the treatment and/or prophylaxis of disorders.

14. Use of a compound according to one of the claims 1 to 5 for producing a pharmaceutical composition for the treatment and/or prophylaxis of disorders.

15. Use according to claim 13 or 14, characterised in that the disorders are caused, mediated and/or propagated by one or more kinases, selected from raf-kinases, Tie-kinases, PDGFR-kinases and VEGFR-kinases.

16. Use according to claim 13, 14 or 15, characterised in that the disorders are selected from the group consisting of hyperproliferative and nonhyperproliferative disorders.

17. Use according to claim 13, 14, 15 or 16, characterised in that the disorder is cancer.

18. Use according to claim 13, 14, 15 or 16, characterised in that the disorder is noncancerous.

19. Use according to claim 13, 14, 15, 16 or 18, characterised in that the disorders are selected from the group consisting of psioarsis, arthritis, inflammation, endometriosis, scarring, Helicobacter pylori infection, Influenza A, begnin prostatic hyperplasia, immunological diseases, autoimmune diseases and immunodeficiency diseases.

20. Use according to one of the claims 13 to 17, characterised in that the disorders are selected from the group consisting of melanoma, brain cancer, lung cancer, squamous cell cancer, bladder cancer, gastric cancer, pancreatic cancer, hepatic cancer, renal cancer, colorectal cancer, breast cancer, head cancer, neck cancer, oesophageal cancer, gynaecological cancer, ovarian cancar, ovary cancer, uterine cancer, prostate cancer, thyroid cancer, lymphoma, chronic leukaemia and acute leukaemia.

21. Use according to one of the claims 13 to 18, characterised in that the disorders are selected from the group consisting of arthritis, restenosis; fibrotic disorders; mesangial cell proliferative disorders, diabetic nephropathy, malignant nephrosclerosis, thrombotic microangiopathy syndromes, organ transplant rejection, glomerulopathies, metabolic disorders, inflammation, solid tumors, rheumatic arthritis, diabetic retinopathy, and neurodegenerative diseases.

22. Use according to one of the claims 13 to 16, characterised in that the disorders are selected from the group consisting of rheumatoid arthritis, inflammation, autoimmune disease, chronic obstructive pulmonary disease, asthma, inflammatory bowel disease, fibrosis, atherosclerosis, restenosis, vascular disease, cardiovascular disease, inflammation, renal disease and angiogenesis disorders.

23. Use of a compound according to one of the claims 1 to 5 as a kinase inhibitor.

24. Use according to claim 23, characterised in that the kinase is selected from the group consisting of from raf-kinases, Tie-kinases, PDGFR-kinases, VEGFR-kinases and p38-kinases.

25. Method for the treatment and/or prophylaxis of disorders, characterised in that one or more compounds according to one of the claims 1 to 5 is administered to a patient in need of such a treatment.

26. Method according to claim 25, characterised in that the one or more compounds according to one of the claims claim 1 to 5 are administered as a pharmaceutical composition according to claim 9 or 10.

27. Method for the treatment and/or prophylaxis of disorders according to claim 26, characterised in that the disorders are as defined in one of the claims 15 to 22.

28. Method for the treatment according to claim 27, characterised in that the disorder is cancerous cell growth mediated by raf-kinase, Tie kinases, PDGFR kinases and/or VEGFR kinases.

29. Method for producing compounds of formula I, characterised in that
a) a compound of formula II, 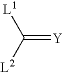
wherein
L¹ and L² either independently from one another represent a leaving group, or together represent a leaving group, and Y is as defined above/below,
is reacted with
b) a compound of formula III 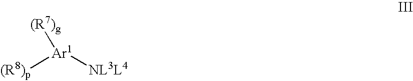
wherein
L³ and L⁴ are independently from one another H or a metal ion, and wherein R⁷, R⁸, g, p and Ar¹ are as defined in claim 1,
and
c) a compound of formula IV, 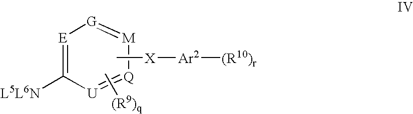
wherein
L⁵ and L⁶ are independently from one another H or a metal ion, and E, G, M, Q, U, R⁹, q, X, Ar², R¹⁰ and rare as defined in claim 1,
and optionally
d) isolating and/or treating the compound of formula I obtained by said reaction with an acid, to obtain the salt thereof.

30. Compound of formula III, 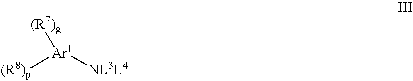
wherein
L³ and L⁴ are independently from one another H or a metal ion, and wherein R⁷, R⁸, g, p and Ar¹ are as defined in claim 1.

31. Compound of formula IV, 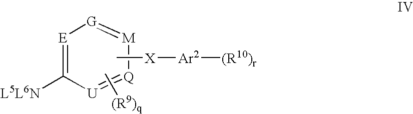
wherein
L⁵ and L⁶ are independently from one another H or a metal ion, and E, G, M, Q, U, R⁹, q, X, Ar², R¹⁰ and r are as defined in claim 1.
